(19) United States
(12) Patent Application Publication
(10) Pub. No.: US 2025/0177503 A1
(43) Pub. Date: Jun. 5, 2025

(54) COMPOSITIONS AND METHODS FOR TREATING AUTOIMMUNE DISORDERS

(71) Applicant: THE REGENTS OF THE UNIVERSITY OF MICHIGAN, Ann Arbor, MI (US)

(72) Inventors: James J. MOON, Ann Arbor, MI (US); Cheng XU, Ann Arbor, MI (US); Hannah DOBSON, Ann Arbor, MI (US); Jin XU, Ann Arbor, MI (US); Fang XIE, Ann Arbor, MI (US); Anna SCHWENDEMAN, Ann Arbor, MI (US)

(21) Appl. No.: 18/840,636

(22) PCT Filed: Feb. 22, 2023
(86) PCT No.: PCT/US2023/063022

§ 371 Date: Aug. 22, 2024

Related U.S. Application Data
(60) Provisional application No. 63/312,452, filed on Feb. 22, 2022.

(51) Int. Cl. A61K 39/00; A61K 9/20; A61K 39/39; A61K 47/24; A61P 37/02

(52) U.S. Cl. CPC A61K 39/001; A61K 9/20; A61K 39/39; A61K 47/24; A61P 37/02; A61K 2039/54; A61K 2039/55533

(57) ABSTRACT

The present invention relates to compositions comprising nanoparticles associated with or without one or more tolerogenic antigens, and compositions comprising an immunomodulatory agent (e.g., interleukin-2 (IL-2) or an IL-2 variant or an IL-2/IC)), and related methods involving co-administration of such compositions for purposes of inducing amplification of regulatory T cells (Tregs) (e.g., antigen-specific regulatory Tregs). The present invention further provides methods of treating autoimmune disorders through administering to a subject a composition comprising nanoparticles associated with or without one or more tolerogenic antigens associated with the autoimmune disorder prior to administering to the subject a composition comprising an immunomodulatory agent.

### CROSS-REFERENCE TO RELATED APPLICATION

The present application claims priority to U.S. Provisional Patent Application Ser. No. 63/312,452, filed Feb. 22, 2022 the contents of which are hereby incorporated by reference in its entirety.

### STATEMENT REGARDING FEDERALLY SPONSORED RESEARCH OR DEVELOPMENT

This invention was made with government support under R01 NS122536 awarded by the National Institutes of Health. The government has certain rights in the invention.

### SEQUENCE LISTING

The text of the computer readable sequence listing filed herewith, titled “40287_601_SequenceListing”, created Feb. 22, 2023, having a file size of 722,461 bytes, is hereby incorporated by reference in its entirety.

### FIELD OF THE INVENTION

The present invention relates to compositions comprising nanoparticles associated with or without one or more tolerogenic antigens, and compositions comprising an immunomodulatory agent (e.g., human interleukin-2 (IL-2), an IL-2 mutein, an IL-2 variant, or an IL-2:anti-IL-2 antibody immune complex (IL-2/IC)), and related methods involving co-administration of such compositions for purposes of inducing amplification of regulatory T cells (Tregs) (e.g., antigen-specific regulatory Tregs). The present invention further provides methods of treating autoimmune disorders through administering to a subject a composition comprising nanoparticles associated with or without one or more tolerogenic antigens associated with the autoimmune disorder prior to administering to the subject a composition comprising an immunomodulatory agent.

### BACKGROUND OF THE INVENTION

Autoimmune disorders are diseases that occur when the body's immune system attacks its own normal tissues, organs or other in vivo components due to immune system abnormalities whose cause cannot be found. These autoimmune disorders are systemic diseases that can occur in almost all parts of the body, including the nervous system, the gastrointestinal system, the endocrine system, the skin, the skeletal system, and the vascular tissue. It is known that autoimmune disorders affect about 5-8% of the world population, but the reported prevalence of autoimmune disorders is lower than the actual level due to limitations in the understanding of autoimmune disorders and a method for diagnosing these diseases.

Improved compositions and methods for treating autoimmune conditions are needed.

The present invention addresses these needs.

### SUMMARY OF THE INVENTION

Experiments conducted during the course of developing embodiments for the present invention resulted in the development of a new strategy for inducing high frequency of antigen-specific Tregs in vivo without ex vivo manipulation of cells. Such experiments resulted in the development and optimization of a new strategy of combining nanodiscs with modified IL-2 for eliciting unprecedented level of antigen-specific Tregs in vivo. Notably, IL-2:anti-IL-2 antibody (clone: JES6-1) immune complex (IL-2/IC) has been shown to selectively induce polyclonal Tregs └14┘. IL-2/IC administered together with free peptide or peptide-tetramer have been reported to induce antigen-specific Tregs [15,16]. However, these previous attempts resulted in rather poor antigen-specific Treg response with less than 0.25% antigen-specific Treg frequency among CD4+ T cell compartment [15,16].

It was envisioned that it may be desirable to deliver peptide antigens to lymphoid tissues to maximize antigen-specific Treg induction together with IL-2/IC. Experiments described herein report for the first time that lymph-targeting nanodisc-mediated delivery of peptide in combination with IL-2/IC therapy resulted in remarkable amplification of antigen-specific Tregs, compared with IL-2/IC alone.

It was also envisioned that the actual method of treatment may be crucial. Such experiments demonstrated that nanodiscs are desirably administered (e.g., subcutaneously) first, followed by systemic administration of IL-2 and/or mutein/engineered IL-2. This ensured that antigen-specific Tregs were primed and generated first, and the subsequent administration of mutein/engineered IL-2 triggered robust proliferation of antigen-specific Tregs. Administration of maintenance doses of nanodiscs and/or mutein/engineered IL-2 for long-term maintenance of antigen-specific Tregs was further envisioned.

Accordingly, the present invention relates to compositions comprising nanoparticles associated (e.g., complexed, conjugated, encapsulated, absorbed, adsorbed, admixed) with or without one or more tolerogenic antigens, and compositions comprising an immunomodulatory agent (e.g., human interleukin-2 (IL-2), an IL-2 mutein, an IL-2 variant, or an IL-2:anti-IL-2 antibody immune complex (IL-2/IC))), and related methods involving co-administration of such compositions for purposes of inducing amplification of regulatory T cells (Tregs) (e.g., antigen-specific regulatory Tregs). The present invention further provides methods of treating autoimmune disorders through administering to a subject a composition comprising nanoparticles associated with or without one or more tolerogenic antigens associated with the autoimmune disorder prior to administering to the subject a composition comprising an immunomodulatory agent.

In certain embodiments, the present invention provides methods for in vivo amplification of regulatory Tregs (e.g., CD4⁺CD25^{high}Foxp3⁺) within a subject (e.g., a human subject suffering from or at risk of suffering from an autoimmune disorder) comprising administering to the subject a composition comprising one or more nanoparticles followed by (e.g., after 1 second, 2 seconds, 1 minute, 1 hour, 1 day, 1 week, 1 month, 1 year, etc.) administering to the subject a composition comprising one or more immunomodulatory agents (e.g., an IL-2, an IL-2 mutein, an IL-2 variant, or an IL-2/IC)) capable of expanding Tregs within the subject. In some embodiments, the composition comprising one or more nanoparticles is associated with (e.g., complexed, conjugated, encapsulated, absorbed, adsorbed, admixed) an mTOR inhibiting agent (e.g., rapamycin or a variant thereof). In some embodiments, the in vivo amplification of antigen-specific regulatory Tregs (e.g., CD4⁺CD25^{high}Foxp3⁺) within a subject is specific to a specific tissue region (e.g., a specific tissue region associated with one or more autoimmune disorders).

In certain embodiments, the present invention provides methods for in vivo amplification of antigen-specific regulatory Tregs (e.g., CD4⁺CD25^{high}Foxp3⁺) within a subject (e.g., a human subject suffering from or at risk of suffering from an autoimmune disorder) comprising administering to the subject a composition comprising one or more nanoparticles associated with one or more tolerogenic antigens followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding antigen-specific Tregs, wherein “antigen-specific” is specific to the one or more tolerogenic antigens associated with the comprising a nanoparticle associated with one or more tolerogenic antigens. In some embodiments, the in vivo amplification of antigen-specific regulatory Tregs (e.g., CD4⁺CD25^{high}Foxp3⁺) within a subject is specific to a specific tissue region (e.g., a specific tissue region associated with one or more autoimmune disorders).

In certain embodiments, the present invention provides methods for facilitating a strong immune tolerance to antigens associated with an autoimmune disorder within a subject (e.g., a human subject suffering from or at risk of suffering from an autoimmune disorder) comprising administering to the subject a composition comprising one or more nanoparticles followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding Tregs (e.g., CD4⁺CD25^{high}Foxp3⁺) within the subject. In some embodiments, the composition comprising one or more nanoparticles is associated with (e.g., complexed, conjugated, encapsulated, absorbed, adsorbed, admixed) an mTOR inhibiting agent (e.g., rapamycin or a variant thereof). In some embodiments, the facilitating a strong immune tolerance to antigens associated with an autoimmune disorder within a subject is specific to a specific tissue region (e.g., a specific tissue region associated with one or more autoimmune disorders).

In certain embodiments, the present invention provides methods for facilitating a strong immune tolerance to antigens associated with an autoimmune disorder within a subject (e.g., a human subject suffering from or at risk of suffering from an autoimmune disorder) comprising administering to the subject a composition comprising one or more nanoparticles associated with one or more tolerogenic antigens followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding antigen-specific Tregs (e.g., CD4⁺CD25^{high}Foxp3⁺), wherein “antigen-specific” is specific to the one or more tolerogenic antigens associated with the comprising a nanoparticle associated with one or more tolerogenic antigens. In some embodiments, the facilitating a strong immune tolerance to antigens associated with an autoimmune disorder within a subject is specific to a specific tissue region (e.g., a specific tissue region associated with one or more autoimmune disorders).

In certain embodiments, the present invention provides methods for increasing the ratio of CD3+FOXP3+ cells to CD3+FOXP3− cells within a population of T cells within a subject (e.g., a human subject suffering from or at risk of suffering from an autoimmune disorder) comprising administering to the subject a composition comprising one or more nanoparticles followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding Tregs (e.g., CD4⁺CD25^{high}Foxp3⁺) within the subject. In some embodiments, the composition comprising one or more nanoparticles is associated with (e.g., complexed, conjugated, encapsulated, absorbed, adsorbed, admixed) an mTOR inhibiting agent (e.g., rapamycin or a variant thereof). In some embodiments, the increasing the ratio of CD3+FOXP3+ cells to CD3+FOXP3− cells within the population of T cells within the subject is specific to a specific tissue region (e.g., a specific tissue region associated with one or more autoimmune disorders).

In certain embodiments, the present invention provides methods for increasing the ratio of CD3+FOXP3+ cells to CD3+FOXP3− cells within a population of T cells within a subject (e.g., a human subject suffering from or at risk of suffering from an autoimmune disorder) comprising administering to the subject a composition comprising one or more nanoparticles associated with one or more tolerogenic antigens followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding antigen-specific Tregs (e.g., CD4⁺CD25^{high}Foxp3±), wherein “antigen-specific” is specific to the one or more tolerogenic antigens associated with the comprising a nanoparticle associated with one or more tolerogenic antigens. In some embodiments, the increasing the ratio of CD3+FOXP3+ cells to CD3+FOXP3− cells within the population of T cells within the subject is specific to a specific tissue.

In certain embodiments, the present invention provides methods for treating, preventing and/or attenuating a disorder comprising administering to a subject (e.g., a human subject suffering from or at risk of suffering from an autoimmune disorder e.g., celiac disease) comprising administering to the subject a composition comprising one or more nanoparticles followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding Tregs (e.g., CD4⁺CD25^{high}Foxp3⁺) within the subject. In some embodiments, the composition comprising one or more nanoparticles is associated with (e.g., complexed, conjugated, encapsulated, absorbed, adsorbed, admixed) an mTOR inhibiting agent (e.g., rapamycin or a variant thereof). In some embodiments, the treating, preventing, and/or attenuating one or more autoimmune disorders in the subject is specific to a specific tissue region (e.g., a specific tissue region associated with the autoimmune disorder).

In certain embodiments, the present invention provides methods for treating, preventing and/or attenuating a disorder comprising administering to a subject (e.g., a human subject suffering from or at risk of suffering from an autoimmune disorder e.g., celiac disease) a composition comprising one or more nanoparticles associated with one or more tolerogenic antigens (e.g., one or more tolerogenic antigens associated with the autoimmune disorder) followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding antigen-specific Tregs (e.g., CD4⁺CD25^{high}Foxp3⁺), wherein “antigen-specific” is specific to the one or more tolerogenic antigens associated with the comprising a nanoparticle associated with one or more tolerogenic antigens. In some embodiments, the treating, preventing, and/or attenuating one or more disorders in the subject is specific to a specific tissue region (e.g., a specific tissue region associated with the disorder).

Such methods are not limited to treating a particular disorder.

In some embodiments, the disorder is an autoimmune disorder. Such methods are not limited to treating a particular autoimmune disorder. Examples of autoimmune disorders include, but are not limited to, multiple sclerosis (MS), celiac disease, rheumatoid arthritis, primary biliary cholangitis, primary sclerosing cholangitis, MOG antibody disease, diabetes (e.g., type 1 diabetes mellitus), autoimmune diseases of the thyroid (e.g., Hashimoto's thyroiditis, Graves' disease), thyroid-associated ophthalmopathy and dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutieres syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polvarthritis/fetal and neonatal anemia, Sepsis, and inflammatory bowel disease.

In some embodiments, the disorder is a transplantation related disorder. In some embodiments, the disorder is one or more allergies. In some embodiments, the disorder is a respiratory condition (e.g., asthma). In some embodiments, the disorder is graft-versus-host-disease (GvHD).

In some embodiments, such methods (e.g., administration of the composition comprising a nanoparticle associated with one or more tolerogenic antigens followed by the administration of the composition comprising an immunomodulatory agent capable of expanding Tregs) is further followed administration of one or more tolerogenic antigens to a specific tissue region (e.g., a specific tissue region associated with one or more autoimmune disorders). In some embodiments, the administration of one or more tolerogenic antigens to the specific tissue region is by injection administration and/or topical administration and/or subcutaneous administration. In some embodiments, the administration of one or more tolerogenic antigens to the specific tissue region prevents immune tolerance within the specific tissue region.

In some embodiments, the nanoparticle is associated with an immunomodulatory agent, and is not associated with a tolerogenic antigen. In some embodiments, the nanoparticle is associated with a tolerogenic antigen and is further associated with an immunomodulatory agent.

The methods described herein are not limited to a particular type or kind of composition comprising one or more immunomodulatory agents capable of expanding Tregs and/or antigen-specific Tregs.

In some embodiments, the composition comprising an immunomodulatory agent capable of expanding Tregs is comprised within a nanoparticle such that the nanoparticle is associated with the immunomodulatory agent capable of expanding Tregs (e.g., thereby providing a composition comprising a nanoparticle associated with an immunomodulatory agent capable of expanding Tregs).

Such embodiments are not limited to specific immunomodulatory agents.

In some embodiments, the one or more immunomodulatory agents is selected from the group comprising fingolimod; rapamycin; 2-(1′H-indole-3′-carbonyl)-thiazole-4-carboxylic acid methyl ester (ITE) or related ligands; Trichostatin A; Suberoylanilide hydroxamic acid (SAHA); statins; mTOR inhibitors; TGF-β signaling agents; TGF-β receptor agonists; histone deacetylase inhibitors; corticosteroids; inhibitors of mitochondrial function; NF-κβ inhibitors; adenosine receptor agonists; prostaglandin E2 agonists (PGE2; phosphodiesterase inhibitors; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors; autophagy inhibitors; aryl hydrocarbon receptor inhibitors; proteasome inhibitor I (PSI); oxidized ATPs IDO; vitamin D3; cyclosporins; aryl hydrocarbon receptor inhibitors; resveratrol; azathiopurine (Aza); 6-mercaptopurine (6-MP); 6-thioguanine (6-TG); FK506; sanglifehrin A; salmeterol; mycophenolate mofetil (MMF); aspirin and other COX inhibitors; niflumic acid; estriol; triptolide; OPN-305, OPN-401; Eritoran (E5564); TAK-242; Cpn10; NI-0101; 1A6; AV411; IRS-954 (DV-1079); IMO-3100; CPG-52363; CPG-52364; OPN-305; ATNC05; NI-0101; IMO-8400; Hydroxychloroquine; CU-CPT22; C29; Ortho-vanillin; SSL3 protein; OPN-305; 5 SsnB; Vizantin; (+)-N-phenethylnoroxymorphone; VB3323; Monosaccharide 3; (+)-Naltrexone and (+)-naloxone; HT52; HTB2; Compound 4a; CNT02424; TH1020; INH-ODN; E6446; AT791; CpG ODN 2088; ODN TTAGGG; COV08-0064; 2R9; GpG oligonucleotides; 2-aminopurine; Amlexanox; Bay11-7082; BX795; CH-223191; Chloroquine; CLI-095; CU-CPT9a; Cyclosporin A; CTY387; Gefitnib; Glybenclamide; H-89; H-131; Isoliquiritigenin; MCC950; MRT67307; OxPAPC; Parthenolide; Pepinh-MYD; Pepinh-TRIF; Polymyxin B; R406; RU.521; VX-765; YM201636; Z-VAD-FMK; and AHR-specific ligands; including but not limited to 2,3,7,8-tetrachloro-dibenzo-p-dioxin (TCDD); tryptamine (TA); and 6 formylindolo[3,2 b]carbazole (FICZ).

In some embodiments, the immunomodulatory agent is a cytokine. In some embodiments, the cytokine is a human cytokine. In some embodiments, the cytokine is selected from TGFβ, IL-1, IL-2, IL-4, IL-6, IL-7, IL-10, IL-12A, IL12B, IL-15, IL-21 and IL-18.

In some embodiments, the immunomodulatory agent is human IL-2. In some embodiments, the immunomodulatory agent is low dose IL-2. In some embodiments, the immunomodulatory agent is PT101 or a variant thereof. In some embodiments, the immunomodulatory agent is mutein IL-2 and/or variations thereof. In some embodiments, the IL-2 is any of the IL-2 cytokines, IL-2 muteins, and/or IL-2 variants as described in U.S. Pat. Nos. 11,091,527, 11,091,526, 11,077,195, 11,077,172, 10,960,079, 10,946,068, 10,766,938, 10,722,460, 10,174,092, 10,174,091; EP Patent No. 3808764, and/or U.S. Patent Application Publication No. US20120315245.

In some embodiments, the immunomodulatory agent is an IL-2:anti-IL-2 antibody immune complex (IL-2/IC).

In some embodiments, IL-2 is an extended pharmacokinetic (PK) IL-2. In some embodiments, the extended-PK IL-2 comprises a fusion protein. In some embodiments, the fusion protein comprises an IL-2 moiety and a moiety selected from the group consisting of an immunoglobulin fragment, human serum albumin, and Fn3. In some embodiments, the fusion protein comprises an IL-2 moiety operably linked to an immunoglobulin Fc domain. In some embodiments, the fusion protein comprises an IL-2 moiety operably linked to human serum albumin. In some embodiments, the extended-PK IL-2 comprises an IL-2 moiety conjugated to a non-protein polymer. In some embodiments, the non-protein polymer is a polyethylene glycol.

In some embodiments, the extended-PK IL-2 is mutated such that it has an altered affinity (e.g., a higher affinity) for the IL-2R alpha receptor compared with unmodified IL-2. Site-directed mutagenesis can be used to isolate IL-2 mutants that exhibit high affinity binding to CD25, i.e., IL-2Rα, as compared to wild-type IL-2. Increasing the affinity of IL-2 for IL-2Rα at the cell surface will increase receptor occupancy within a limited range of IL-2 concentration, as well as raise the local concentration of IL-2 at the cell surface.

In some embodiments, IL-2 mutants are provided, which may be, but are not necessarily, substantially purified and which can function as high affinity CD25 binders. IL-2 is a T cell growth factor that induces proliferation of antigen-activated T cells and stimulation of NK cells. Exemplary IL-2 mutants which are high affinity binders include those described in WO2013/177187A2. Further exemplary IL-2 mutants with increased affinity for CD25 are disclosed in U.S. Pat. No. 7,569,215, the contents of which are incorporated herein by reference.

In certain embodiments, the composition comprising an immunomodulatory agent (e.g., human cytokine (e.g., an IL-2, an IL-2 mutein, an IL-2 variant, or an IL-2/IC)) is capable of expanding Treg cells within a subject or sample. Indeed, such compositions comprising an immunomodulatory agent are capable of increasing the ratio of Tregs to non-regulatory T cells. The ratio may be measured by determining the ratio of CD3+FOXP3+ cells to CD3+FOXP3− cells within the population of T cells. The typical Treg frequency in human blood is 5-10% of total CD4+CD3+ T cells, however, in autoimmune disorders the percentage may be lower or higher. In preferred embodiments, the percentage of Treg increases at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1000%. Maximal fold increases in Treg may vary for particular diseases; however, a maximal Treg frequency that might be obtained through IL-2 mutein treatment is 50% or 60% of total CD4+CD3+ T cells. In certain embodiments, a composition comprising an immunomodulatory agent (e.g., human cytokine (e.g., an IL-2, an IL-2 mutein, an IL-2 variant, or an IL-2/IC)) is administered to a subject and the ratio of regulatory T cells (Tregs) to non-regulatory T cells within peripheral blood of a subject increases.

The methods described herein are not limited to a particular composition comprising one or more nanoparticles associated with or not associated with one or more tolerogenic antigens.

Such compositions are not limited to a particular nanoparticle. In some embodiments, the nanoparticle is a sHDL nanoparticle. In some embodiments, the average size of the nanoparticle is between 6 to 500 nm (e.g., 7 to 20 nm, 21 to 50 nm, 51 to 100 nm, 101 to 200 nm, 201 to 300 nm, 301 to 400 nm, and 401 to 500 nm). In some embodiments, the average particle size of the sHDL nanoparticle is between 6-70 nm (e.g., 7 to 10 nm, 11 to 20 nm, 21 to 30 nm, 31 to 40 nm, 41 to 50 nm, 51 to 60 nm, and 61 to 70 nm).

In some embodiments, the phospholipid is selected from the group consisting of 1,2-dilauroyl-sn-glycero-3-phosphocholine; 1,2-dimyristoyl-sn-glycero-3-phosphocholine; 1,2-dipalmitoyl-sn-glycero-3-phosphocholine; 1,2-distearoyl-sn-glycero-3-phosphocholine; 1,2-diarachidoyl-sn-glycero-3-phosphocholine; 1,2-dibehenoyl-sn-glycero-3-phosphocholine; 1,2-dilignoceroyl-sn-glycero-3-phosphocholine; 1,2-dimyristoleoyl-sn-glycero-3-phosphocholine; 1,2-dimyristelaidoyl-sn-glycero-3-phosphocholine; 1,2-dipalmitoleoyl-sn-glycero-3-phosphocholine; 1,2-dipalmitelaidoyl-sn-glycero-3-phosphocholine; 1,2-dipetroselenoyl-sn-glycero-3-phosphocholine; 1,2-dioleoyl-sn-glycero-3-phosphocholine; 1,2-dielaidoyl-sn-glycero-3-phosphocholine; 1,2-dieicosenoyl-sn-glycero-3-phosphocholine; 1,2-dinervonoyl-sn-glycero-3-phosphocholine; 1,2-dilauroyl-sn-glycero-3-phosphoethanolamine; 1,2-dimyristoyl-sn-glycero-3-phosphoethanolamine; 1,2-dipentadecanoyl-sn-glycero-3-phosphoethanolamine; 1,2-dipalmitoyl-sn-glycero-3-phosphoethanolamine; 1,2-distearoyl-sn-glycero-3-phosphoethanolamine; 1,2-dipalmitoleoyl-sn-glycero-3-phosphoethanolamine; 1,2-dielaidoyl-sn-glycero-3-phosphoethanolamine; 1,2-dioleoyl-sn-glycero-3-phosphoethanolamine; dioleoyl-sn-glycero-3-phosphoethanolamine-N-[3-(2-pyridyldithio) propionate]; 1,2-dipalmitoyl-sn-glycero-3-phosphothioethanol; 1,2-di-(9Z-octadecenoyl)-sn-glycero-3-phosphoethanolamine-N-[4-(p-maleimidophenyl)butyramide]; 1,2-dihexadecanoyl-sn-glycero-3-phosphoethanolamine-N-[4-(p-maleimidophenyl)butyramide]; 1,2-dihexadecanoyl-sn-glycero-3-phosphoethanolamine-N-[4-(p-maleimidomethyl)cyclohexane-carboxamide]; 1,2-di-(9Z-octadecenoyl)-sn-gly cero-3-phosphoethanolamine-N-[4-(p-maleimidomethyl)cyclohexane-carboxamide]; N-[(3-Maleimide-1-oxopropyl)aminopropyl polyethyleneglycol-carbamyl]distearoylphosphatidyl-ethanolamine; N-[(3-Maleimide-1-oxopropyl)aminopropyl polyethyleneglycol-carbamyl]distearoylphosphatidyl-ethanolamine; N-(3-Maleimide-1-oxopropyl)-L-α-phosphatidylethanolamine, Distearoyl; N-[(3-Maleimide-1-oxopropyl)aminopropyl polyethyleneglycol-carbamyl]distearoylphosphatidyl-ethanolamine; N-(3-Maleimide-1-oxopropyl)-L-α-phosphatidylethanolamine, Dimyristoy; N-(3-Maleimide-1-oxopropyl)-L-α-phosphatidylethanolamine, Dioleoyl; N-(3-Maleimide-1-oxopropyl)-L-α-phosphatidylethanolamine, Dipalmitoyl; N-(3-Maleimide-1-oxopropyl)-L-α-phosphatidylethanolamine, 1-Palmitoyl-2-oleoyl; phosphatidylcholine; phosphatidylinositol; phosphatidylserine; phosphatidylethanolamine; N-(Succinimidyloxy-glutaryl)-L-α-phosphatidylethanolamine, Distearoyl; N-(Succinimidyloxy-glutaryl)-L-α-phosphatidylethanolamine, Dioleoyl; N-(Succinimidyloxy-glutaryl)-L-α-phosphatidylethanolamine, 1-Palmitoyl-2-oleoyl; N-(Succinimidyloxy-glutaryl)-L-α-phosphatidylethanolamine, Dipalmitoyl; N-(Succinimidyloxy-glutaryl)-L-α-phosphatidylethanolamine, Dimyristoyl; 3-(N-succinimidyloxyglutaryl)aminopropyl, and polyethyleneglycol-carbamyl distearoylphosphatidyl-ethanolamine; N-(3-oxopropoxy polyethylenegly col)carbamyl-distearoyl-ethanolamine.

In some embodiments, the HDL apolipoprotein component is selected from the group consisting of apolipoprotein A-I (apo A-I), apolipoprotein A-II (apo A-II), apolipoprotein A-II xxx (apo A-II-xxx), apolipoprotein A4 (apo A4), apolipoprotein Cs (apo Cs), apolipoprotein E (apo E), apolipoprotein A-I milano (apo A-I-milano), apolipoprotein A-I paris (apo A-I-paris), apolipoprotein M (apo M), an HDL apolipoprotein mimetic, preproapoliprotein, preproApoA-T, proApoA T, preproApoA-II, proApoA 11, preproApoA-V, proApoA4V, ApoA-V, preproApoE, proApoE, preproApoA I_{Milano}, prOApoA-I_{Milano}, preproApoA-I_{Paris}, proApoA-I_{Paris}, and mixtures thereof.

In some embodiments, the ApoA-I mimetic is
described by any of SEQ ID NOs: 1-336 and
(SEQ ID NO: 341)
WDRVKDLATVYVDVLKDSGRDYVSQF,
(SEQ ID NO: 342)
LKLLDNWDSVTSTFSKLREOL,
(SEQ ID NO: 343)
PVTOEFWDNLEKETEGLROEMS,
(SEQ ID NO: 344)
KDLEEVKAKVQ,
(SEQ ID NO: 345)
KDLEEVKAKVO,
(SEQ ID NO: 346)
PYLDDFQKKWQEEMELYRQKVE,
(SEQ ID NO: 347)
PLRAELQEGARQKLHELOEKLS,
(SEQ ID NO: 348)
PLGEEMRDRARAHVDALRTHLA,
(SEQ ID NO: 349)
PYSDELRQRLAARLEALKENGG,
(SEQ ID NO: 350)
ARLAEYHAKATEHLSTLSEKAK,
(SEQ ID NO: 351)
PALEDLROGLL,
(SEQ ID NO: 352)
PVLESFKVSFLSALEEYTKKLN,
(SEQ ID NO: 353)
PVLESFVSFLSALEEYTKKLN,
(SEQ ID NO: 352)
PVLESFKVSFLSALEEYTKKLN,
(SEQ ID NO: 354)
TVLLLTICSLEGALVRRQAKEPCV,
(SEQ ID NO: 355)
QTVTDYGKDLME,
(SEQ ID NO: 356)
KVKSPELOAEAKSYFEKSKE,
(SEQ ID NO: 357)
VLTLALVAVAGARAEVSADOVATV,
(SEQ ID NO: 358)
NNAKEAVEHLOKSELTOOLNAL,
(SEQ ID NO: 359)
LPVLVWLSIVLEGPAPAOGTPDVSS,
(SEQ ID NO: 360)
LPVLVVVLSIVLEGPAPAQGTPDVSS,
(SEQ ID NO: 361)
ALDKLKEFGNTLEDKARELIS,
(SEQ ID NO: 362)
VVALLALLASARASEAEDASLL,
(SEQ ID NO: 363)
KLRKLRKRLLRDADDLQKRLAVYOA,
(SEQ ID NO: 364)
AQAWGERLRARMEEMGSRTRDR,
(SEQ ID NO: 365)
LDEVKEQVAEVRAKLEEQAQ,
(SEQ ID NO: 236)
DWLKAFYDKVAEKLKEAF,
(SEQ ID NO: 366)
DWLKAFYDKVAEKLKEAFPDWAKAAYDKAAEKAKEAA,
(SEQ ID NO: 367)
PVLDLFRELLNELLEALKQKL,
(SEQ ID NO: 368)
PVLDLFRELLNELLEALKQKLA,
(SEQ ID NO: 4)
PVLDLFRELLNELLEALKQKLK,
(SEQ ID NO: 369)
PVLDLFRELLNELLEALKQKLA,
(SEQ ID NO: 370)
PVLDLFRELLNELLEALKKLLK,
(SEQ ID NO: 371)
PVLDLFRELLNELLEALKKLLA,
(SEQ ID NO: 372)
PLLDLFRELLNELLEALKKLLA,
and
(SEQ ID NO: 373)
EVRSKLEEWFAAFREFAEEFLARLKS.

In some embodiments, the one or more tolerogenic antigens is a plurality of tolerogenic antigens.

In some embodiments, the plurality of tolerogenic antigens are tolerogenic antigens including between 3 amino acids and 50 amino acids in length (e.g., e.g., about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 amino acids in length).

In some embodiments, the plurality of tolerogenic antigens are tolerogenic antigens including a polypeptide including an amino acid sequence of any one of SEQ ID NOs: 375-796.

In some embodiments, the plurality of tolerogenic antigens are human allograft transplantation antigens. In some embodiments, the human allograft transplantation antigens are selected from subunits of the various MHC class I and MHC class II haplotype proteins, and single-amino-acid polymorphisms on minor blood group antigens including RhCE, Kell, Kidd, Duffy and Ss.

In some embodiments, the plurality of tolerogenic antigens are specific for type 1 diabetes mellitus. In some embodiments, the type 1 diabetes mellitus tolerogenic antigens are selected from insulin, proinsulin, preproinsulin, glutamic acid decarboxylase-65 (GAD-65), GAD-67, insulinoma-associated protein 2 (IA-2), insulinoma-associated protein 2P (IA-20), ICA69, ICA12 (SOX-13), carboxypeptidase H, Imogen 38, GLIMA 38, chromogranin-A, HSP-60, caboxypeptidase E, peripherin, glucose transporter 2, hepatocarcinoma-intestine-pancreas/pancreatic associated protein, S1000, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5.

In some embodiments, the tolerogenic antigens are specific for one or more of the following autoimmune disorders: rheumatoid arthritis, multiple sclerosis, primary biliary cholangitis, primary sclerosing cholangitis, MOG antibody disease, diabetes (e.g., type 1 diabetes mellitus), autoimmune diseases of the thyroid (e.g., Hashimoto's thyroiditis, Graves' disease), thyroid-associated ophthalmopathy and dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutières syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polvarthritis/fetal and neonatal anemia, Sepsis, and inflammatory bowel disease.

In some embodiments, the plurality of tolerogenic antigens include one or more of tolerogenic antigens selected from thyroglobulin (TG), thyroid peroxidase (TPO), thyrotropin receptor (TSHR), sodium iodine symporter (NIS), megalin, thyroid autoantigens including TSHR, insulin-like growth factor 1 receptor, calcium sensitive receptor, 21-hydroxylase, 17α-hydroxylase, and P450 side chain cleavage enzyme (P450scc), ACTH receptor, P450c21, P450c17, FSH receptor, α-enolase, pituitary gland-specific protein factor (PGSF) 1a and 2, and type 2 iodothyronine deiodinase, myelin basic protein, myelin oligodendrocyte glycoprotein, proteolipid protein, collagen II, H^{|}, K^{|}-ATPase, tissue transglutaminase and gliadin, tyrosinase, tyrosinase related protein 1 and 2, acetylcholine receptor, desmoglein 3, 1 and 4, pemphaxin, desmocollins, plakoglobin, perplakin, desmoplakins, acetylcholine receptor, BP180, BP230, plectin, laminin 5, endomysium, tissue transglutaminase, collagen VII, matrix metalloproteinase 1 and 3, the collagen-specific molecular chaperone heat-shock protein 47, fibrillin-1, PDGF receptor, Scl-70, U1 RNP, Th/To, Ku, Jol, NAG-2, centromere proteins, topoisomerase I, nucleolar proteins, RNA polymerase I, II and III, PM-Slc, fibrillarin, B23, UlsnRNP, nuclear antigens SS-A and SS-B, fodrin, poly(ADP-ribose) polymerase, topoisomerase, nuclear proteins including SS-A, high mobility group box 1 (HMGB1), nucleosomes, histone proteins, double-stranded DNA, glomerular basement membrane proteins including collagen IV, cardiac myosin, aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and type 1 interferons interferon alpha, beta and omega.

Such compositions are not limited to specific tolerogenic antigens. In some embodiments, the tolerogenic antigen is a foreign antigen against which a patient has developed an unwanted immune response. In some embodiments, the plurality of tolerogenic antigens are specific for celiac disease. In some embodiments, the tolerogenic antigens are selected form gliadin, glutenin, and fragments thereof capable of inducing an immune response. In some embodiments, the tolerogenic antigens are selected from gliadin (e.g., α-, γ-, and ω-gliadin) or fragments thereof. In some embodiments, the tolerogenic antigens are selected from the group consisting of α, γ, and ω gliadins or fragments thereof. In some embodiments, the tolerogenic antigen includes a polypeptide having at least 90% (e.g., at least 91%, at least 92%, at least 93%, at least 94%, at least 95%, at least 96%, at least 97%, at least 98%, at least 99%) sequence identity to the polypeptide sequence of any one of SEQ ID NOs: 375-580. In some embodiments, tolerogenic antigen includes a polypeptide having at least 95% (e.g., 96%, 97%, 98%, 99%, or 100%) sequence identity to any one of the polypeptide sequences of SEQ ID NOs: 375-580. In some embodiments, the tolerogenic antigen includes a polypeptide having the polypeptide sequence of any one of SEQ ID NOs: 375-580. In some embodiments, the tolerogenic antigen includes two or more (e.g., two, three, four, five, and six) polypeptide sequences having the sequence of any one of SEQ ID NOs: 375-580.

In some embodiments, the tolerogenic antigen is a self antigen against which a subject (e.g., a human patient) has developed an autoimmune response or may develop an autoimmune response. Examples include proinsulin (e.g., for subjects suffering from or at risk of suffering from diabetes), collagens (e.g., for subjects suffering from or at risk of suffering from rheumatoid arthritis), and myelin basic protein (e.g., for subjects suffering from or at risk of suffering from multiple sclerosis). There are many proteins that are human autoimmune proteins, a term referring to various autoimmune diseases wherein the protein or proteins causing the disease are known or can be established by routine testing. Embodiments include testing a patient to identify an autoimmune protein and creating an antigen for use in a molecular fusion and creating immunotolerance to the protein. Embodiments include an antigen, or choosing an antigen from, one or more of the following proteins. In type 1 diabetes mellitus, several main antigens have been identified: insulin, proinsulin, preproinsulin, glutamic acid decarboxylase-65 (GAD-65), GAD-67, insulinoma-associated protein 2 (IA-2), and insulinoma-associated protein 2p (IA-2B); other antigens include ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, S1003, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5. In autoimmune diseases of the thyroid, including Hashimoto's thyroiditis and Graves' disease, main antigens include thyroglobulin (TG), thyroid peroxidase (TPO) and thyrotropin receptor (TSHR); other antigens include sodium iodine symporter (NIS) and megalin. In thyroid-associated ophthalmopathy and dermopathy, in addition to thyroid autoantigens including TSHR, an antigen is insulin-like growth factor 1 receptor. In hypoparathyroidism, a main antigen is calcium sensitive receptor. In Addison's disease, main antigens include 21-hydroxylase, 17α-hydroxylase, and P450 side chain cleavage enzyme (P450scc); other antigens include ACTH receptor, P450c21 and P450c17. In premature ovarian failure, main antigens include FSH receptor and α-enolase. In autoimmune hypophysitis, or pituitary autoimmune disease, main antigens include pituitary gland-specific protein factor (PGSF) 1a and 2; another antigen is type 2 iodothyronine deiodinase. In multiple sclerosis, main antigens include myelin basic protein, myelin oligodendrocyte glycoprotein and proteolipid protein. In rheumatoid arthritis, a main antigen is collagen II. In immunogastritis, a main antigen is H⁺, K⁺-ATPase. In pernicious angemis, a main antigen is intrinsic factor. In celiac disease, main antigens are tissue transglutaminase and gliadin. In vitiligo, a main antigen is tyrosinase, and tyrosinase related protein 1 and 2. In myasthenia gravis, a main antigen is acetylcholine receptor. In pemphigus vulgaris and variants, main antigens are desmoglein 3, 1 and 4; other antigens include pemphaxin, desmocollins, plakoglobin, perplakin, desmoplakins, and acetylcholine receptor. In bullous pemphigoid, main antigens include BP180 and BP230; other antigens include plectin and laminin 5. In dermatitis herpetiformis Duhring, main antigens include endomysium and tissue transglutaminase. In epidermolysis bullosa acquisita, a main antigen is collagen VII. In systemic sclerosis, main antigens include matrix metalloproteinase 1 and 3, the collagen-specific molecular chaperone heat-shock protein 47, fibrillin-1, and PDGF receptor; other antigens include Scl-70, U1 RNP, Th/To, Ku, Jol, NAG-2, centromere proteins, topoisomerase I, nucleolar proteins, RNA polymerase I, II and III, PM-Slc, fibrillarin, and B23. In mixed connective tissue disease, a main antigen is U1snRNP. In Sjogren's syndrome, the main antigens are nuclear antigens SS-A and SS-B; other antigens include fodrin, poly(ADP-ribose) polymerase and topoisomerase. In systemic lupus erythematosus, main antigens include nuclear proteins including SS-A, high mobility group box 1 (HMGB1), nucleosomes, histone proteins and double-stranded DNA. In Goodpasture's syndrome, main antigens include glomerular basement membrane proteins including collagen IV. In rheumatic heart disease, a main antigen is cardiac myosin. Other autoantigens revealed in autoimmune polyglandular syndrome type 1 include aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta and omega.

In some cases, the tolerogenic antigen is a foreign antigen against which a patient has developed an unwanted immune response. Examples are food antigens. Embodiments include testing a patient to identify foreign antigen and creating a molecular fusion that includes the antigen and treating the patient to develop immunotolerance to the antigen or food. Examples of such foods and/or antigens are provided. Examples are from peanut: conarachin (Ara h 1), allergen II (Ara h 2), arachis agglutinin, conglutin (Ara h 6); from apple: 31 kda major allergen/disease resistance protein homolog (Mal d 2), lipid transfer protein precursor (Mal d 3), major allergen Mal d 1.03D (Mal d 1): from milk: A-lactalbumin (ALA), lactotransferrin; from kiwi: actinidin (Act c 1, Act d 1), phytocystatin, thaumatin-like protein (Act d 2), kiwellin (Act d 5); from mustard: 2S albumin (Sin a 1), 11S globulin (Sin a 2), lipid transfer protein (Sin a 3), profilin (Sin a 4); from celery: profilin (Api g 4), high molecular weight glycoprotein (Api g 5); from shrimp: Pen a 1 allergen (Pen a 1), allergen Pen m 2 (Pen m 2), tropomyosin fast isoform; from wheat and/or other cereals: high molecular weight glutenin, low molecular weight glutenin, alpha- and gamma-gliadin, hordein, secalin, avenin; from strawberry: major strawberry allergy Fra a 1-E (Fra a 1), from banana: profilin (Mus xp 1).

In some embodiments, the tolerogenic antigens are multimeric tolerogenic antigens including the following N-terminal-to-C-terminal structure

(P₄-L₄)ₙ₄-(P₃-L₃)ₙ₃-P₂-(L₁-P₁)ₙ₁
where P₁, P₂, P₃, and P₄ are each independently a tolerogenic antigen;
L₁, L₃, and L₄ are each independently a linker; and
n₁, n₃, and n₄ are each independently 0 or 1, wherein at least one of n₁, n₃, and n₄ are 1.

In some embodiments, n₁ is 1, n₃ is 0, and n₄ is 0, and the tolerogenic antigen includes the following N-terminal-to-C-terminal structure:

P₂-L₁-P₁.

In some embodiments, L₁ is a peptide linker including between 2 and 200 amino acids (e.g., between 5 and 50 (e.g., between 5 and 20, 15 and 30, 25 and 40, or 35 and 50), between 45 and 100 (e.g., between 45 and 60, 55 and 70, 65 and 80, 75 and 90, or 85 and 100), 95 and 150 (e.g., between 95 and 110, 105 and 120, 115 and 130, 125 and 140, or 135 and 150), or 145 and 200 amino acids (e.g., between 145 and 160, 155 and 170, 165 and 180, 175 and 190, or 185 and 200). In some embodiments, L₁ is a peptide linker including glycine (G) and serine (S) residues. In some embodiments, L₁ is a peptide linker including the amino acid sequence of (GS)ₓ, (GGS)ₓ, or (GGGGS)ₓ, where x is an integer from 1 to 10 (e.g., 1, 2, 3, 4, 5, 6, 7, 8, 9, or 10). In some embodiments, P₁ and P₂ each include different tolerogenic antigens. In some embodiments, P₁ and P₂ each include identical tolerogenic antigens.

In some embodiments, n₁ is 1, n₃ is 1, and n₄ is 0, and the tolerogenic antigen includes the following N-terminal-to-C-terminal structure:

P₃-L₃-P₂-L₁-P₁.

In some embodiments, L₁ and L₃ are each an independently selected peptide linker including between 2 and 200 amino acids (e.g., between 5 and 50 (e.g., between 5 and 20, 15 and 30, 25 and 40, or 35 and 50), between 45 and 100 (e.g., between 45 and 60, 55 and 70, 65 and 80, 75 and 90, or 85 and 100), 95 and 150 (e.g., between 95 and 110, 105 and 120, 115 and 130, 125 and 140, or 135 and 150), or 145 and 200 amino acids (e.g., between 145 and 160, 155 and 170, 165 and 180, 175 and 190, or 185 and 200). In some embodiments, L₁ and L₃ are each an independently selected peptide linker including glycine (G) and serine (S) residues. In some embodiments, L₁ and L₃ are each an independently selected peptide linker including the amino acid sequence of (GS)ₓ, (GGS)ₓ, or (GGGGS)ₓ, where x is an integer from 1 to 10 (e.g., 1, 2, 3, 4, 5, 6, 7, 8, 9, or 10). In some embodiments, P₁, P₂, and/or P₃ each include different tolerogenic antigens. In some embodiments, P₁, P₂, and P₃ each include identical tolerogenic antigens.

In some embodiments, n₁ is 1, n₃ is 1, and n₄ is 1, and the tolerogenic antigen includes the following N-terminal-to-C-terminal structure:

P₄-L₄-P₃-L₃-P₂-L₁-P₁.

In certain embodiments, L₁ and L₂ are each an independently selected peptide linker including between 2 and 200 amino acids (e.g., between 5 and 50 (e.g., between 5 and 20, 15 and 30, 25 and 40, or 35 and 50), between 45 and 100 (e.g., between 45 and 60, 55 and 70, 65 and 80, 75 and 90, or 85 and 100), 95 and 150 (e.g., between 95 and 110, 105 and 120, 115 and 130, 125 and 140, or 135 and 150), or 145 and 200 amino acids (e.g., between 145 and 160, 155 and 170, 165 and 180, 175 and 190, or 185 and 200). In some embodiments, L₁, L₂, and L₃ are each an independently selected peptide linker including glycine (G) and serine (S) residues. In some embodiments, L₁, L₂, and L₃ are each an independently selected peptide linker including the amino acid sequence of (GS)ₓ, (GGS)ₓ, or (GGGGS (SEQ ID NO: 219))ₓ, where x is an integer from 1 to 10. In some embodiments, P₁, P₂, P₃, and/or P₄ each include different tolerogenic antigens. In some embodiments, P₁, P₂, P₃, and P₄ each include identical tolerogenic antigens.

In some embodiments, the number of tolerogenic antigens associated with a specific nanoparticle includes a population of between 1 and 30 (e.g., 1-10, 9 to 15, 12 to 18, 15 to 22, 18 to 25, 20 to 27, 22 to 28, or 25 to 30) tolerogenic antigens per nanoparticle. In some embodiments, the number of tolerogenic antigens associated with a specific nanoparticle includes a population of 6 tolerogenic antigens per particle. In other embodiments, the number of tolerogenic antigens associated with a specific nanoparticle includes a population of 8 tolerogenic antigens per particle. In some embodiments, the population of tolerogenic antigens associated with a specific nanoparticle are the same antigen. In some embodiments, the population of tolerogenic antigens associated with a specific nanoparticle includes between 1 and 5 (e.g., 2, 3, 4, and 5) different tolerogenic antigens. In some embodiments, the population of tolerogenic antigens associated with a specific nanoparticle include 3 to 4 different tolerogenic antigens. In some embodiments, the population of tolerogenic antigens are specific to between 1 and 3 different diseases. In certain embodiments, the population of tolerogenic antigens are specific to the same disease.

In some embodiments, the population of tolerogenic antigens associated with a specific nanoparticle includes (i) a first polypeptide population including the amino acid sequence of any one of SEQ ID NOs: 406-588, or a biologically active fragment or variant thereof, (ii) a second polypeptide population including the amino acid sequence of any one of SEQ ID NOs: 406-588, or biologically active fragment or variant thereof, and (iii) a third polypeptide population including the amino acid sequence of any one of SEQ ID NOs: 406-588, or a biologically active fragment or variant thereof.

In some embodiments, the first polypeptide population includes the amino acid sequence of SEQ ID NO: 474, or a biologically active fragment or variant thereof, (ii) the second polypeptide population includes the amino acid sequence of any one of SEQ ID NOs: 406-588, or biologically active fragment or variant thereof, and (iii) the third polypeptide population includes the amino acid sequence of any one of SEQ ID NOs: 406-588, or a biologically active fragment or variant thereof.

In some embodiments, the population of tolerogenic antigens associated with a specific nanoparticle includes (i) the first polypeptide population includes the amino acid sequence of SEQ ID NO: 474, or a biologically active fragment or variant thereof, (ii) the second polypeptide population includes the amino acid sequence of SEQ ID NO: 475, or biologically active fragment or variant thereof, and (iii) the third polypeptide population includes the amino acid sequence of any one of SEQ ID NOs: 406-588, or a biologically active fragment or variant thereof. In some embodiments, the third polypeptide population includes the amino acid sequence of SEQ ID NO: 476, or a biologically active fragment or variant thereof. In some embodiments, the second polypeptide population includes the amino acid sequence of SEQ ID NO: 477, or a biologically active fragment or variant thereof, and/or the third polypeptide population includes the amino acid sequence of SEQ ID NO: 478, or a biologically active fragment or variant thereof.

In some embodiments, the tolerogenic antigen includes a polypeptide having at least 90% (e.g., at least 91%, at least 92%, at least 93%, at least 94%, at least 95%, at least 96%, at least 97%, at least 98%, at least 99%) sequence identity to the polypeptide sequence of SEQ ID NO: 374. In some embodiments, the tolerogenic antigen includes a polypeptide sequence of SEQ ID NO: 374. In some embodiments, the tolerogenic antigen includes a fragment of SEQ ID NO: 373, including 6-12 (e.g., 7, 8, 9, 10, 11, and 12) amino acid residues in length.

In some embodiments, the tolerogenic antigen includes an amide group at the C-terminus. In certain embodiments, the tolerogenic antigen includes a pyroglutamic acid residue at the N-terminus. In another embodiment, the tolerogenic antigen includes an acetyl group at the N-terminus. In some embodiments, the tolerogenic antigen includes a pyroglutamic acid residue at the N-terminus and an amide group at the C-terminus. In some embodiments, the tolerogenic antigen includes an acetyl group at the N-terminus and an amide group at the C-terminus. In certain embodiments, the tolerogenic antigen includes an N-terminus or a C-terminus modified with a cysteine residue bound to a linker. In some embodiments, the tolerogenic antigen includes an N-terminus and a C-terminus modified with cysteine residues bound to a linker.

In some embodiments of any one of the compositions described herein, the population of tolerogenic antigens are conjugated with the nanoparticle phospholipid in such a manner that facilitates strong immune tolerance upon administration to a subject (e.g., a human subject suffering from or at risk of suffering from an autoimmune disorder e.g., MS, celiac disease, rheumatoid arthritis, primary biliary cholangitis, primary sclerosing cholangitis, MOG antibody disease, diabetes (e.g., type 1 diabetes mellitus), autoimmune diseases of the thyroid (e.g., Hashimoto's thyroiditis, Graves' disease), thyroid-associated ophthalmopathy and dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutières syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polvarthritis/fetal and neonatal anemia, Sepsis, or inflammatory bowel disease).

In some embodiments, the plurality of tolerogenic antigens are conjugated with the nanoparticle phospholipid via a thiol-reactive and reduction-insensitive linkage between the tolerogenic antigen and the nanoparticle phospholipid. Indeed, a thiol-reactive and reduction-insensitive linkage between the tolerogenic antigen and the nanoparticle phospholipid facilitates strong immune tolerance. In some embodiments, the phospholipid is N-(3-Maleimide-1-oxopropyl)-L-α-phosphatidylethanolamine.

In some embodiments, the tolerogenic antigen is conjugated with the nanoparticle phospholipid via an amine-mediated interaction (e.g., N-(Succinimidyloxy-glutaryl)-L-α-phosphatidylethanolamine, Dioleoyl (DOPE-NHS)). In some embodiments, the amine-mediated interaction is N-(Succinimidyloxy-glutaryl)-L-α-phosphatidylethanolamine, Dioleoyl (DOPE-NHS)). In some embodiments, the amine-mediated interaction is through an amine-reactive phospholipid with self-immolative linkage (e.g., linkers including o-dithiobenzyl, p-dithiobenzyl, beta-dithiobenzyl carbamate moieties, 2,2-dimethyl-4-mercapto-butyric acid, or Disulfide-carbonate-based traceless linker).

In some embodiments, the composition does not contain an adjuvant.

In certain embodiments, compositions comprising one or more immunomodulatory agents are provided.

Additional embodiments will be apparent to persons skilled in the relevant art based on the teachings contained herein.

### BRIEF DESCRIPTION OF THE DRAWINGS

FIG. 1. HDL nanodiscs were analyzed by dynamic light scattering. Shown are the hydrodynamic size of nanodiscs before and after loading of OVA-II peptide, polydispersity index (PDI), and intensity and volume profiles.

FIG. 2. Nanodisc-OVA-II were analyzed by LC-MS. Shown are chromatograms of DOPE-MAL before and after conjugation to OVA-II peptide, the conjugation efficiency of DOPE-MAL to OVA-II peptide, and the loading efficiency of DOPE-OVA-II into Nanodisc.

FIG. 3. Blank Nanodisc and Nanodisc-OVA-II were analyzed by GPC. Shown are their chromatograms.

FIG. 4. Schematic illustration of treatment regimen for ND-OVA-II combined with IL-2/IC therapy.

FIG. 5. PBMCs were analyzed by flow cytometry on day 7. Shown are representative scatter plots. Numbers in plots represent frequencies of parent gate.

FIG. 6A-F. PBMCs were analyzed by flow cytometry on day 7. Shown are a) % CD4⁺ T cells among PBMCs, b) % OT-II Tetramer+ T cells among CD4⁺ T cells in PBMCs, c) % Foxp3⁺CD25⁺ T_{regs} among CD4+ T cells, d) % OT-II Tetramer+Foxp3⁺CD25⁺ T_{regs} among CD4+ T cells, e) mean fluorescence intensity (MFI) of CD25 among Foxp3⁺CD25⁺ T_{regs}, and f) MFI of CD25 among OT-II Tetramer^{|}Foxp3^{|}CD25^{|} T_{regs}.

FIG. 7. PBMCs were analyzed by flow cytometry on day 14. Shown are representative scatter plots. Numbers in plots represent frequencies of parent gate.

FIG. 8A-F. PBMCs were analyzed by flow cytometry on day 14. Shown are a) % and number of CD4⁺ T cells among PBMCs, b) % and number of OT-II Tetramer⁺ T cells among CD4⁺ T cells in PBMCs, c) % and number of Foxp3⁺CD25⁺ T_{regs} among CD4⁺ T cells, d) % and number of OT-II Tetramer⁺Foxp3⁺CD25⁺ T_{regs} among CD4⁺ T cells, e) MFI of CD25 and f) GITR among Foxp3⁺CD25⁺ T_{regs} and OT-II Tetramer⁺Foxp3⁺CD25⁺ T_{regs}.

FIG. 9A-C. Shown are the changes of a) total Foxp3⁺CD25⁺ T_{regs}, b) OT-11 Tetramer⁺Foxp3⁺CD25⁺ T_{regs}, and c) OT-II Tetramer⁺Foxp3-CD25⁺ T_{conv} among peripheral CD4⁺ T cells on days 7 and 14.

FIG. 10. Schematic illustration of treatment regimen for ACT+ND-OVA-II combined with IL-2/IC therapy, followed by antigen challenge in ears on day 25.

FIG. 11A-C. Twenty four hours post intra-dermal challenge with MOG or OVA-II peptide at the left or right ear, respectively, ear tissues were analyzed by flow cytometry. Shown are a) number of CD4⁺ cells among live cells, b) number of CD4⁺CD25⁺Foxp3⁺ cells, and c) number of CD25⁺Foxp3⁺OT-II Tetramer⁺ cells.

FIG. 12. Schematic illustration of treatment regimens for ND-OVA-II combined with wild-type IL-2 (wtIL-2) or IL-2/IC. One dose of ND-OVA-II was first administered on Day −4, −3, or −1, and three doses of Il-2/IC or five doses of wtIL-2 were given daily, starting from Day 0. Same regimens were repeated ten days after the initial dates in each group, respectively.

FIG. 13A-D. CD4⁺ T cells from peripheral blood were analyzed by flow cytometry on Day 5, 10, and 15 after IL-2 or IL-2/IC treatment. Shown are a) frequency of CD4⁺ cells among CD3⁺ T cells, b) frequency of CD25⁺Foxp3⁺ Treg cells among CD4⁺ T cells, c) frequency of OT-II Tetramer⁺CD44^{hi} cells among CD4⁺ T cells, and d) the frequency of OT-II Tetramer⁺CD44^{hi} cells among CD25⁺Foxp3⁺ Treg cells.

FIG. 14A-F. CD8⁺ T cells and NK cells from peripheral blood were analyzed by flow cytometry on Day 5, 10, and 15 after IL-2 or IL-2/IC treatment. Shown are a) frequency of CD8⁺ cells among CD3⁺ T cells, c) frequency of CD44^{hi} cells among CD8⁺ T cells, e) frequency of SSC^{low}CD49b⁺ NK cells among CD3⁻ cells, number of b) CD8⁺ T cells, d) CD8⁺CD44^{hi} T cells, and f) SSC^{low}CD49b⁺ NK cells in 2 mL blood.

FIG. 15A-C. A) Schematic illustration of treatment regimen for ACT+ND combined with IL-2/IC therapy. 6 days after the last IL-2 treatment, the mice were transferred with 3 million preactivated BDC splenocytes and 3 million preactivated NY8.3 splenocytes via retro-orbital injection. Diabetes incidence was monitored via OneTouch Ultra 2. B) Mice were treated with p31-ND or p31-ND+IL-2/IC combo. C) Mice were treated with InsB-ND and InsC-ND or InsB-ND+InsC-ND+IL-2/IC combo.

### DEFINITIONS

The term “about” is used herein to mean a value that is ±10% of the recited value.

As used herein, by “administering” is meant a method of giving a dosage of a composition described herein (e.g., a nanoparticle or a nanoparticle associated with an antigen) (e.g., a immunomodulatory agent) to a subject. The compositions utilized in the methods described herein can be administered by any suitable route, including, for example, by inhalation, nebulization, aerosolization, intranasally, intratracheally, intrabronchially, orally, parenterally (e.g., intravenously, subcutaneously, or intramuscularly), orally, nasally, rectally, topically, or buccally. The compositions utilized in the methods described herein can also be administered locally or systemically. The preferred method of administration can vary depending on various factors (e.g., the components of the composition being administered, and the severity of the condition being treated).

As used herein, the term “associated with” refers to the state of two or more entities (e.g., nanoparticles and one or more immunomodulatory agent) which are linked by a direct or indirect covalent or non-covalent interaction. In some embodiments, an association is covalent. In some embodiments, a covalent association is mediated by a linker moiety. In some embodiments, an association is non-covalent (e.g., charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, etc.). For example, in some embodiments, an immunomodulatory agent is admixed with a nanoparticle. In some embodiments, an immunomodulatory agent is conjugated with a nanoparticle. In some embodiments, an immunomodulatory agent is encapsulated within a nanoparticle. In some embodiments, an immunomodulatory agent is absorbed into a nanoparticle. In some embodiments, an immunomodulatory agent is adsorbed onto a nanoparticle. In some embodiments, an immunomodulatory agent is admixed with a nanoparticle.

As used herein, the term “absorbed” refers to a biomacromolecule agent (e.g., antigen) that is taken into and stably retained in the interior, that is, internal to the outer surface, of a nanoparticle and/or microparticle.

As used herein, the term “admixed” refers to a biomacromolecule agent (e.g., antigen) that is dissolved, dispersed, or suspended in a nanoparticle and/or microparticle. In some cases, the biomacromolecule agent may be uniformly admixed in the nanoparticle and/or microparticle.

As used herein, the term “adsorbed” refers to the attachment of a biomacromolecule agent (e.g., antigen) to the external surface of a nanoparticle and/or microparticle. Such adsorption preferably occurs by electrostatic attraction. Electrostatic attraction is the attraction or bonding generated between two or more oppositely charged or ionic chemical groups. Generally, the adsorption is typically reversible.

As used herein, the term “mutein” is intended to include proteins and polypeptides with an altered amino acid sequence and which arise as a result of a mutation or a recombinant DNA procedure. As used herein, the term “IL-2 mutein molecule” or “IL-2 mutein” refers to an IL-2 variant that preferentially activates Treg cells.

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein can be the full-length, 33-mer polypeptide from α-gliadin (SEQ ID NO: 374) or any fragment thereof or any of the polypeptides disclosed in Table 3 (SEQ ID NOs: 375-405) as epitopes recognized by CD₄⁺ T-cells. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

As used herein, the terms “autoimmune disorder” and “autoimmune disease”, used herein interchangeably, refers to a medical condition in which a subject's immune system mistakenly attacks the subject's own body.

As used herein, a “combination therapy” or “administered in combination” means that two or more (e.g., 2, 3, 4, 5, 6, 7, 8, 9, 10 or more) different agents or treatments are administered to a subject as part of a defined treatment regimen for a particular disease or condition (e.g., an autoimmune disorder (e.g., MS or celiac disease)). The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder, is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be affected by any appropriate route including, but not limited to, by inhalation, nebulization, aerosolization, intranasally, intratracheally, intrabronchially, orally, parenterally (e.g., intravenously, subcutaneously, or intramuscularly), orally, nasally, rectally, topically, buccally, or by direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the term “complexed” as used herein relates to the non-covalent interaction of a biomacromolecule agent (e.g., antigen) with a nanoparticle and/or microparticle.

As used herein, the term “conjugated” as used herein indicates a covalent bond association between a biomacromolecule agent (e.g., antigen) and a nanoparticle and/or microparticle.

As used herein, the term “drug” or “therapeutic agent” is meant to include any molecule, molecular complex, or substance administered to an organism for diagnostic or therapeutic purposes, including medical imaging, monitoring, contraceptive, cosmetic, nutraceutical, pharmaceutical, and prophylactic applications. The term drug is further meant to include any such molecule, molecular complex, or substance that is chemically modified and/or operatively attached to a biologic or biocompatible structure.

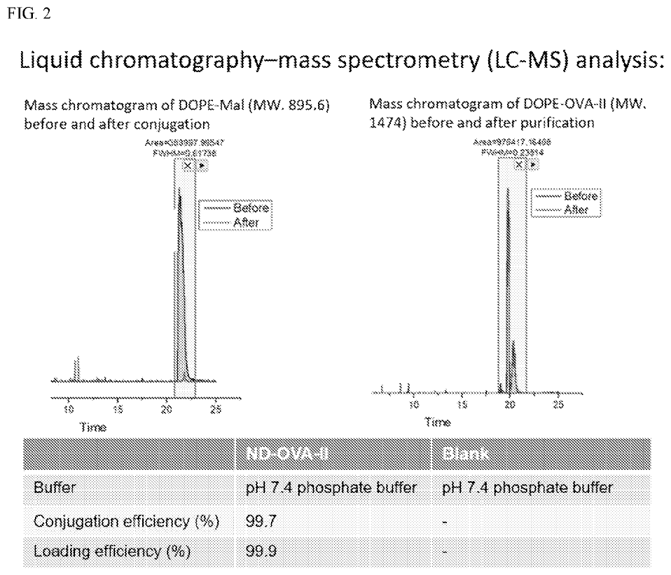

The term “T regulatory cells” (also called “Tregs” or “Treg cells”) has its general meaning in the art and is intended to describe the subpopulation of T cells that are been characterized to “suppress” activity of effector T cells in vitro and/or in vivo. Treg cells thus represent an important component of the healthy immune system. Regulatory T cells are involved in keeping effector T cells in check, which modulate the immune system, maintain tolerance to self-antigens, and abrogate autoimmune and/or inflammatory disease. Tregs have numerous acknowledged biomarkers known in the art. Regulatory T cells comprise two subsets that distinguish each other by the expression of CD45RA defined as “naïve Tregs” that express FOXP3 and CD45RA and “effector Tregs” that also express FOXP3 and not CD45RA. Cells that are suitable for expansion are naïve Tregs since they are highly proliferative under stimulation conditions and in the presence of IL-2 while effector Tregs are poorly proliferative under such conditions. Thus, FoxP3+CD4+ T cells can be divided in (1) naïve/resting Treg cells with a CD127lowCD25++CD45RA+FoxP3low phenotype (naïve Treg) and (2) effector Treg with CD127lowCD25+++CD45RA-FoxP3high phenotype (“effector Treg cells”), both of which being highly suppressive in vitro, and (3) CD4+ T cells that are not suppressive with a CD127lowCD25++CD45RA-FoxP3low phenotype. Thus, the term “effector Treg cells” (also called “eTreg cells”) refers to active Treg cell which display regulatory function or suppressive function of effector T cells (acting literally as the “effector” of Treg cells). The demonstration of regulatory/suppressive function of eTreg cells may be determined by any suitable method known in the art (see Miyara, M. et al. Functional Delineation and Differentiation Dynamics of Human CD4(+) T Cells Expressing the FoxP3 Transcription Factor. Immunity 30, 899-911 (2009)). In particular, examples of such tests are set out in the example section. Specifically, the tests embodied in example and FIG. 2 are regarded as standards in vitro tests for the assessment of regulatory T cell function.

As used herein, the term “fragment” refers to less than 100% of the amino acid sequence of a full-length reference protein (e.g., 99%, 90%, 80%, 70%, 60%, 50%, 40%, 30%, 20%, 10%, of the full-length sequence etc.), but including, e.g., 5, 10, 20, 25, 30, 35, 40, 45, 50, 100, 150, 200, 250, 300, 350, or more amino acids. A fragment can be of sufficient length such that a desirable function of the full-length protein is maintained. For example, the regulation of the alternative complement pathway in the fluid phase by fragments of, for example, factor H, is maintained. Such fragments are “biologically active fragments.”

As used herein, the term “expanding” refers to increasing the number of cells (e.g., Tregs) in a cell population or sample due to cell replication.

As used herein, the term “HDL” or “high density lipoprotein” refers to high-density lipoprotein. HDL comprises a complex of lipids and proteins in approximately equal amounts that functions as a transporter of cholesterol in the blood. HDL is mainly synthesized in and secreted from the liver and epithelial cells of the small intestine. Immediately after secretion, HDL is in a form of a discoidal particle containing apolipoprotein A-I (also called apoA-I) and phospholipid as its major constituents and is also called nascent HDL. This nascent HDL receives, in blood, free cholesterol from cell membranes of peripheral cells or produced in the hydrolysis course of other lipoproteins, and forms mature spherical HDL while holding, at its hydrophobic center, cholesterol ester converted from said cholesterol by the action of LCAT (lecithin cholesterol acyltransferase). HDL plays an extremely important role in a lipid metabolism process called “reverse cholesterol transport”, which takes, in blood, cholesterol out of peripheral tissues and transports it to the liver. High levels of HDL are associated with a decreased risk of atherosclerosis and coronary heart disease (CHD) as the reverse cholesterol transport is considered one of the major mechanisms for HDL's prophylactic action on atherosclerosis.

As used herein, the term “immunomodulatory agent” refers to a compound that stimulates or suppresses the immune system. Immunomodulatory agents include, but are not limited to, statins; mTOR inhibitors, such as rapamycin or a rapamycin analog; TGF-β signaling agents; TGF-β receptor agonists; histone deacetylase inhibitors, such as Trichostatin A; corticosteroids; inhibitors of mitochondrial function, such as rotenone; P38 inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors, such as TGX-221; autophagy inhibitors, such as 3-Methyladenine; aryl hydrocarbon receptor inhibitors; proteasome inhibitor I (PSI); and oxidized ATPs, such as P2X receptor blockers. In some embodiments, the immunomodulatory agent is an immunosuppressive agent. Examples of immunosuppressive agents include, but are not limited to, IDO, vitamin D3, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol; triptolide; OPN-305, OPN-401; Eritoran (E5564); TAK-242; Cpn10; NI-0101; 1A6; AV411; IRS-954 (DV-1079); IMO-3100; CPG-52363; CPG-52364; OPN-305; ATNC05; NI-0101; IMO-8400; Hydroxychloroquine; CU-CPT22; C29; Ortho-vanillin; SSL3 protein; OPN-305; 5 SsnB; Vizantin; (+)-N-phenethylnoroxymorphone; VB3323; Monosaccharide 3; (+)-Naltrexone and (+)-naloxone; HT52; HTB2; Compound 4a; CNT02424; TH1020; INH-ODN; E6446; AT791; CpG ODN 2088; ODN TTAGGG; COV08-0064; 2R9; GpG oligonucleotides; 2-aminopurine; Amlexanox; Bay11-7082; BX795; CH-223191; Chloroquine; CLI-095; CU-CPT9a; Cyclosporin A; CTY387; Gefitnib; Glybenclamide; H-89; H-131; Isoliquiritigenin; MCC950; MRT67307; OxPAPC; Parthenolide; Pepinh-MYD; Pepinh-TRIF; Polymyxin B; R406; RU.521; VX-765; YM201636; Z-VAD-FMK; and AHR-specific ligands; including but not limited to 2,3,7,8-tetrachloro-dibenzo-p-dioxin (TCDD); tryptamine (TA); and 6 formylindolo[3,2 b]carbazole (FICZ). In particular embodiments, the immunosuppressant is FTY720 (also known as fingolimod) (Chung and Harung, Clin. Neuropharmacol 33: 91-101, 2010), AhR activation by 2-(1′H-indole-3′-carbonyl)-thiazole-4-carboxylic acid methyl ester (ITE) or related ligands (Yeste A, et al. Proc. Natl. Acad. Sci. USA 109: 11270-11275, 2012; Quintana F. J., et al Proc. Natl. Acad. Sci. USA 107: 20768-20773, 2010), Trichostatin A (TSA) (Reilly C. M. et al. J Autoimmun 31: 123-130. 2008). Suberoylanilide hydroxamic acid (SAHA), a histone deacetylase inhibitor, (Lucas J. L., et al. Cell Immunol 257: 97-104, 2009) and/or Rapamycin (Rapa) (Maldonado, R. A., et al Proc. Natl. Acad Sci. USA 112:E156-165, 2015). In embodiments, the immunosuppressant may comprise any of the agents provided herein.

As used herein, the term “nucleic acid” may be DNA or RNA, such as mRNA. In embodiments, the compositions comprise a complement, such as a full-length complement, or a degenerate (due to degeneracy of the genetic code) of any of the nucleic acids provided herein. In embodiments, the nucleic acid is an expression vector that can be transcribed when transfected into a cell line. In embodiments, the expression vector may comprise a plasmid, retrovirus, or an adenovirus amongst others. Nucleic acids can be isolated or synthesized using standard molecular biology approaches, for example by using a polymerase chain reaction to produce a nucleic acid fragment, which is then purified and cloned into an expression vector. Additional techniques useful in the practice of this invention may be found in Current Protocols in Molecular Biology 2007 by John Wiley and Sons, Inc.; Molecular Cloning: A Laboratory Manual (Third Edition) Joseph Sambrook, Peter MacCallum Cancer Institute, Melbourne, Australia; David Russell, University of Texas Southwestern Medical Center, Dallas, Cold Spring Harbor.

As used herein, the term “in vitro” refers to an artificial environment and to processes or reactions that occur within an artificial environment. In vitro environments can consist of, but are not limited to, test tubes and cell culture.

The term “in vivo” refers to the natural environment (e.g., an animal or a cell) and to processes or reaction that occur within a natural environment.

As used here, the term “lipids” or “lipid molecules” refer to fatty substances that are insoluble in water and include fats, oils, waxes, and related compounds. They may be either made in the blood (endogenous) or ingested in the diet (exogenous). Lipids are essential for normal body function and whether produced from an exogenous or endogenous source, they must be transported and then released for use by the cells. The production, transportation, and release of lipids for use by the cells is referred to as lipid metabolism. While there are several classes of lipids, two major classes are cholesterol and triglycerides. Cholesterol may be ingested in the diet and manufactured by the cells of most organs and tissues in the body, primarily in the liver. Cholesterol can be found in its free form or, more often, combined with fatty acids forming what is known as cholesterol esters. As used herein, “lipid” or “lipid molecule” refers to any lipophilic compound. Non-limiting examples of lipid compounds include fatty acids, cholesterol, phospholipids, complex lipids, and derivatives or analogs thereof. They are usually divided into at least three classes: (1) “simple lipids,” which include fats and oils as well as waxes; (2) “compound lipids,” which include phospholipids and glycolipids; and (3) “derived lipids” such as steroids. Lipids or lipid molecules suitable for use in the present invention include both membrane-forming lipids and non-membrane-forming lipids.

As used herein the term, “lipoproteins” refer to compounds that are structured so that water-insoluble lipids are contained in a partially water-soluble shell. Depending on the type of lipoprotein, the contents include varying amounts of free and esterified cholesterol, triglycerides, and apoproteins or apolipoproteins. There are five major types of lipoproteins, which differ in function and in their lipid and apoprotein content and are classified according to increasing density: (i) chylomicrons and chylomicron remnants, (ii) very low density lipoproteins (“VLDL”), (iii) intermediate-density lipoproteins (“IDL”), (iv) low-density lipoproteins (“LDL”), and (v) high-density lipoproteins (“HDL”). Cholesterol circulates in the bloodstream as particles associated with lipoproteins.

The term “non-naturally occurring amino acid,” as used herein, means an alpha amino acid that is not naturally produced or found in a mammal. Examples of non-naturally occurring amino acids include D-amino acids; an amino acid having an acetylaminomethyl group attached to a sulfur atom of a cysteine; a pegylated amino acid; the omega amino acids of the formula NH₂(CH₂)ₙCOOH where n is 2-6, neutral nonpolar amino acids, such as sarcosine, t-butyl alanine, t-butyl glycine, N-methyl isoleucine, and norleucine; oxymethionine; phenylglycine; citrulline; methionine sulfoxide; cysteic acid; omithine; diaminobutyric acid; 3-aminoalanine; 3-hydroxy-D-proline; 2,4-diaminobutyric acid; 2-aninopentanoic acid; 2-aminooctanoic acid, 2-carboxy piperazine; piperazine-2-carboxylic acid, 2-amino-4-phenylbutanoic acid; 3-(2-naphthyl)alanine, and hydroxyproline. Other amino acids are α-aminobutyric acid, α-amino-α-methylbutyrate, aminocyclopropane-carboxylate, aminoisobutyric acid, aminonorbomyl-carboxylate, L-cyclohexylalanine, cyclopentylalanine, L-N-methylleucine, L-N-methylmethionine, L-N-methylnorvaline, L-N-methylphenylalanine, L-N-methylproline, L-N-methylserine, L-N-methyltryptophan, D-omithine, L-N-methylethylglycine, L-norleucine, α-methyl-aminoisobutyrate, α-methylcyclohexylalanine, D-α-methylalanine, D-α-methylarginine, D-α-methylasparagine, D-α-methylaspartate, D-α-methylcysteine, D-α-methylglutamine, D-α-methylhistidine, D-α-methylisoleucine, D-α-methylleucine, D-α-methyllysine, D-α-methylmethionine, D-α-methylomithine, D-α-methylphenylalanine, D-α-methylproline, D-α-methylserine, D-N-methylserine, D-α-methylthreonine, D-α-methyltryptophan, D-α-methyltyrosine, D-α-methylvaline, D-N-methylalanine, D-N-methylarginine, D-N-methylasparagine, D-N-methylaspartate, D-N-methylcysteine, D-N-methylglutamine, D-N-methylglutamate, D-N-methylhistidine, D-N-methylisoleucine, D-N-methylleucine, D-N-methyllysine, N-methylcyclohexylalanine, D-N-methylomithine, N-methylglycine, N-methylaminoisobutyrate, N-(1-methylpropyl)glycine, N-(2-methylpropyl)glycine, D-N-methyltryptophan, D-N-methyltvrosine, D-N-methylvaline, γ-aminobutyric acid, L-t-butylglycine, L-ethylglycine, L-homophenylalanine, L-α-methylarginine, L-α-methylaspartate, L-α-methylcysteine, L-α-methylglutamine, L-α-methylhistidine, L-α-methylisoleucine, L-α-methylleucine, L-α-methylmethionine, L-α-methylnorvaline, L-α-methylphenylalanine, L-α-methylserine, L-α-methyltryptophan, L-α-methylvaline, N—(N-(2,2-diphenylethyl) carbamylmethylglycine, 1-carboxy-1-(2,2-diphenyl-ethylamino) cyclopropane, 4-hydroxyproline, ornithine, 2-aminobenzoyl (anthraniloyl), D-cyclohexylalanine, 4-phenyl-phenylalanine, L-citrulline, α-cyclohexylglycine, L-1,2,3,4-tetrahydroisoquinoline-3-carboxylic acid, L-thiazolidine-4-carboxylic acid, L-homotyrosine, L-2-furylalanine, L-histidine (3-methyl), N-(3-guanidinopropyl)glycine, O-methyl-L-tyrosine, O-glycan-serine, meta-tyrosine, nor-tyrosine, L-N,N′,N″-trimethyllysine, homolysine, norlysine, N-glycan asparagine, 7-hydroxy-1,2,3,4-tetrahydro-4-fluorophenylalanine, 4-methylphenylalanine, bis-(2-picolyl)amine, pentafluorophenylalanine, indoline-2-carboxylic acid, 2-aminobenzoic acid, 3-amino-2-naphthoic acid, asymmetric dimethylarginine, L-tetrahydroisoquinoline-1-carboxylic acid, D-tetrahydroisoquinoline-1-carboxylic acid, 1-amino-cyclohexane acetic acid, D/L-allylglycine, 4-aminobenzoic acid, 1-amino-cyclobutane carboxylic acid, 2 or 3 or 4-aminocyclohexane carboxylic acid, 1-amino-1-cyclopentane carboxylic acid, 1-aminoindane-1-carboxylic acid, 4-amino-pyrrolidine-2-carboxylic acid, 2-aminotetraline-2-carboxylic acid, azetidine-3-carboxylic acid, 4-benzyl-pyrolidine-2-carboxylic acid, tert-butylglycine, b-(benzothiazolyl-2-yl)-alanine, b-cyclopropyl alanine, 5,5-dimethyl-1,3-thiazolidine-4-carboxylic acid, (2R,4S)4-hydroxypiperidine-2-carboxylic acid, (2S,4S) and (2S,4R)-4-(2-naphthylmethoxy)-pyrolidine-2-carboxylic acid, (2S,4S) and (2S,4R)4-phenoxy-pyrrolidine-2-carboxylic acid, (2R,5S) and (2S,5R)-5-phenyl-pyrrolidine-2-carboxylic acid, (2S,4S)-4-amino-1-benzoyl-pyrrolidine-2-carboxylic acid, t-butylalanine, (2S,5R)-5-phenyl-pyrrolidine-2-carboxylic acid, 1-aminomethyl-cyclohexane-acetic acid, 3,5-bis-(2-amino)ethoxy-benzoic acid, 3,5-diamino-benzoic acid, 2-methylamino-benzoic acid, N-methylanthranylic acid, L-N-methylalanine, L-N-methylarginine, L-N-methylasparagine, L-N-methylaspartic acid, L-N-methylcysteine, L-N-methylglutamine, L-N-methylglutamic acid. L-N-methylhistidine, L-N-methylisoleucine, L-N-methyllysine, L-N-methylnorleucine, L-N-methylomithine, L-N-methylthreonine, L-N-methyltyrosine, L-N-methylvaline, L-N-methyl-t-butylglycine, L-norvaline, α-methyl-γ-aminobutyrate, 4,4′-biphenylalanine, α-methylcylcopentylalanine, α-methyl-α-napthylalanine, α-methylpenicillamine, N-(4-aminobutyl)glycine, N-(2-aminoethyl)glycine, N-(3-aminopropyl)glycine, N-amino-α-methylbutyrate, α-napthylalanine, N-benzylglycine, N-(2-carbamylethyl)glycine, N-(carbamylmethyl)glycine, N-(2-carboxyethyl)glycine, N-(carboxymethyl)glycine, N-cyclobutylglycine, N-cyclodecylglycine, N-cycloheptylglycine, N-cyclohexylglycine, N-cyclodecylglycine, N-cylcododecylglycine, N-cyclooctylglycine, N-cyclopropylglycine, N-cycloundecylglycine, N-(2,2-diphenylethyl)glycine, N-(3,3-diphenylpropyl)glycine, N-(3-guanidinopropyl)glycine, N-(1-hydroxyethyl)glycine, N-(hydroxyethyl))glycine, N-(imidazolylethyl))glycine, N-(3-indolylyethyl)glycine, N-methyl-γ-aminobutyrate, D-N-methylmethionine, N-methylcyclopentylalanine, D-N-methylphenylalanine, D-N-methylproline, D-N-methylthreonine, N-(1-methylethyl)glycine, N-methyl-napthylalanine, N-methylpenicillamine, N-(p-hydroxyphenyl)glycine, N-(thiomethyl)glycine, penicillamine, L-α-methylalanine, L-α-methylasparagine, L-α-methyl-t-butylglycine, L-methylethylglycine, L-α-methylglutamate, L-α-methylhomophenylalanine, N-(2-methylthioethyl)glycine, L-α-methyllysine, L-α-methylnorleucine, L-α-methylomithine, L-α-methylproline, L-α-methylthreonine, L-α-methyltyrosine, L-N-methyl-homophenylalanine, N—(N-(3,3-diphenylpropyl) carbamylmethylglycine, L-pyroglutamic acid, D-pyroglutamic acid, O-methyl-L-serine, O-methyl-L-homoserine, 5-hydroxylysine, α-carboxyglutamate, phenylglycine, L-pipecolic acid (homoproline), L-homoleucine, L-lysine (dimethyl), L-2-naphthylalanine, L-dimethyldopa or L-dimethoxy-phenvlalanine, L-3-pyridylalanine, L-histidine (benzoyloxymethyl), N-cycloheptylglvcine, L-diphenylalanine, O-methyl-L-homotyrosine, L-β-homolysine, O-glycan-threoine, Ortho-tyrosine, L-N,N′-dimethyllysine, L-homoarginine, neotryptophan, 3-benzothienylalanine, isoquinoline-3-carboxylic acid, diaminopropionic acid, homocysteine, 3,4-dimethoxyphenylalanine, 4-chlorophenylalanine, L-1,2,3,4-tetrahydronorharman-3-carboxylic acid, adamantylalanine, symmetrical dimethylarginine, 3-carboxythiomorpholine, D-1,2,3,4-tetrahydronorharman-3-carboxylic acid, 3-aminobenzoic acid, 3-amino-1-carboxymethyl-pyridin-2-one, 1-amino-1-cyclohexane carboxylic acid, 2-aminocyclopentane carboxylic acid, 1-amino-1-cyclopropane carboxylic acid, 2-aminoindane-2-carboxylic acid, 4-amino-tetrahydrothiopyran-4-carboxylic acid, azetidine-2-carboxylic acid, b-(31unction3131ole-2-yl)-alanine, neopentylglycine, 2-carboxymethyl piperidine, b-cyclobutyl alanine, allylglycine, diaminopropionic acid, homo-cyclohexyl alanine, (2S,4R)-4-hydroxypiperidine-2-carboxylic acid, octahydroindole-2-carboxylic acid, (2S,4R) and (2S,4R)-4-(2-naphthyl), pyrrolidine-2-carboxylic acid, nipecotic acid, (2S,4R) and (2S,4S)-4-(4-phenylbenzyl) pyrrolidine-2-carboxylic acid, (3S)-1-pyrrolidine-3-carboxylic acid, (2S,4S)-4-tritvlmercapto-pyrrolidine-2-carboxylic acid, (2S,4S)-4-mercaptoproline, t-butylglycine, N,N-bis(3-aminopropyl)glycine, 1-amino-cyclohexane-1-carboxylic acid, N-mercaptoethylglycine, and selenocysteine. In some embodiments, amino acid residues may be charged or polar. Charged amino acids include alanine, lysine, aspartic acid, or glutamic acid, or non-naturally occurring analogs thereof. Polar amino acids include glutamine, asparagine, histidine, serine, threonine, tyrosine, methionine, or tryptophan, or non-naturally occurring analogs thereof. It is specifically contemplated that in some embodiments, a terminal amino group in the amino acid may be an amido group or a carbamate group.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “protein” refers to polymers of amino acids (e.g., naturally occurring amino acids and non-natural amino acids) of any length. The terms also encompass an amino acid polymer that has been modified; for example, disulfide bond formation, glycosylation, acetylation, phosphorylation, lipidation, or conjugation with a labeling component.

As used herein, the term “peptide” refers to a polymer in which the monomers are amino acids covalently attached together through amide bonds. Peptides are two or often more amino acids monomers long.

By “pharmaceutical composition” is meant any composition that contains a therapeutically or biologically active agent (e.g., a nanoparticle containing 1-30 (e.g., 2-30, 3-30, 4-30, 5-30, 6-30, 7-30, or 8-30 tolerogenic antigens)) that is suitable for administration to a subject. The biologically active agent includes a nanoparticle that contains 1-30 (e.g., 8-30 tolerogenic antigens per nanoparticle). The 1-30 tolerogenic antigens associated with a specific nanoparticle may all have the same sequence identities, or the 1-30 tolerogenic antigens associated with a specific nanoparticle may contain between 1 and 5 different populations of tolerogenic antigens having different sequence identities. Any of these formulations can be prepared by well-known and accepted methods in the art. See, for example, Remington: The Science and Practice of Pharmacy (21^{st} ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

By “pharmaceutically acceptable diluent, excipient, carrier, or adjuvant” is meant a diluent, excipient, carrier, or adjuvant which is physiologically acceptable to the subject while retaining the therapeutic properties of the pharmaceutical composition with which it is administered.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals, and industrial samples. Such examples are not however to be construed as limiting the sample types applicable to the present invention.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the terms “synthetic HDL,” “sHDL,” “reconstituted HDL”, and “rHDL” refer to a particle structurally analogous to native HDL, composed of a lipid or lipids in association with at least one of the proteins of HDL, preferably ApoA-I, or a mimetic thereof. Typically, the components of sHDL may be derived from blood or produced by recombinant technology.

By “therapeutically effective amount” is meant the amount of a composition administered to improve, inhibit, or ameliorate a condition of a subject, or a symptom of a disorder or disease, e.g., celiac disease, in a clinically relevant manner. Any improvement in the subject is considered sufficient to achieve treatment. Preferably, an amount sufficient to treat is an amount that reduces, inhibits, or prevents the occurrence or one or more symptoms of the disease or disorder (e.g., celiac disease) or is an amount that reduces the severity of, or the length of time during which a subject suffers from one or more symptoms of the disease or disorder, for example, celiac disease, (e.g., by at least about 10%, about 20%, or about 30%, more preferably by at least about 50%, about 60%, or about 70%, and most preferably by at least about 80%, about 90%, about 95%, about 99%, or more, relative to a control subject that is not treated with a composition described herein). An effective amount of the pharmaceutical composition used to practice the methods described herein (e.g., the treatment of celiac disease) varies depending upon the manner of administration and the age, body weight, and general health of the subject being treated. A physician or researcher can decide the appropriate amount and dosage regimen.

As used herein, the term “tolerogenic antigen” refers to a molecule that is capable of binding to an antibody or to an antigen receptor on a T cell, especially one that induces an immune response.

As used herein, the term “solvent” refers to a medium in which a reaction is conducted. Solvents may be liquid but are not limited to liquid form. Solvent categories include but are not limited to nonpolar, polar, protic, and aprotic.

### DETAILED DESCRIPTION OF THE INVENTION

Regulatory T cells (Tregs, CD4⁺CD25^{high}Foxp3⁺) play crucial roles in immune tolerance against autoimmune diseases [1]. Numerous studies have shown that adoptive transfer of Tregs can alleviate inflammation and restore immune tolerance in preclinical models. However, it remains unknown how to effectively induce and maintain high frequency of Tregs in vivo without ex vivo manipulation of Tregs, such as CAR-Tregs. Tregs require exogenous IL-2 via high-affinity IL2R complex for survival and proliferation [2]. While low dose IL-2 has been shown to induce Tregs in vivo, IL-2 can also promote effector T cells and NK cells that may exacerbate inflammation [3,4].

Recently, engineered IL-2 and mutein IL-2, such as PT101, have been developed selectively trigger Tregs without inducing effector T cells and NK cells [5-8]. In a clinical study, PT101 has been reported to induce polyclonal Tregs in humans [9]. However, prior studies have shown that, compared with polyclonal Tregs, antigen-specific Tregs have significantly superior therapeutic potential in a targeted manner for immune tolerance [10-13]. Thus, new approaches are needed to induce high frequency of antigen-specific Tregs in vivo.

Experiments conducted during the course of developing embodiments for the present invention resulted in the development of a new strategy for inducing high frequency of antigen-specific Tregs in vivo without ex vivo manipulation of cells. Such experiments resulted in the development and optimization of a new strategy of combining nanodiscs with modified IL-2 for eliciting unprecedented level of antigen-specific Tregs in vivo. Notably, IL-2:anti-IL-2 antibody (clone: JES6-1) immune complex (IL-2/IC) has been shown to selectively induce polyclonal Tregs [14]. IL-2/IC administered together with free peptide or peptide-tetramer have been reported to induce antigen-specific Tregs [15,16]. However, these previous attempts resulted in rather poor antigen-specific Treg response with less than 0.25% antigen-specific Treg frequency among CD4⁺ T cell compartment [15,16].

It was envisioned that it would be crucial to properly deliver peptide antigens to lymphoid tissues to maximize antigen-specific Treg induction together with IL-2/IC. Experiments described herein report for the first time that lymph-targeting nanodisc-mediated delivery of peptide in combination with IL-2/IC therapy resulted in remarkable amplification of antigen-specific Tregs, compared with IL-2/IC alone.

It was also envisioned that the actual method of treatment may be crucial. Such experiments demonstrated that nanodiscs are desirably administered (e.g., subcutaneously) first, followed by systemic administration of IL-2 and/or mutein/engineered IL-2. This ensured that antigen-specific Tregs were primed and generated first, and the subsequent administration of mutein/engineered IL-2 triggered robust proliferation of antigen-specific Tregs. Administration of maintenance doses of nanodiscs and/or mutein/engineered IL-2 for long-term maintenance of antigen-specific Tregs was further envisioned.

Compared with CAR-Tregs or other cellular therapies, the nanodiscs described herein are synthetic, well-characterized, and easy to manufacture [17,18]. Thus, this combination approach opens new doors for immunotherapy against various autoimmune diseases in a targeted manner. More broadly, it is envisioned that the nanodiscs described herein can be combined with mutein IL-2, other endogenous or engineered cytokines, growth factors, or antibodies to achieve synergy and induce Tregs.

Accordingly, the present invention relates to compositions comprising nanoparticles associated with or without one or more tolerogenic antigens, and compositions comprising an immunomodulatory agent (e.g., interleukin-2 (IL-2) or an IL-2 variant or an IL-2/IC)), and related methods involving co-administration of such compositions for purposes of inducing amplification of regulatory T cells (Tregs) (e.g., antigen-specific regulatory Tregs). The present invention further provides methods of treating autoimmune disorders through administering to a subject a composition comprising nanoparticles associated with or without one or more tolerogenic antigens associated with the autoimmune disorder prior to administering to the subject a composition comprising an immunomodulatory agent.

In certain embodiments, the present invention provides methods for in vivo amplification of regulatory Tregs (e.g., CD4±CD25^{high}Foxp3±) within a subject (e.g., a human subject suffering from or at risk of suffering from an autoimmune disorder) comprising administering to the subject a composition comprising one or more nanoparticles followed by (e.g., after 1 second, 2 seconds, 1 minute, 1 hour, 1 day, 1 week, 1 month, 1 year, etc.) administering to the subject a composition comprising one or more immunomodulatory agents (e.g., an IL-2, an IL-2 mutein, an IL-2 variant, or an IL-2/IC)) capable of expanding Tregs within the subject. In some embodiments, the composition comprising one or more nanoparticles is associated with (e.g., complexed, conjugated, encapsulated, absorbed, adsorbed, admixed) an mTOR inhibiting agent (e.g., rapamycin or a variant thereof). In some embodiments, the in vivo amplification of antigen-specific regulatory Tregs (e.g., CD4⁺CD25^{high}Foxp3⁺) within a subject is specific to a specific tissue region (e.g., a specific tissue region associated with one or more autoimmune disorders).

In certain embodiments, the present invention provides methods for in vivo amplification of antigen-specific regulatory Tregs (e.g., CD4⁺CD25^{high}Foxp3⁺) within a subject (e.g., a human subject suffering from or at risk of suffering from an autoimmune disorder) comprising administering to the subject a composition comprising one or more nanoparticles associated with one or more tolerogenic antigens followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding antigen-specific Tregs, wherein “antigen-specific” is specific to the one or more tolerogenic antigens associated with the comprising a nanoparticle associated with one or more tolerogenic antigens. In some embodiments, the in vivo amplification of antigen-specific regulatory Tregs (e.g., CD4⁺CD25^{high}Foxp3⁺) within a subject is specific to a specific tissue region (e.g., a specific tissue region associated with one or more autoimmune disorders).

In certain embodiments, the present invention provides methods for facilitating a strong immune tolerance to antigens associated with an autoimmune disorder within a subject (e.g., a human subject suffering from or at risk of suffering from an autoimmune disorder) comprising administering to the subject a composition comprising one or more nanoparticles followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding Tregs (e.g., CD4⁺CD25^{high}Foxp3⁺) within the subject. In some embodiments, the composition comprising one or more nanoparticles is associated with (e.g., complexed, conjugated, encapsulated, absorbed, adsorbed, admixed) an mTOR inhibiting agent (e.g., rapamycin or a variant thereof). In some embodiments, the facilitating a strong immune tolerance to antigens associated with an autoimmune disorder within a subject is specific to a specific tissue region (e.g., a specific tissue region associated with one or more autoimmune disorders).

In certain embodiments, the present invention provides methods for facilitating a strong immune tolerance to antigens associated with an autoimmune disorder within a subject (e.g., a human subject suffering from or at risk of suffering from an autoimmune disorder) comprising administering to the subject a composition comprising one or more nanoparticles associated with one or more tolerogenic antigens followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding antigen-specific Tregs (e.g., CD4⁺CD25^{high}Foxp3*), wherein “antigen-specific” is specific to the one or more tolerogenic antigens associated with the comprising a nanoparticle associated with one or more tolerogenic antigens. In some embodiments, the facilitating a strong immune tolerance to antigens associated with an autoimmune disorder within a subject is specific to a specific tissue region (e.g., a specific tissue region associated with one or more autoimmune disorders).

In certain embodiments, the present invention provides methods for increasing the ratio of CD3+FOXP3+ cells to CD3+FOXP3− cells within a population of T cells within a subject (e.g., a human subject suffering from or at risk of suffering from an autoimmune disorder) comprising administering to the subject a composition comprising one or more nanoparticles followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding Tregs (e.g., CD4⁺CD25^{high}Foxp3⁺) within the subject. In some embodiments, the composition comprising one or more nanoparticles is associated with (e.g., complexed, conjugated, encapsulated, absorbed, adsorbed, admixed) an mTOR inhibiting agent (e.g., rapamycin or a variant thereof). In some embodiments, the increasing the ratio of CD3+FOXP3+ cells to CD3+FOXP3− cells within the population of T cells within the subject is specific to a specific tissue region (e.g., a specific tissue region associated with one or more autoimmune disorders).

In certain embodiments, the present invention provides methods for increasing the ratio of CD3+FOXP3+ cells to CD3+FOXP3− cells within a population of T cells within a subject (e.g., a human subject suffering from or at risk of suffering from an autoimmune disorder) comprising administering to the subject a composition comprising one or more nanoparticles associated with one or more tolerogenic antigens followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding antigen-specific Tregs (e.g., CD4⁺CD25^{high}Foxp3⁺), wherein “antigen-specific” is specific to the one or more tolerogenic antigens associated with the comprising a nanoparticle associated with one or more tolerogenic antigens. In some embodiments, the increasing the ratio of CD3+FOXP3+ cells to CD3+FOXP3− cells within the population of T cells within the subject is specific to a specific tissue region (e.g., a specific tissue region associated with one or more autoimmune disorders).

In certain embodiments, the present invention provides methods for treating, preventing and/or attenuating a disorder comprising administering to a subject (e.g., a human subject suffering from or at risk of suffering from an autoimmune disorder e.g., celiac disease) comprising administering to the subject a composition comprising one or more nanoparticles followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding Tregs (e.g., CD4⁺CD25^{high}Foxp3⁺) within the subject. In some embodiments, the composition comprising one or more nanoparticles is associated with (e.g., complexed, conjugated, encapsulated, absorbed, adsorbed, admixed) an mTOR inhibiting agent (e.g., rapamycin or a variant thereof). In some embodiments, the treating, preventing, and/or attenuating one or more autoimmune disorders in the subject is specific to a specific tissue region (e.g., a specific tissue region associated with the autoimmune disorder).

In certain embodiments, the present invention provides methods for treating, preventing and/or attenuating a disorder comprising administering to a subject (e.g., a human subject suffering from or at risk of suffering from an autoimmune disorder e.g., celiac disease) a composition comprising one or more nanoparticles associated with one or more tolerogenic antigens (e.g., one or more tolerogenic antigens associated with the autoimmune disorder) followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding antigen-specific Tregs (e.g., CD4⁺CD25^{high}Foxp3⁺), wherein “antigen-specific” is specific to the one or more tolerogenic antigens associated with the comprising a nanoparticle associated with one or more tolerogenic antigens. In some embodiments, the treating, preventing, and/or attenuating one or more disorders in the subject is specific to a specific tissue region (e.g., a specific tissue region associated with the disorder).

Such methods are not limited to treating a particular disorder.

In some embodiments, the disorder is an autoimmune disorder. Examples of autoimmune disorders include, but are not limited to, multiple sclerosis (MS), celiac disease, rheumatoid arthritis, primary biliary cholangitis, primary sclerosing cholangitis, MOG antibody disease, diabetes (e.g., type 1 diabetes mellitus), autoimmune diseases of the thyroid (e.g., Hashimoto's thyroiditis, Graves' disease), thyroid-associated ophthalmopathy and dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutieres syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polyarthritis/fetal and neonatal anemia, Sepsis, and inflammatory bowel disease.

In some embodiments, the disorder is a transplantation related disorder. In some embodiments, the disorder is one or more allergies. In some embodiments, the disorder is a respiratory condition (e.g., asthma). In some embodiments, the disorder is graft-versus-host-disease (GvHD).

In some embodiments, such methods (e.g., administration of the composition comprising a nanoparticle associated with one or more tolerogenic antigens followed by the administration of the composition comprising an immunomodulatory agent capable of expanding Tregs) is further followed administration of one or more tolerogenic antigens to a specific tissue region (e.g., a specific tissue region associated with one or more autoimmune disorders). In some embodiments, the administration of one or more tolerogenic antigens to the specific tissue region is by injection administration and/or topical administration and/or subcutaneous administration. In some embodiments, the administration of one or more tolerogenic antigens to the specific tissue region prevents immune tolerance within the specific tissue region.

In some embodiments, the nanoparticle is associated with an immunomodulatory agent, and is not associated with a tolerogenic antigen. In some embodiments, the nanoparticle is associated with a tolerogenic antigen and is further associated with an immunomodulatory agent.

In some embodiments, the composition comprising an immunomodulatory agent capable of expanding Tregs is comprised within a nanoparticle such that the nanoparticle is associated with the immunomodulatory agent capable of expanding Tregs (e.g., thereby providing a composition comprising a nanoparticle associated with an immunomodulatory agent capable of expanding Tregs).

### Immunomodulatory Agents

The present invention provides compositions comprising one or more immunomodulatory agents. Such compositions are not limited to specific immunomodulatory agents.

In some embodiments the one or more immunomodulatory agents are selected from fingolimod; rapamycin; 2-(1′H-indole-3′-carbonyl)-thiazole-4-carboxylic acid methyl ester (ITE) or related ligands; Trichostatin A; Suberoylanilide hydroxamic acid (SAHA); statins; mTOR inhibitors; TGF-β signaling agents; TGF-β receptor agonists; histone deacetylase inhibitors; corticosteroids; inhibitors of mitochondrial function; NF-κβ inhibitors; adenosine receptor agonists; prostaglandin E2 agonists (PGE2; phosphodiesterase inhibitors; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors; autophagy inhibitors; aryl hydrocarbon receptor inhibitors; proteasome inhibitor I (PSI); oxidized ATPs IDO; vitamin D3; cyclosporins; aryl hydrocarbon receptor inhibitors; resveratrol; azathiopurine (Aza); 6-mercaptopurine (6-MP); 6-thioguanine (6-TG); FK506; sanglifehrin A; salmeterol; mycophenolate mofetil (MMF); aspirin and other COX inhibitors; niflumic acid; estriol; triptolide; OPN-305, OPN-401; Eritoran (E5564); TAK-242; Cpn10; NI-0101; 1A6; AV411; IRS-954 (DV-1079); IMO-3100; CPG-52363; CPG-52364; OPN-305; ATNC05; NI-0101; IMO-8400; Hydroxychloroquine; CU-CPT22; C29; Ortho-vanillin; SSL3 protein; OPN-305; 5 SsnB; Vizantin; (+)-N-phenethylnoroxymorphone; VB3323; Monosaccharide 3; (+)-Naltrexone and (+)-naloxone; HT52; HTB2; Compound 4a; CNT02424; TH1020; INH-ODN; E6446; AT791; CpG ODN 2088; ODN TTAGGG; COV08-0064; 2R9; GpG oligonucleotides; 2-aminopurine; Amlexanox; Bay 11-7082; BX795; CH-223191; Chloroquine; CLI-095; CU-CPT9a; Cyclosporin A; CTY387; Gefitnib; Glybenclamide; H-89; H-131; Isoliquiritigenin; MCC950; MRT67307; OxPAPC; Parthenolide; Pepinh-MYD; Pepinh-TRIF; Polymyxin B; R406; RU.521; VX-765; YM201636; Z-VAD-FMK; and AHR-specific ligands; including but not limited to 2,3,7,8-tetrachloro-dibenzo-p-dioxin (TCDD); tryptamine (TA); and 6 formylindolo[3,2 b]carbazole (FICZ))).

In some embodiments, the immunomodulatory agent is a cytokine. In some embodiments, the cytokine is a human cytokine. In some embodiments, the cytokine is selected from TGFβ, IL-1, IL-2, IL-4, IL-6, IL-7, IL-10, IL-12A, IL12B, IL-15, IL-21 and IL-18.

In some embodiments, the immunomodulatory agent is human IL-2. In some embodiments, the immunomodulatory agent is low dose IL-2. In some embodiments, the immunomodulatory agent is PT101 or a variant thereof. In some embodiments, the immunomodulatory agent is mutein IL-2 and/or variations thereof. In some embodiments, the IL-2 is any of the IL-2 cytokines, IL-2 muteins, and/or IL-2 variants as described in U.S. Pat. Nos. 11,091,527, 11,091,526, 11,077,195, 11,077,172, 10,960,079, 10,946,068, 10,766,938, 10,722,460, 10,174,092, 10,174,091; EP Patent No. 3808764, and/or U.S. Patent Application Publication No. US20120315245.

In some embodiments, the immunomodulatory agent is an IL-2:anti-IL-2 antibody (clone: JES6-1) immune complex (IL-2/IC).

In some embodiments, IL-2 is Proleukin. In some embodiments, IL-2 is administered at a dose of less than 14 MIU/m², less than 12 MIU/m², less than 10 MIU/m², less than 8 MIU/m², less than 6 MIU/m², less than 4 MIU/m², or less than 2 MIU/m².

In some embodiments, IL-2 is an extended pharmacokinetic (PK) IL-2. In some embodiments, the extended-PK IL-2 comprises a fusion protein. In some embodiments, the fusion protein comprises an IL-2 moiety and a moiety selected from the group consisting of an immunoglobulin fragment, human serum albumin, and Fn3. In some embodiments, the fusion protein comprises an IL-2 moiety operably linked to an immunoglobulin Fc domain. In some embodiments, the fusion protein comprises an IL-2 moiety operably linked to human serum albumin. In some embodiments, the extended-PK IL-2 comprises an IL-2 moiety conjugated to a non-protein polymer. In some embodiments, the non-protein polymer is a polyethylene glycol.

In certain embodiments, the extended-PK IL-2 is mutated such that it has an altered affinity (e.g., a higher affinity) for the IL-2R alpha receptor compared with unmodified IL-2. Site-directed mutagenesis can be used to isolate IL-2 mutants that exhibit high affinity binding to CD25, i.e., IL-2Rα, as compared to wild-type IL-2. Increasing the affinity of IL-2 for IL-2Rα at the cell surface will increase receptor occupancy within a limited range of IL-2 concentration, as well as raise the local concentration of IL-2 at the cell surface.

In certain embodiments, IL-2 mutants are provided, which may be, but are not necessarily, substantially purified and which can function as high affinity CD25 binders. IL-2 is a T cell growth factor that induces proliferation of antigen-activated T cells and stimulation of NK cells. Exemplary IL-2 mutants which are high affinity binders include those described in WO2013/177187A2. Further exemplary IL-2 mutants with increased affinity for CD25 are disclosed in U.S. Pat. No. 7,569,215, the contents of which are incorporated herein by reference.

IL-2 mutants can be at least or about 50%, at least or about 65%, at least or about 70%, at least or about 80%, at least or about 85%, at least or about 87%, at least or about 90%, at least or about 95%, at least or about 97%, at least or about 98%, or at least or about 99% identical in amino acid sequence to wild-type IL-2 (in its precursor form or, preferably, the mature form). The mutation can consist of a change in the number or content of amino acid residues. For example, the IL-2 mutants can have a greater or a lesser number of amino acid residues than wild-type IL-2. Alternatively, or in addition, IL-2 mutants can contain a substitution of one or more amino acid residues that are present in the wild-type IL-2.

The 42unction42es that are required to make IL-2 mutants are routine in the art, and can be performed without resort to undue experimentation by one of ordinary skill in the art. For example, a mutation that consists of a substitution of one or more of the amino acid residues in IL-2 can be created using a PCR-assisted mutagenesis technique (e.g., as known in the art and/or described herein for the creation of IL-2 mutants). Mutations that consist of deletions or additions of amino acid residues to an IL-2 polypeptide can also be made with standard recombinant techniques. In the event of a deletion or addition, the nucleic acid molecule encoding IL-2 is simply digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

In addition to generating IL-2 mutants via expression of nucleic acid molecules that have been altered by recombinant molecular biological techniques, IL-2 mutants can be chemically synthesized. Chemically synthesized polypeptides are routinely generated by those of skill in the art.

As noted above, IL-2 can also be prepared as fusion or chimeric polypeptides that include IL-2 and a heterologous polypeptide (i.e., a polypeptide that is not IL-2). The heterologous polypeptide can increase the circulating half-life of the chimeric polypeptide in vivo, and may, therefore, further enhance the properties of IL-2.

In certain embodiments, the chimeric polypeptide can include IL-2 and a polypeptide that functions as an antigenic tag, such as a FLAG sequence. FLAG sequences are recognized by biotinylated, highly specific, anti-FLAG antibodies, as described herein (see also Blanar et al., Science 256: 1014, 1992; LeClair et al., Proc. Natl. Acad. Sci. USA 89:8145, 1992). In certain embodiments, the chimeric polypeptide further comprises a C-terminal c-myc epitope tag.

Chimeric polypeptides can be constructed using no more than conventional molecular biological techniques, which are well within the ability of those of ordinary skill in the art to perform.

In certain embodiments, the composition comprising an immunomodulatory agent (e.g., human cytokine (e.g., an IL-2, an IL-2 mutein, an IL-2 variant, or an IL-2/IC)) is capable of expanding Treg cells within a subject or sample. Indeed, such compositions comprising an immunomodulatory agent are capable of increasing the ratio of Tregs to non-regulatory T cells. The ratio may be measured by determining the ratio of CD3+FOXP3+ cells to CD3+FOXP3− cells within the population of T cells. The typical Treg frequency in human blood is 5-10% of total CD4+CD3+ T cells, however, in autoimmune disorders the percentage may be lower or higher. In preferred embodiments, the percentage of Treg increases at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1000%. Maximal fold increases in Treg may vary for particular diseases; however, a maximal Treg frequency that might be obtained through IL-2 mutein treatment is 50% or 60% of total CD4+CD3+ T cells. In certain embodiments, a composition comprising an immunomodulatory agent (e.g., human cytokine (e.g., an IL-2, an IL-2 mutein, an IL-2 variant, or an IL-2/IC)) is administered to a subject and the ratio of regulatory T cells (Tregs) to non-regulatory T cells within peripheral blood of a subject increases.

Because such compositions comprising an immunomodulatory agent (e.g., human cytokine (e.g., an IL-2, an IL-2 mutein, an IL-2 variant, or an IL-2/IC)) preferentially expand Tregs over other cell types, they also are useful for increasing the ratio of regulatory T cells (Tregs) to natural killer (NK) cells within the peripheral blood of a subject. The ratio may be measured by determining the ratio of CD3+FOXP3+ cells to CD16+ and/or CD56+ lymphocytes that are CD19− and CD3−.

### Nanoparticles

The present invention is not limited to specific types or kinds of nanoparticles associated with (e.g., complexed, conjugated, encapsulated, absorbed, adsorbed, admixed) or not associated with tolerogenic antigens for treating, preventing, or ameliorating various types of autoimmune disorders (e.g., celiac disease).

Examples of nanoparticles include, but are not limited to, fullerenes (a.k.a. C₆₀, C₇₀, C₇₆, C₈₀, C₈₄), endohedral metallofullerenes (EMI's) buckyballs, which contain additional atoms, ions, or clusters inside their fullerene cage), trimetallic nitride templated endohedral metallofullerenes (TNT EMEs, high-symmetry four-atom molecular cluster endohedrals, which are formed in a trimetallic nitride template within the carbon cage), single-walled and multi-walled carbon nanotubes, branched and dendritic carbon nanotubes, gold nanorods, silver nanorods, single-walled and multi-walled boron/nitrate nanotubes, carbon nanotube peapods (nanotubes with internal metallo-fullerenes and/or other internal chemical structures), carbon nanohorns, carbon nanohorn peapods, liposomes, nanoshells, dendrimers, quantum dots, superparamagnetic nanoparticles, nanorods, and cellulose nanoparticles. The particle embodiment can also include microparticles with the capability to enhance effectiveness or selectivity. Other non-limiting exemplary nanoparticles include glass and polymer micro- and nano-spheres, biodegradable PLGA micro- and nano-spheres, gold, silver, carbon, and iron nanoparticles.

In some embodiments, the nanoparticle is a modified micelle. In these embodiments, the modified micelle comprises polyol polymers modified to contain a hydrophobic polymer block. The term “hydrophobic polymer block” as used in the present disclosure indicates a segment of the polymer that on its own would be hydrophobic. The term “micelle” as used herein refers to an aggregate of molecules dispersed in a liquid. A typical micelle in aqueous solution forms an aggregate with the hydrophilic “head” regions in contact with surrounding solvent, sequestering the hydrophobic single tail regions in the micelle centre. In some embodiments the head region may be, for example, a surface region of the polyol polymer while the tail region may be, for example, the hydrophobic polymer block region of the polyol polymer.

The invention further encompasses use of particles on the micrometer scale in addition to the nanometer scale. Where microparticles are used, it is preferred that they are relatively small, on the order of 1-50 micrometers. For ease of discussion, the use herein of “nanoparticles” encompasses true nanoparticles (sizes of from about 1 nm to about 1000 nm), microparticles (e.g., from about 1 micrometer to about 50 micrometers), or both.

Examples of nanoparticles include, by way of example and without limitation, paramagnetic nanoparticles, superparamagnetic nanoparticles, metal nanoparticles, fullerene-like materials, inorganic nanotubes, dendrimers, dendrimers with covalently attached metal chelates, nanofibers, nanohorns, nano-onions, nanorods, nanoropes, and quantum dots. In some embodiments, a nanoparticle is a metal nanoparticle (for example, a nanoparticle of gold, palladium, platinum, silver, copper, nickel, cobalt, iridium, or an alloy of two or more thereof). Nanoparticles can include a core or a core and a shell, as in core-shell nanoparticles.

In some embodiments, the nanoparticles are sHDL nanoparticles. Generally, sHDL nanoparticles are composed of a mixture of HDL apolipoprotein and an amphipathic lipid.

The present invention is not limited to use of a particular type or kind of HDL apolipoprotein. HDL apolipoproteins include, for example apolipoprotein A-I (apo A-I), apolipoprotein A-II (apo A-II), apolipoprotein A4 (apo A4), apolipoprotein Cs (apo Cs), apolipoprotein M (apo M), and apolipoprotein E (apo E). In some embodiments, the HDL apolipoprotein is selected from preproapoliprotein, preproApoA-I, proApoA-I, ApoA-I, preproApoA-II, proApoA-II, ApoA-II, apolipoprotein A-II xxx (apo A-II-xxx), preproApoA-IV, proApoA-lV, ApoA-IV, ApoA-V, preproApoE, proApoE, ApoE, preproApoA-lMilano, proApoA-Imilano, ApoA-lMilano, preproApoA-Iparis, proApoA-Iparis, ApoA-Iparis, and peptide mimetics of these proteins mixtures thereof. Preferably, the carrier particles are composed of ApoA-I or ApoA-II, however the use of other lipoproteins including apolipoprotein A4, apolipoprotein Cs or apolipoprotein E may be used alone or in combination to formulate carrier particle mixtures for delivery of therapeutic agents. In some embodiments, mimetics of such HDL apolipoproteins are used.

ApoA-I is synthesized by the liver and small intestine as preproapolipoprotein which is secreted as a proprotein that is rapidly cleaved to generate a mature polypeptide having 243 amino acid residues. ApoA-I consists mainly of 6 to 8 different 22 amino acid repeats, and 2 different 11 amino acid repeats, each of which has the helical wheel signature of an amphipathic α helix, spaced by a linker moiety which is often proline, and, in some cases, consists of a stretch made up of several residues. ApoA-I forms three types of stable complexes with lipids: small, lipid-poor complexes referred to as pre-beta-1 HDL; flattened discoidal particles containing polar lipids (phospholipid and cholesterol) referred to as pre-beta-2 HDL; and spherical particles containing both polar and nonpolar lipids, referred to as spherical or mature HDL (HDL3 and HDL2). Most HDL in the circulating population contain both ApoA-I and ApoA-II (the second major HDL protein).

In some embodiments, ApoA-I agonists or mimetics are provided. In some embodiments, such ApoA-I mimetics are capable of forming amphipathic α-helices that mimic the activity of ApoA-I, and have specific activities approaching or exceeding that of the native molecule. In some, the ApoA-I mimetics are peptides or peptide analogues that: form amphipathic helices (in the presence of lipids), bind lipids, form pre-p-like or HDL-like complexes, activate lecithin: cholesterol acyltransferase (LCAT), increase serum levels of HDL fractions, and promote cholesterol efflux.

The present invention is not limited to use of a particular ApoA-I mimetic. In some embodiments, any of the ApoA-I mimetics described in Srinivasa, et al., 2014 Curr. Opinion Lipidology Vol. 25(4): 304-308 are utilized. In some embodiments, any of the ApoA-I mimetics described in U.S. Patent Application Publication Nos. 20110046056 and 20130231459 are utilized.

In some embodiments, the “22A” ApoA-I mimetic is used (PVLDLFRELLNELLEALKQKLK) (SEQ ID NO: 4) (see, e.g., U.S. Pat. No. 7,566,695). In some embodiments, any of the following ApoA-I mimetics shown in Table 1 as described in U.S. Pat. No. 7,566,695 are utilized:

TABLE 1
ApoA-I mimetics

| SEQ ID NO | AMINO ACID SEQUENCE |
|---|---|
| (SEQ ID NO: 1) | PVLDLFRELLNELLEZLKQKLK |
| (SEQ ID NO: 2) | GVLDLFRELLNELLEALKQKLKK |
| (SEQ ID NO: 3) | PVLDLFRELLNELLEWLKQKLK |
| (SEQ ID NO: 4) | PVLDLFRELLNELLEALKQKLK |
| (SEQ ID NO: 5) | PVLDLFRELLNELLEALKQKLKK |
| (SEQ ID NO: 6) | PVLDLFRELLNEXLEALKQKLK |
| (SEQ ID NO: 7) | PVLDLFKELLNELLEALKQKLK |
| (SEQ ID NO: 8) | PVLDLFRELLNEGLEALKQKLK |
| (SEQ ID NO: 9) | PVLDLFRELGNELLEALKQKLK |
| (SEQ ID NO: 10) | PVLDLFRELLNELLEAZKQKLK |
| (SEQ ID NO: 11) | PVLDLFKELLQELLEALKQKLK |
| (SEQ ID NO: 12) | PVLDLFRELLNELLEAGKQKLK |
| (SEQ ID NO: 13) | GVLDLFRELLNEGLEALKQKLK |
| (SEQ ID NO: 14) | PVLDLFRELLNELLEALOQOLO |
| (SEQ ID NO: 15) | PVLDLFRELWNELLEALKQKLK |
| (SEQ ID NO: 16) | PVLDLLRELLNELLEALKQKLK |
| (SEQ ID NO: 17) | PVLELFKELLQELLEALKQKLK |
| (SEQ ID NO: 18) | GVLDLFRELLNELLEALKQKLK |
| (SEQ ID NO: 19) | PVLDLFRELLNEGLEALKQKLK |
| (SEQ ID NO: 20) | PVLDLFREGLNELLEALKQKLK |
| (SEQ ID NO: 21) | PVLDLFRELLNELLEALKQKLK |
| (SEQ ID NO: 22) | PVLDLFRELLNELLEGLKQKLK |
| (SEQ ID NO: 23) | PLLELFKELLQELLEALKQKLK |
| (SEQ ID NO: 24) | PVLDLFRELLNELLEALQKKLK |
| (SEQ ID NO: 25) | PVLDFFRELLNEXLEALKQKLK |
| (SEQ ID NO: 26) | PVLDLFRELLNELLELLKQKLK |
| (SEQ ID NO: 27) | PVLDLFRELLNELZEALKQKLK |
| (SEQ ID NO: 28) | PVLDLFRELLNELWEALKQKLK |
| (SEQ ID NO: 29) | AVLDLFRELLNELLEALKQKLK |
| (SEQ ID NO: 30) | PVLDLPRELLNELLEALKQKLK¹ |
| (SEQ ID NO: 31) | PVLDLFLELLNEXLEALKQKLK |
| (SEQ ID NO: 32) | XVLDLFRELLNELLEALKQKLK |
| (SEQ ID NO: 33) | PVLDLFREKLNELLEALKQKLK |
| (SEQ ID NO: 34) | PVLDZFRELLNELLEALKQKLK |
| (SEQ ID NO: 35) | PVLDWFRELLNELLEALKQKLK |
| (SEQ ID NO: 36) | PLLELLKELLQELLEALKQKLK |
| (SEQ ID NO: 37) | PVLDLFREWLNELLEALKQKLK |
| (SEQ ID NO: 38) | PVLDLFRELLNEXLEAWKQKLK |
| (SEQ ID NO: 39) | PVLDLFRELLEELLKALKKKLK |
| (SEQ ID NO: 40) | PVLDLFNELLRELLEALQKKLK |
| (SEQ ID NO: 41) | PVLDLWRELLNEXLEALKQKLK |
| (SEQ ID NO: 42) | PVLDEFREKLNEXWEALKQKLK |
| (SEQ ID NO: 43) | PVLDEFREKLWEXLEALKQKLK |
| (SEQ ID NO: 44) | PVLDEFREKLNEXLEALKQKLK |
| (SEQ ID NO: 45) | PVLDEFREKLNEXLEALKQKLK |
| (SEQ ID NO: 46) | PVLDLFREKLNEXLEALKQKLK |
| (SEQ ID NO: 47) | ~VLDLFRELLNEGLEALKQKLK |
| (SEQ ID NO: 48) | PVLDLFRELLNELLEALKQKLK |
| (SEQ ID NO: 49) | PVLDLFRNLLEKLLEALEQKLK |
| (SEQ ID NO: 50) | PVLDLFRELLWEXLEALKQKLK |
| (SEQ ID NO: 51) | PVLDLFWELLNEXLEALKQKLK |
| (SEQ ID NO: 52) | PVWDEFREKLNEXLEALKQKLK |
| (SEQ ID NO: 53) | VVLDLFRELLNELLEALKQKLK |
| (SEQ ID NO: 54) | PVLDLFRELLNEWLEALKQKLK |
| (SEQ ID NO: 55) | P~~~LFRELLNELLEALKQKLK |
| (SEQ ID NO: 56) | PVLDLFRELLNELLEALKQKKK |
| (SEQ ID NO: 57) | PVLDLFRNLLEELLKALEQKLK |
| (SEQ ID NO: 58) | PVLDEFREKLNEXLEALKQKL~ |
| (SEQ ID NO: 59) | LVLDLFRELLNELLEALKQKLK |
| (SEQ ID NO: 60) | PVLDLFRELLNELLEALKQ~~~ |
| (SEQ ID NO: 61) | PVLDEFRWKLNEXLEALKQKLK |
| (SEQ ID NO: 62) | PVLDEWREKLNEXLEALKQKLK |
| (SEQ ID NO: 63) | PVLDFFREKLNEXLEALKQKLK |
| (SEQ ID NO: 64) | PWLDEFREKLNEXLEALKQKLK |
| (SEQ ID NO: 65) | ~VLDEFREKLNEXLEALKQKLK |
| (SEQ ID NO: 66) | PVLDLFRNLLEELLEALQKKLK |
| (SEQ ID NO: 67) | ~VLDLFRELLNELLEALKQKLK |
| (SEQ ID NO: 68) | PVLDEFRELLKEXLEALKQKLK |
| (SEQ ID NO: 69) | PVLDEFRKKLNEXLEALKQKLK |
| (SEQ ID NO: 70) | PVLDEFRELLYEXLEALKQKLK |
| (SEQ ID NO: 71) | PVLDEFREKLNELXEALKQKLK |
| (SEQ ID NO: 72) | PVLDLFRELLNEXLWALKQKLK |
| (SEQ ID NO: 73) | PVLDEFWEKLNEXLEALKQKLK |
| (SEQ ID NO: 74) | PVLDKFREKLNEXLEALKQKLK |
| (SEQ ID NO: 75) | PVLDEFREKLNEELEALKQKLK |
| (SEQ ID NO: 76) | PVLDEFRELLFEXLEALKQKLK |
| (SEQ ID NO: 77) | PVLDEFREKLNKXLEALKQKLK |
| (SEQ ID NO: 78) | PVLDEFRDKLNEXLEALKQKLK |
| (SEQ ID NO: 79) | PVLDEFRELLNELLEALKQKLK |
| (SEQ ID NO: 80) | PVLDLFERLLNELLEALQKKLK |
| (SEQ ID NO: 81) | PVLDEFREKLNWXLEALKQKLK |
| (SEQ ID NO: 82) | ~~LDEFREKLNEXLEALKQKLK |
| (SEQ ID NO: 83) | PVLDEFREKLNEXLEALWQKLK |
| (SEQ ID NO: 84) | PVLDEFREKLNELLEALKQKLK |
| (SEQ ID NO: 85) | P~LDLFRELLNELLEALKQKLK |
| (SEQ ID NO: 86) | PVLELFERLLDELLNALQKKLK |
| (SEQ ID NO: 87) | PLLELLKELLQELLEALKQKLK |
| (SEQ ID NO: 88) | PVLDKFRELLNEXLEALKQKLK |
| (SEQ ID NO: 89) | PVLDEFREKLNEXLWALKQKLK |
| (SEQ ID NO: 90) | ~~~DEFREKLNEXLEALKQKLK |
| (SEQ ID NO: 91) | PVLDEFRELLNEXLEALKQKLK |
| (SEQ ID NO: 92) | PVLDEFRELYNEXLEALKQKLK |
| (SEQ ID NO: 93) | PVLDEFREKLNEXLKALKQKLK |
| (SEQ ID NO: 94) | PVLDEFREKLNEALEALKQKLK |
| (SEQ ID NO: 95) | PVLDLFRELLNLXLEALKQKLK |
| (SEQ ID NO: 96) | PVLDLFRELLNEXLEALKQKLK |
| (SEQ ID NO: 97) | PVLDLFRELLNELLE~~~~~~~ |
| (SEQ ID NO: 98) | PVLDLFRELLNEELEALKQKLK |
| (SEQ ID NO: 99) | KLKQKLAELLENLLERFLDLVP |
| (SEQ ID NO: 100) | PVLDLFRELLNELLEALKQKLK |
| (SEQ ID NO: 101) | PVLDLFRELLNWXLEALKQKLK |
| (SEQ ID NO: 102) | PVLDLFRELLNLXLEALKEKLK |
| (SEQ ID NO: 103) | PVLDEFRELLNEELEALKQKLK |
| (SEQ ID NO: 104) | P~~~~~~~LLNELLEALKQKLK |
| (SEQ ID NO: 105) | PAADAFREAANEAAEAAKQKAK |
| (SEQ ID NO: 106) | PVLDLFREKLNEELEALKQKLK |
| (SEQ ID NO: 107) | KLKQKLAELLENLLERFLDLVP |
| (SEQ ID NO: 108) | PVLDLFRWLLNEXLEALKQKLK |
| (SEQ ID NO: 109) | PVLDEFREKLNERLEALKQKLK |
| (SEQ ID NO: 110) | PVLDEFREKLNEXXEALKQKLK |
| (SEQ ID NO: 111) | PVLDEFREKLWEXWEALKQKLK |
| (SEQ ID NO: 112) | PVLDEFREKLNEXSEALKQKLK |
| (SEQ ID NO: 113) | PVLDEFREKLNEPLEALKQKLK |
| (SEQ ID NO: 114) | PVLDEFREKLNEXMEALKQKLK |
| (SEQ ID NO: 115) | PKLDEFREKLNEXLEALKQKLK |
| (SEQ ID NO: 116) | PHLDEFREKLNEXLEALKQKLK |
| (SEQ ID NO: 117) | PELDEFREKLNEXLEALKQKLK |
| (SEQ ID NO: 118) | PVLDEFREKLNEXLEALEQKLK |
| (SEQ ID NO: 119) | PVLDEFREKLNEELEAXKQKLK |
| (SEQ ID NO: 120) | PVLDEFREKLNEELEXLKQKLK |
| (SEQ ID NO: 121) | PVLDEFREKLNEELEALWQKLK |
| (SEQ ID NO: 122) | PVLDEFREKLNEELEWLKQKLK |
| (SEQ ID NO: 123) | QVLDLFRELLNELLEALKQKLK |
| (SEQ ID NO: 124) | PVLDLFOELLNELLEALOQOLO |
| (SEQ ID NO: 125) | NVLDLFRELLNELLEALKQKLK |
| (SEQ ID NO: 126) | PVLDLFRELLNELGEALKQKLK |
| (SEQ ID NO: 127) | PVLDLFRELLNELLELLKQKLK |
| (SEQ ID NO: 128) | PVLDLFRELLNELLEFLKQKLK |
| (SEQ ID NO: 129) | PVLELFNDLLRELLEALQKKLK |
| (SEQ ID NO: 130) | PVLELFNDLLRELLEALKQKLK |
| (SEQ ID NO: 131) | PVLELFKELLNELLDALRQKLK |
| (SEQ ID NO: 132) | PVLDLFRELLENLLEALQKKLK |
| (SEQ ID NO: 133) | PVLELFERLLEDLLQALNKKLK |
| (SEQ ID NO: 134) | PVLELFERLLEDLLKALNOKLK |
| (SEQ ID NO: 135) | DVLDLFRELLNELLEALKQKLK |
| (SEQ ID NO: 136) | PALELFKDLLQELLEALKQKLK |
| (SEQ ID NO: 137) | PVLDLFRELLNEGLEAZKQKLK |
| (SEQ ID NO: 138) | PVLDLFRELLNEGLEWLKQKLK |
| (SEQ ID NO: 139) | PVLDLFRELWNEGLEALKQKLK |
| (SEQ ID NO: 140) | PVLDLFRELLNEGLEALOQOLO |
| (SEQ ID NO: 141) | PVLDFFRELLNEGLEALKQKLK |
| (SEQ ID NO: 142) | PVLELFRELLNEGLEALKQKLK |
| (SEQ ID NO: 143) | PVLDLFRELLNEGLEALKQKLK* |
| (SEQ ID NO: 144) | PVLELFENLLERLLDALQKKLK |
| (SEQ ID NO: 145) | GVLELFENLLERLLDALQKKLK |
| (SEQ ID NO: 146) | PVLELFENLLERLLDALQKKLK |
| (SEQ ID NO: 147) | PVLELFENLLERLFDALQKKLK |
| (SEQ ID NO: 148) | PVLELFENLLERLGDALQKKLK |
| (SEQ ID NO: 149) | PVLELFENLWERLLDALQKKLK |
| (SEQ ID NO: 150) | PLLELFENLLERLLDALQKKLK |
| (SEQ ID NO: 151) | PVLELFENLGERLLDALQKKLK |
| (SEQ ID NO: 152) | PVFELFENLLERLLDALQKKLK |
| (SEQ ID NO: 153) | AVLELFENLLERLLDALQKKLK |
| (SEQ ID NO: 154) | PVLELFENLLERGLDALQKKLK |
| (SEQ ID NO: 155) | PVLELFLNLWERLLDALQKKLK |
| (SEQ ID NO: 156) | PVLELFLNLLERLLDALQKKLK |
| (SEQ ID NO: 157) | PVLEFFENLLERLLDALQKKLK |
| (SEQ ID NO: 158) | PVLELFLNLLERLLDWLQKKLK |
| (SEQ ID NO: 159) | PVLDLFENLLERLLDALQKKLK |
| (SEQ ID NO: 160) | PVLELFENLLERLLDWLQKKLK |
| (SEQ ID NO: 161) | PVLELFENLLERLLEALQKKLK |
| (SEQ ID NO: 162) | PVLELFENWLERLLDALQKKLK |
| (SEQ ID NO: 163) | PVLELFENLLERLWDALQKKLK |
| (SEQ ID NO: 164) | PVLELFENLLERLLDAWQKKLK |
| (SEQ ID NO: 165) | PVLELFENLLERLLDLLQKKLK |
| (SEQ ID NO: 166) | PVLELFLNLLEKLLDALQKKLK |
| (SEQ ID NO: 167) | PVLELFENGLERLLDALQKKLK |
| (SEQ ID NO: 168) | PVLELFEQLLEKLLDALQKKLK |
| (SEQ ID NO: 169) | PVLELFENLLEKLLDALQKKLK |
| (SEQ ID NO: 170) | PVLELFENLLEOLLDALQOOLO |
| (SEQ ID NO: 171) | PVLELFENLLEKLLDLLQKKLK |
| (SEQ ID NO: 172) | PVLELFLNLLERLGDALQKKLK |
| (SEQ ID NO: 173) | PVLDLFDNLLDRLLDLLNKKLK |
| (SEQ ID NO: 174) | PVLELFENLLERLLDALQKKLK |
| (SEQ ID NO: 175) | PVLELFENLLERLLELLNKKLK |
| (SEQ ID NO: 176) | PVLELWENLLERLLDALQKKLK |
| (SEQ ID NO: 177) | GVLELFLNLLERLLDALQKKLK |
| (SEQ ID NO: 178) | PVLELFDNLLEKLLEALQKKLR |
| (SEQ ID NO: 179) | PVLELFDNLLERLLDALQKKLK |
| (SEQ ID NO: 180) | PVLELFDNLLDKLLDALQKKLR |
| (SEQ ID NO: 181) | PVLELFENLLERWLDALQKKLK |
| (SEQ ID NO: 182) | PVLELFENLLEKLLEALQKKLK |
| (SEQ ID NO: 183) | PLLELFENLLEKLLDALQKKLK |
| (SEQ ID NO: 184) | PVLELFLNLLERLLDAWQKKLK |
| (SEQ ID NO: 185) | PVLELFENLLERLLDALQOOLO |
| (SEQ ID NO: 186) | PVLELFEQLLERLLDALQKKLK |
| (SEQ ID NO: 187) | PVLELFENLLERLLDALNKKLK |
| (SEQ ID NO: 188) | PVLELFENLLDRLLDALQKKLK |
| (SEQ ID NO: 189) | DVLELFENLLERLLDALQKKLK |
| (SEQ ID NO: 190) | PVLEFWDNLLDKLLDALQKKLR |
| (SEQ ID NO: 191) | PVLDLLRELLEELKQKLK* |
| (SEQ ID NO: 192) | PVLDLFKELLEELKQKLK* |
| (SEQ ID NO: 193) | PVLDLFRELLEELKQKLK* |
| (SEQ ID NO: 194) | PVLELFRELLEELKQKLK* |
| (SEQ ID NO: 195) | PVLELFKELLEELKQKLK* |
| (SEQ ID NO: 196) | PVLDLFRELLEELKNKLK* |
| (SEQ ID NO: 197) | PLLDLFRELLEELKQKLK* |
| (SEQ ID NO: 198) | GVLDLFRELLEELKQKLK* |
| (SEQ ID NO: 199) | PVLDLFRELWEELKQKLK* |
| (SEQ ID NO: 200) | NVLDLFRELLEELKQKLK* |
| (SEQ ID NO: 201) | PLLDLFKELLEELKQKLK* |
| (SEQ ID NO: 202) | PALELFKDLLEELRQKLR* |
| (SEQ ID NO: 203) | AVLDLFRELLEELKQKLK* |
| (SEQ ID NO: 204) | PVLDFFRELLEELKQKLK* |
| (SEQ ID NO: 205) | PVLDLFREWLEELKQKLK* |
| (SEQ ID NO: 206) | PLLELLKELLEELKQKLK* |
| (SEQ ID NO: 207) | PVLELLKELLEELKQKLK* |
| (SEQ ID NO: 208) | PALELFKDLLEELRQRLK* |
| (SEQ ID NO: 209) | PVLDLFRELLNELLQKLK |
| (SEQ ID NO: 210) | PVLDLFRELLEELKQKLK |
| (SEQ ID NO: 211) | PVLDLFRELLEELOQOLO* |
| (SEQ ID NO: 212) | PVLDLFOELLEELOQOLK* |
| (SEQ ID NO: 213) | PALELFKDLLEEFRQRLK* |
| (SEQ ID NO: 214) | PVLDLFRELLEELKQKLK* |
| (SEQ ID NO: 215) | PVLDLFRELLEEWKQKLK* |
| (SEQ ID NO: 216) | PVLELFKELLEELKQKLK |
| (SEQ ID NO: 217) | PVLDLFRELLELLKQKLK |
| (SEQ ID NO: 218) | PVLDLFRELLNELLQKLK* |
| (SEQ ID NO: 219) | PVLDLFRELLNELWQKLK |
| (SEQ ID NO: 220) | PVLDLFRELLEELQKKLK |
| (SEQ ID NO: 221) | DVLDLFRELLEELKQKLK* |
| (SEQ ID NO: 222) | PVLDAFRELLEALLQLKK |
| (SEQ ID NO: 223) | PVLDAFRELLEALAQLKK |
| (SEQ ID NO: 224) | PVLDLFREGWEELKQKLK |
| (SEQ ID NO: 225) | PVLDAFRELAEALAQLKK |
| (SEQ ID NO: 226) | PVLDAFRELGEALLQLKK |
| (SEQ ID NO: 227) | PVLDLFRELGEELKQKLK* |
| (SEQ ID NO: 228) | PVLDLFREGLEELKQKLK* |
| (SEQ ID NO: 229) | PVLDLFRELLEEGKQKLK* |
| (SEQ ID NO: 230) | PVLELFERLLEDLQKKLK |
| (SEQ ID NO: 231) | PVLDLFRELLEKLEQKLK |
| (SEQ ID NO: 232) | PLLELFKELLEELKQKLK* |
| (SEQ ID NO: 233) | LDDLLQKWAEAFNQLLKK |
| (SEQ ID NO: 234) | EWLKAFYEKVLEKLKELF* |
| (SEQ ID NO: 235) | EWLEAFYKKVLEKLKELF* |
| (SEQ ID NO: 236) | DWLKAFYDKVAEKLKEAF* |
| (SEQ ID NO: 237) | DWFKAFYDKVFEKFKEFF |
| (SEQ ID NO: 238) | GIKKFLGSIWKFIKAFVG |
| (SEQ ID NO: 239) | DWFKAFYDKVAEKFKEAF |
| (SEQ ID NO: 240) | DWLKAFYDKVAEKLKEAF |
| (SEQ ID NO: 241) | DWLKAFYDKVFEKFKEFF |
| (SEQ ID NO: 242) | EWLEAFYKKVLEKLKELP |
| (SEQ ID NO: 243) | DWFKAFYDKFFEKFKEFF |
| (SEQ ID NO: 244) | EWLKAFYEKVLEKLKELF |
| (SEQ ID NO: 245) | EWLKAEYEKVEEKLKELF* |
| (SEQ ID NO: 246) | EWLKAEYEKVLEKLKELF* |
| (SEQ ID NO: 247) | EWLKAFYKKVLEKLKELF* |
| (SEQ ID NO: 248) | PVLDLFRELLEQKLK* |
| (SEQ ID NO: 249) | PVLDLFRELLEELKQK* |
| (SEQ ID NO: 250) | PVLDLFRELLEKLKQK* |
| (SEQ ID NO: 251) | PVLDLFRELLEKLOK* |
| (SEQ ID NO: 252) | PVLDLFRELLEALKQK* |
| (SEQ ID NO: 253) | PVLDLFENLLERLKQK* |
| (SEQ ID NO: 254) | PVLDLFRELLNELKQK* |

*indicates peptides that are N-terminal acetylated and
C-terminal amidated; indicates peptides that are N-terminal
dansylated; sp indicates peptides that exhibited solubility
problems under the experimental conditions; X is Aib; Z is
Nal; O is Orn; and ~ indicates deleted amino acids.

In some embodiments, an ApoA-I mimetic having the following sequence as described in U.S. Pat. No. 6,743,778 is utilized: Asp Trp Leu Lys Ala Phe Tyr Asp Lys Val Ala Glu Lys Leu Lys Glu Ala Phe (SEQ ID NO: 255).

In some embodiments, any of the following ApoA-I mimetics shown in Table 2 as described in U.S. Patent Application Publication No. 2003/0171277 are utilized:

TABLE 2
ApoA-I mimetics

| SEQ ID NO | AMINO ACID SEQUENCE |
|---|---|
| (SEQ ID NO: 256) | D-W-L-K-A-F-Y-D-K-V-A-E-K-L-K-E- |
| | A-F |
| (SEQ ID NO: 257) | Ac-D-W-L-K-A-F-Y-D-K-V-A-E-K-L- |
| | K-E-A-F-NH₂ |
| (SEQ ID NO: 258) | Ac-D-W-F-K-A-F-Y-D-K-V-A-E-K-L- |
| | K-E-A-F-NH₂ |
| (SEQ ID NO: 259) | Ac-D-W-L-K-A-F-Y-D-K-V-A-E-K-F- |
| | K-E-A-F-NH₂ |
| (SEQ ID NO: 260) | Ac-D-W-F-K-A-F-Y-D-K-V-A-E-K-F- |
| | K-E-A-F-NH₂ |
| (SEQ ID NO: 261) | Ac-D-W-L-K-A-F-Y-D-K-V-F-E-K-F- |
| | K-E-F-F-NH₂ |
| (SEQ ID NO: 262) | Ac-D-W-L-K-A-F-Y-D-K-F-F-E-K-F- |
| | K-E-F-F-NH₂ |
| (SEQ ID NO: 263) | Ac-D-W-F-K-A-F-Y-D-K-F-F-E-K-F- |
| | K-E-F-F-NH₂ |
| (SEQ ID NO: 264) | Ac-D-W-L-K-A-F-Y-D-K-V-A-E-K-L- |
| | K-E-F-F-NH₂ |
| (SEQ ID NO: 265) | Ac-D-W-L-K-A-F-Y-D-K-V-F-E-K-F- |
| | K-E-A-F-NH₂ |
| (SEQ ID NO: 266) | Ac-D-W-L-K-A-F-Y-D-K-V-F-E-K-L- |
| | K-E-F-F-NH₂ |
| (SEQ ID NO: 267) | Ac-D-W-L-K-A-F-Y-D-K-V-A-E-K-F- |
| | K-E-F-F-NH₂ |
| (SEQ ID NO: 268) | Ac-D-W-L-K-A-F-Y-D-K-V-F-E-K-F- |
| | K-E-F-F-NH₂ |
| (SEQ ID NO: 269) | Ac-E-W-L-K-L-F-Y-E-K-V-L-E-K-F- |
| | K-E-A-F-NH₂ |
| (SEQ ID NO: 270) | Ac-E-W-L-K-A-F-Y-D-K-V-A-E-K-F- |
| | K-E-A-F-NH₂ |
| (SEQ ID NO: 271) | Ac-E-W-L-K-A-F-Y-D-K-V-A-E-K-L- |
| | K-E-F-F-NH₂ |
| (SEQ ID NO: 272) | Ac-E-W-L-K-A-F-Y-D-K-V-F-E-K-F- |
| | K-E-A-F-NH₂ |
| (SEQ ID NO: 273) | Ac-E-W-L-K-A-F-Y-D-K-V-F-E-K-L- |
| | K-E-F-F-NH₂ |
| (SEQ ID NO: 274) | Ac-E-W-L-K-A-F-Y-D-K-V-A-E-K-F- |
| | K-E-F-F-NH₂ |
| (SEQ ID NO: 275) | Ac-E-W-L-K-A-F-Y-D-K-V-F-E-K-F- |
| | K-E-F-F-NH₂ |
| (SEQ ID NO: 276) | Ac-A-F-Y-D-K-V-A-E-K-L-K-E-A-F-NH₂ |
| (SEQ ID NO: 277) | Ac-A-F-Y-D-K-V-A-E-K-F-K-E-A-F-NH₂ |
| (SEQ ID NO: 278) | Ac-A-F-Y-D-K-V-A-E-K-F-K-E-A-F-NH₂ |
| (SEQ ID NO: 279) | Ac-A-F-Y-D-K-F-F-E-K-F-K-E-F-F-NH₂ |
| (SEQ ID NO: 280) | Ac-A-F-Y-D-K-F-F-E-K-F-K-E-F-F-NH₂ |
| (SEQ ID NO: 281) | Ac-A-F-Y-D-K-V-A-E-K-F-K-E-A-F-NH₂ |
| (SEQ ID NO: 282) | Ac-A-F-Y-D-K-V-A-E-K-L-K-E-F-F-NH₂ |
| (SEQ ID NO: 283) | Ac-A-F-Y-D-K-V-F-E-K-F-K-E-A-F-NH₂ |
| (SEQ ID NO: 284) | Ac-A-F-Y-D-K-V-F-E-K-L-K-E-F-F-NH₂ |
| (SEQ ID NO: 285) | Ac-A-F-Y-D-K-V-A-E-K-F-K-E-F-F-NH₂ |
| (SEQ ID NO: 286) | Ac-K-A-F-Y-D-K-V-F-E-K-F-K-E-F-NH₂ |
| (SEQ ID NO: 287) | Ac-L-F-Y-E-K-V-L-E-K-F-K-E-A-F-NH₂ |
| (SEQ ID NO: 288) | Ac-A-F-Y-D-K-V-A-E-K-F-K-E-A-F-NH₂ |
| (SEQ ID NO: 289) | Ac-A-F-Y-D-K-V-A-E-K-L-K-E-F-F-NH₂ |
| (SEQ ID NO: 290) | Ac-A-F-Y-D-K-V-F-E-K-F-K-E-A-F-NH₂ |
| (SEQ ID NO: 291) | Ac-A-F-Y-D-K-V-F-E-K-L-K-E-F-F-NH₂ |
| (SEQ ID NO: 292) | Ac-A-F-Y-D-K-V-A-E-K-F-K-E-F-F-NH₂ |
| (SEQ ID NO: 293) | Ac-A-F-Y-D-K-V-F-E-K-F-K-E-F-F-NH₂ |
| (SEQ ID NO: 294) | Ac-D-W-L-K-A-L-Y-D-K-V-A-E-K-L-K- |
| | E-A-L-NH₂ |
| (SEQ ID NO: 295) | Ac-D-W-F-K-A-F-Y-E-K-V-A-E-K-L-K- |
| | E-F-F-NH₂ |
| (SEQ ID NO: 296) | Ac-D-W-F-K-A-F-Y-E-K-F-F-E-K-F-K- |
| | E-F-F-NH₂ |
| (SEQ ID NO: 297) | Ac-E-W-L-K-A-L-Y-E-K-V-A-E-K-L-K- |
| | E-A-L-NH₂ |
| (SEQ ID NO: 298) | Ac-E-W-L-K-A-F-Y-E-K-V-A-E-K-L-K- |
| | E-A-F-NH₂ |
| (SEQ ID NO: 299) | Ac-E-W-F-K-A-F-Y-E-K-V-A-E-K-L-K- |
| | E-F-F-NH₂ |
| (SEQ ID NO: 300) | Ac-E-W-L-K-A-F-Y-E-K-V-F-E-K-F-K- |
| | E-F-F-NH₂ |
| (SEQ ID NO: 301) | Ac-E-W-L-K-A-F-Y-E-K-F-F-E-K-F-K- |
| | E-F-F-NH₂ |
| (SEQ ID NO: 302) | Ac-E-W-F-K-A-F-Y-E-K-F-F-E-K-F-K- |
| | E-F-F-NH₂ |
| (SEQ ID NO: 303) | Ac-D-F-L-K-A-W-Y-D-K-V-A-E-K-L-K- |
| | E-A-W-NH₂ |
| (SEQ ID NO: 304) | Ac-E-F-L-K-A-W-Y-E-K-V-A-E-K-L-K- |
| | E-A-W-NH₂ |
| (SEQ ID NO: 305) | Ac-D-F-W-K-A-W-Y-D-K-V-A-E-K-L-K- |
| | E-W-W-NH₂ |
| (SEQ ID NO: 306) | Ac-E-F-W-K-A-W-Y-E-K-V-A-E-K-L-K- |
| | E-W-W-NH₂ |
| (SEQ ID NO: 307) | Ac-D-K-L-K-A-F-Y-D-K-V-F-E-W-A-K- |
| | E-A-F-NH₂ |
| (SEQ ID NO: 308) | Ac-D-K-W-K-A-V-Y-D-K-F-A-E-A-F-K- |
| | E-F-L-NH₂ |
| (SEQ ID NO: 309) | Ac-E-K-L-K-A-F-Y-E-K-V-F-E-W-A-K- |
| | E-A-F-NH₂ |
| (SEQ ID NO: 310) | Ac-E-K-W-K-A-V-Y-E-K-F-A-E-A-F-K- |
| | E-F-L-NH₂ |
| (SEQ ID NO: 311) | Ac-D-W-L-K-A-F-V-D-K-F-A-E-K-F-K- |
| | E-A-Y-NH₂ |
| (SEQ ID NO: 312) | Ac-E-K-W-K-A-V-Y-E-K-F-A-E-A-F-K- |
| | E-F-L-NH₂ |
| (SEQ ID NO: 313) | Ac-D-W-L-K-A-F-V-Y-D-K-V-F-K-L-K- |
| | E-F-F-NH₂ |
| (SEQ ID NO: 314) | Ac-E-W-L-K-A-F-V-Y-E-K-V-F-K-L-K- |
| | E-F-F-NH₂ |
| (SEQ ID NO: 315) | Ac-D-W-L-R-A-F-Y-D-K-V-A-E-K-L-K- |
| | E-A-F-NH₂ |
| (SEQ ID NO: 316) | Ac-E-W-L-R-A-F-Y-E-K-V-A-E-K-L-K- |
| | E-A-F-NH₂ |
| (SEQ ID NO: 317) | Ac-D-W-L-K-A-F-Y-D-R-V-A-E-K-L-K- |
| | E-A-F-NH₂ |
| (SEQ ID NO: 318) | Ac-E-W-L-K-A-F-Y-E-R-V-A-E-K-L-K- |
| | E-A-F-NH₂ |
| (SEQ ID NO: 319) | Ac-D-W-L-K-A-F-Y-D-K-V-A-E-R-L-K- |
| | E-A-F-NH₂ |
| (SEQ ID NO: 320) | Ac-E-W-L-K-A-F-Y-E-K-V-A-E-R-L-K- |
| | E-A-F-NH₂ |
| (SEQ ID NO: 321) | Ac-D-W-L-K-A-F-Y-D-K-V-A-E-K-L-R- |
| | E-A-F-NH₂ |
| (SEQ ID NO: 322) | Ac-E-W-L-K-A-F-Y-E-K-V-A-E-K-L-R- |
| | E-A-F-NH₂ |
| (SEQ ID NO: 323) | Ac-D-W-L-K-A-F-Y-D-R-V-A-E-R-L-K- |
| | E-A-F-NH₂ |
| (SEQ ID NO: 324) | Ac-E-W-L-K-A-F-Y-E-R-V-A-E-R-L-K- |
| | E-A-F-NH₂ |
| (SEQ ID NO: 325) | Ac-D-W-L-R-A-F-Y-D-K-V-A-E-K-L-R- |
| | E-A-F-NH₂ |
| (SEQ ID NO: 326) | Ac-E-W-L-R-A-F-Y-E-K-V-A-E-K-L-R- |
| | E-A-F-NH₂ |
| (SEQ ID NO: 327) | Ac-D-W-L-R-A-F-Y-D-R-V-A-E-K-L-K- |
| | E-A-F-NH₂ |
| (SEQ ID NO: 328) | Ac-E-W-L-R-A-F-Y-E-R-V-A-E-K-L-K- |
| | E-A-F-NH₂ |
| (SEQ ID NO: 329) | Ac-D-W-L-K-A-F-Y-D-K-V-A-E-R-L-R- |
| | E-A-F-NH₂ |
| (SEQ ID NO: 330) | Ac-E-W-L-K-A-F-Y-E-K-V-A-E-R-L-R- |
| | E-A-F-NH₂ |
| (SEQ ID NO: 331) | Ac-D-W-L-R-A-F-Y-D-K-V-A-E-R-L-K- |
| | E-A-F-NH₂ |
| (SEQ ID NO: 332) | Ac-E-W-L-R-A-F-Y-E-K-V-A-E-R-L-K- |
| | E-A-F-NH₂ |

In some embodiments, an Apo A-I mimetic having the following sequence as described in U.S. Patent Application Publication No. 2006/0069030 is utilized: F-A-E-K-F-K-E-A-V-K-D-Y-F-A-K-F-W-D (SEQ ID NO:333).

In some embodiments, an Apo A-I mimetic having the following sequence as described in U.S. Patent Application Publication No. 2009/0081293 is utilized:

(SEQ ID NO: 334)
DWFKAFYDKVAEKFKEAF;
(SEQ ID NO: 335)
DWLKAFYDKVAEKLKEAFL
(SEQ ID NO: 336)
PALEDLRQGLLPVLESFKVFLSALEEYTKKLNTQ.

In some embodiments, an Apo A-I mimetic having one of the following sequences is utilized:

(SEQ ID NO: 341)
WDRVKDLATVYVDVLKDSGRDYVSQF,
(SEQ ID NO: 342)
LKLLDNWDSVTSTFSKLREOL,
(SEQ ID NO: 343)
PVTOEFWDNLEKETEGLROEMS,
(SEQ ID NO: 344)
KDLEEVKAKVQ,
(SEQ ID NO: 345)
KDLEEVKAKVO,
(SEQ ID NO: 346)
PYLDDFQKKWQEEMELYRQKVE,
(SEQ ID NO: 347)
PLRAELQEGARQKLHELOEKLS,
(SEQ ID NO: 348)
PLGEEMRDRARAHVDALRTHLA,
(SEQ ID NO: 349)
PYSDELRQRLAARLEALKENGG,
(SEQ ID NO: 350)
ARLAEYHAKATEHLSTLSEKAK,
(SEQ ID NO: 351)
PALEDLOGLL,
(SEQ ID NO: 352)
PVLESFKVSFLSALEEYTKKLN,
(SEQ ID NO 353)
PVLESFVSFLSALEEYTKKLN,
(SEQ ID NO: 352)
PVLESFKVSFLSALEEYTKKLN,
(SEQ ID NO: 354)
TVLLLTICSLEGALVRRQAKEPCV,
(SEQ ID NO: 355)
QTVTDYGKDLME,
(SEQ ID NO: 356)
KVKSPELOAEAKSYFEKSKE,
(SEQ ID NO: 357)
VLTLALVAVAGARAEVSADOVATV,
(SEQ ID NO: 358)
NNAKEAVEHLOKSELTOOLNAL,
(SEQ ID NO: 359)
LPVLVWLSIVLEGPAPAOGTPDVSS,
(SEQ ID NO: 360)
LPVLVVVLSIVLEGPAPAQGTPDVSS,
(SEQ ID NO: 361)
ALDKLKEFGNTLEDKARELIS,
(SEQ ID NO: 362)
VVALLALLASARASEAEDASLL,
(SEQ ID NO: 363)
HLRKLRKRLLRDADDLQKRLAVYOA,
(SEQ ID NO: 364)
AQAWGERLRARMEEMGSRTRDR,
(SEQ ID NO: 365)
LDEVKEQVAEVRAKLEEQAQ,
(SEQ ID NO: 366)
SWLKAFYDKVAEKLKEAFPDWAKAAYDKAAEKAKEAA,
(SEQ ID NO: 367)
PVLDLFRELLNELLEALKQKL,
(SEQ ID NO: 368)
PVLDLFRELLNELLEALKQKLA,
(SEQ ID NO: 4)
PVLDLFRELLNELLEALKQKLK,
(SEQ ID NO: 369)
PVLDLFRELLNELLEALKQKLA,
(SEQ ID NO: 370)
PVLDLFRELLNELLEALKKLLK,
(SEQ ID NO: 371)
PVLDLFRELLNELLEALKKLLA,
(SEQ ID NO: 372)
PLLDLFRELLNELLEALKKLLA,
and
(SEQ ID NO: 373)
EVRSKLEEWFAAFREFAEEFLARLKS.

Amphipathic lipids include, for example, any lipid molecule which has both a hydrophobic and a hydrophilic moiety. Examples include phospholipids or glycolipids. Examples of phospholipids which may be used in the sHDL-tolerogenic antigen nanoparticles include but are not limited to 1,2-dilauroyl-sn-glycero-3-phosphocholine; 1,2-dimyristoyl-sn-glycero-3-phosphocholine; 1,2-dipalmitoyl-sn-glycero-3-phosphocholine; 1,2-distearoyl-sn-glycero-3-phosphocholine; 1,2-diarachidoyl-sn-glycero-3-phosphocholine; 1,2-dibehenoyl-sn-glycero-3-phosphocholine; 1,2-dilignoceroyl-sn-glycero-3-phosphocholine; 1,2-dimyristoleoyl-sn-glycero-3-phosphocholine; 1,2-dimyristelaidoyl-sn-glycero-3-phosphocholine; 1,2-dipalmitoleoyl-sn-glycero-3-phosphocholine; 1,2-dipalmitelaidoyl-sn-glycero-3-phosphocholine; 1,2-dipetroselenoyl-sn-glycero-3-phosphocholine; 1,2-dioleoyl-sn-glycero-3-phosphocholine; 1,2-dielaidoyl-sn-glycero-3-phosphocholine; 1,2-dieicosenoyl-sn-glycero-3-phosphocholine; 1,2-dinervonoyl-sn-glycero-3-phosphocholine; 1,2-dilauroyl-sn-glycero-3-phosphoethanolamine; 1,2-dimyristoyl-sn-glycero-3-phosphoethanolamine; 1,2-dipentadecanoyl-sn-glycero-3-phosphoethanolamine; 1,2-dipalmitoyl-sn-glycero-3-phosphoethanolamine; 1,2-distearoyl-sn-glycero-3-phosphoethanolamine; 1,2-dipalmitoleoyl-sn-glycero-3-phosphoethanolamine; 1,2-dielaidoyl-sn-glycero-3-phosphoethanolamine; 1,2-dioleoyl-sn-glycero-3-phosphoethanolamine; dioleoyl-sn-glycero-3-phosphoethanolamine-N-[3-(2-pyridyldithio) propionate]; 1,2-dipalmitoyl-sn-glycero-3-phosphothioethanol; 1,2-di-(9Z-octadecenoyl)-sn-glycero-3-phosphoethanolamine-N-[4-(p-maleimidophenyl)butyramide]; 1,2-dihexadecanoyl-sn-glycero-3-phosphoethanolamine-N-[4-(p-maleimidophenyl)butyramide]; 1,2-dihexadecanoyl-sn-glycero-3-phosphoethanolamine-N-[4-(p-maleimidomethyl)cyclohexane-carboxamide]; 1,2-di-(9Z-octadecenoyl)-sn-glycero-3-phosphoethanolamine-N-[4-(p-maleimidomethyl)cyclohexane-carboxamide]; N-[(3-Maleimide-1-oxopropyl)aminopropyl polyethyleneglycol-carbamyl]distearoylphosphatidyl-ethanolamine; N-[(3-Maleimide-1-oxopropyl)aminopropyl polyethylenegly col-carbamyl]distearoylphosphatidyl-ethanolamine; N-(3-Maleimide-1-oxopropyl)-L-α-phosphatidylethanolamine, Distearoyl; N-[(3-Maleimide-1-oxopropyl)aminopropyl polyethyleneglycol-carbamyl]distearoylphosphatidyl-ethanolamine; N-(3-Maleimide-1-oxopropyl)-L-α-phosphatidylethanolamine, Dimyristoy; N-(3-Maleimide-1-oxopropyl)-L-α-phosphatidylethanolamine, Dioleoyl; N-(3-Maleimide-1-oxopropyl)-L-α-phosphatidylethanolamine, Dipalmitoyl; N-(3-Maleimide-1-oxopropyl)-L-α-phosphatidylethanolamine, 1-Palmitoyl-2-oleoyl; phosphatidylcholine; phosphatidylinositol; phosphatidylserine; phosphatidylethanolamine; N-(Succinimidyloxy-glutaryl)-L-α-phosphatidylethanolamine, Distearoyl; N-(Succinimidyloxy-glutaryl)-L-α-phosphatidylethanolamine, Dioleoyl; N-(Succinimidyloxy-glutaryl)-L-α-phosphatidylethanolamine, 1-Palmitoyl-2-oleoyl; N-(Succinimidyloxy-glutaryl)-L-α-phosphatidylethanolamine, Dipalmitoyl; N-(Succinimidyloxy-glutaryl)-L-α-phosphatidylethanolamine, Dimyristoyl; 3-(N-succinimidyloxyglutaryl)aminopropyl, and polyethyleneglycol-carbamyl distearoylphosphatidyl-ethanolamine; N-(3-oxopropoxy polyethylenegly col)carbamyl-distearoyl-ethanolamine.

In some embodiments, the sHDL nanoparticles have a molar ratio of phospholipid/HDL apolipoprotein from 2 to 250 (e.g., 10 to 200, 20 to 100, 20 to 50, 30 to 40).

Generally, the sHDL nanoparticles so formed are spherical or discoidal and have a diameter of from about 5 nm to about 20 nm (e.g., 4-75 nm, 4-60 nm, 4-50 nm, 4-22 nm, 6-18 nm, 8-15 nm, 8-10 nm, etc.). In some embodiments, the sHDL nanoparticles are subjected to size exclusion chromatography to yield a more homogeneous preparation.

Such compositions are not limited to specific tolerogenic antigens implicated in autoimmune disease (e.g., MS or celiac disease).

### Tolerogenic Antigens

The present invention includes compositions and methods for treating autoimmune disease (e.g., MS or celiac disease) including nanoparticles associated with a plurality of tolerogenic antigens (e.g., between 1-30 tolerogenic antigens (e.g., 8-30 tolerogenic antigens per nanoparticle) implicated in autoimmune disease (e.g., MS or celiac disease), as well as methods utilizing such nanoparticles. In the present invention, the tolerogenic antigens are antigens that have been identified to play a role in autoimmune disease (e.g., MS or celiac disease). In some embodiments, the tolerogenic antigen is between about 3 amino acids and about 50 amino acids in length (e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length). In some embodiments, the tolerogenic antigen is a single tolerogenic antigen between about 3 and about 50 amino acids in length.

In celiac disease, the main antigens are tissue transglutaminase and gliadin (e.g., α-, γ-, and ω-gliadin). Any antigen identified as a tissue transglutaminase or a gliadin antigen may be used.

In some embodiments, the antigen associated with the nanoparticle includes a gliadin polypeptide, such as the full-length gliadin polypeptide or any epitopes of the gliadin polypeptide. In some embodiments, the antigen associated with the nanoparticle includes a 33-mer polypeptide from α2-gliadin. In some embodiments, the 33-mer gliadin polypeptide has at least 90% (at least 91%, at least 92%, at least 93%, at least 94%, at least 95%, at least 96%, at least 97%, at least 98%, at least 99%, or 100%) sequence identity to the polypeptide sequence of LQLQPFPQPELPYPQPELPYPQPELPYPQPQPF (SEQ ID NO: 374). In some embodiments, the antigen associated with the nanoparticles includes an epitope of the 33-mer gliadin polypeptide. The epitope of the 33-mer gliadin polypeptide may be a polypeptide of any length shorter than the 33-mer polypeptide, for example the epitope may include between 25 and 3 (e.g., 24, 23, 22, 21, 20, 19, 18, 17, 16, 15, 14, 13, 12, 11, 10, 9, 8, 7, 6, 5, 4 or 3) amino acid residues, between 20 and 5 (e.g., 19, 18, 17, 16, 15, 14, 13, 12, 11, 10, 9, 8, 7, 6, or 5) amino acids residues, between 12 and 6 (e.g., 12, 11, 10, 9, 8, 7 or 6) amino acid residues, or 9 amino acids in length. Further examples of epitopes of the 33-gliadin that may be associated with the nanoparticles include any one of the epitopes described in Table 3, including SEQ ID Nos: 375-405. In some embodiments, the tolerogenic antigen associated with the nanoparticle may include any one of the antigens described in Table 4, including SEQ ID Nos: 406-580. In some embodiments, the antigen associated with the nanoparticles includes a polypeptide sequence having at least 85% (e.g., at least 86%, at least 87%, at least 88%, at least 89%, at least 90%, at least 95%, or 100%) sequence identity to any one of SEQ ID Nos: 375-580. In some embodiments, the tolerogenic antigen associated with the nanoparticle may include an antigen including two or more (e.g., 2, 3, 4, 5, or 6) polypeptides having the polypeptide sequences of any two of SEQ ID Nos: 375-580. In some embodiments, the plurality of tolerogenic antigens (e.g., between 1-30 (e.g., 6-30, or 8-30 (e.g., 8, 9, 10, 11, 12, 13, 14, 15, 16, 17, 18, 19, 20, 21, 22, 23, 24, 25, 26, 27, 28, 29, or 30)) tolerogenic antigens per nanoparticle) associated with the nanoparticles have the same identity as every other tolerogenic antigen associated with the nanoparticle. In some embodiments, the plurality of tolerogenic antigens associated with the nanoparticles includes a population of between 2-10 (e.g., 2, 3, 4, 5, 6, 7, 8, 9, or 10) different antigen sequences implicated in the same disease; for example, the nanoparticles may be associated with between 3-8 (e.g., 3, 4, 5, 6, 7, or 8), 4-6 (e.g., 4, 5, or 6), or 3-4 different polypeptide antigen sequences. In some embodiments, the nanoparticles may be associated with (i) a first polypeptide population comprising the amino acid sequence of any one of SEQ ID Nos: 406-580, or a biologically active fragment or variant thereof, (ii) a second polypeptide population comprising the amino acid sequence of any one of SEQ ID Nos: 406-580, or biologically active fragment or variant thereof, and (iii) a third polypeptide population comprising the amino acid sequence of any one of SEQ ID Nos: 406-580, or a biologically active fragment or variant thereof. In some instances, the first, second, and third polypeptide populations have different amino acid sequences. In some embodiments, the nanoparticles may be associated with (i) a first polypeptide comprising the amino acid sequence LQPFPQPELPYPQPQ (SEQ ID NO: 474), or a biologically active fragment or variant thereof, (ii) a second polypeptide comprising the amino acid sequence QPFPQPEQPFPWQP (SEQ ID NO: 475), or a biologically active fragment or variant thereof, and (iii) a third polypeptide comprising the amino acid sequence PEQPIPEQPQPYPQQ (SEQ ID NO: 476), or a biologically active fragment or variant thereof. In some embodiments, the nanoparticles may be associated with (i) a first polypeptide comprising the amino acid sequence LQPFPQPELPYPQPQ (SEQ ID NO: 474), or a biologically active fragment or variant thereof, (ii) a second polypeptide comprising the amino acid sequence PQQPFPQPEQPFPWQP (SEQ ID NO: 477), or a biologically active fragment or variant thereof, and (iii) a third polypeptide comprising the amino acid sequence FPEQPIPEQPQPYPQQ (SEQ ID NO: 478), or a biologically active fragment or variant thereof. In some embodiments, the nanoparticles may be associated with (i) a first polypeptide comprising the amino acid sequence ELQPFPQPELPYPQPQ (SEQ ID NO: 506), or a biologically active fragment or variant thereof, (ii) a second polypeptide comprising the amino acid sequence EQPFPQPEQPFPWQP (SEQ ID NO: 507), or a biologically active fragment or variant thereof, and (iii) a third polypeptide comprising the amino acid sequence EPEQPIPEQPQPYPQQ (SEQ ID NO: 508), or a biologically active fragment or variant thereof. In some embodiments, the tolerogenic antigens having polypeptide sequences of SEQ ID Nos: 506, 507, and 508 include an N-terminus pyroglutamic acid (pyroE). In some embodiments described herein, the tolerogenic antigens having polypeptide sequences of SEQ ID Nos: 506, 507, and 508 include a C-terminus amide group. In some embodiments described herein, the tolerogenic antigens having polypeptide sequences of SEQ ID Nos: 506, 507, and 508 include a N-terminus pyroE residue and a C-terminus amide group. In some embodiments, the nanoparticles may be associated with (i) a first polypeptide comprising the amino acid sequence QLQPFPQPELPYPQPQ (SEQ ID NO: 509), or a biologically active fragment or variant thereof, (ii) a second polypeptide comprising the amino acid sequence QQPFPQPEQPFPWQP (SEQ ID NO: 510), or a biologically active fragment or variant thereof, and (iii) a third polypeptide comprising the amino acid sequence FPEQPIPEQPQPYPQQ (SEQ ID NO: 511), or a biologically active fragment or variant thereof. In some embodiments, the tolerogenic antigens having polypeptide sequences of SEQ ID Nos: 509, 510, and 511 include an N-terminus acetyl group. In some embodiments described herein, the tolerogenic antigens having polypeptide sequences of SEQ ID Nos: 509, 510, and 511 include a C-terminus amide group. In some embodiments described herein, the tolerogenic antigens having polypeptide sequences of SEQ ID Nos: 509, 510, and 511 include a N-terminus acetyl group and a C-terminus amide group. In any of the embodiments described herein, the population of antigens associated with the nanoparticle may be fully or partially deamidated. In some embodiments described herein, the tolerogenic antigens associated with the nanoparticle may include an N-terminus pyroglutamic acid (pyroE). In some embodiments described herein, the tolerogenic antigens associated with the nanoparticle may include an N-terminus acetyl group. In some embodiments described herein, the tolerogenic antigens associated with the nanoparticle may include an N-terminus amide group. In some embodiments described herein, the tolerogenic antigens associated with the nanoparticle may include a C-terminus amide group.

TABLE 3
Celiac Disease Relevant T-cell epitopes
recognized by CD4⁺ T cells

| SEQ ID NO: | Sequence |
|---|---|
| 375 | PFPQPELPY |
| 376 | PYPQPELPY |
| 377 | PQPELPYPQ |
| 378 | FRPEQPYPQ |
| 379 | PQQSFPEQQ |
| 380 | IQPEQPAQL |
| 381 | QQPEQPYPQ |
| 382 | SQPEQEFPQ |
| 383 | PQPEQEFPQ |
| 384 | QQPEQPFPQ |
| 385 | PQPEQPFCQ |
| 386 | QQPFPEQPQ |
| 387 | PFPQPEQPF |
| 388 | PQPEQPFPW |
| 389 | PFSEQEQPV |
| 390 | FSQQQESPF |
| 391 | QQPIPEQPQ |
| 392 | PQPEQPFPQ |
| 393 | PIPEQPQPY |
| 394 | EQPIPEQPQ |
| 395 | PQPEQPFPQ |
| 396 | PYPEQEEPF |
| 397 | PYPEQEQPF |
| 398 | PFSEQEQPV |
| 399 | EGSFQPSQE |
| 400 | EQPQQPFPQ |
| 401 | EQPQQPYPE |
| 402 | QGYYPTSPQ |
| 403 | EGSFQPSQE |
| 404 | PQQSFPEQE |
| 405 | QGYYPTSPQ |

TABLE 4
Tolerogenic Antigens

| SEQ | | SEQ | |
|---|---|---|---|
| ID NO: | Sequence | ID NO: | Sequence |
| 406 | QPFPQPQQPFPW | 498 | YSPYQPQQPFPQ |
| 407 | QPFPQPQQPIPV | 499 | QLPLQPQQPFPQ |
| 408 | QPFPQPEQPFPW | 500 | QQPQQPFPLQPQQPVP |
| 409 | PFPQPEQPIPV | 501 | IIPQQPQQPFPL |
| 410 | QPFPQPELPFPQ | 502 | PEQIIPQQPQQP |
| 411 | LPYPQPQLPYPQ | 503 | FLLQPQQPFSQ |
| 412 | LPYPQPELPYPQ | 504 | IISQQPQQPFPL |
| 413 | QPFPQPQLPYPQ | 505 | PFPQRPQQPFPQ |
| 414 | QPFPQPELPYPQ | 506 | ELQPFPQPELPYPQPQ |
| 415 | QPFPQPQQPFSQ | 507 | EQPFPQPEQPFPWQP |
| 416 | QPFPQPEQPFSQ | 508 | EPEQPIPEQPQPYPQQ |
| 417 | QPFPQPQQPFCQ | 509 | QLQPFPQPELPYPQPQ |
| 418 | QPFPQPEQPFCQ | 510 | QQPFPQPEQPFPWQP |
| 419 | QPFPQPQLPYSQ | 511 | FPEQPIPEQPQPYPQQ |
| 420 | QPFPQPELPYSQ | 512 | PELP |
| 421 | LQQQCSPVAMPQRLAR | 513 | QPELPYP |
| 422 | QPFPQPQLPYLQ | 514 | PQPELPY |
| 423 | QPFPQPELPYLQ | 515 | FPQPELP |
| 424 | QQFIQPQQPFPQ | 516 | PELPYPQP |
| 425 | QQFIQPEQPFPQ | 517 | QPELPYPQ |
| 426 | LERPWQQQPLPP | 518 | PQPELPYP |
| 427 | LERPWQEQPLPP | 519 | FPQPELPY |
| 428 | PIPQQPEQPFPL | 520 | PFPQPELP |
| 429 | QGQQGYYPISPQQSGQ | 521 | PELPYPQPQ |
| 430 | QGQPGYYPTSPQQIGQ | 522 | QPELPYPQP |
| 431 | PGQGQSGYYPTSPQQS | 523 | FPQPELPYP |
| 432 | PQQTFPQQPQLP | 524 | PFPQPELPY |
| 433 | PQQTFPEQPQLP | 525 | QPFPQPELP |
| 434 | GQGQSGYYPTSPQQSG | 526 | QPELPYPQPQ |
| 435 | QYEVIRSLVLRTLPNM | 527 | PQPELPYPQP |
| 436 | QVDPSGQVQWPQ | 528 | FPQPELPYPQ |
| 437 | QVDPSGEVQWPQ | 529 | PFPQPELPYP |
| 438 | QPFPQPQQPFPL | 530 | QPFPQPELPY |
| 439 | QPFPQPEQPFPL | 531 | LQPFPQPELP |
| 440 | QPFPQPQQPIPY | 532 | PQPELPYPQPQ |
| 441 | QPFPQPEQPIPY | 533 | FPQPELPYPQP |
| 442 | PQQPVPQQPQPY | 534 | PFPQPELPYPQ |
| 443 | PQQPVPEQPQPY | 535 | QPFPQPELPYP |
| 444 | PQPFPQQPIPQQPQPY | 536 | LQPFPQPELPY |
| 445 | QQPIPQQPQPY | 537 | FPQPELPYPQPQ |
| 446 | QQPIPEQPQPY | 538 | PFPQPELPYPQP |
| 447 | QQFPQPQQPFPQ | 539 | LQPFPQPELPYP |
| 448 | QQFPQPEQPFPQ | 540 | PFPQPELPYPQPQ |
| 449 | PQQPIPQQPQPYPQQP | 541 | LQPFPQPELPYPQ |
| 450 | QQPFPQQPFPQQPQPY | 542 | QPEQPF |
| 451 | QPFPQPQQPFSW | 543 | QPEQPFP |
| 452 | QPFPQPEQPFSW | 544 | PQPEQPF |
| 453 | PQQPFPQQPQPYPQQP | 545 | QPEQPFPW |
| 454 | QPFPQPQQPIPQ | 546 | PQPEQPFP |
| 455 | QPFPQPEQPIPQ | 547 | FPQPEQPF |
| 456 | QPFPQPQQPFPQ | 548 | QPEQPFPWQ |
| 457 | QPFPQPEQPFPQ | 549 | FPQPEQPFP |
| 458 | QPFPQPQQPTPI | 550 | QPEQPFPWQP |
| 459 | QPFPQPEQPTPI | 551 | PQPEQPFPWQ |
| 460 | PAPIQPQQPFPQ | 552 | FPQPEQPFPW |
| 461 | PAPIQPEQPFPQ | 553 | PFPQPEQPFP |
| 462 | PQQPFPQQPEQI | 554 | QPFPQPEQPF |
| 463 | PQQPFPEQPEQI | 555 | PQPEQPFPWQP |
| 464 | PQQPFPQQPQQI | 556 | FPQPEQPFPWQ |
| 465 | PQQPFPEQPQQI | 557 | PFPQPEQPFPW |
| 466 | PFPQQPEQIISQ | 558 | QPFPQPEQPFP |
| 467 | PFPQQPEQIISQ | 559 | FPQPEQPFPWQP |
| 468 | PFPQQPEQIIPQ | 560 | PFPQPEQPFPWQ |
| 469 | PFPQQPEQIIPQ | 561 | PFPQPEQPFPWQP |
| 470 | QPFPQPQQQLPL | 562 | QPFPQPEQPFPWQ |
| 471 | QPFPQPEQQLPL | 563 | PIPEQPQ |
| 472 | LFPLPQQPFPQ | 564 | PIPEQPQP |
| 473 | LFPLPEQPFPQ | 565 | QPIPEQPQ |
| 474 | LQPFPQPELPYPQPQ | 566 | QPIPEQPQP |
| 475 | QPFPQPEQPFPWQP | 567 | PIPEQPQPYP |
| 476 | PEQPIPEQPQPYPQQ | 568 | QPIPEQPQPY |
| 477 | PQQPFPQPEQPFPWQP | 569 | EQPIPEQPQP |
| 478 | FPEQPIPEQPQPYPQQ | 570 | PEQPIPEQPQ |
| 479 | PEQPIPEQPQPYPQQ | 571 | PIPEQPQPYPQQ |
| 480 | PQPFLPQLPYPQ | 572 | QPIPEQPQPYPQ |
| 481 | QAFPQPQQTFPH | 573 | EQPIPEQPQPYP |
| 482 | TPIQPQQPFPQ | 574 | PEQPIPEQPQPY |
| 483 | PFPLQPQQPFPQ | 575 | QPIPEQPQPYPQQ |
| 484 | PFTQPQQPTPI | 576 | EQPIPEQPQPYPQ |
| 485 | QPFPQLQQPQQP | 577 | PEQPIPEQPQPYP |
| 486 | VAHAIIMHQQQQQQQE | 578 | EQPIPEQPQPYPQQ |
| 487 | SYPVQPQQPFPQ | 579 | PEQPIPEQPQPYPQ |
| 488 | PQQPQPFPQQPVPQQP | 580 | PDLP |
| 489 | PFPWQPQQPFPQ | 581 | PELPYPQ |
| 490 | PFPLQPQQPFPQ | 582 | QPFPQPELPYPQP |
| 491 | QQPFQPQQPFPQ | 583 | QPFPQPELPYPQPQ |
| 492 | NPLQPQQPFPLQPQPP | 584 | LQPFPQPELPYPQP |
| 493 | PLQPQQPFPLQPQPPQ | 585 | PIPEQPQPYPQ |
| 494 | PNPLQPQQPFPLQ | 586 | QPIPEQPQPYP |
| 495 | TIPQQPQQPFPL | 587 | EQPIPEQPQPY |
| 496 | SFSQQPQQPFPL | 588 | PEQPIPEQPQP |
| 497 | SFSEQPQQPFPL | | |

In some embodiments, the tolerogenic antigen is a biologically active fragment of SEQ ID NO: 474. In some instances, the biologically active fragment of SEQ ID NO: 474 includes a polypeptide comprising the sequence of SEQ ID NO: 512. In some instances, the biologically active fragment of SEQ ID NO: 474 includes a polypeptide comprising the sequence of SEQ ID NO: 580.

In some instances, the tolerogenic antigen is a biologically active fragment of SEQ ID NO: 475. In some instances, the biologically active fragment of SEQ ID NO: 475 includes a polypeptide comprising the sequence of SEQ ID NO: 542.

In some embodiments, the tolerogenic antigen is a biologically active fragment of SEQ ID NO: 476. In some instances, the biologically active fragment of SEQ ID NO: 476 includes a polypeptide comprising the sequence of SEQ ID NO: 563.

In some embodiments, the tolerogenic antigen comprises a polypeptide comprising the amino acid sequence PFPQPELPY (SEQ ID NO: 375). In some embodiments, the tolerogenic antigen comprises a polypeptide comprising the amino acid sequence PYPQPELPY (SEQ ID NO: 376). In some embodiments, the tolerogenic antigen comprises a polypeptide comprising the amino acid sequence PQPELPYPQ (SEQ ID NO: 377). In some embodiments, the tolerogenic antigen comprises a polypeptide comprising the amino acid sequence FRPEQPYPQ (SEQ ID NO: 378). In some embodiments, the tolerogenic antigen comprises a polypeptide comprising the amino acid sequence PQQSFPEQQ (SEQ ID NO: 379). In some embodiments, the tolerogenic antigen comprises a polypeptide comprising the amino acid sequence IQPEQPAQL (SEQ ID NO: 380). In some embodiments, the tolerogenic antigen comprises a polypeptide comprising the amino acid sequence QQPEQPYPQ (SEQ ID NO: 381). In some embodiments, the tolerogenic antigen comprises a polypeptide comprising the amino acid sequence SQPEQEFPQ (SEQ ID NO: 382). In some embodiments, the tolerogenic antigen comprises a polypeptide comprising the amino acid sequence PQPEQEFPQ (SEQ ID NO: 383). In some embodiments, the tolerogenic antigen comprises a polypeptide comprising the amino acid sequence QQPEQPFPQ (SEQ ID NO: 384). In some embodiments, the tolerogenic antigen comprises a polypeptide comprising the amino acid sequence PQPEQPFCQ (SEQ ID NO: 385). In some embodiments, the tolerogenic antigen comprises a polypeptide comprising the amino acid sequence QQPFPEQPQ (SEQ ID NO: 386). In some embodiments, the tolerogenic antigen comprises a polypeptide comprising the amino acid sequence PFPQPEQPF (SEQ ID NO: 387). In some embodiments, the tolerogenic antigen comprises a polypeptide comprising the amino acid sequence PQPEQPFPW (SEQ ID NO: 388). In some embodiments, the tolerogenic antigen comprises a polypeptide comprising the amino acid sequence PFSEQEQPV (SEQ ID NO: 389). In some embodiments, the tolerogenic antigen comprises a polypeptide comprising the amino acid sequence FSQQQESPF (SEQ ID NO: 390). In some embodiments, the tolerogenic antigen comprises a polypeptide comprising the amino acid sequence QQPIPEQPQ (SEQ ID NO: 391). In some embodiments, the tolerogenic antigen comprises a polypeptide comprising the amino acid sequence PQPEQPFPQ (SEQ ID NO: 392). In some embodiments, the tolerogenic antigen comprises a polypeptide comprising the amino acid sequence PIPEQPQPY (SEQ ID NO: 393). In some embodiments, the tolerogenic antigen comprises a polypeptide comprising the amino acid sequence EQPIPEQPQ (SEQ ID NO: 394). In some embodiments, the tolerogenic antigen comprises a polypeptide comprising the amino acid sequence PQPEQPFPQ (SEQ ID NO: 395). In some embodiments, the tolerogenic antigen comprises a polypeptide comprising the amino acid sequence PYPEQEEPF (SEQ ID NO: 396). In some embodiments, the tolerogenic antigen comprises a polypeptide comprising the amino acid sequence PYPEQEQPF (SEQ ID NO: 397). In some embodiments, the tolerogenic antigen comprises a polypeptide comprising the amino acid sequence PFSEQEQPV (SEQ ID NO: 398). In some embodiments, the tolerogenic antigen comprises a polypeptide comprising the amino acid sequence EGSFQPSQE (SEQ ID NO: 399). In some embodiments, the tolerogenic antigen comprises a polypeptide comprising the amino acid sequence EQPQQPFPQ (SEQ ID NO: 400). In some embodiments, the tolerogenic antigen comprises a polypeptide comprising the amino acid sequence EQPQQPYPE (SEQ ID NO: 401). In some embodiments, the tolerogenic antigen comprises a polypeptide comprising the amino acid sequence QGYYPTSPQ (SEQ ID NO: 402). In some embodiments, the tolerogenic antigen comprises a polypeptide comprising the amino acid sequence EGSFQPSQE (SEQ ID NO: 403). In some embodiments, the tolerogenic antigen comprises a polypeptide comprising the amino acid sequence PQQSFPEQE (SEQ ID NO: 404). In some embodiments, the tolerogenic antigen comprises a polypeptide comprising the amino acid sequence QGYYPTSPQ (SEQ ID NO: 405). In some embodiments, the tolerogenic antigen comprises a polypeptide comprising the amino acid sequence QPFPQPQQPFPW (SEQ ID NO: 406). In some embodiments, the tolerogenic antigen comprises a polypeptide comprising the amino acid sequence QPFPQPQQPIPV (SEQ ID NO: 407). In some embodiments, the tolerogenic antigen comprises a polypeptide comprising the amino acid sequence QPFPQPEQPFPW (SEQ ID NO: 408). In some embodiments, the tolerogenic antigen comprises a polypeptide comprising the amino acid sequence PFPQPEQPIPV (SEQ ID NO: 409). In some embodiments, the tolerogenic antigen comprises a polypeptide comprising the amino acid sequence QPFPQPELPFPQ (SEQ ID NO: 410). In some embodiments, the tolerogenic antigen comprises a polypeptide comprising the amino acid sequence LPYPQPQLPYPQ (SEQ ID NO: 411). In some embodiments, the tolerogenic antigen comprises a polypeptide comprising the amino acid sequence LPYPQPELPYPQ (SEQ ID NO: 412). In some embodiments, the tolerogenic antigen comprises a polypeptide comprising the amino acid sequence QPFPQPQLPYPQ (SEQ ID NO: 413). In some embodiments, the tolerogenic antigen comprises a polypeptide comprising the amino acid sequence QPFPQPELPYPQ (SEQ ID NO: 414). In some embodiments, the tolerogenic antigen comprises a polypeptide comprising the amino acid sequence QPFPQPQQPFSQ (SEQ ID NO: 415). In some embodiments, the tolerogenic antigen comprises a polypeptide comprising the amino acid sequence QPFPQPEQPFSQ (SEQ ID NO: 416). In some embodiments, the tolerogenic antigen comprises a polypeptide comprising the amino acid sequence QPFPQPQQPFCQ (SEQ ID NO: 417). In some embodiments, the tolerogenic antigen comprises a polypeptide comprising the amino acid sequence QPFPQPEQPFCQ (SEQ ID NO: 418). In some embodiments, the tolerogenic antigen comprises a polypeptide comprising the amino acid sequence QPFPQPQLPYSQ (SEQ ID NO: 419). In some embodiments, the tolerogenic antigen comprises a polypeptide comprising the amino acid sequence QPFPQPELPYSQ (SEQ ID NO: 420). In some embodiments, the tolerogenic antigen comprises a polypeptide comprising the amino acid sequence LQQQCSPVAMPQRLAR (SEQ ID NO: 421). In some embodiments, the tolerogenic antigen comprises a polypeptide comprising the amino acid sequence QPFPQPQLPYLQ (SEQ ID NO: 422). In some embodiments, the tolerogenic antigen comprises a polypeptide comprising the amino acid sequence QPFPQPELPYLQ (SEQ ID NO: 423). In some embodiments, the tolerogenic antigen comprises a polypeptide comprising the amino acid sequence QQFIQPQQPFPQ (SEQ ID NO: 424). In some embodiments, the tolerogenic antigen comprises a polypeptide comprising the amino acid sequence QQFIQPEQPFPQ (SEQ ID NO: 425). In some embodiments, the tolerogenic antigen comprises a polypeptide comprising the amino acid sequence LERPWQQQPLPP (SEQ ID NO: 426). In some embodiments, the tolerogenic antigen comprises a polypeptide comprising the amino acid sequence LERPWQEQPLPP (SEQ ID NO: 427). In some embodiments, the tolerogenic antigen comprises a polypeptide comprising the amino acid sequence PIPQQPEQPFPL (SEQ ID NO: 428). In some embodiments, the tolerogenic antigen comprises a polypeptide comprising the amino acid sequence QGQQGYYPISPQQSGQ (SEQ ID NO: 429). In some embodiments, the tolerogenic antigen comprises a polypeptide comprising the amino acid sequence QGQPGYYPTSPQQIGQ (SEQ ID NO: 430). In some embodiments, the tolerogenic antigen comprises a polypeptide comprising the amino acid sequence PGQGQSGYYPTSPQQS (SEQ ID NO: 431). In some embodiments, the tolerogenic antigen comprises a polypeptide comprising the amino acid sequence PQQTFPQQPQLP (SEQ ID NO: 432). In some embodiments, the tolerogenic antigen comprises a polypeptide comprising the amino acid sequence PQQTFPEQPQLP (SEQ ID NO: 433). In some embodiments, the tolerogenic antigen comprises a polypeptide comprising the amino acid sequence GQGQSGYYPTSPQQSG (SEQ ID NO: 434). In some embodiments, the tolerogenic antigen comprises a polypeptide comprising the amino acid sequence QYEVIRSLVLRTLPNM (SEQ ID NO: 435). In some embodiments, the tolerogenic antigen comprises a polypeptide comprising the amino acid sequence QVDPSGQVQWPQ (SEQ ID NO: 436). In some embodiments, the tolerogenic antigen comprises a polypeptide comprising the amino acid sequence QVDPSGEVQWPQ (SEQ ID NO: 437). In some embodiments, the tolerogenic antigen comprises a polypeptide comprising the amino acid sequence QPFPQPQQPFPL (SEQ ID NO: 438). In some embodiments, the tolerogenic antigen comprises a polypeptide comprising the amino acid sequence QPFPQPEQPFPL (SEQ ID NO: 439). In some embodiments, the tolerogenic antigen comprises a polypeptide comprising the amino acid sequence QPFPQPQQPIPY (SEQ ID NO: 440). In some embodiments, the tolerogenic antigen comprises a polypeptide comprising the amino acid sequence QPFPQPEQPIPY (SEQ ID NO: 441). In some embodiments, the tolerogenic antigen comprises a polypeptide comprising the amino acid sequence PQQPVPQQPQPY (SEQ ID NO: 442). In some embodiments, the tolerogenic antigen comprises a polypeptide comprising the amino acid sequence PQQPVPEQPQPY (SEQ ID NO: 443). In some embodiments, the tolerogenic antigen comprises a polypeptide comprising the amino acid sequence PQPFPQQPIPQQPQPY (SEQ ID NO: 444). In some embodiments, the tolerogenic antigen comprises a polypeptide comprising the amino acid sequence QQPIPQQPQPY (SEQ ID NO: 445). In some embodiments, the tolerogenic antigen comprises a polypeptide comprising the amino acid sequence QQPIPEQPQPY (SEQ ID NO: 446). In some embodiments, the tolerogenic antigen comprises a polypeptide comprising the amino acid sequence QQFPQPQQPFPQ (SEQ ID NO: 447). In some embodiments, the tolerogenic antigen comprises a polypeptide comprising the amino acid sequence QQFPQPEQPFPQ (SEQ ID NO: 448). In some embodiments, the tolerogenic antigen comprises a polypeptide comprising the amino acid sequence PQQPIPQQPQPYPQQP (SEQ ID NO: 449). In some embodiments, the tolerogenic antigen comprises a polypeptide comprising the amino acid sequence QQPFPQQPFPQQPQPY (SEQ ID NO: 450). In some embodiments, the tolerogenic antigen comprises a polypeptide comprising the amino acid sequence QPFPQPQQPFSW (SEQ ID NO: 451). In some embodiments, the tolerogenic antigen comprises a polypeptide comprising the amino acid sequence QPFPQPEQPFSW (SEQ ID NO: 452). In some embodiments, the tolerogenic antigen comprises a polypeptide comprising the amino acid sequence PQQPFPQQPQPYPQQP (SEQ ID NO: 453). In some embodiments, the tolerogenic antigen comprises a polypeptide comprising the amino acid sequence QPFPQPQQPIPQ (SEQ ID NO: 454). In some embodiments, the tolerogenic antigen comprises a polypeptide comprising the amino acid sequence QPFPQPEQPIPQ (SEQ ID NO: 455). In some embodiments, the tolerogenic antigen comprises a polypeptide comprising the amino acid sequence QPFPQPQQPFPQ (SEQ ID NO: 456). In some embodiments, the tolerogenic antigen comprises a polypeptide comprising the amino acid sequence QPFPQPEQPFPQ (SEQ ID NO: 457). In some embodiments, the tolerogenic antigen comprises a polypeptide comprising the amino acid sequence QPFPQPQQPTPI (SEQ ID NO: 458). In some embodiments, the tolerogenic antigen comprises a polypeptide comprising the amino acid sequence QPFPQPEQPTPI (SEQ ID NO: 459). In some embodiments, the tolerogenic antigen comprises a polypeptide comprising the amino acid sequence PAPIQPQQPFPQ (SEQ ID NO: 460). In some embodiments, the tolerogenic antigen comprises a polypeptide comprising the amino acid sequence PAPIQPEQPFPQ (SEQ ID NO: 461). In some embodiments, the tolerogenic antigen comprises a polypeptide comprising the amino acid sequence PQQPFPQQPEQI (SEQ ID NO: 462). In some embodiments, the tolerogenic antigen comprises a polypeptide comprising the amino acid sequence PQQPFPEQPEQI (SEQ ID NO: 463). In some embodiments, the tolerogenic antigen comprises a polypeptide comprising the amino acid sequence PQQPFPQQPQQI (SEQ ID NO: 464). In some embodiments, the tolerogenic antigen comprises a polypeptide comprising the amino acid sequence PQQPFPEQPQQI (SEQ ID NO: 465). In some embodiments, the tolerogenic antigen comprises a polypeptide comprising the amino acid sequence PFPQQPEQIISQ (SEQ ID NO: 466). In some embodiments, the tolerogenic antigen comprises a polypeptide comprising the amino acid sequence PFPQQPEQIISQ (SEQ ID NO: 467). In some embodiments, the tolerogenic antigen comprises a polypeptide comprising the amino acid sequence PFPQQPEQIIPQ (SEQ ID NO: 468). In some embodiments, the tolerogenic antigen comprises a polypeptide comprising the amino acid sequence PFPQQPEQIIPQ (SEQ ID NO: 469). In some embodiments, the tolerogenic antigen comprises a polypeptide comprising the amino acid sequence QPFPQPQQQLPL (SEQ ID NO: 470). In some embodiments, the tolerogenic antigen comprises a polypeptide comprising the amino acid sequence QPFPQPEQQLPL (SEQ ID NO: 471). In some embodiments, the tolerogenic antigen comprises a polypeptide comprising the amino acid sequence LFPLPQQPFPQ (SEQ ID NO: 472). In some embodiments, the tolerogenic antigen comprises a polypeptide comprising the amino acid sequence LFPLPEQPFPQ (SEQ ID NO: 473). In some embodiments, the tolerogenic antigen comprises a polypeptide comprising the amino acid sequence LQPFPQPELPYPQPQ (SEQ ID NO: 474). In some embodiments, the tolerogenic antigen comprises a polypeptide comprising the amino acid sequence QPFPQPEQPFPWQP (SEQ ID NO: 475). In some embodiments, the tolerogenic antigen comprises a polypeptide comprising the amino acid sequence PEQPIPEQPQPYPQQ (SEQ ID NO: 476). In some embodiments, the tolerogenic antigen comprises a polypeptide comprising the amino acid sequence PQQPFPQPEQPFPWQP (SEQ ID NO: 477). In some embodiments, the tolerogenic antigen comprises a polypeptide comprising the amino acid sequence FPEQPIPEQPQPYPQQ (SEQ ID NO: 478). In some embodiments, the tolerogenic antigen comprises a polypeptide comprising the amino acid sequence PEQPIPEQPQPYPQQ (SEQ ID NO: 479). In some embodiments, the tolerogenic antigen comprises a polypeptide comprising the amino acid sequence PQPFLPQLPYPQ (SEQ ID NO: 480). In some embodiments, the tolerogenic antigen comprises a polypeptide comprising the amino acid sequence QAFPQPQQTFPH (SEQ ID NO: 481). In some embodiments, the tolerogenic antigen comprises a polypeptide comprising the amino acid sequence TPIQPQQPFPQ (SEQ ID NO: 482). In some embodiments, the tolerogenic antigen comprises a polypeptide comprising the amino acid sequence PFPLQPQQPFPQ (SEQ ID NO: 483). In some embodiments, the tolerogenic antigen comprises a polypeptide comprising the amino acid sequence PFTQPQQPTPI (SEQ ID NO: 484). In some embodiments, the tolerogenic antigen comprises a polypeptide comprising the amino acid sequence QPFPQLQQPQQP (SEQ ID NO: 485). In some embodiments, the tolerogenic antigen comprises a polypeptide comprising the amino acid sequence VAHAIIMHQQQQQQQE (SEQ ID NO: 486). In some embodiments, the tolerogenic antigen comprises a polypeptide comprising the amino acid sequence SYPVQPQQPFPQ (SEQ ID NO: 487). In some embodiments, the tolerogenic antigen comprises a polypeptide comprising the amino acid sequence PQQPQPFPQQPVPQQP (SEQ ID NO: 488). In some embodiments, the tolerogenic antigen comprises a polypeptide comprising the amino acid sequence PFPWQPQQPFPQ (SEQ ID NO: 489). In some embodiments, the tolerogenic antigen comprises a polypeptide comprising the amino acid sequence PFPLQPQQPFPQ (SEQ ID NO: 490). In some embodiments, the tolerogenic antigen comprises a polypeptide comprising the amino acid sequence QQPFQPQQPFPQ (SEQ ID NO: 491). In some embodiments, the tolerogenic antigen comprises a polypeptide comprising the amino acid sequence NPLQPQQPFPLQPQPP (SEQ ID NO: 492). In some embodiments, the tolerogenic antigen comprises a polypeptide comprising the amino acid sequence PLQPQQPFPLQPQPPQ (SEQ ID NO: 493). In some embodiments, the tolerogenic antigen comprises a polypeptide comprising the amino acid sequence PNPLQPQQPFPLQ (SEQ ID NO: 494). In some embodiments, the tolerogenic antigen comprises a polypeptide comprising the amino acid sequence TIPQQPQQPFPL (SEQ ID NO: 495). In some embodiments, the tolerogenic antigen comprises a polypeptide comprising the amino acid sequence SFSQQPQQPFPL (SEQ ID NO: 496). In some embodiments, the tolerogenic antigen comprises a polypeptide comprising the amino acid sequence SFSEQPQQPFPL (SEQ ID NO: 497). In some embodiments, the tolerogenic antigen comprises a polypeptide comprising the amino acid sequence YSPYQPQQPFPQ (SEQ ID NO: 498). In some embodiments, the tolerogenic antigen comprises a polypeptide comprising the amino acid sequence QLPLQPQQPFPQ (SEQ ID NO: 499). In some embodiments, the tolerogenic antigen comprises a polypeptide comprising the amino acid sequence QQPQQPFPLQPQQPVP (SEQ ID NO: 500). In some embodiments, the tolerogenic antigen comprises a polypeptide comprising the amino acid sequence IIPQQPQQPFPL (SEQ ID NO: 501). In some embodiments, the tolerogenic antigen comprises a polypeptide comprising the amino acid sequence PEQIIPQQPQQP (SEQ ID NO: 502). In some embodiments, the tolerogenic antigen comprises a polypeptide comprising the amino acid sequence FLLQPQQPFSQ (SEQ ID NO: 503). In some embodiments, the tolerogenic antigen comprises a polypeptide comprising the amino acid sequence IISQQPQQPFPL (SEQ ID NO: 504). In some embodiments, the tolerogenic antigen comprises a polypeptide comprising the amino acid sequence PFPQRPQQPFPQ (SEQ ID NO: 505). In some embodiments, the tolerogenic antigen comprises a polypeptide comprising the amino acid sequence ELQPFPQPELPYPQPQ (SEQ ID NO: 506). In some embodiments, the tolerogenic antigen comprises a polypeptide comprising the amino acid sequence EQPFPQPEQPFPWQP (SEQ ID NO: 507). In some embodiments, the tolerogenic antigen comprises a polypeptide comprising the amino acid sequence EPEQPIPEQPQPYPQQ (SEQ ID NO: 508). In some embodiments, the tolerogenic antigen comprises a polypeptide comprising the amino acid sequence QLQPFPQPELPYPQPQ (SEQ ID NO: 509). In some embodiments, the tolerogenic antigen comprises a polypeptide comprising the amino acid sequence QQPFPQPEQPFPWQP (SEQ ID NO: 510). In some embodiments, the tolerogenic antigen comprises a polypeptide comprising the amino acid sequence FPEQPIPEQPQPYPQQ (SEQ ID NO: 511). In some embodiments, the tolerogenic antigen comprises a polypeptide comprising the amino acid sequence PELP (SEQ ID NO: 512). In some embodiments, the tolerogenic antigen comprises a polypeptide comprising the amino acid sequence QPELPYP (SEQ ID NO: 513). In some embodiments, the tolerogenic antigen comprises a polypeptide comprising the amino acid sequence PQPELPY (SEQ ID NO: 514). In some embodiments, the tolerogenic antigen comprises a polypeptide comprising the amino acid sequence FPQPELP (SEQ ID NO: 515). In some embodiments, the tolerogenic antigen comprises a polypeptide comprising the amino acid sequence PELPYPQP (SEQ ID NO: 516). In some embodiments, the tolerogenic antigen comprises a polypeptide comprising the amino acid sequence QPELPYPQ (SEQ ID NO: 517). In some embodiments, the tolerogenic antigen comprises a polypeptide comprising the amino acid sequence PQPELPYP (SEQ ID NO: 518). In some embodiments, the tolerogenic antigen comprises a polypeptide comprising the amino acid sequence FPQPELPY (SEQ ID NO: 519). In some embodiments, the tolerogenic antigen comprises a polypeptide comprising the amino acid sequence PFPQPELP (SEQ ID NO: 520). In some embodiments, the tolerogenic antigen comprises a polypeptide comprising the amino acid sequence PELPYPQPQ (SEQ ID NO: 521). In some embodiments, the tolerogenic antigen comprises a polypeptide comprising the amino acid sequence QPELPYPQP (SEQ ID NO: 522). In some embodiments, the tolerogenic antigen comprises a polypeptide comprising the amino acid sequence FPQPELPYP (SEQ ID NO: 523). In some embodiments, the tolerogenic antigen comprises a polypeptide comprising the amino acid sequence PFPQPELPY (SEQ ID NO: 524). In some embodiments, the tolerogenic antigen comprises a polypeptide comprising the amino acid sequence QPFPQPELP (SEQ ID NO: 525). In some embodiments, the tolerogenic antigen comprises a polypeptide comprising the amino acid sequence QPELPYPQPQ (SEQ ID NO: 526). In some embodiments, the tolerogenic antigen comprises a polypeptide comprising the amino acid sequence PQPELPYPQP (SEQ ID NO: 527). In some embodiments, the tolerogenic antigen comprises a polypeptide comprising the amino acid sequence FPQPELPYPQ (SEQ ID NO: 528). In some embodiments, the tolerogenic antigen comprises a polypeptide comprising the amino acid sequence PFPQPELPYP (SEQ ID NO: 529). In some embodiments, the tolerogenic antigen comprises a polypeptide comprising the amino acid sequence QPFPQPELPY (SEQ ID NO: 530). In some embodiments, the tolerogenic antigen comprises a polypeptide comprising the amino acid sequence LQPFPQPELP (SEQ ID NO: 531). In some embodiments, the tolerogenic antigen comprises a polypeptide comprising the amino acid sequence PQPELPYPQPQ (SEQ ID NO: 532). In some embodiments, the tolerogenic antigen comprises a polypeptide comprising the amino acid sequence FPQPELPYPQP (SEQ ID NO: 533). In some embodiments, the tolerogenic antigen comprises a polypeptide comprising the amino acid sequence PFPQPELPYPQ (SEQ ID NO: 534). In some embodiments, the tolerogenic antigen comprises a polypeptide comprising the amino acid sequence QPFPQPELPYP (SEQ ID NO: 535). In some embodiments, the tolerogenic antigen comprises a polypeptide comprising the amino acid sequence LQPFPQPELPY (SEQ ID NO: 536). In some embodiments, the tolerogenic antigen comprises a polypeptide comprising the amino acid sequence FPQPELPYPQPQ (SEQ ID NO: 537). In some embodiments, the tolerogenic antigen comprises a polypeptide comprising the amino acid sequence PFPQPELPYPQP (SEQ ID NO: 538). In some embodiments, the tolerogenic antigen comprises a polypeptide comprising the amino acid sequence LQPFPQPELPYP (SEQ ID NO: 539). In some embodiments, the tolerogenic antigen comprises a polypeptide comprising the amino acid sequence PFPQPELPYPQPQ (SEQ ID NO: 540). In some embodiments, the tolerogenic antigen comprises a polypeptide comprising the amino acid sequence LQPFPQPELPYPQ (SEQ ID NO: 541). In some embodiments, the tolerogenic antigen comprises a polypeptide comprising the amino acid sequence QPEQPF (SEQ ID NO: 542). In some embodiments, the tolerogenic antigen comprises a polypeptide comprising the amino acid sequence QPEQPFP (SEQ ID NO: 543). In some embodiments, the tolerogenic antigen comprises a polypeptide comprising the amino acid sequence PQPEQPF (SEQ ID NO: 544). In some embodiments, the tolerogenic antigen comprises a polypeptide comprising the amino acid sequence QPEQPFPW (SEQ ID NO: 545). In some embodiments, the tolerogenic antigen comprises a polypeptide comprising the amino acid sequence PQPEQPFP (SEQ ID NO: 546). In some embodiments, the tolerogenic antigen comprises a polypeptide comprising the amino acid sequence FPQPEQPF (SEQ ID NO: 547). In some embodiments, the tolerogenic antigen comprises a polypeptide comprising the amino acid sequence QPEQPFPWQ (SEQ ID NO: 548). In some embodiments, the tolerogenic antigen comprises a polypeptide comprising the amino acid sequence FPQPEQPFP (SEQ ID NO: 549). In some embodiments, the tolerogenic antigen comprises a polypeptide comprising the amino acid sequence QPEQPFPWQP (SEQ ID NO: 550). In some embodiments, the tolerogenic antigen comprises a polypeptide comprising the amino acid sequence PQPEQPFPWQ (SEQ ID NO: 551). In some embodiments, the tolerogenic antigen comprises a polypeptide comprising the amino acid sequence FPQPEQPFPW (SEQ ID NO: 552). In some embodiments, the tolerogenic antigen comprises a polypeptide comprising the amino acid sequence PFPQPEQPFP (SEQ ID NO: 553). In some embodiments, the tolerogenic antigen comprises a polypeptide comprising the amino acid sequence QPFPQPEQPF (SEQ ID NO: 554). In some embodiments, the tolerogenic antigen comprises a polypeptide comprising the amino acid sequence PQPEQPFPWQP (SEQ ID NO: 555). In some embodiments, the tolerogenic antigen comprises a polypeptide comprising the amino acid sequence FPQPEQPFPWQ (SEQ ID NO: 556). In some embodiments, the tolerogenic antigen comprises a polypeptide comprising the amino acid sequence PFPQPEQPFPW (SEQ ID NO: 557). In some embodiments, the tolerogenic antigen comprises a polypeptide comprising the amino acid sequence QPFPQPEQPFP (SEQ ID NO: 558). In some embodiments, the tolerogenic antigen comprises a polypeptide comprising the amino acid sequence FPQPEQPFPWQP (SEQ ID NO: 559). In some embodiments, the tolerogenic antigen comprises a polypeptide comprising the amino acid sequence PFPQPEQPFPWQ (SEQ ID NO: 560). In some embodiments, the tolerogenic antigen comprises a polypeptide comprising the amino acid sequence PFPQPEQPFPWQP (SEQ ID NO: 561). In some embodiments, the tolerogenic antigen comprises a polypeptide comprising the amino acid sequence QPFPQPEQPFPWQ (SEQ ID NO: 562). In some embodiments, the tolerogenic antigen comprises a polypeptide comprising the amino acid sequence PIPEQPQ (SEQ ID NO: 563). In some embodiments, the tolerogenic antigen comprises a polypeptide comprising the amino acid sequence PIPEQPQP (SEQ ID NO: 564). In some embodiments, the tolerogenic antigen comprises a polypeptide comprising the amino acid sequence QPIPEQPQ (SEQ ID NO: 565). In some embodiments, the tolerogenic antigen comprises a polypeptide comprising the amino acid sequence QPIPEQPQP (SEQ ID NO: 566). In some embodiments, the tolerogenic antigen comprises a polypeptide comprising the amino acid sequence PIPEQPQPYP (SEQ ID NO: 567). In some embodiments, the tolerogenic antigen comprises a polypeptide comprising the amino acid sequence QPIPEQPQPY (SEQ ID NO: 568). In some embodiments, the tolerogenic antigen comprises a polypeptide comprising the amino acid sequence EQPIPEQPQP (SEQ ID NO: 569). In some embodiments, the tolerogenic antigen comprises a polypeptide comprising the amino acid sequence PEQPIPEQPQ (SEQ ID NO: 570). In some embodiments, the tolerogenic antigen comprises a polypeptide comprising the amino acid sequence PIPEQPQPYPQQ (SEQ ID NO: 571). In some embodiments, the tolerogenic antigen comprises a polypeptide comprising the amino acid sequence QPIPEQPQPYPQ (SEQ ID NO: 572). In some embodiments, the tolerogenic antigen comprises a polypeptide comprising the amino acid sequence EQPIPEQPQPYP (SEQ ID NO: 573). In some embodiments, the tolerogenic antigen comprises a polypeptide comprising the amino acid sequence PEQPIPEQPQPY (SEQ ID NO: 574). In some embodiments, the tolerogenic antigen comprises a polypeptide comprising the amino acid sequence QPIPEQPQPYPQQ (SEQ ID NO: 575). In some embodiments, the tolerogenic antigen comprises a polypeptide comprising the amino acid sequence EQPIPEQPQPYPQ (SEQ ID NO: 576). In some embodiments, the tolerogenic antigen comprises a polypeptide comprising the amino acid sequence PEQPIPEQPQPYP (SEQ ID NO: 577). In some embodiments, the tolerogenic antigen comprises a polypeptide comprising the amino acid sequence EQPIPEQPQPYPQQ (SEQ ID NO: 578). In some embodiments, the tolerogenic antigen comprises a polypeptide comprising the amino acid sequence PEQPIPEQPQPYPQ (SEQ ID NO: 579). In some embodiments, the tolerogenic antigen comprises a polypeptide comprising the amino acid sequence PDLP (SEQ ID NO: 580). In some embodiments, the tolerogenic antigen comprises a polypeptide comprising the amino acid sequence PELPYPQ (SEQ ID NO: 581). In some embodiments, the tolerogenic antigen comprises a polypeptide comprising the amino acid sequence QPFPQPELPYPQP (SEQ ID NO: 582). In some embodiments, the tolerogenic antigen comprises a polypeptide comprising the amino acid sequence QPFPQPELPYPQPQ (SEQ ID NO: 583). In some embodiments, the tolerogenic antigen comprises a polypeptide comprising the amino acid sequence LQPFPQPELPYPQP (SEQ ID NO: 584). In some embodiments, the tolerogenic antigen comprises a polypeptide comprising the amino acid sequence PIPEQPQPYPQ (SEQ ID NO: 585). In some embodiments, the tolerogenic antigen comprises a polypeptide comprising the amino acid sequence QPIPEQPQPYP (SEQ ID NO: 586). In some embodiments, the tolerogenic antigen comprises a polypeptide comprising the amino acid sequence EQPIPEQPQPY (SEQ ID NO: 587). In some embodiments, the tolerogenic antigen comprises a polypeptide comprising the amino acid sequence PEQPIPEQPQP (SEQ ID NO: 588).

In some embodiments, such tolerogenic antigens include human allograft transplantation antigens. Examples of such human allograft transplantation antigens include, but are not limited to, the subunits of the various MHC class I and MHC class II haplotype proteins, and single-amino-acid polymorphisms on minor blood group antigens including RhCE, Kell, Kidd, Duffy and Ss.

In some embodiments, the tolerogenic antigen is a self antigen against which a subject (e.g., a human patient) has developed an autoimmune response or may develop an autoimmune response. Examples include proinsulin (e.g., for subjects suffering from or at risk of suffering from diabetes), collagens (e.g., for subjects suffering from or at risk of suffering from rheumatoid arthritis), and myelin basic protein (e.g., for subjects suffering from or at risk of suffering from multiple sclerosis). There are many proteins that are human autoimmune proteins, a term referring to various autoimmune diseases wherein the protein or proteins causing the disease are known or can be established by routine testing. Embodiments include testing a patient to identify an autoimmune protein and creating an antigen for use in a molecular fusion and creating immunotolerance to the protein. Embodiments include an antigen, or choosing an antigen from, one or more of the following proteins. In type 1 diabetes mellitus, several main antigens have been identified: insulin, proinsulin, preproinsulin, glutamic acid decarboxylase-65 (GAD-65), GAD-67, insulinoma-associated protein 2 (IA-2), and insulinoma-associated protein 2β (IA-2β); other antigens include ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, S100β, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5. In autoimmune diseases of the thyroid, including Hashimoto's thyroiditis and Graves' disease, main antigens include thyroglobulin (TG), thyroid peroxidase (TPO) and thyrotropin receptor (TSHR); other antigens include sodium iodine symporter (NIS) and megalin. In thyroid-associated ophthalmopathy and dermopathy, in addition to thyroid autoantigens including TSHR, an antigen is insulin-like growth factor 1 receptor. In hypoparathyroidism, a main antigen is calcium sensitive receptor. In Addison's disease, main antigens include 21-hydroxylase, 17α-hydroxylase, and P450 side chain cleavage enzyme (P450scc); other antigens include ACTH receptor, P450c21 and P450c17. In premature ovarian failure, main antigens include FSH receptor and α-enolase. In autoimmune hypophysitis, or pituitary autoimmune disease, main antigens include pituitary gland-specific protein factor (PGSF) 1a and 2; another antigen is type 2 iodothyronine deiodinase. In multiple sclerosis, main antigens include myelin basic protein, myelin oligodendrocyte glycoprotein and proteolipid protein. In rheumatoid arthritis, a main antigen is collagen II. In immunogastritis, a main antigen is H⁺, K⁺-ATPase. In pernicious angemis, a main antigen is intrinsic factor. In celiac disease, main antigens are tissue transglutaminase and gliadin. In vitiligo, a main antigen is tyrosinase, and tyrosinase related protein 1 and 2. In myasthenia gravis, a main antigen is acetylcholine receptor. In pemphigus vulgaris and variants, main antigens are desmoglein 3, 1 and 4; other antigens include pemphaxin, desmocollins, plakoglobin, perplakin, desmoplakins, and acetylcholine receptor. In bullous pemphigoid, main antigens include BP180 and BP230; other antigens include plectin and laminin 5. In dermatitis herpetiformis Duhring, main antigens include endomysium and tissue transglutaminase. In epidermolysis bullosa acquisita, a main antigen is collagen VII. In systemic sclerosis, main antigens include matrix metalloproteinase 1 and 3, the collagen-specific molecular chaperone heat-shock protein 47, fibrillin-1, and PDGF receptor; other antigens include Scl-70, U1 RNP, Th/To, Ku, Jol, NAG-2, centromere proteins, topoisomerase I, nucleolar proteins, RNA polymerase I, II and III, PM-Slc, fibrillarin, and B23. In mixed connective tissue disease, a main antigen is U1snRNP. In Sjogren's syndrome, the main antigens are nuclear antigens SS-A and SS-B; other antigens include fodrin, poly(ADP-ribose) polymerase and topoisomerase. In systemic lupus erythematosus, main antigens include nuclear proteins including SS-A, high mobility group box 1 (HMGB1), nucleosomes, histone proteins and double-stranded DNA. In Goodpasture's syndrome, main antigens include glomerular basement membrane proteins including collagen IV. In rheumatic heart disease, a main antigen is cardiac myosin. Other autoantigens revealed in autoimmune polyglandular syndrome type 1 include aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta and omega.

In some cases, the tolerogenic antigen is a foreign antigen against which a patient has developed an unwanted immune response. Examples are food antigens. Embodiments include testing a patient to identify foreign antigen and creating a molecular fusion that comprises the antigen and treating the patient to develop immunotolerance to the antigen or food. Examples of such foods and/or antigens are provided. Examples are from peanut: conarachin (Ara h 1), allergen II (Ara h 2), arachis agglutinin, conglutin (Ara h 6); from apple: 31 kDa major allergen/disease resistance protein homolog (Mal d 2), lipid transfer protein precursor (Mal d 3), major allergen Mal d 1.03D (Mal d 1): from milk: α-lactalbumin (ALA), lactotransferrin; from kiwi: actinidin (Act c 1, Act d 1), phytocystatin, thaumatin-like protein (Act d 2), kiwellin (Act d 5); from mustard: 2S albumin (Sin a 1), 11S globulin (Sin a 2), lipid transfer protein (Sin a 3), profilin (Sin a 4); from celery: profilin (Api g 4), high molecular weight glycoprotein (Api g 5); from shrimp: Pen a 1 allergen (Pen a 1), allergen Pen m 2 (Pen m 2), tropomyosin fast isoform; from wheat and/or other cereals: high molecular weight glutenin, low molecular weight glutenin, alpha- and gamma-gliadin, hordein, secalin, avenin; from strawberry: major strawberry allergy Fra a 1-E (Fra a 1), from banana: profilin (Mus xp 1). In some embodiments, the tolerogenic antigens are antigenic peptides of any one of SEQ ID Nos: 589-742 (Table 5).

TABLE 5
Tolerogenic Antigens cont.

| SEQ ID NO: | Sequence | Origin |
|---|---|---|
| 589 | Ser Gln Gln Pro Tyr Leu Gln Leu | Gliadin |
| | Gln | |
| 590 | Ala Leu Ala Leu Val Arg Met Leu Ile | ATP-binding casset transporter multidrug |
| | | resistance-associate protein 7 |
| 591 | Tyr Leu Leu Pro Arg Arg Gly Pro | HCV core |
| | Arg Leu | |
| 592 | Ala Ile Ser Pro Arg Thr Leu Asn Ala | p24 protein of HIV-1 |
| 593 | Val Met Ala Pro Arg Thr Leu Ile Leu | (HCMV) UL40 or HLA-Cw3 |
| 594 | Ser Gln Ala Pro Leu Pro Cys Val Leu | Epstein-Barr virus (EBC) BZLF-1 |
| 595 | Gln Met Arg Pro Val Ser Arg Val | Leader sequence of human HSP60 |
| | Leu | |
| 596 | His Ala Val Ser Glu Gly Thr Lys Ala | Histone H2B type 1-L |
| | Val Thr Lys Tyr Thr Ser Ser | |
| 597 | Pro Ala Glu Thr Ala Thr Pro Ala Pro | Histone H1.5 |
| | Val Glu Lys Ser Pro Ala Lys | |
| 598 | Ala Tyr Val Arg Leu Ala Pro Asp | 60S ribosomal protein L23a |
| | Tyr Asp Ala Leu Asp Val Ala Asn | |
| 599 | His Ala Val Ser Glu Gly Thr Lys Ala | Histone H2B type 1-J |
| | Val Thr Lys Tyr Thr Ser Ala | |
| 600 | Ala Val Ser Asp Gly Val Ile Lys Val | Cofilin-1 |
| | Phe Asn Asp Met Lys Val Arg | |
| 601 | His Ala Val Ser Glu Gly Thr Lys Ala | Histone H2B type 1-L |
| | Val Thr Lys Tyr Thr Ser Ser | |
| 602 | Gln Leu Leu Gln Ala Asn Pro Ile Leu | Myoson-9 |
| | Glu Ala Phe Gly Asn Ala Lys | |
| 603 | Lys Ser Ala Asp Thr Leu Trp Asp Ile | L-lactate dehydrogenase B chain |
| | Gln Lys Asp Leu Lys Asp Leu | |
| 604 | Ala Tyr Val Arg Leu Ala Pro Asp | 60S ribosomal protein L23a |
| | Tyr Asp Ala Leu Asp Val Ala Asn | |
| 605 | His Ala Val Ser Glu Gly Thr Lys Ala | Histone H2B type 1-J |
| | Val Thr Lys Tyr Thr Ser Ala | |
| 606 | Thr Gly Leu Ile Lys Gly Ser Gly Thr | Pyruvate kinase isozymes M1/M2 |
| | Ala Glu Val Glu Leu Lys Lys | |
| 607 | Val Ser Asp Gly Val Ile Lys Val Phe | Cofilin-1 |
| | Asn Asp Met Lys Val Arg Lys | |
| 608 | Ala Ser Gly Asn Tyr Ala Thr Val Ile | 60S ribosomal protein L8 |
| | Ser His Asn Pro Glu Thr Lys | |
| 609 | Thr Ala Glu Ile Leu Glu Leu Ala Gly | Histone H2A type 1-D |
| | Asn Ala Ala Arg Asp Asn Lys | |
| 610 | His Ala Val Ser Glu Gly Thr Lys Ala | Histone H2A type 1-J |
| | Val Thr Lys Tyr Thr Ser Ala | |
| 611 | Pro Ala Pro Val Glu Lys Ser Pro Ala | Histone H1.5 |
| | Lys Lys Lys Ala Thr Lys | |
| 612 | Ser Ala Asp Thr Leu Trp Asp Ile Gln | L-lactate dehydrogenase B chain |
| | Lys Asp Leu Lys Asp Leu | |
| 613 | Thr Gly Leu Ile Lys Gly Ser Gly Thr | Pyruvate kinase isozymes M1/M2 |
| | Ala Glu Val Glu Leu Lys | |
| 614 | Lys Ser Ala Asp Thr Leu Trp Gly Ile | L-lactate dehydrogenase A chain |
| | Gln Lys Glu Leu Gln Phe | |
| 615 | His Gly Ser Tyr Glu Asp Ala Val His | T-complex protein 1 subunit alpha |
| | Ser Gly Ala Leu Asn Asp | |
| 616 | Ser Asp Gly Val Ile Lys Val Phe Asn | Cofilin-1 |
| | Asp Met Lys Val Arg Lys | |
| 617 | Ala Gly Asn Leu Gly Gly Gly Val | 60S ribosomal protein L22 |
| | Val Thr Ile Glu Arg Ser Lys | |
| 618 | Ala Gln Ala Ala Ala Pro Ala Ser Val | 60S ribosomal protein L29 |
| | Pro Ala Gln Ala Pro Lys | |
| 619 | Pro Arg Lys Ile Glu Glu Ile Lys Asp | 60S ribosomal protein L38 |
| | Phe Leu Leu Thr Ala Arg | |
| 620 | Ser Glu Gly Thr Lys Ala Val Thr Lys | Histone H2B type 1-L |
| | Tyr Thr Ser Ser Lys | |
| 621 | Val Leu Lys Gln Val His Pro Asp | Histone H2B type 1-L |
| | Thr Gly Ile Ser Ser Lys | |
| 622 | Ser Trp Thr Ala Ala Asp Thr Ala Ala | HLA class 1 histocompatibility antigen, |
| | Gln Ile Thr Gln Arg | Cw-1 alpha chain |
| 623 | Phe Ile Ser Val Gly Tyr Val Asp Asp | HLA class 1 histocompatibility antigen, |
| | Thr Gln Phe Val Arg | Cw-1 alpha chain |
| 624 | Asn Ile Asp Asp Gly Thr Ser Asp | 60S ribosomal protein L27 |
| | Arg Pro Tyr Ser His Ala | |
| 625 | Val Leu Lys Gln Val His Pro Asp | Histone H2B type 1-J |
| | Thr Gly Ile Ser Ser Lys | |
| 626 | Arg Lys Thr Val Thr Ala Met Asp | Histone H4 |
| | Val Val Tyr Ala Leu Lys | |
| 627 | Ser Ala Asp Thr Leu Trp Gly Ile Gln | L-lactate dehydrogenase A chain |
| | Lys Glu Leu Gln Phe | |
| 628 | Ala Ser Ala Glu Thr Val Asp Pro Ala | Fascin |
| | Ser Leu Trp Glu Tyr | |
| 629 | Thr Val Val Asn Lys Asp Val Phe | 60S ribosomal protein L27 |
| | Arg Asp Pro Ala Leu | |
| 630 | Lys Thr Val Thr Ala Met Asp Val | Histone H4 |
| | Val Tyr Ala Leu Lys | |
| 631 | Glu Gly Ile Pro Ala Leu Asp Asn Phe | Elongation factor 2 |
| | Leu Asp Lys Leu | |
| 632 | Arg Val Thr Ile Met Pro Lys Asp Ile | Histone H3.3C |
| | Gln Leu Ala Arg | |
| 633 | Pro Val Ala Val Met Ala Glu Ser Ala | COP9 signalosome complex subunit 8 |
| | Phe Ser Phe Lys | |
| 634 | Gln Thr Val Ala Val Gly Val Ile Lys | Elongation factor 1-alpha 1 |
| | Ala Val Asp Lys | |
| 635 | Ile Leu Glu Leu Ala Gly Asn Ala Ala | Histone H2A type 1-D |
| | Arg Asp Asn Lys | |
| 636 | Gly Thr Gly Ala Ser Gly Ser Phe Lys | Histone H1.5 |
| | Leu Asn Lys | |
| 637 | Lys Gln Val His Pro Asp Thr Gly Ile | Histone H2B Type 1-J |
| | Ser Ser Lys | |
| 638 | Val Gly Gly Thr Ser Asp Val Glu Val | 60 kDa heat shock protein, mitochondrial |
| | Asn Glu Lys | |
| 639 | Asn Ser Val Val Glu Ala Ser Glu Ala | 14-3-3 protein eta |
| | Ala Tyr Lys | |
| 640 | Ala Leu Arg Tyr Pro Met Ala Val | 60S ribosomal protein L36 |
| | Gly Leu Asn Lys | |
| 641 | Ser Leu Val Ser Lys Gly Thr Leu Val | Histone H1.5 |
| | Gln Thr Lys | |
| 642 | Pro Glu Leu Ala Lys Ser Ala Pro Ala | Histone H2B type 1-L |
| | Pro Lys | |
| 643 | Ser Glu Met Glu Val Gln Asp Ala | Proliferation-associated protein 2G4 |
| | Glu Leu Lys | |
| 644 | Gln Thr Tyr Ser Thr Glu Pro Asn | 60S ribosomal protein L28 |
| | Asn Leu Lys | |
| 645 | Pro Met Phe Ile Val Asn Thr Asn Val | Macrophage migration inhibitory factor |
| | Pro Arg | |
| 646 | Ala Gly Phe Ala Gly Asp Asp Ala | Actin, cytoplasmic 1 |
| | Pro Arg | |
| 647 | Arg Val Asn Ala Gly Thr Leu Ala | von Willebrand factor A domain- |
| | Val Leu | containing protein 8 |
| 648 | Ile Gly Gln Ser Lys Val Phe Phe Arg | Myoson-9 |
| 649 | Thr Ala Glu Ile Leu Glu Leu Ala Gly | Histone H2A type 1-D |
| | Asn Ala Ala Arg Asp Asn Lys | |
| 650 | Ile Leu Glu Leu Ala Gly Asn Ala Ala | Histone H2A type 1-D |
| | Arg Asp Asn Lys | |
| 651 | Ala Leu Ala Gly Cys His Leu Glu | Polyamine-modulated factor 1-binding |
| | Asp Thr Gln Arg Lys Leu Gln Lys | protein 1 |
| | Gly | |
| 652 | Met Gln Leu Ile Thr Arg Gly Lys Gly | Isthmin-1 |
| | Ala Gly Thr Pro Asn Leu Ile | |
| 653 | Lys Met Lys Leu Arg Asn Thr Val | Taste receptor type 2 member 50 |
| | His Leu Ser Tyr Leu Thr Val | |
| 654 | Cys Arg Ala Ser Gln Thr Ile Ser Ser | Ig kappa chain V-I region OU |
| | Tyr Leu Asp Trp Tyr Gln | |
| 655 | Pro Ala Ala Leu Thr Asn Lys Gly | Intraflagellar transport protein 88 |
| | Asn Thr Val Phe Ala | homologue |
| 656 | Trp Thr Pro Gly Pro Ser Ala Gly Val | Mucin-19 |
| | Thr Gly Ile Ala | |
| 657 | Ile Leu Arg Thr Ile Gly Lys Glu Ala | Trafficking protein particle complex |
| | Phe | subunit 8 |
| 658 | Arg Ser Cys Gly Tyr Ala Cys Thr | Isthmin-1 |
| | Ala | |
| 659 | Phe Pro Asn Gly Phe Ser Phe Ile His | von Willebrand factor A, EGF, and |
| | | pentraxin domain-containing protein 1 |
| 660 | Ser His Gly Pro Tyr Ile Lys Leu Ile | Major facilitator superfamily domain- |
| | | containing protein 2A |
| 661 | Ala Gln Ala Ala Ala Pro Ala Ser Val | 60S ribosomal protein L29 |
| | Pro Ala Gln Ala Pro Lys | |
| 662 | Ala Tyr Val Arg Leu Ala Pro Asp | 60S ribosomal protein L23a |
| | Tyr Asp Ala Leu Asp Val Ala Asn | |
| | Lys | |
| 663 | Ala Tyr Val Arg Leu Ala Pro Asp | 60S ribosomal protein L23a |
| | Tyr Asp Ala Leu Asp Val Ala Asn | |
| 664 | Ala Ser Gly Asn Tyr Ala Thr Val Ile | 60S ribosomal protein L8 |
| | Ser His Asn Pro Glu Thr Lys | |
| 665 | Ala Gly Asn Leu Gly Gly Gly Val | 60S ribosomal protein L22 |
| | Val Thr Ile Glu Arg Ser Lys | |
| 666 | Pro Arg Lys Ile Glu Glu Ile Lys Asp | 60S ribosomal protein L38 |
| | Phe Leu Leu Thr Ala Arg | |
| 667 | Asn Ile Asp Asp Gly Thr Ser Asp | 60S ribosomal protein L27 |
| | Arg Pro Tyr Ser His Ala | |
| 668 | Thr Val Val Asn Lys Asp Val Phe | 60S ribosomal protein L27 |
| | Arg Asp Pro Ala Leu | |
| 669 | Ala Leu Arg Tyr Pro Met Ala Val | 60S ribosomal protein L36 |
| | Gly Leu Asn Lys | |
| 670 | Gln Thr Tyr Ser Thr Glu Pro Asn | 60S ribosomal protein L28 |
| | Asn Leu Lys | |
| 671 | Pro Glu Leu Ala Lys Ser Ala Pro Ala | Histone H2B type 1-L |
| | Pro Lys | |
| 672 | Lys Gln Val His Pro Asp Thr Gly Ile | Histone H2B type 1-J |
| | Ser Ser Lys | |
| 673 | Val Leu Lys Gln Val His Pro Asp | Histone H2B type 1-J |
| | Thr Gly Ile Ser Ser Lys | |
| 674 | Ser Glu Gly Thr Lys Ala Val Thr Lys | Histone H2B type 1-L |
| | Tyr Thr Ser Ser Lys | |
| 675 | His Ala Val Ser Glu Gly Thr Lys Ala | Histone H2B type 1-J |
| | Val Thr Lys Tyr Thr Ser Ala | |
| 676 | His Ala Val Ser Glu Gly Thr Lys Ala | Histone H2B type 1-L |
| | Val Thr Lys Tyr Thr Ser Ser | |
| 677 | His Ala Val Ser Glu Gly Thr Lys Ala | Histone H2B type 1-J |
| | Val Thr Lys Tyr Thr Ser Ala Lys | |
| 678 | His Ala Val Ser Glu Gly Thr Lys Ala | Histone H2B type 1-L |
| | Val Thr Lys Tyr Thr Ser Ser Lys | |
| 679 | His Ala Val Ser Glu Gly Thr Lys Ala | Histone H2B type 1-J |
| | Val Thr Lys Tyr Thr Ser Ala | |
| 680 | Ala Gly Phe Ala Gly Asp Asp Ala | Actin, cytoplasmic 1 |
| | Pro Arg | |
| 681 | Ser Gln Ala Pro Leu Pro Cys Val Leu | Epstein-Barr virus (EBC) BZLF-1 |
| 682 | Val Met Ala Pro Arg Thr Leu Phe | HLA-G leader peptide |
| | Leu | |
| 683 | Pro Lys Lys Thr Glu Ser His His Lys | Histone H2A type 3 |
| | Ala Lys Gly Lys | |
| 684 | Ala Ala Val Leu Glu Tyr Leu | Histone H2A type 2-B |
| 685 | Ala Gln Ala Ala Ala Pro Ala Ser Val | 60S ribosomal protein L29 |
| | Pro Ala Gln Ala Pro Lys Arg Thr Gln | |
| | Ala Pro Thr Lys Ala Ser Glu | |
| 686 | Lys Leu Glu Lys Glu Glu Glu Glu | High mobility group protein HMG- |
| | Gly Ile Ser Gln Glu Ser Ser Glu Glu | I/HMG-Y |
| | Glu Gln | |
| 687 | Gly Asp Arg Ser Glu Asp Phe Gly | Annexin Al |
| | Val Asn Glu Asp Leu Ala Asp Ser | |
| | Asp Ala Arg | |
| 688 | Val Ala Pro Glu Glu His Pro Val Leu | Actin, cytoplasmic 1 |
| | Leu Thr Glu Ala Pro Leu Asn Pro | |
| | Lys | |
| 689 | Ser Thr Ala Gly Asp Thr His Leu Gly | Heat shock cognate 71 kDa protein |
| | Gly Glu Asp Phe Asp Asn Arg | |
| 690 | Lys Val Pro Gln Val Ser Thr Pro Thr | Serum albumin |
| | Leu Val Glu Val Ser Arg | |
| 691 | Pro Asp Pro Ala Lys Ser Ala Pro Ala | Histone H2B type 1-H |
| | Pro Lys Lys Gly Ser Lys | |
| 692 | Leu Gln Ala Glu Ile Glu Gly Leu Lys | Keratin, type II cytoskeletal 8 |
| | Gly Gln Arg | |
| 693 | Pro Asp Pro Ala Lys Ser Ala Pro Ala | Histone H2B type 1-H |
| | Pro Lys | |
| 694 | Pro Glu Leu Ala Lys Ser Ala Pro Ala | Histone H2B type 1-L |
| | Pro Lys | |
| 695 | Pro Glu Pro Val Lys Ser Ala Pro Val | Histone H2B Type 1-M |
| | Pro Lys | |
| 696 | Ala Ala Pro Ala Thr Arg Ala Ala Leu | Solute carrier family 15 member 4 |
| 697 | Ser Ala Pro Ser Arg Ala Thr Ala Leu | BTB/POZ domain-containing protein |
| | | KCTD18 |
| 698 | Ile Leu Asn Phe Pro Pro Pro Pro | Caprin-2 |
| 699 | Ile Ala Pro Thr Gly His Ser Leu | Septin-6 |
| 700 | Ile Ser Pro His Gly Asn Ala Leu | ATP-dependent Clp protease ATP- |
| | | binding subunit clpx-like, mitochondrial |
| 701 | Pro Asp Pro Ala Lys Ser Ala Pro Ala | Histone H2B type 1-H |
| | Pro Lys Lys Gly Ser Lys | |
| 702 | Pro Asp Pro Ala Lys Ser Ala Pro Ala | Histone H2B type 1-H |
| | Pro Lys | |
| 703 | Pro Glu Leu Ala Lys Ser Ala Pro Ala | Histone H2B type 1-L |
| | Pro Lys | |
| 704 | Pro Glu Pro Val Lys Ser Ala Pro Val | Histone H2B type 1-M |
| | Pro Lys | |
| 705 | Val Ala Pro Glu Glu His Pro Val Leu | Actin, cytoplasmic 1 |
| | Leu Thr Glu Ala Pro Leu Asn Pro | |
| | Lys | |
| 706 | Pro Leu Leu Ala Leu Leu Ala Leu | Insulin |
| | Trp Gly Pro Asp Pro | |
| 707 | Pro Lys Thr Arg Arg Glu Ala Glu | Insulin |
| | Val Gly Gln | |
| 708 | Arg Arg Glu Ala Glu Asp Leu Gln | Insulin |
| | Gly Ser Leu | |
| 709 | Arg Arg Glu Ala Glu Asp Leu Glu | Insulin |
| | Gly Ser Leu | |
| 710 | Asp Leu Gln Val Gly Gln Val Glu | Insulin |
| | Leu Gly Gly Gly Pro | |
| 711 | Asp Leu Gln Val Gly Glu Val Glu | Insulin |
| | Leu Gly Gly Gly Pro | |
| 712 | Cys Gly Ser His Leu Val Glu Ala | Insulin |
| | Leu Tyr Leu Val Cys | |
| 713 | Met Asn Ile Leu Leu Glu Tyr Val Val | Insulin |
| | Lys Ser | |
| 714 | Asn Met Phe Thr Tyr Glu Ile Ala Pro | Insulin |
| | Val Phe Val Leu Leu Glu Tyr Val | |
| 715 | Asn Val Cys Phe Trp Tyr Ile Pro Pro | Insulin |
| | Ser Leu Arg Thr Leu Glu Asp Asn | |
| 716 | Phe Asn Gln Leu Ser Thr Gly Leu | Insulin |
| | Asp Met Val Gly Leu Ala Ala Asp | |
| | Trp | |
| 717 | Gly Arg Thr Gly Thr Tyr Ile Leu Ile | Insulin |
| | Asp Met Val Leu Asn Arg Met Ala | |
| 718 | Pro Lys Ala Ala Arg Pro Pro Val Thr | Insulin |
| | Pro Val Leu Leu Glu Lys Lys Ser | |
| 719 | Leu Leu Ala Leu Leu Ala Leu Trp | Insulin |
| | Gly Pro Asp | |
| 720 | Lys Lys Lys Lys Tyr Val Ser Ile Asp | Thyroid Stimulating Hormone Receptor |
| | Val Thr Leu Gln Gln Leu Glu Ser His | |
| | Lys Lys Lys | |
| 721 | Gly Leu Lys Met Phe Pro Asp Leu | Thyroid Stimulating Hormone Receptor |
| | Thr Lys Val Tyr Ser Thr Asp | |
| 722 | H-Pro-Arg-His-Arg-Asp-Thr-Gly-Ile- | Myelin Basic Protein |
| | Leu-Asp-Ser-Ile- Gly-Arg-Phe-NH2 | |
| 723 | H-Glu-Asn-Pro-Val-Val-His-Phe- | Myelin Basic Protein |
| | Phe-Lys-Asn-Ile-Val- Thr-Pro-Arg- | |
| | Thr-Pro-NH2 | |
| 724 | H-Ala-Ser-Asp-Tyr-Lys-Ser-Ala-His- | Myelin Basic Protein |
| | Lys-Gly-Phe-Lys- Gly-Val-Asp-NH2 | |
| 725 | H-Gly-Phe-Lys-Gly-Val-Asp-Ala- | Myelin Basic Protein |
| | Gln-Gly-Thr-Leu-Ser- Lys-Ile-Phe- | |
| | NH2 | |
| 726 | Thr Gln Gln Ile Arg Leu Gln Ala Glu | Apo E |
| | Ile Phe Gln Ala Arg | |
| 727 | Ala Gln Gln Ile Arg Leu Gln Ala Glu | Apo E |
| | Ala Phe Gln Ala Arg | |
| 728 | Thr Ala Gln Ile Arg Leu Gln Ala Glu | Apo E |
| | Ile Phe Gln Ala Arg | |
| 729 | Thr Gln Ala Ile Arg Leu Gln Ala Glu | Apo E |
| | Ile Phe Gln Ala Arg | |
| 730 | Thr Gln Gln Ala Arg Leu Gln Ala | Apo E |
| | Glu Ile Phe Gln Ala Arg | |
| 731 | Thr Gln Gln Ile Ala Leu Gln Ala Glu | Apo E |
| | Ile Phe Gln Ala Arg | |
| 732 | Gln Thr Gln Gln Ile Arg Leu Gln Ala | Apo E |
| | Glu Ile Phe Gln Ala Arg | |
| 733 | Gln Gln Ile Arg Leu Gln Ala Glu Ile | Apo E |
| | Phe Gln Ala Arg | |
| 734 | Asn Ile Asp Ala Leu Asn Glu Asn | β-lactoglobulin |
| | Lys | |
| 735 | Asn Val Leu Val Leu Asp Thr Asp | β-lactoglobulin |
| | Tyr Lys Lys | |
| 736 | Asn Thr Pro Glu Val Asp Asp Glu | β-lactoglobulin |
| | Ala Leu Glu Lys Phe Asp Lys | |
| 737 | Leu Glu Asp Ala Arg Arg Leu Lys | λ cI repressor protein |
| | Ala Ile Tyr Glu Lys Lys Lys | |
| 738 | His Ser Leu Gly Lys Trp Leu Gly His | Myelin proteolipid protein |
| | Pro Asp Lys Phe | |
| 739 | Asn Thr Trp Thr Thr Cys Gln Ser Ile | Myelin proteolipid protein |
| | Ala Phe Pro Ser Lys | |
| 740 | Ile Ala Ala Thr Tyr Asn Phe Ala Val | Myelin proteolipid protein |
| | Leu Lys Leu Met Gly Arg Gly | |
| 741 | Val His Phe Phe Lys Asn Ile Val Thr | Major basic protein |
| | Pro Arg Thr Pro | |
| 742 | Asp Glu Gly Gly Tyr Thr Cys Phe | Myelin oligodendrocyte glycoprotein |
| | Phe Arg Asp His Ser Tyr Gln | |

In some instances, the autoimmune disease is Type 1 diabetes and the tolerogenic antigen is derived from Carboxypeptidase H, Chromagranin A, Glutamate decarboxylase, Imogen-38, Insulin, Insulinoma antigen-2 and 2β, Islet-specific glucose-6-phosphatase catalytic subunit related protein (IGRP), pancreatic beta-cell antigens or Proinsulin.

In some instances, the autoimmune disease is MS and the tolerogenic antigen is derived from α-enolase, aquaporin-4, β-arrestin, myelin basic protein, myelin oligodendrocytic glycoprotein, proteolipid protein, or S100-β.

In some instances, the autoimmune disease is rheumatoid arthritis and the tolerogenic antigen is derived from citrullinated protein, collagen II, heat shock proteins, gp130-RAPS, or human cartilage glycoprotein 39.

In some instances, the autoimmune disease is systemic lupus erythematosus and the tolerogenic antigen is derived from La antigen, nucleosome histones and ribonucleoproteins (snRNP), phospholipid-β-2 glycoprotein I complex, poly(ADP-ribose) polymerase, glycoprotein gp70, or Sm antigens of U-1 small ribonucleoprotein complex.

In some instances, the autoimmune disease is scleroderma and the tolerogenic antigen is derived from fibrillarin or small nucleolar protein (snoRNP).

In some embodiments, the autoimmune disease is Graves' disease and the tolerogenic antigen is derived from thyroid stimulating factor receptor (TSH-R).

In some instances, the autoimmune disease is biliary cirrhosis and the tolerogenic antigen is derived from pyruvate dehydrogenase dihydrolipoamide acetyltransferase (PCD-E2).

In some embodiments, the autoimmune disease is alopecia areata and the tolerogenic antigen is derived from hair follicle antigens.

In some instances, the autoimmune disease is ulcerative colitis and the tolerogenic antigen is derived from human tropomyosin isoform 5 (hTM5).

In some instances, the tolerogenic antigen is derived from an antigen selected from a group consisting of 17-hydroxylase, 21-hydroxylase, ADAMTS13, Annexin A5, apoH, AQP4, aromatic acid carboxylase, Basement Membrane Collagen Type IV, BP-1, BP-2, carbonic anhydrase, carboxypeptidase H, cardiolipin, cardiolipin, chromogranin A, complement component 3, Desmoglein 3, enolase, epidermal transglutaminase, GD1a, gliadin, glutamate receptor, Glutamic acid decarboxylase, glycoproteins lib-IIIa or Ib-IX, GMCSF, gpIIb-IIIa or 1b-IX, GQ1b, GQ1b, histidine-tRNA, histones, HPA-1a, HPA-5b, HSP60, HSP70, HSP90, Hu, IA-2beta, IAPP, ICA69, IFN-gamma, IGRP, IL-1, insulin, insulinoma antigen-2, interferon omega, Jol, keratin, Kir4.1, LA, LKM-1, LKM-1, LKM-2, LKM-3, LP, major peripheral myelin protein P0, Mi-2, muscarinic acetylcholine receptor Ml, MuSK protein; hypocretin, myelin associated glycoprotein (MAG), myelin basic protein (MBP), myelin oligodendrocyte glycoprotein (MOG), myelin-associated oligodendrocytic basic protein cardiac myosin, myeloperoxidase, neurofilaments, nicotinic acetylcholine receptor, orexin, outer surface protein (OSP), p62, phosphatidylserine, proteolipid protein (PLP), pyruvate dehydrogenase, Q-type calcium channel, Ro, sc170, signal recognition peptide, SMA, soluble liver antigen, sp100, synaptogagmin, thyroglobulin, thyroid peroxidase, tissue transglutaminase, TNF-alpha, topoisomerase, transglutaminase, type XVII collagen, U1-RNP, voltage-gated calcium channels, Yo, ZnT8, β2 glycoprotein I, or β2 glycoprotein I.

Tolerogenic antigens may further include, but are not limited to, hInsB₁₀₋₁₈ (HLVEALYLV (SEQ ID NO: 743)), hIGRP₂₂₈₋₂₃₆ (LNIDLLWSV (SEQ ID NO: 744)), hIGRP₂₆₅₋₂₇₃ (VLFGLGFAI (SEQ ID NO: 745)), IGRP₂₀₆₋₂₁₄ (VYLKTNVFL (SEQ ID NO: 746)), NRP-A7 (KYNKANAFL (SEQ ID NO: 747)), NRP-14 (KYNIANVFL (SEQ ID NO: 748)), NRP-V7 (KYNKANVFL (SEQ ID NO: 749)), YAI/Db (FQDENYLYL (SEQ ID NO: 750)) and/or INS B₁₅₋₂₃ (LYLVCGERG (SEQ ID NO: 751)), as well as peptides and proteins disclosed in U.S. Publication 20050202032.

In certain aspects, a peptide antigen for use in the treatment of type 1 diabetes is GAD65₁₁₄₁₂₃, VMNILLQYVV (SEQ ID NO: 752); GAD65₅₃₆₋₅₄₅, RMMEYGTTMV (SEQ ID NO: 753); GFAP₁₄₃₋₁₅₁, NLAQTDLATV (SEQ ID NO: 754); GFAP₂₁₄₋₂₂₂, QLARQQVHV (SEQ ID NO: 755); IA-2₁₇₂₋₁₈₀, SLSPLQAEL (SEQ ID NO: 756); IA-2₄₈₂₋₄₉₀, SLAAGVKLL (SEQ ID NO: 757); IA-2₈₀₅₋₈₁₃, VIVMLTPLV (SEQ ID NO: 758); ppIAPP₅₋₁₃, KLQVFLIVL (SEQ ID NO: 759); ppIAPP₉₋₁₇, FLIVLSVAL (SEQ ID NO: 760); IGRP₁₅₂₋₁₆₀, FLWSVFMLI (SEQ ID NO: 761); IGRP₂₁₁₋₂₁₉, NLFLFLFAV (SEQ ID NO: 762); IGRP₂₁₅₋₂₂₃, FLFAVGFYL (SEQ ID NO: 763); IGRP₂₂₂₋₂₃₀, YLLLRVLNI (SEQ ID NO: 764); IGRP₂₂₈₋₂₃₆, LNIDLLWSV (SEQ ID NO: 744); IGRP₂₆₅₋₂₇₃, VLFGLGFAI (SEQ ID NO: 745); IGRP₂₉₃₋₃₀₁, RLLCALTSL (SEQ ID NO: 765); Pro-insulin_{L2-10}, ALWMRLLPL (SEQ ID NO: 766); Pro-insulin_{L3-11}, LWMRLLPLL (SEQ ID NO: 767); Pro-insulin_{L6-14}, RLLPLLALL (SEQ ID NO: 768); Pro-insulin_{B5-14}, HLCGSHLVEA (SEQ ID NO: 769); Pro-insulin_{B10-18}, HLVEALYLV (SEQ ID NO: 743); Proinsulin_{B14-22}, ALYLVCGER (SEQ ID NO: 770); Pro-insulin_{B15-24}, LYLVCGERGF (SEQ ID NO: 771); Pro-insulinB₁₇₋₂₅, LVCGERGFF (SEQ ID NO: 772); Pro-insulinB₁₈₋₂₇, VCGERGFFYT (SEQ ID NO: 773); Pro-insulin_{B20-27}, GERGFFYT (SEQ ID NO: 774); Pro-insulin_{B21-29}, ERGFFYTPK (SEQ ID NO: 775); Pro-insulin_{B25-C1}, FYTPKTRRE (SEQ ID NO: 776); Proinsulin_{B27-C5}, TPKTRREAEDL (SEQ ID NO: 777); Pro-insulin_{C20-28}, SLQPLALEG (SEQ ID NO: 778); Pro-insulin_{C25-33}, ALEGSLQKR (SEQ ID NO: 779); Pro-insulin_{C29-A5}, SLQKRGIVEQ (SEQ ID NO: 780); Pro-insulin_{A1-10}, GIVEQCCTSI (SEQ ID NO: 781); Pro-insulin_{A2-10}, IVEQCCTSI (SEQ ID NO: 782); Pro-insulin_{A12-20}, SLYQLENYC (SEQ ID NO: 783), or combinations thereof.

In still further aspects, tolerogenic antigens associated with multiple sclerosis (MS) can be used and include: MAG₂₈₇₋₂₉₅, SLLLELEEV (SEQ ID NO: 784); MAG₅₀₉₋₅₁₇, LMWAKIGPV (SEQ ID NO: 785); MAG₅₅₆₋₅₆₄, VLFSSDFRI (SEQ ID NO: 786); MBP₁₁₀₋₁₁₈, SLSRFSWGA (SEQ ID NO: 787); MOG₁₁₄₋₁₂₂, KVEDPFYWV (SEQ ID NO: 788); MOG₁₆₆₋₁₇₅, RTFDPHFLRV (SEQ ID NO: 789); MOG₁₇₂₋₁₈₀, FLRVPCWKI (SEQ ID NO: 790); MOG₁₇₉₋₁₈₈, KITLFVIVPV (SEQ ID NO: 791); MOG₁₈₈₋₁₉₆, VLGPLVALI (SEQ ID NO: 792); MOG₁₈₁₋₁₈₉, TLFVIVPVL (SEQ ID NO: 793); MOG₂₀₅₋₂₁₄, RLAGQFLEEL (SEQ ID NO: 794); PLP₈₀₋₈₈, FLYGALLLA (SEQ ID NO: 795), or combinations thereof.

In some instances, tolerogenic antigens associated with systemic lupus erythematosus can be used including, but not limited to, FIEWNKLRFRQGLEW (SEQ ID NO: 796). In some instances, the tolerogenic antigen comprising a polypeptide having the sequence of SEQ ID NO: 796 includes at least one amino acid moiety that is a D-amino acid.

### Multimeric Tolerogenic Antigens

In certain embodiments, a tolerogenic antigen provided herein is a multimeric tolerogenic antigen. In one example, a multimeric tolerogenic antigen includes two or more tolerogenic antigens (e.g., a tolerogenic antigen described herein) connected by way or a linker (e.g., a peptide linker). In some instances, the tolerogenic antigen includes the following N-terminal-to-C-terminal structure:

(P₄-L₄)n₄-(P₃-L₃)n₃-P₂-(L₁-P₁)n₁

Wherein P₁, P₂, P₃, and P₄ are each independently selected from any tolerogenic antigen described herein (e.g., any tolerogenic antigen from tables 3-5); L₁, L₃, and L₄ are each independently a linker; and n₁, n₃, and n₄ are each independently 0 or 1, wherein at least one of n₁, n₃, and n₄ are 1.

In some instances, n₁ is 1, n₃ is 0, and n₄ is 0, and the tolerogenic antigen includes the following N-terminal-to-C-terminal structure:

P₂-L₁-P₁.

In some instances, the peptide linker includes between 2 and 200 amino acids (e.g., between 5 and 50 (e.g., between 5 and 20, 15 and 30, 25 and 40, or 35 and 50), between 45 and 100 (e.g., between 45 and 60, 55 and 70, 65 and 80, 75 and 90, or 85 and 100), 95 and 150 (e.g., between 95 and 110, 105 and 120, 115 and 130, 125 and 140, or 135 and 150), or 145 and 200 amino acids (e.g., between 145 and 160, 155 and 170, 165 and 180, 175 and 190, or 185 and 200)). In some instances, the peptide linker comprises glycine (Gly) and serine (Ser) amino acids. In some instances, the peptide linker includes the amino acid sequence of any one of (GS)ₓ, (GGS)ₓ, (GGGGS (SEQ ID NO: 797))ₓ, (GGSG)ₓ, (SGGG)ₓ, wherein x is an integer from 1 to 10. In certain embodiments the linker includes the amino acid sequence of (GGGGS (SEQ ID NO: 797))ₓ, wherein x is an integer from 2-5. In some instances, P₂ and P₁ are different tolerogenic antigens. In some instances, P₂ and P₁ are identical tolerogenic antigens.

In some instances, n₁ is 1, n₃ is 1, and n₄ is 0, and the tolerogenic antigen comprises the following N-terminal-to-C-terminal structure:

P₃-L₃-P₂-L₁-P₁.

In some instances, each peptide linker independently includes between 2 and 200 amino acids (e.g., between 5 and 50 (e.g., between 5 and 20, 15 and 30, 25 and 40, or 35 and 50), between 45 and 100 (e.g., between 45 and 60, 55 and 70, 65 and 80, 75 and 90, or 85 and 100), 95 and 150 (e.g., between 95 and 110, 105 and 120, 115 and 130, 125 and 140, or 135 and 150), or 145 and 200 amino acids (e.g., between 145 and 160, 155 and 170, 165 and 180, 175 and 190, or 185 and 200)). In some instances, the peptide linker comprises glycine (Gly) and serine (Ser) amino acids. In some instances, the peptide linker includes the amino acid sequence of any one of (GS)ₓ, (GGS)ₓ, (GGGGS (SEQ ID NO: 797))ₓ, (GGSG)ₓ, (SGGG)ₓ, wherein x is an integer from 1 to 10. In certain embodiments the linker includes the amino acid sequence of (GGGGS (SEQ ID NO: 797))ₓ, wherein x is an integer from 2-5. In some instances, P₃, P₂, and/or P₁ are different tolerogenic antigens. In some instances, P₃, P₂, and/or P₁ are identical tolerogenic antigens.

In some instances, n₁ is 1, n₃ is 1, and n₄ is 1, and the tolerogenic antigen comprises the following N-terminal-to-C-terminal structure:

P₄-L₄-P₃-L₃-P₂-L₁-P₁.

In some instances, each peptide linker independently includes between 2 and 200 amino acids (e.g., between 5 and 50 (e.g., between 5 and 20, 15 and 30, 25 and 40, or 35 and 50), between 45 and 100 (e.g., between 45 and 60, 55 and 70, 65 and 80, 75 and 90, or 85 and 100), 95 and 150 (e.g., between 95 and 110, 105 and 120, 115 and 130, 125 and 140, or 135 and 150), or 145 and 200 amino acids (e.g., between 145 and 160, 155 and 170, 165 and 180, 175 and 190, or 185 and 200)). In some instances, the peptide linker comprises glycine (Gly) and serine (Ser) amino acids. In some instances, the peptide linker includes the amino acid sequence of any one of (GS)ₓ, (GGS)ₓ, (GGGGS (SEQ ID NO: 797))ₓ, (GGSG)ₓ, (SGGG)ₓ, wherein x is an integer from 1 to 10. In certain embodiments the linker includes the amino acid sequence of (GGGGS (SEQ ID NO: 797))ₓ, wherein x is an integer from 2-5. In some instances, P₄, P₃, P₂, and/or P₁ are different tolerogenic antigens. In some instances, P₄, P₃, P₂, and/or P₁ are identical tolerogenic antigens.

In some embodiments, the tolerogenic antigen is conjugated with the nanoparticle phospholipid in such a manner that facilitates strong immune tolerance upon administration to a subject (e.g., a human subject suffering from or at risk of suffering from an autoimmune disorder (e.g., MS or celiac disease)).

In some embodiments, the tolerogenic antigen is conjugated with the nanoparticle phospholipid via a thiol-reactive and reduction-insensitive linkage between the tolerogenic antigen and the nanoparticle phospholipid. Indeed, a thiol-reactive and reduction-insensitive linkage between the tolerogenic antigen and the nanoparticle phospholipid facilitates strong immune tolerance. In some embodiments, the phospholipid is e.g., N-(3-Maleimide-1-oxopropyl)-L-α-phosphatidylethanolamine.

In some embodiments, the tolerogenic antigen is conjugated with the nanoparticle phospholipid via an amine-mediated interaction. For example, in some embodiments, the amine-mediated interaction is through an amine-reactive phospholipid (e.g., N-(Succinimidyloxy-glutaryl)-L-α-phosphatidylethanolamine, Dioleoyl (DOPE-NHS)). In some embodiments, the amine-mediated interaction is through an amine-reactive phospholipid with self-immolative linkage (e.g., linkers including o-dithiobenzyl, p-dithiobenzyl, beta-dithiobenzyl carbamate moieties, 2,2-dimethyl-4-mercapto-butyric acid, or Disulfide-carbonate-based traceless linker).

In some embodiments, the number of tolerogenic antigens associated with a specific nanoparticle is any amount that facilitates strong immune tolerance upon administration to a subject (e.g., a human subject suffering from or at risk of suffering from an autoimmune disorder (e.g., MS or celiac disease). In some embodiments, the amount of tolerogenic antigens associated with a specific nanoparticle is between 1 and 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30).

Tolerogenic antigens can be prepared by a number of techniques known in the art, depending on that nature of the molecule. Polynucleotide, polypeptide, and carbohydrate antigens can be isolated from cells of the species to be treated in which they are enriched. Short peptides are conveniently prepared by amino acid synthesis. Longer proteins of known sequence can be prepared by synthesizing an encoding sequence or PCR-amplifying an encoding sequence from a natural source or vector, and then expressing the encoding sequence in a suitable bacterial or eukaryotic host cell.

In some embodiments of the composition described herein, the nanoparticle with a plurality of tolerogenic antigens includes a linker between the tolerogenic antigens and the nanoparticle. In some embodiments, the linker refers to a covalent linkage or connection between the tolerogenic antigen and a phospholipid group of the nanoparticle. In some embodiments, the N-terminus and/or C-terminus of the tolerogenic antigen is modified with a terminal cysteine residue, which is attached to a linker. In some embodiments, the N-terminus and/or C-terminus of the tolerogenic antigen is modified with a terminal C(S)ₙ polypeptide, wherein n is between 1-10 (e.g., 2, 3, 4, 5, 6, 7, 8, 9, or 10) serine residues, and wherein the terminal serine residue is attached to a linker. In some embodiments, the N-terminus and/or C-terminus of the tolerogenic antigen is modified with a terminal CSS polypeptide, which is attached to a linker. In some embodiments, the linker is a thiol reactive crosslinker. In some embodiments, the linker is a maleimide linker. In some embodiments, the linker is a pyridyl linker. In some embodiments, the N-terminus and/or C-terminus of the tolerogenic antigen is modified with a terminal cysteine residue, which is attached to a maleimide linker. In some embodiments, the N-terminus and/or C-terminus of the tolerogenic antigen is modified with a terminal cysteine residue, which is attached to a pyridyl linker. In some embodiments, the N-terminus and/or C-terminus of the tolerogenic antigen is modified with a terminal CSS polypeptide, which is attached to a maleimide linker. In some embodiments, the N-terminus and/or C-terminus of the tolerogenic antigen is modified with a terminal CSS polypeptide, which is attached to a pyridyl linker. The linker may be attached to a modified nucleoside or nucleotide (e.g., Cys and Ser) on the nucleobase or sugar moiety at a first end and to a payload, e.g., a lipid e.g., a phospholipid, at a second end.

Linkers may be chemical linkers, which are known to one of skill in the art. Linkers may alternately be peptide linkers. The linker may be of sufficient length as to not interfere with a polypeptide sequence or a lipid moiety. Examples of chemical groups that can be incorporated into the linker include, but are not limited to, alkyl, alkenyl, alkynyl, amido, amino, ether, thioether, ester, alkylene, heteroalkylene, aryl, or heterocyclyl groups, each of which can be optionally substituted. A linker may include a synthetic group derived from, e.g., a synthetic polymer (e.g., a polyethylene glycol (PEG) polymer). In some embodiments, a linker may include one or more amino acid residues, such as D- or L-amino acid residues. Further examples of useful linkers include those that contain electrophiles, such as Michael acceptors (e.g., maleimides), activated esters, electron-deficient carbonyl compounds, and aldehydes, among others, suitable for reaction with nucleophilic substituents present within antibodies, antigen-binding fragments, proteins, peptides, and small molecules, such as amine and thiol moieties.

In the present invention, a linker between multimeric tolerogenic antigens (e.g. L₁, L₃, and/or L₄) can be polypeptide including between 2 and 200 amino acids (e.g., between 5 and 50 (e.g., between 5 and 20, 15 and 30, 25 and 40, or 35 and 50), between 45 and 100 (e.g., between 45 and 60, 55 and 70, 65 and 80, 75 and 90, or 85 and 100), 95 and 150 (e.g., between 95 and 110, 105 and 120, 115 and 130, 125 and 140, or 135 and 150), or 145 and 200 amino acids (e.g., between 145 and 160, 155 and 170, 165 and 180, 175 and 190, or 185 and 200)). In some embodiments, a linker between multimeric tolerogenic antigens (e.g. L₁, L₃, and/or L₄) is a polypeptide containing at least 12 amino acids, such as 12-200 amino acids (e.g., 12-200, 12-180, 12-160, 12-140, 12-120, 12-100, 12-90, 12-80, 12-70, 12-60, 12-50, 12-40, 12-30, 12-20, 12-19, 12-18, 12-17, 12-16, 12-15, 12-14, or 12-13 amino acids) (e.g., 14-200, 16-200, 18-200, 20-200, 30-200, 40-200, 50-200, 60-200, 70-200, 80-200, 90-200, 100-200, 120-200, 140-200, 160-200, 180-200, or 190-200 amino acids). In some embodiments, a linker between multimeric tolerogenic antigens (e.g. L₁, L₃, and/or L₄) is a polypeptide containing 12-30 amino acids (e.g., 12, 13, 14, 15, 16, 17, 18, 19, 20, 21, 22, 23, 24, 25, 26, 27, 28, 29, or 30 amino acids).

Suitable peptide linkers are known in the art, and include, for example, peptide linkers containing flexible amino acid residues such as glycine and serine. In certain embodiments, a linker can contain motifs, e.g., multiple or repeating motifs, of GS, GGS, GGGGS (SEQ ID NO: 797), GGSG (SEQ ID NO: 798), or SGGG (SEQ ID NO: 799). In certain embodiments, a linker can contain 2 to 12 amino acids including motifs of GS, e.g., GS, GSGS (SEQ ID NO: 800), GSGSGS (SEQ ID NO: 801), GSGSGSGS (SEQ ID NO: 802), GSGSGSGSGS (SEQ ID NO: 803), or GSGSGSGSGSGS (SEQ ID NO: 804). In certain other embodiments, a linker can contain 3 to 12 amino acids including motifs of GGS, e.g., GGS, GGSGGS (SEQ ID NO: 805), GGSGGSGGS (SEQ ID NO: 806), and GGSGGSGGSGGS (SEQ ID NO: 807). In yet other embodiments, a linker can contain 4 to 12 amino acids including motifs of GGSG (SEQ ID NO: 808), e.g., GGSGGGSG (SEQ ID NO: 809), or GGSGGGSGGGSG (SEQ ID NO: 810). In other embodiments, a linker can contain motifs of GGGGS (SEQ ID NO: 797), e.g., GGGGSGGGGSGGGGS (SEQ ID NO:811). In certain embodiments, a linker is SGGGSGGGSGGGSGGGSGGG (SEQ ID NO: 812).

In preferred embodiments, a peptide linker (e.g., L₁, L₃, and/or L₄) is a peptide linker including the amino acid sequence of any one of (GS)ₓ, (GGS)ₓ, (GGGGS)ₓ, (GGSG)ₓ, (SGGG)ₓ, wherein x is an integer from 1 to 50 (e.g., 1-40, 1-30, 1-20, 1-10, or 1-5). In preferred embodiments, the peptide linker has the amino acid sequence (GGGGS)ₓ, wherein x is an integer selected from 1, 2, 3, 4, 5, 6, 7, 8, 9, or 10.

In some embodiments, a peptide linker contains only glycine residues, e.g., at least 4 glycine residues (e.g., 4-200, 4-180, 4-160, 4-140, 4-40, 4-100, 4-90, 4-80, 4-70, 4-60, 4-50, 4-40, 4-30, 4-20, 4-19, 4-18, 4-17, 4-16, 4-15, 4-14, 4-13, 4-12, 4-11, 4-10, 4-9, 4-8, 4-7, 4-6 or4-5 glycine residues) (e.g., 4-200, 6-200, 8-200, 10-200, 12-200, 14-200, 16-200, 18-200, 20-200, 30-200, 40-200, 50-200, 60-200, 70-200, 80-200, 90-200, 100-200, 120-200, 140-200, 160-200, 180-200, or 190-200 glycine residues). In certain embodiments, a linker has 4-30 glycine residues (e.g., 4, 5, 6, 7, 8, 9, 10, 11, 12, 13, 14, 15, 16, 17, 18, 19, 20, 21, 22, 23, 24, 25, 26, 27, 28, 29, or 30 glycine residues). In some embodiments, a linker containing only glycine residues may not be glycosylated (e.g., O-linked glycosylation, also referred to as O-glycosylation) or may have a decreased level of glycosylation (e.g., a decreased level of O-glycosylation) (e.g., a decreased level of O-glycosylation with glycans such as xylose, mannose, sialic acids, fucose (Fuc), and/or galactose (Gal) (e.g., xylose)) as compared to, e.g., a linker containing one or more serine residues.

In some embodiments, a linker containing only glycine residues may not be 0-glycosylated (e.g., O-xylosylation) or may have a decreased level of O-glycosylation (e.g., a decreased level of O-xylosylation) as compared to, e.g., a linker containing one or more serine residues.

In some embodiments, a linker containing only glycine residues may not undergo proteolysis or may have a decreased rate of proteolysis as compared to, e.g., a linker containing one or more serine residues.

In certain embodiments, a linker can contain motifs of GGGG (SEQ ID NO: 813), e.g., GGGGGGGG (SEQ ID NO: 814). GGGGGGGGGGGG (SEQ ID NO: 815), GGGGGGGGGGGGGGGG (SEQ ID NO: 816), or GGGGGGGGGGGGGGGGGGGG (SEQ ID NO: 817). In certain embodiments, a linker can contain motifs of GGGGG (SEQ ID NO: 818), e.g., GGGGGGGGGG (SEQ ID NO: 819), GGGGGGGGGGGGGGG (SEQ ID NO: 820, or GGGGGGGGGGGGGGGGGGGG (SEQ ID NO: 821). In certain embodiments, a linker is GGGGGGGGGGGGGGGGGGGG (SEQ ID NO: 822).

In other embodiments, a linker can also contain amino acids other than glycine and serine, e.g., GENLYFQSGG (SEQ ID NO:823), SACYCELS (SEQ ID NO: 824), RSIAT (SEQ ID NO: 825), RPACKIPNDLKQKVMNH (SEQ ID NO:826), GGSAGGSGSGSSGGSSGASGTGTAGGTGSGSGTGSG (SEQ ID NO: 827), AAANSSIDLISVPVDSR (SEQ ID NO: 828), or GGSGGGSEGGGSEGGGSEGGGSEGGGSEGGGSGGGS (SEQ ID NO: 829).

### Tolerogenic Antigen Variants

In certain embodiments, amino acid sequence variants of the tolerogenic antigens of the invention are contemplated. For example, it may be desirable to improve the tolerogenic antigenicity and/or other biological properties of the tolerogenic antigens. Amino acid sequence variants of a tolerogenic antigen may be prepared by introducing appropriate modifications into the nucleotide sequence encoding the tolerogenic antigen, or by peptide synthesis. Such modifications include, for example, deletions from, and/or insertions into and/or substitutions of residues within the amino acid sequences of the tolerogenic antigens. Any combination of deletion, insertion, and substitution can be made to arrive at the final construct, provided that the final construct possesses the desired characteristics, for example, inducing antigen tolerance.

In certain embodiments, tolerogenic antigen variants having one or more amino acid substitutions are provided. Conservative substitutions are shown in Table 6 under the heading of “preferred substitutions.” More substantial changes are provided in Table 6 under the heading of “exemplary substitutions,” and as further described below in reference to amino acid side chain classes. Amino acid substitutions may be introduced into a tolerogenic antigen of interest and the products screened for a desired activity, for example, retained/improved tolerogenic antigenicity.

TABLE 6
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg I | Lys; Gln; Asn | Lys |
| Asn (N) | Gln; His; Asp, Lys; Arg | Gln |
| Asp (D) | Glu; Asn | Glu |
| Cys I | Ser; Ala | Ser |
| Gln (Q) | Asn; Glu | Asn |
| Glu I | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

A useful method for identification of residues or regions of a tolerogenic antigen that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, And Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Tolerogenic antigen variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues.

In some embodiments, the tolerogenic antigen includes an amide group at the C-terminus. In certain embodiments, the tolerogenic antigen includes a pyroglutamic acid residue at the N-terminus. In another embodiment, the tolerogenic antigen includes an acetyl group at the N-terminus. In some embodiments, the tolerogenic antigen includes a pyroglutamic acid residue at the N-terminus and an amide group at the C-terminus. In some embodiments, the tolerogenic antigen includes an acetyl group at the N-terminus and an amide group at the C-terminus. In certain embodiments, the tolerogenic antigen includes an N-terminus or a C-terminus modified with a cysteine residue bound to a linker. In some embodiments, the tolerogenic antigen includes an N-terminus and a C-terminus modified with cysteine residues bound to a linker.

In some embodiments of any one of the compositions described herein, the population of tolerogenic antigens are conjugated with the nanoparticle phospholipid in such a manner that facilitates strong immune tolerance upon administration to a subject (e.g., a human subject suffering from or at risk of suffering from an autoimmune disorder e.g., MS, celiac disease, rheumatoid arthritis, primary biliary cholangitis, primary sclerosing cholangitis, MOG antibody disease, diabetes (e.g., type 1 diabetes mellitus), autoimmune diseases of the thyroid (e.g., Hashimoto's thyroiditis, Graves' disease), thyroid-associated ophthalmopathy and dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutieres syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polvarthritis/fetal and neonatal anemia, Sepsis, or inflammatory bowel disease).

In some embodiments, the plurality of tolerogenic antigens are conjugated with the nanoparticle phospholipid via a thiol-reactive and reduction-insensitive linkage between the tolerogenic antigen and the nanoparticle phospholipid. Indeed, a thiol-reactive and reduction-insensitive linkage between the tolerogenic antigen and the nanoparticle phospholipid facilitates strong immune tolerance. In some embodiments, the phospholipid is N-(3-Maleimide-1-oxopropyl)-L-α-phosphatidylethanolamine.

In some embodiments, the tolerogenic antigen is conjugated with the nanoparticle phospholipid via an amine-mediated interaction (e.g., N-(Succinimidyloxy-glutaryl)-L-α-phosphatidylethanolamine, Dioleoyl (DOPE-NHS)). In some embodiments, the amine-mediated interaction is N-(Succinimidyloxy-glutaryl)-L-α-phosphatidylethanolamine, Dioleoyl (DOPE-NHS)). In some embodiments, the amine-mediated interaction is through an amine-reactive phospholipid with self-immolative linkage (e.g., linkers including o-dithiobenzyl, p-dithiobenzyl, beta-dithiobenzyl carbamate moieties, 2,2-dimethyl-4-mercapto-butyric acid, or Disulfide-carbonate-based traceless linker).

### Nanoparticle Characterization

The nanoparticles of the present invention may be characterized for size and uniformity by any suitable analytical techniques. These include, but are not limited to, atomic force microscopy (AFM), electrospray-ionization mass spectroscopy, MALDI-TOF mass spectroscopy, LC-MS/MS, ¹³C nuclear magnetic resonance spectroscopy, high performance liquid chromatography (HPLC), size exclusion chromatography (SEC) (equipped with multi-angle laser light scattering, dual UV and refractive index detectors), capillary electrophoresis, and get electrophoresis. These analytical methods assure the uniformity of the sHDL nanoparticle population and are important in the production quality control for eventual use in in vivo applications.

In some embodiments, gel permeation chromatography (GPC), which can separate sHDL nanoparticles from liposomes and free ApoA-I mimetic peptide, is used to analyze the sHDL-Tolerogenic antigen nanoparticles. In some embodiments, the size distribution and zeta-potential are determined by dynamic light scattering (DLS) using, for example, a Malven Nanosizer instrument.

### Methods of Use

In certain embodiments, the present invention provides methods for in vivo amplification of regulatory Tregs (e.g., CD4⁺CD25^{high}Foxp3⁺) within a subject (e.g., a human subject suffering from or at risk of suffering from an autoimmune disorder) comprising administering to the subject a composition comprising one or more nanoparticles followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding Tregs within the subject. In some embodiments, the composition comprising one or more nanoparticles is associated with (e.g., complexed, conjugated, encapsulated, absorbed, adsorbed, admixed) an mTOR inhibiting agent (e.g., rapamycin or a variant thereof).

In certain embodiments, the present invention provides methods for in vivo amplification of antigen-specific regulatory Tregs (e.g., CD4⁺CD25^{high}Foxp3⁺) within a subject (e.g., a human subject suffering from or at risk of suffering from an autoimmune disorder) comprising administering to the subject a composition comprising one or more nanoparticles associated with one or more tolerogenic antigens followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding antigen-specific Tregs, wherein “antigen-specific” is specific to the one or more tolerogenic antigens associated with the comprising a nanoparticle associated with one or more tolerogenic antigens.

In certain embodiments, the present invention provides methods for facilitating a strong immune tolerance to antigens associated with an autoimmune disorder within a subject (e.g., a human subject suffering from or at risk of suffering from an autoimmune disorder) comprising administering to the subject a composition comprising one or more nanoparticles followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding Tregs (e.g., CD4⁺CD25^{high}Foxp3⁺) within the subject. In some embodiments, the composition comprising one or more nanoparticles is associated with (e.g., complexed, conjugated, encapsulated, absorbed, adsorbed, admixed) an mTOR inhibiting agent (e.g., rapamycin or a variant thereof).

In certain embodiments, the present invention provides methods for facilitating a strong immune tolerance to antigens associated with an autoimmune disorder within a subject (e.g., a human subject suffering from or at risk of suffering from an autoimmune disorder) comprising administering to the subject a composition comprising one or more nanoparticles associated with one or more tolerogenic antigens followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding antigen-specific Tregs (e.g., CD4⁺CD25^{high}Foxp3⁺), wherein “antigen-specific” is specific to the one or more tolerogenic antigens associated with the comprising a nanoparticle associated with one or more tolerogenic antigens.

In certain embodiments, the present invention provides methods for increasing the ratio of CD3+FOXP3+ cells to CD3+FOXP3− cells within a population of T cells within a subject (e.g., a human subject suffering from or at risk of suffering from an autoimmune disorder) comprising administering to the subject a composition comprising one or more nanoparticles followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding Tregs (e.g., CD4⁺CD25^{high}Foxp3⁺) within the subject. In some embodiments, the composition comprising one or more nanoparticles is associated with (e.g., complexed, conjugated, encapsulated, absorbed, adsorbed, admixed) an mTOR inhibiting agent (e.g., rapamycin or a variant thereof).

In certain embodiments, the present invention provides methods for increasing the ratio of CD3+FOXP3+ cells to CD3+FOXP3− cells within a population of T cells within a subject (e.g., a human subject suffering from or at risk of suffering from an autoimmune disorder) comprising administering to the subject a composition comprising one or more nanoparticles associated with one or more tolerogenic antigens followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding antigen-specific Tregs (e.g., CD4⁺CD25^{high}Foxp3⁺), wherein “antigen-specific” is specific to the one or more tolerogenic antigens associated with the comprising a nanoparticle associated with one or more tolerogenic antigens.

In certain embodiments, the present invention provides methods for treating, preventing and/or attenuating a disorder comprising administering to a subject (e.g., a human subject suffering from or at risk of suffering from an autoimmune disorder e.g., celiac disease) comprising administering to the subject a composition comprising one or more nanoparticles followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding Tregs (e.g., CD4⁺CD25^{high}Foxp3⁺) within the subject. In some embodiments, the composition comprising one or more nanoparticles is associated with (e.g., complexed, conjugated, encapsulated, absorbed, adsorbed, admixed) an mTOR inhibiting agent (e.g., rapamycin or a variant thereof). In some embodiments, the treating, preventing, and/or attenuating one or more autoimmune disorders in the subject is specific to a specific tissue region (e.g., a specific tissue region associated with the autoimmune disorder).

In certain embodiments, the present invention provides methods for treating, preventing and/or attenuating a disorder comprising administering to a subject (e.g., a human subject suffering from or at risk of suffering from an autoimmune disorder e.g., celiac disease) a composition comprising one or more nanoparticles associated with one or more tolerogenic antigens (e.g., one or more tolerogenic antigens associated with the autoimmune disorder) followed by (e.g., after 1 second, 2 seconds, 1 minute, 1 hour, 1 day, 1 week, 1 month, 1 year, etc.) administering to the subject a composition comprising one or more immunomodulatory agents (e.g., human cytokine (e.g., an IL-2, an IL-2 mutein, an IL-2 variant, or an IL-2/IC)) capable of expanding antigen-specific Tregs (e.g., CD4⁺CD25^{high}Foxp3⁺), wherein “antigen-specific” is specific to the one or more tolerogenic antigens associated with the comprising a nanoparticle associated with one or more tolerogenic antigens. In some embodiments, the treating, preventing, and/or attenuating one or more disorders in the subject is specific to a specific tissue region (e.g., a specific tissue region associated with the disorder).

The immune system can be classified into 101unctionnal subsystems: the innate and the acquired immune system. The innate immune system is the first line of defense against infections, and most potential pathogens are rapidly neutralized by this system before they can cause, for example, a noticeable infection. The acquired immune system reacts to molecular structures, referred to as antigens, of the intruding organism. There are two types of acquired immune reactions, which include the humoral immune reaction and the cell-mediated immune reaction. In the humoral immune reaction, antibodies secreted by B cells into bodily fluids bind to pathogen-derived antigens, leading to the elimination of the pathogen through a variety of mechanisms, e.g. complement-mediated lysis. In the cell-mediated immune reaction, T-cells capable of destroying other cells are activated. For example, if proteins associated with a disease (e.g., MS or celiac disease) are present in a cell, they are fragmented proteolytically to peptides within the cell. Specific cell proteins then attach themselves to the antigen or peptide formed in this manner and transport them to the surface of the cell, where they are presented to the molecular defense mechanisms, in particular T-cells, of the body. Cytotoxic T cells recognize these antigens and kill the cells that harbor the antigens.

The molecules that transport and present peptides on the cell surface are referred to as proteins of the major histocompatibility complex (MHC), which is known as the human leukocyte antigen (HLA) complex in humans. MHC proteins are classified into two types, referred to as MHC class I and MHC class II. The structures of the proteins of the two MHC classes are very similar; however, they have very different functions. Proteins of MHC class I are present on the surface of almost all cells of the body, including most tumor cells. MHC class I proteins are loaded with antigens that usually originate from endogenous proteins or from pathogens present inside cells and are then presented to I or cytotoxic T-lymphocytes (CTLs). MHC class II proteins are present on dendritic cells, B-lymphocytes, macrophages, and other antigen-presenting cells. They mainly present peptides, which are processed from external antigen sources, i.e. outside of the cells, to T-helper (Th) cells. Most of the peptides bound by the MHC class I proteins originate from cytoplasmic proteins produced in the healthy host cells of an organism itself and do not normally stimulate an immune reaction. Accordingly, cytotoxic T-lymphocytes that recognize such self-peptide-presenting MHC molecules of class I are deleted in the thymus (central tolerance) or, after their release from the thymus, are deleted or inactivated, i.e. tolerized (peripheral tolerance). MHC molecules are capable of stimulating an immune reaction when they present peptides to non-tolerized T-lymphocytes. Cytotoxic T-lymphocytes have both T-cell receptors (TCR) and CD8 molecules on their surface. T-Cell receptors are capable of recognizing and binding peptides complexed with the molecules of MHC class I. Each cytotoxic T-lymphocyte expresses a unique T-cell receptor which is capable of binding specific MHC/peptide complexes.

Experiments conducted during the course of developing embodiments for the present invention resulted in the development of a new strategy for inducing high frequency of antigen-specific Tregs in vivo without ex vivo manipulation of cells. Such experiments resulted in the development and optimization of a new strategy of combining nanodiscs with modified IL-2 for eliciting unprecedented level of antigen-specific Tregs in vivo. Notably, IL-2:anti-IL-2 antibody (clone: JES6-1) immune complex (IL-2/IC) has been shown to selectively induce polyclonal Tregs [14]. IL-2/IC administered together with free peptide or peptide-tetramer have been reported to induce antigen-specific Tregs [15,16]. However, these previous attempts resulted in rather poor antigen-specific Treg response with less than 0.25% antigen-specific Treg frequency among CD4+ T cell compartment [15,16].

It was envisioned that it would be crucial to properly deliver peptide antigens to lymphoid tissues to maximize antigen-specific Treg induction together with IL-2/IC. Experiments described herein report for the first time that lymph-targeting nanodisc-mediated delivery of peptide in combination with IL-2/IC therapy resulted in remarkable amplification of antigen-specific Tregs, compared with IL-2/IC alone.

It was also envisioned that the actual method of treatment is crucial. Such experiments demonstrated that nanodiscs should be administered subcutaneously first, followed by systemic administration of IL-2 and/or mutein/engineered IL-2. This ensured that antigen-specific Tregs were primed and generated first, and the subsequent administration of mutein/engineered IL-2 triggered robust proliferation of antigen-specific Tregs. Administration of maintenance doses of nanodiscs and/or mutein/engineered IL-2 for long-term maintenance of antigen-specific Tregs was further envisioned.

Such methods are not limited to treating a particular disorder.

In some embodiments, the disorder is an autoimmune disorder. Such methods are not limited to treating a particular autoimmune disorder. Examples of autoimmune disorders include, but are not limited to, multiple sclerosis (MS), celiac disease, rheumatoid arthritis, primary biliary cholangitis, primary sclerosing cholangitis, MOG antibody disease, diabetes (e.g., type 1 diabetes mellitus), autoimmune diseases of the thyroid (e.g., Hashimoto's thyroiditis, Graves' disease), thyroid-associated ophthalmopathy and dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutieres syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polyarthritis/fetal and neonatal anemia, Sepsis, and inflammatory bowel disease.

In some embodiments, the disorder is a transplantation related disorder. In some embodiments, the disorder is one or more allergies. In some embodiments, the disorder is a respiratory condition (e.g., asthma). In some embodiments, the disorder is graft-versus-host-disease (GvHD).

In some embodiments, such methods for treating or preventing autoimmune disorders further comprise co-administering (e.g., simultaneously or at different times) additional therapeutic agents. Examples of such therapeutic agents include, but are not limited to, disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), glucocorticoids (e.g., prednisone, methylprednisone), TNF-α inhibitors (e.g., adalimumab, certolizumab pegol, etanercept, golimumab, infliximab), IL-1 inhibitors, and metalloprotease inhibitors. In some embodiments, the therapeutic agents include, but are not limited to, infliximab, adalimumab, etanercept, or parenteral gold or oral gold. In some instances, the therapeutic agent is an immunomodulatory agent or immunosuppressant (e.g., statins; mTOR inhibitors, such as rapamycin or a rapamycin analog; TGF-β signaling agents; TGF-β receptor agonists; histone deacetylase inhibitors, such as Trichostatin A; corticosteroids; inhibitors of mitochondrial function, such as rotenone; P38 inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors, such as TGX-221; autophagy inhibitors, such as 3-Methyladenine; aryl hydrocarbon receptor inhibitors; proteasome inhibitor I (PSI); and oxidized ATPs, such as P2X receptor blockers. Immunosuppressants also include IDO, vitamin D3, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol, triptolide; OPN-305, OPN-401; Eritoran (E5564); TAK-242; Cpn10; NI-0101; 1A6; AV411; IRS-954 (DV-1079); IMO-3100; CPG-52363; CPG-52364; OPN-305; ATNC05; NI-0101; IMO-8400; Hydroxychloroquine; CU-CPT22; C29; Ortho-vanillin; SSL3 protein; OPN-305; 5 SsnB; Vizantin; (+)-N-phenethylnoroxymorphone; VB3323; Monosaccharide 3; (+)-Naltrexone and (+)-naloxone; HT52; HTB2; Compound 4a; CNT02424; TH1020; INH-ODN; E6446; AT791; CpG ODN 2088; ODN TTAGGG; COV08-0064; 2R9; GpG oligonucleotides; 2-aminopurine; Amlexanox; Bay11-7082; BX795; CH-223191; Chloroquine; CLI-095; CU-CPT9a; Cyclosporin A; CTY387; Gefitnib; Glybenclamide; H-89; H-131; Isoliquiritigenin; MCC950; MRT67307; OxPAPC; Parthenolide; Pepinh-MYD; Pepinh-TRIF; Polymyxin B; R406; RU.521; VX-765; YM201636; Z-VAD-FMK; and AHR-specific ligands; including but not limited to 2,3,7,8-tetrachloro-dibenzo-p-dioxin (TCDD); tryptamine (TA); and 6 formylindolo[3,2 b]carbazole (FICZ)). In particular embodiments, the immunosuppressant is fingolimod; rapamycin; 2-(1′H-indole-3′-carbonyl)-thiazole-4-carboxylic acid methyl ester (ITE) or related ligands; Trichostatin A; and/or Suberoylanilide hydroxamic acid (SAHA).

Such methods are not limited to a particular manner of administering the composition comprising nanoparticles associated or not associated with tolerogenic antigens and/or the composition comprising an immunomodulatory agent (e.g., human cytokine (e.g., an IL-2, an IL-2 mutein, an IL-2 variant, or an IL-2/IC)). Indeed, any acceptable method known to one of ordinary skill in the art may be used to administer either composition to the subject. The administration may be localized (i.e., to a particular region, physiological system, tissue, organ, or cell type) or systemic. Such compositions can be administered by a number of routes including, but not limited to oral, inhalation (nasal or pulmonary), intravenous, intraperitoneal, intramuscular, transdermal, subcutaneous, topical, subcutaneous, sublingual, or rectal means. Injections can be e.g., intravenous, intradermal, subcutaneous, intramuscular, or intraperitoneal. In some embodiments, the injections can be given at multiple locations.

Administration of the formulations may be accomplished by any acceptable method which allows an effective amount of either of the compositions to achieve its desired effect. The particular mode selected will depend upon factors such as the particular formulation, the severity of the state of the subject being treated, and the dosage required to induce an effective immune response. As generally used herein, an “effective amount” is that amount which is able to induce in vivo amplification of antigen-specific regulatory Tregs (e.g., CD4⁺CD25^{high}Foxp3⁺), and/or facilitate a strong immune tolerance to antigens associated with an autoimmune disorder, and/or induce an immune response in the treated subject. The actual effective amounts of either of such compositions can vary according to the specific antigen or combination thereof being utilized, the particular composition formulated, the mode of administration, and the age, weight, condition of the individual being vaccinated, as well as the route of administration and the disease or disorder.

### Pharmaceutical Compositions

Where clinical applications are contemplated, in some embodiments of the present invention, the compositions comprising nanoparticles associated or not associated with tolerogenic antigens and/or the compositions comprising one or more immunomodulatory agents (e.g., human cytokine (e.g., an IL-2, an IL-2 mutein, an IL-2 variant, or an IL-2/IC)) are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a straight composition comprising nanoparticles associated or not associated with tolerogenic antigens and/or composition comprising an immunomodulatory agent (e.g., human cytokine (e.g., an IL-2, an IL-2 mutein, an IL-2 variant, or an IL-2/IC)) may be administered using one or more of the routes described herein.

In preferred embodiments, the compositions are used in conjunction with appropriate salts and buffers to render delivery of the compositions in a stable manner to allow for uptake by target cells. Buffers also are employed when either of the compositions are introduced into a patient.

Aqueous compositions comprise an effective amount of the sHDL nanoparticles to cells dispersed in a pharmaceutically acceptable carrier or aqueous medium. Such compositions also are referred to as inocula.

The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with the vectors or cells of the present invention, its use in therapeutic compositions is contemplated. Supplementary active ingredients may also be incorporated into the compositions.

In some embodiments of the present invention, the active compositions include classic pharmaceutical preparations. Administration of these compositions according to the present invention is via any common route so long as the target tissue is available via that route. This includes oral, nasal, buccal, rectal, vaginal, subcutaneous, or topical. Alternatively, administration may be by orthotopic, intradermal, subcutaneous, intramuscular, intraperitoneal, or intravenous injection.

The active compositions may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating either of the compositions in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, either of the compositions are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous, and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present invention, the active particles or agents are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used. Suppositories are solid dosage forms of various weights and shapes, usually medicated, for insertion into the rectum, vagina, or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications are available in a variety of physical forms, e.g., creams, gels, or liquids, which depart from the classical concept of suppositories. The compositions also may be formulated as inhalants.

### Kits

In some embodiments, the present invention also provides kits comprising a composition comprising one or more nanoparticles associated or not associated with one or more tolerogenic antigens and/or a composition comprising one or more immunomodulatory agents (e.g., human cytokine (e.g., an IL-2, an IL-2 mutein, an IL-2 variant, or an IL-2/IC)) as described herein. In some embodiments, the kits comprise one or more of the reagents and tools necessary to generate either composition, and methods of using either of such compositions.

### EXAMPLES

The following example is provided in order to demonstrate and further illustrate certain preferred embodiments and aspects of the present invention and are not to be construed as limiting the scope thereof. Use of pronouns such as, “we”, “our,” and “I” refer to the inventive entity.

### Example I

### Preparation of Antigen-Loaded Nanodiscs:

DMPC(1,2-dimyristoyl-sn-glycero-3-phosphocholine) and DOPE-MAL (N-(3-Maleimide-1-oxopropyl)-L-α-phosphatidylethanolamine) were purchased from NOF AMERICA CORPORATION. DMPC and ApoA1-mimetic peptide 22A (PVLDLFRELLBELLEALKQKLK) powder were mixed and hydrated in 10 mM sodium phosphate buffer (DMPC: 22A=2:1, mass ratio), which was then subjected to heating and cooling cycles to obtain blank synthetic HDL Nanodisc, followed by sonication for 5 min at room temperature. To load antigen peptide into blank Nanodisc, cysteine-terminated antigen peptide was first conjugated with DOPE-MAL (antigen peptide:DOPE-MAL=2:1, molar ratio). Then DOPE-peptide was added to blank Nanodisc (22A:antigen peptide=5:1, mass ratio) and incubated at room temperature with gentle shaking on an orbital shaker for 1 h. Unreacted antigen peptides were removed by using Zeba Spin desalting columns (Pierce). Antigen peptide used in this study was OVA-II (OVA323-339) peptide, CSS-ISQAVHAAHAEINEAGR. The peptide loading efficiency was measured by LC-MS. Nanodisc-OVA-II was analyzed by gel permeation chromatography (GPC) equipped with TSKgel G3000SWxl column (7.8 mm ID×30 cm, Tosoh Bioscience LLC). The hydrodynamic sizes and zeta potentials of Nanodisc samples were measured by dynamic light scattering (DLS, Zetasizer Nano ZSP).

### Preparation of Mouse IL-2:Anti-IL-2 Immune Complex (QL-2/IC):

IL-2/anti-IL-2 mAb immune complex (IL-2/IC) was prepared as previously reported [15]. 0.1 mg anti-IL-2 mAb (JES6-1A12, InVivoMAb, BioX-Cell, Lebanon, NH) was mixed in vitro with recombinant murine IL-2 (20 μg, PeproTech, Rocky Hill, NJ) in 2 mL HBSS (Mediatech, Herndon, VA) and incubated at room temperature for 10 min. Then the resulting IL-2/IC was administered in 0.1 mL single injection dose containing 1 μg murine IL-2 and 5 g IL-2 mAb.

### Animal Study

Animals were cared for following federal, state, and local guidelines. All work performed on animals was in accordance with and approved by Institutional Animal Care & Use Committee (IACUC) at University of Michigan, Ann Arbor. Seven-week-old female C57BL/6 mice were randomly divided into 5 groups with 5 mice in each group. On Day 0, Day 7 and Day 14, mice in groups 1, 2 and 3 were injected subcutaneously with 0.1 mL Nanodisc-OVA-II (contain 0.1 mg OVA-II peptide) at the tail base. In addition, group 1 received intraperitoneal (i.p.) administration of 0.1 mL as-prepared IL-2/IC solution (described above) on days 1, 2, and 3 after each Nanodisc-OVA-II injection. Group 2 received intraperitoneal (i.p.) administration of 0.1 mL as-prepared IL-2/IC solution (described above) on days 3, 4, and 5 after each Nanodisc-OVA-II injection. Group 3 mice did not receive IL-2/IC. Group 4 mice received IL-2/IC alone on days 1, 2, and 3 for week 0, 1, and 2 (similar to the group 1 without Nanodisc-OVA-II injection). Group 5 received PBS. On Day 7, 14, 21 and 28, peripheral blood was collected for flow cytometry analysis.

### Flow Cytometry Analysis

At indicated time points, 150-200 uL of blood was collected from individual mice into EDTA coated tubes. The blood volumes were transferred to 1.5 mL Eppendorf tubes for red blood cell lysis. Red blood cells were lysed by adding 1 mL ACK lysis buffer to each blood volume, and tubes were allowed to shake at room temperature for five minutes. After five minutes, the tubes were spun at 600 g for 5 minutes and supernatants were discarded. Pellets were suspended again in 1 mL of ACK lysis buffer and immediately spun at 600 g for 5 minutes. Supernatants were discarded, and PBMCs were re-suspended in PBS and transferred to a 96 well plate and washed once with PBS before proceeding to FACS staining. For surface staining, cells were first incubated with eBioscience Fixable Viability Dye eFluor450 in PBS for 10 minutes at room temperature in the dark. PBMCs were washed with 200 uL PBS and spun at 600 g for five minutes. Supernatants were discarded, and the samples were suspended in Fc Block (anti-CD16/32) in FACS buffer (PBS+1% BSA). Blocking was allowed to proceed for 10 minutes at room temperature in the dark before OT-II tetramer was added to the wells at a 1:40 final dilution in FACS buffer. OT-II tetramer (NIH Tetramer Core Facility, Atlanta, GA) was allowed to incubate with PBMCs for 1 hour at room temperature in the dark. 20 minutes prior to the end of the OT-II tetramer incubation, surface antibodies were added to the samples at a 1:100 final dilution. Following tetramer and surface incubation, PBMCs were washed with 200 uL FACs buffer and spun at 600 g for 5 minutes. For Foxp3 intracellular staining, eBioscience™ Foxp3/Transcription Factor Staining Buffer Set was used according to the kit's instructions. To measure total cell counts, 50 uL of Life Technologies Absolute Counting Beads were added to fully stained PBMC samples prior to collection on the BioRad ZE5 Analyzer. FSC files were analyzed using FlowJo.

### Characterization of Antigen-Loaded Nanodiscs:

### Synthesis and Characterization of HDL Nanodisc-OVA-II

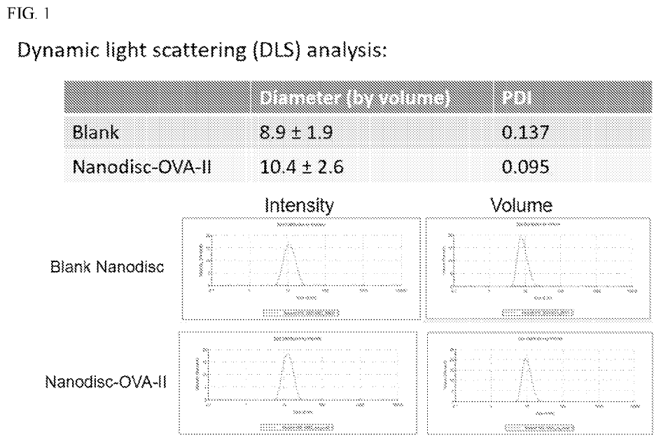

Blank HDL Nanodisc exhibited an average diameter of 8.9±2.1 nm, (PDI=0.137), while OVA-II-loaded HDL Nanodisc exhibited an average diameter of 10.4±2.6 nm (PDI=0.095, FIG. 1). The loading efficiency of OVA-II in sHDL Nanodisc was ˜99% as quantified by LC-MS (FIG. 2). And the as-synthesized blank Nanodisc and Nanodisc-OVA-II was further characterized by Gel permeation chromatography (GPC) analysis, Nanodisc-OVA-II showed a single uniform peak with a shorter retention time in GPC analysis (0.8 mL/min, PBS as mobile phase, column: TSKgel G3000SWxl, TOSOH Bioscience).

### Analysis of Antigen-Specific T Cells in Mice:

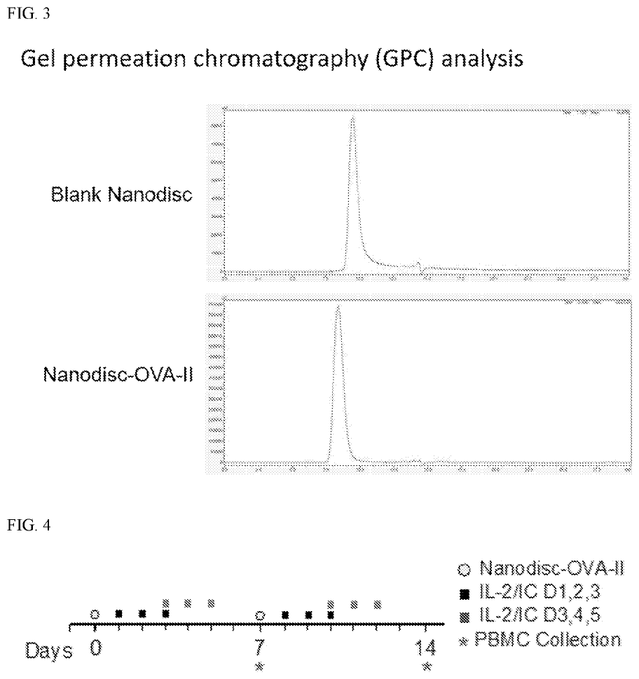

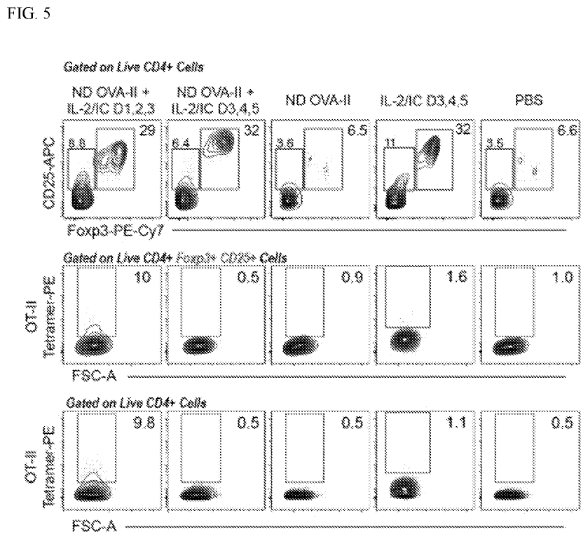

C57BL/6 mice were administered s.c. with Nanodisc-OVA-II (ND-OVA-II) and treated with IL-2/IC via i.p. route using the regimen shown in FIG. 4. Control groups included either ND-OVA-II, IL-2/IC, or PBS treatments alone. Systemic immune responses were measured by flow cytometry on days 7 and 14 post initial ND-OVA-II administration.

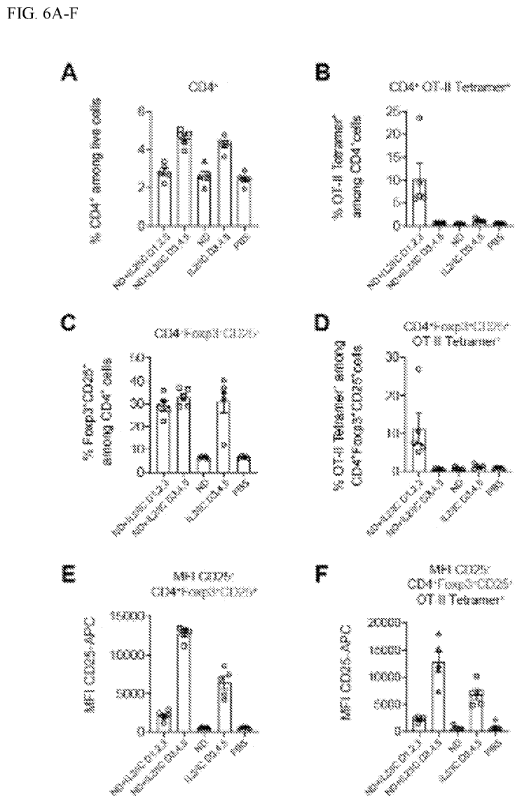

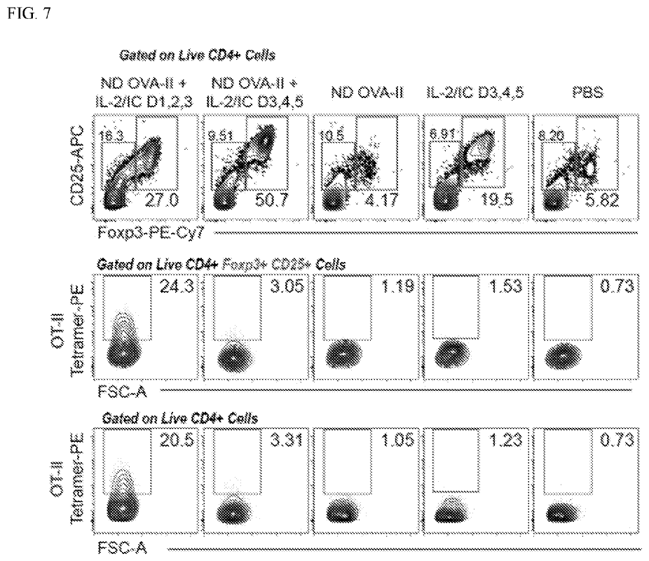

By day 7, animals in groups 2 and 4 that received ND-OVA-II in combination with IL-2/IC on days 3, 4, and 5 (D3,4,5) or IL-2/IC exhibited increased frequency of CD4⁺ T cells among PBMCs (FIG. 5, FIG. 6a). Animals in group 1 that received ND-OVA-IT in combination with IL-2/IC on days 1, 2, and 3 (D1,2,3) induced significantly higher frequency of OT-II Tetramer⁺ antigen-specific CD4⁺ T cells (FIG. 5, FIG. 6b). Mice in groups 1, 2, and 4 that received ND-OVA-II in combination with IL-2/IC or IL-2/IC alone exhibited a 5-fold expansion of total CD4⁺CD25⁺Foxp3⁺ T regulatory cells (T_{regs}), compared to mice in groups 3 and 5 treated with ND-OVA-IT or PBS (FIG. 6c). Importantly, ND-OVA-II+IL-2/IC (D1,2,3) induced robust expansion of OT-II Tetramer⁺CD25⁺Foxp3⁺ T_{regs} (FIG. 6d); these OVA-specific T_{regs} represented about 10% of total T_{regs}. Although mice in group 2 that received ND-OVA-II+IL-2/IC (D3,4,5) treatments expanded fewer antigen-specific T_{regs} (FIG. 6d), these cells showed significantly higher expression of CD25 among total CD25mFoxp3⁺ T_{regs} and OT-IT Tetramer⁺CD25⁺Foxp3⁺ T_{regs} (FIG. 6e,f), compared with T_{regs} induced by ND-OVA-II+IL-2/IC (D1,2,3) or IL-2/IC alone.

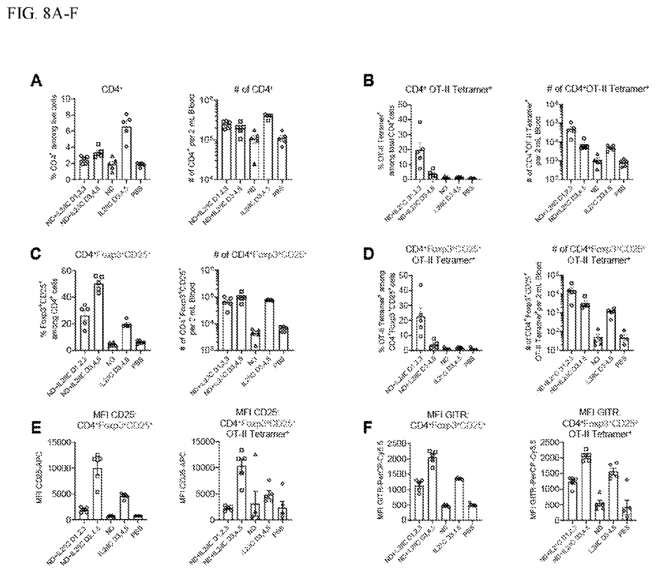

By day 14, mice that received IL-2/IC (D1,2,3) had increased CD4⁺ T cells among PBMCs, compared with other groups (FIG. 7, FIG. 8a). ND-OVA-II in combination with IL-2/IC on days 3, 4, and 5 (D3,4,5) or IL-2/IC exhibited increased frequency of CD4⁺ T cells among PBMCs (FIG. 7, FIG. 8a). Like day 7, animals treated with ND-OVA-II+IL-2/IC (D1,2,3) showed further expansion of antigen-specific OT-II Tetramer⁺CD4⁺ T cells, reaching ˜20% of CD4⁺ T cells (FIG. 7, FIG. 8b). Total T_{regs} were increased by 5-fold and 10-fold in ND-OVA-II+IL-2/IC (D1,2,3) and ND-OVA-II+IL-2/IC (D3,4,5), respectively, compared to ND-OVA-II and PBS controls (FIG. 7, 8c). Like day 7, animals treated with ND-OVA-II+IL-2/IC (D1,2,3) showed further expansion of antigen-specific T_{regs}, with now ˜22% of total T_{regs} staining positive for OT-II Tetramer (FIG. 8d).

For day 14 samples, in addition to assessing cell population frequencies, Life Technologies Absolute Counting Beads were added the flow samples to measure cell counts. ND-OVA-II+IL-2/IC (D3,4,5) animals showed the highest total number of T_{regs}, with an average of about 99,000 CD4⁺CD25⁺Foxp3⁺ cells counted per 2 mL of blood (FIG. 8c). ND-OVA-II+IL-2/IC (D1,2,3) mice had the highest number of antigen specific T_{regs}, with about 16,400 CD4⁺CD25⁺Foxp3⁺ OT-II Tetramer⁺ cells counted per 2 mL of blood (FIG. 8d). In addition, CD25 and GITR expression was measured on total T_{regs} and OT-II Tetramer⁺ T_{regs}. Similar to data from day 7, ND-OVA-II+IL-2/IC (D3,4,5) treatment resulted in the highest expression of both CD25 and GITR among total and OT-II Tetramer⁺ T_{regs}, compared to all other groups (FIG. 8e,f).

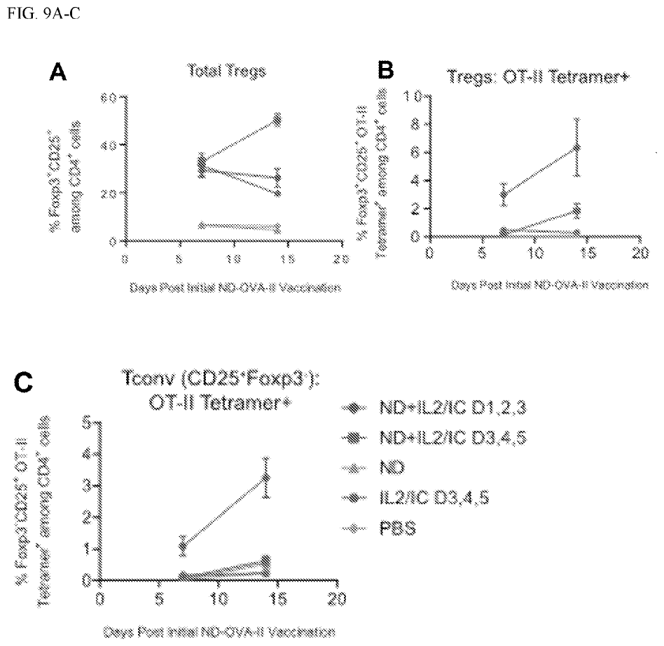

The summary of these findings is shown in FIG. 9. ND-OVA-II+IL-2/IC (D1,2,3) treatment expanded total T_{regs} to ˜30% of CD4⁺ T cells in PBMCs both on day 7 and 14 (FIG. 9a). ND-OVA-II+IL-2/IC (D1,2,3) treatment increased antigen-specific OT-II Tetramer⁺Foxp3⁺CD25⁺ T_{regs} from ˜3% to ˜6.5% among CD4⁺ T cells in PBMCs on day 7 and 14, respectively (FIG. 9b), which represent a 6-fold and 20-fold improvement over IL-2/IC treatment alone. Notably, ND-OVA-II+IL-2/IC (D1,2,3) treatment also expanded antigen-specific OT-II Tetramer⁺Foxp3-CD25⁺ T_{conv} among total CD4⁺ T cells from ˜1% to ˜3.3% on day 7 and 14, respectively (FIG. 9c).

On the other hand, ND-OVA-II+IL-2/IC (D3,4,5) treatment expanded total T_{regs} to ˜35% on day 7 to ˜50% on day 14 among CD4⁺ T cells in PBMCs (FIG. 9a). ND-OVA-II+IL-2/IC (D3,4,5) treatment increased antigen-specific OT-II Tetramer⁺Foxp3⁺CD25⁺ T_{regs} from ˜0.2% to ˜2% among CD4⁺ T cells in PBMCs on day 7 and 14, respectively (FIG. 9b), which represented a 6-fold improvement over IL-2/IC treatment alone by day 14. ND-OVA-II+II-2/IC (D3,4,5) treatment also expanded antigen-specific OT-II Tetramer⁺Foxp3-CD25⁺ T_{conv} among total CD4⁺ T cells from ˜0.1% to ˜0.6% on day 7 and 14, respectively (FIG. 9c).

Although ND-OVA-II+IL-2/IC (D3,4,5) animals acquired a smaller number and percentage of antigen specific T_{regs} (FIG. 9b), they showed significant expansion of total T_{regs} among CD4⁺ cells (FIG. 9a) and higher expression of CD25 and GITR (FIG. 6e,f, FIG. 8 e,f), compared to ND-OVA-II+IL-2/IC (D1,2,3) and IL-2/IC (D3,4,5) only groups. Increased expression of CD25 and GITR suggest that T_{regs} stimulated by ND-OVA-II+IL-2/IC (D3,4,5) therapy may be more functional.

### Example II

Tregs are potent mediators of immune tolerance in inflamed tissues. However, it is remains unknown how to promote and control Treg migration into certain tissues. It was envisioned that circulating Tregs can be recruited into certain tissues by applying their cognate antigen on local tissues. We anticipate that peptide-MHC-II complex displayed on antigen-presenting cells in local tissues will trigger T cell responses, thereby leading to the secretion of chemokines and recruitment of Tregs into the tissues. Thus, this approach may be beneficial to recruit Tregs into local tissues and mediate local immune tolerance.

Furthermore, this approach may be broadly applicable even in autoimmune diseases with un-identified autoantigens. While autoantigens are well-defined in a subset of autoimmune diseases, many autoimmune diseases have undefined autoantigens. Therefore, it would be beneficial to develop an antigen non-specific approach to induce Tregs in the general circulation and promote their tissue infiltration into certain tissues or organs.

In Example I, we have reported that antigen-loaded nanodiscs, in combination with IL-2/IC treatments, led to robust induction of antigen-specific Tregs in the blood circulation. In this example (Example II), it is shown that antigen-specific Tregs in the blood circulation can be recruited into ear dermal tissues applied with the antigen. Thus, it is envisioned that antigen-nanodisc in combination with IL-2/IC or mutein IL-2 treatments, followed by application of autoantigens (either by injection or topical application) can drive Treg infiltration into certain tissues and organs.

### Preparation of Antigen-Loaded Nanodiscs:

DMPC(1,2-dimyristoyl-sn-glycero-3-phosphocholine) and DOPE-MAL (N-(3-Maleimide-1-oxopropyl)-L-α-phosphatidylethanolamine) were purchased from NOF AMERICA CORPORATION. DMPC and ApoA1-mimetic peptide 22A (PVLDLFRELLBELLEALKQKLK) powder were mixed and hydrated in 10 mM sodium phosphate buffer (DMPC: 22A=2:1, mass ratio), which was then subjected to heating and cooling cycles to obtain blank synthetic HDL Nanodisc, followed by sonication for 5 min at room temperature. To load antigen peptide into blank Nanodisc, cysteine-terminated antigen peptide was first conjugated with DOPE-MAL (antigen peptide:DOPE-MAL=2:1, molar ratio). Then DOPE-peptide was added to blank Nanodisc (22A:antigen peptide=5:1, mass ratio) and incubated at room temperature with gentle shaking on an orbital shaker for 1 h. Unreacted antigen peptides were removed by using Zeba Spin desalting columns (Pierce). Antigen peptide used in this study was OVA-II (OVA323-339) peptide, CSS-ISQAVHAAHAEINEAGR. The peptide loading efficiency was measured by LC-MS. Nanodisc-OVA-II was analyzed by gel permeation chromatography (GPC) equipped with TSKgel G3000SWxl column (7.8 mm ID×30 cm, Tosoh Bioscience LLC). The hydrodynamic sizes and zeta potentials of Nanodisc samples were measured by dynamic light scattering (DLS, Zetasizer Nano ZSP).

### Preparation of Mouse IL-2:Anti-IL-2 Immune Complex (IL-2 IC):

IL-2/anti-IL-2 mAb immune complex (IL-2/IC) was prepared as previously reported [1]. 0.1 mg anti-IL-2 mAb (JES6-1A12, InVivoMAb, BioX-Cell, Lebanon, NH) was mixed in vitro with recombinant murine IL-2 (20 μg, PeproTech, Rocky Hill, NJ) in 2 mL HBSS (Mediatech, Herndon, VA) and incubated at room temperature for 10 min. Then the resulting IL-2/IC was administered in 0.1 mL single injection dose containing 1 μg murine IL-2 and 5 g IL-2 mAb.

### Animal Study

Animals were cared for following federal, state, and local guidelines. All work performed on animals was in accordance with and approved by Institutional Animal Care & Use Committee (IACUC) at University of Michigan, Ann Arbor. Seven-week-old female C57BL/6 mice were randomly divided into 3 groups with 5 mice in each group. On Day 0, mice received adoptive cells transfer (ACT) of 2.8×10⁶ CD4⁺ T cells from 9-12-week-old OT-II transgenic mice by retro-orbital injections. Next, on Days 0, 7 and 14, mice in groups 1 and 2 were injected subcutaneously with 0.1 mL Nanodisc-OVA-II (containing 0.1 mg OVA-II peptide) at the tail base. In addition, group 1 received intraperitoneal (i.p.) administration of 0.1 mL as-prepared IL-2/IC solution (described above) on days 3, 4, and 5 after each Nanodisc-OVA-II injection. Mice in group 2 received ACT+Nanodisc-OVA-II treatments alone, and Mice in group 3 received ACT+PBS as a control group. On day 25, mice were anesthetized by inhalation of isoflurane, and left and right ears were administered intradermally with 10 mg of either OVA-II peptide or irrelevant MOG35-55 peptide in PBS, respectively, using 29G-needle syringe (BD SAFETYGLIDE 0.5 ML INSULIN SYRINGE 29G X 0.5″). After 24 hrs, mice were euthanized, and cell suspensions from the ears were prepared. Briefly, the ears were removed and split into dorsal and ventral halves, and cartilage was removed. The skins were cut into small pieces and digested for 90 min at 37° C. in a solution of RPMI 1640 plus 5 mg/ml DNase I (Sigma-Aldrich) and 3 mg/ml collagenase type III (Sigma-Aldrich) containing 2% FBS. The cell suspensions were passed over 70-um strainer and washed before flow cytometric analysis.

### Flow Cytometric Analysis

For surface staining, total cells from each ear digestion were transferred to a 96 well plate and incubated with eBioscience Fixable Viability Dye eFluor450 in PBS for 10 minutes at room temperature in the dark. Samples were washed with 200 uL PBS and spun at 600 g for five minutes. Supernatants were discarded, and cells were suspended in Fc Block (anti-CD16/32) in FACS buffer (PBS+1% BSA). Blocking was allowed to proceed for 10 minutes at room temperature in the dark before OT-II tetramer was added to the wells at a 1:40 final dilution in FACS buffer. OT-II tetramer (NIH Tetramer Core Facility, Atlanta, GA) was allowed to incubate with FACs samples for 1 hour at room temperature in the dark. 20 minutes prior to the end of the OT-II tetramer incubation, surface antibodies were added to the samples at a 1:100 final dilution. Following tetramer and surface incubation, cells were washed with 200 uL FACs buffer and spun at 600 g for 5 minutes. For Foxp3 intracellular staining, eBioscience™ Foxp3/Transcription Factor Staining Buffer Set was used according to the kit's instructions. To measure total cell counts, 25 μL of Life Technologies Absolute Counting Beads were added to fully stained samples prior to collection on the BioRad ZE5 Analyzer. FSC files were analyzed using FlowJo.

Analysis of Antigen-Specific T Cells after Antigen Challenge in Ears.

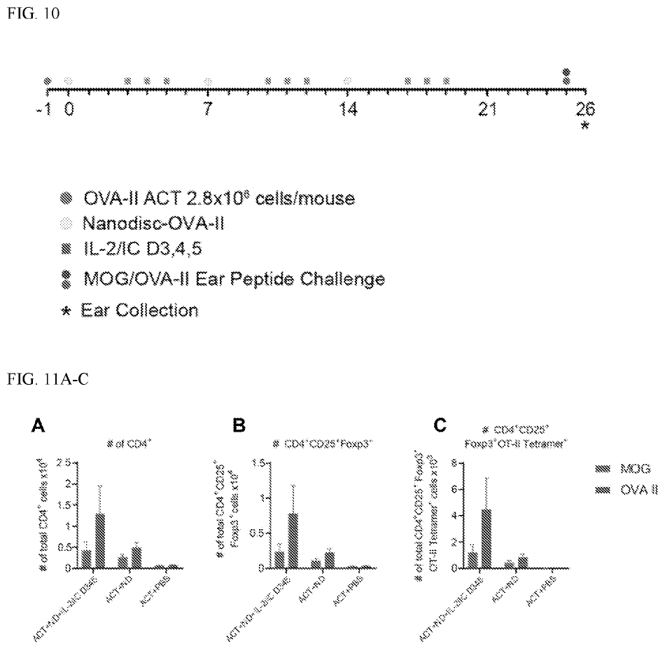

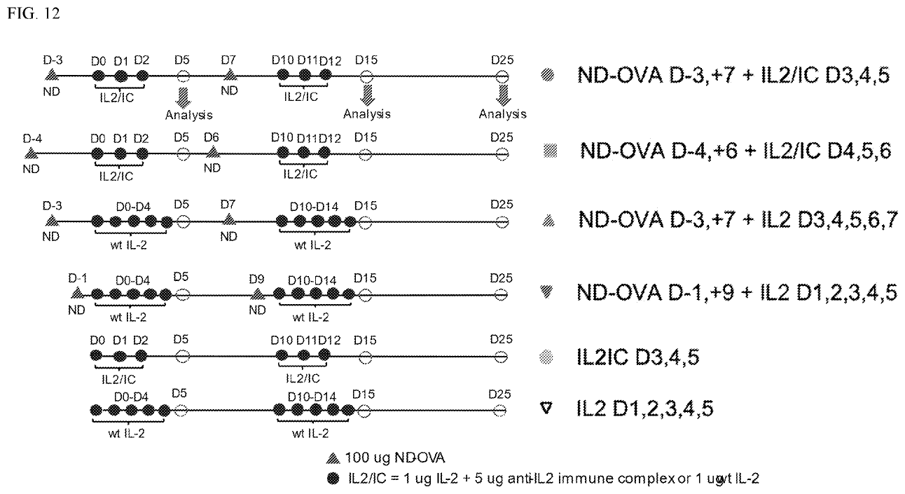

C57BL/6 mice were given 2.8×10⁶ CD4⁺ OVA-II T cells (ACT) via retro-orbital i.v. injection. One day later, Nanodisc-OVA-II (ND-OVA-II) was administered s.c. and mice were treated with IL-2/IC via i.p. route using the regimen shown in FIG. 10. Control groups included either ACT+ND-OVA-II or ACT+PBS treatments. On day 25 post initial ND vaccination, mice were challenged via intra-dermal injection of 10 ug of OVA-II peptide in the right ear or irrelevant MOG peptide in the left ear. After 24 hours, left and right ears were excised, processed into single cell suspensions, and stained for flow cytometric analysis. In mice treated with ACT+ND-OVA-II+IL-2/IC (D3,4,5), ears challenged with OVA-II specific peptide contained on average 13,000 total CD4⁺ T cells, compared to only 4,300 found in MOG-challenge ears (FIG. 11A). In addition, OVA-II peptide challenged ears showed increased numbers of total CD4⁺CD25⁺Foxp3⁺ T_{regs} and antigen-specific OT-II-Tetramer⁺CD4⁺CD25⁺Foxp3⁺ T_{regs} in mice treated with treated with either ACT+ND-OVA-II+IL-2/IC (D3,4,5) or ACT+ND-OVA-II (FIG. 11B,C). In mice treated with ACT+ND-OVA-II+IL-2/IC (D3,4,5), numbers of CD4⁺CD25⁺Foxp3⁺ T_{regs} and OT-IT Tetramer⁺ T_{regs} were 3.2 and 3.8-fold increased, respectively, in OVA-II challenged ears, compared to irrelevant MOG peptide challenged ears (FIG. 11B,C). When compared to ACT+ND-OVA-II or ACT+PBS controls challenged with OVA-II peptide, ACT+ND-OVA-II+IL-2/IC (D3,4,5) mice on average showed a 5 or 1000-fold increase in the total number of antigen-specific T_{regs} migrating to ear tissues (FIG. 11C).

### Example III

### Materials and Methods

### Preparation of Antigen-Loaded Nanodiscs

DMPC(1,2-dimyristoyl-sn-glycero-3-phosphocholine) and DOPE-MAL (N-(3-Maleimide-1-oxopropyl)-L-α-phosphatidylethanolamine) were purchased from NOF AMERICA CORPORATION and Avanti Polar Lipids, respectively. DMPC and ApoA1-mimetic peptide 22A (PVLDLFRELLBELLEALKQKLK) powder were mixed and hydrated in 10 mM sodium phosphate buffer (DMPC: 22A=2:1, mass ratio), which was then subjected to heating and cooling cycles to obtain blank synthetic HDL Nanodisc (ND), followed by sonication for 2 min at room temperature. To load antigen peptide into blank Nanodisc, cysteine-terminated antigen peptide was first conjugated with DOPE-MAL (antigen peptide:DOPE-MAL=1.5:1, molar ratio). Then DOPE-peptide was added to blank Nanodisc (22A:antigen peptide=5:1, mass ratio) and incubated at room temperature with gentle shaking on an orbital shaker for 1 h. Unreacted antigen peptides were removed by using Zeba Spin desalting columns (Pierce). Antigen peptide used in this study was OVA-II (OVA323-339) peptide, ISQAVHAAHAEINEAGR. The peptide loading efficiency was measured by LC-MS.

### Preparation of Mouse IL-2:Anti-IL-2 Immune Complex (IL-2/IC)

IL-2/anti-IL-2 mAb immune complex (IL-2/IC) was prepared as previously reported [15]. 1 mg anti-IL-2 mAb (JES6-1A12, InVivoMAb, BioX-Cell, Lebanon, NH) was mixed in vitro with 0.2 mg recombinant murine IL-2 (PeproTech, Rocky Hill, NJ) in 20 mL HBSS (Mediatech, Herndon, VA) and incubated at room temperature for 10 min. Then the resulting IL-2/IC was administered in 0.1 mL single injection dose containing 1 μg murine IL-2 and 5 g IL-2 mAb to the experimental animals.

### Animal Study

Animals were cared for following federal, state, and local guidelines. All work performed on animals was in accordance with and approved by Institutional Animal Care & Use Committee (IACUC) at the University of Michigan, Ann Arbor. Seven-week-old female C57BL/6 mice were randomly divided into 6 groups with 5 mice in each group. For Group 1 (ND-OVA_D−3,+7+IL2/IC_D3,4,5): 0.1 mL of ND-OVA (containing 0.1 mg OVA-II peptide) was injected into each mouse subcutaneously at the tail base at Day −3 and Day 7; 0.1 mL IL2/IC (containing 1 μg murine IL-2 and 5 g IL-2 mAb) was injected into each mouse intraperitoneally (i.p.) at Days 0, 1, 2, and Days 10, 11, 12. For Group 2 (ND-OVA_D−4,+6+IL2/IC_D4,5,6): 0.1 mL of ND-OVA (containing 0.1 mg OVA-II peptide) was injected into each mouse subcutaneously at the tail base at Day −4 and Day 6; 0.1 mL IL2/IC (containing 1 g murine IL-2 and 5 μg IL-2 mAb) was injected into each mouse intraperitoneally (i.p.) at Days 0, 1, 2, and Days 10, 11, 12. For Group 3 (ND-OVA_D−3,+7+IL2/IC_D3,4,5,6,7): 0.1 mL of ND-OVA (containing 0.1 mg OVA-II peptide) was injected into each mouse subcutaneously at the tail base at Day −3 and Day 7; 0.1 mL IL2 (containing 1 μg murine IL-2) was injected into each mouse intraperitoneally (i.p.) at Days 0, 1, 2, 3, 4 and Days 10, 11, 12, 13, 14. For Group 4 (ND-OVA_D−1,+9+IL2/IC_D1,2,3,4,5): 0.1 mL of ND-OVA (containing 0.1 mg OVA-II peptide) was injected into each mouse subcutaneously at the tail base at Day −1 and Day 9; 0.1 mL IL2 (containing 1 μg murine IL-2) was injected into each mouse intraperitoneally (i.p.) at Days 0, 1, 2, 3, 4 and Days 10, 11, 12, 13, 14. For Group 5 (IL2/IC_D3,4,5): 0.1 mL IL2/IC (containing 1 μg murine IL-2 and 5 g IL-2 mAb) was injected into each mouse intraperitoneally (i.p.) at Days 0, 1, 2, and Days 10, 11, 12. For Group 6 (IL2/IC_D1,2,3,4,5): 0.1 mL IL2 (containing 1 μg murine IL-2) was injected into each mouse intraperitoneally (i.p.) at Days 0, 1, 2, 3, 4 and Days 10, 11, 12, 13, 14, as shown in FIG. 12. The injection used 29G-needle syringe (BD SAFETYGLIDE 0.5 ML INSULIN SYRINGE 29G X 0.5″). On Days 5, 15 and 25, mouse peripheral blood was collected for flow cytometry analysis.

### Flow Cytometric Analysis

At indicated time points, 150-200 uL of blood was collected from individual mice into EDTA coated tubes. The blood volumes were transferred to 1.5 mL Eppendorf tubes for red blood cell lysis. Red blood cells were lysed by adding 1 mL ACK lysis buffer to each blood volume, and tubes were allowed to shake at room temperature for five minutes. After five minutes, the tubes were spun at 600 g for 5 minutes and supernatants were discarded. Pellets were suspended again in 1 mL of ACK lysis buffer and immediately spun at 600 g for 5 minutes. Supernatants were discarded, and PBMCs were re-suspended in PBS and transferred to a 96-well plate and washed once with PBS before proceeding to FACS staining. For surface staining, cells were first incubated with eBioscience Fixable Viability Dye eFluor450 in PBS for 10 minutes at room temperature in the dark. PBMCs were washed with 200 uL PBS and spun at 600 g for five minutes. Supernatants were discarded, and the samples were suspended in Fc Block (anti-CD16/32) in FACS buffer (PBS+1% BSA). Blocking was allowed to proceed for 10 minutes at room temperature in the dark before OT-II tetramer was added to the wells at a 1:40 final dilution in FACS buffer. OT-II tetramer (NIH Tetramer Core Facility, Atlanta, GA) was allowed to incubate with PBMCs for 1 hour at room temperature in the dark. 20 minutes prior to the end of the OT-II tetramer incubation, surface antibodies were added to the samples at a 1:100 final dilution. Following tetramer and surface incubation, PBMCs were washed with 200 uL FACs buffer and spun at 600 g for 5 minutes. For Foxp3 intracellular staining, eBioscience™ Foxp3/Transcription Factor Staining Buffer Set was used according to the kit's instructions. To measure total cell counts, 50 uL of Life Technologies Absolute Counting Beads were added to fully stained PBMC samples prior to collection on the BioRad ZE5 Analyzer. FSC files were analyzed using FlowJo.

### Results

### Optimization of the Dosing Regimen and Comparison of ND+wtIL2 vs ND+IL2/IC

C57BL/6 mice were administered Nanodisc-OVA-II (ND-OVA) s.c. and treated with IL-2 immune complex (IL-2/IC) or wild type IL-2 protein (IL-2) via i.p. route using the regimen shown in FIG. 12. We found in Example I that IL-2/IC injections given closer to initial ND-OVA vaccination can expand OT-II Tetramer+Foxp3−CD25+ Tconv cells (Example I, FIG. 9c). To optimize the ND vaccination and IL-2/IC combination therapy, we tested two regimens: delivering IL-2/IC for three consecutive days starting three-(group 1) or four-days (group 2) post vaccination. We compared these two regimens to ND vaccination plus wtIL-2 protein therapy. The half-life of IL-2 protein in vivo is significantly shorter than IL-2/IC. Therefore, we included five doses of IL-2 beginning three-(group 3) or one-day (group 4) post ND-OVA vaccination. Control groups included IL-2/IC (group 5) and IL-2 (group 6) treatments alone (FIG. 12). Systemic immune responses were measured by flow cytometry on days 5, 15 and 25 post the first injection of IL-2/IC or IL-2.

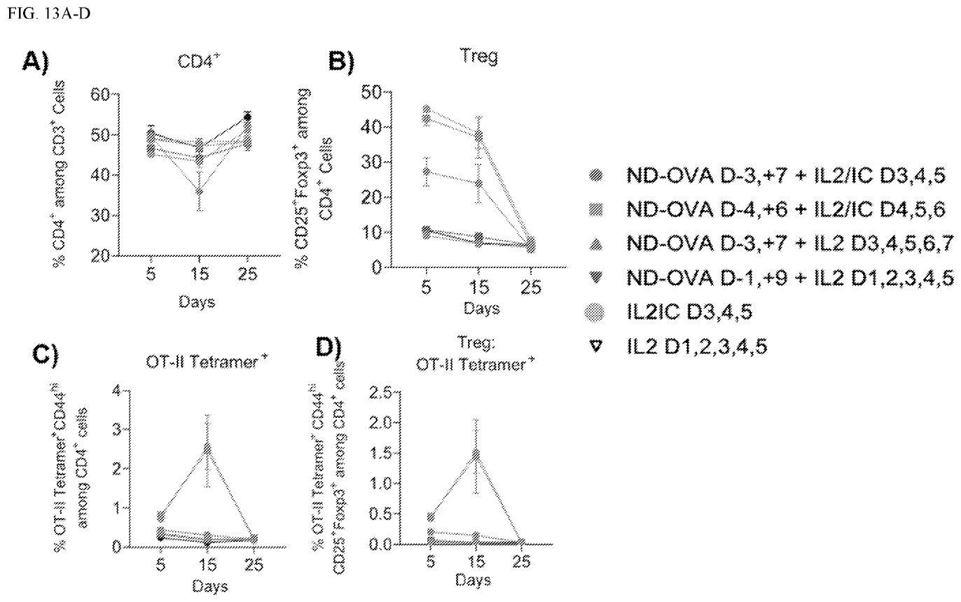

Over the course of the vaccine regimen, the frequencies of CD4+ T cells among lymphocytes was similar among the vaccine groups, with only IL-2/IC alone controls showing a drop in CD4 T cells on day 15 (FIG. 13a). Mice in groups 1 and 2 that received ND-OVA+IL-2/IC on D3,4,5 or D4,5,6 demonstrated almost identical CD4+ T cell responses among PBMCs.

On days 5 and 15, animals that received ND-OVA+IL-2/IC on D3,4,5 or D4,5,6 exhibited a 4.5-fold expansion of CD4+CD25+Foxp3+T regulatory cells (T_{regs}) compared to animals in group 3 and 4 that received ND-OVA+IL-2 (FIG. 13b). Importantly, combination of ND-OVA+IL-2/IC expanded ˜1.6-fold more total T_{regs} compared to IL-2/IC alone, while ND-OVA+IL-2 and IL-2 alone failed to increase frequencies of T_{regs} above 10% (FIG. 13b). Ten days following the last vaccine cycle, T_{regs} in animals treated with IL-2/IC had returned to the baseline (FIG. 13b).

In addition to total T_{regs}, animals in groups 1 and 2 elicited robust antigen-specific OT-II responses compared to all other groups. After one vaccine cycle, total OT-II Tetramer+ and OT-II Tetramer+Foxp3+CD25+T_{regs} cells represented ˜0.76% and ˜0.44% of total CD4+ cells, respectively (FIG. 13c,d). A second round of vaccination further increased total and T_{reg} antigen-specific cells to ˜2.5% and ˜1.5% among total CD4⁺ cells, respectively, representing a 3.3-fold increase from day 5 (FIG. 13c,d). Notably by day 15, antigen-specific OT-11 T_{regs} in ND-OVA+IL-2/IC mice were increased ˜55-fold, compared to mice treated with ND-OVA+IL-2 or IL-2 alone (FIG. 13d).

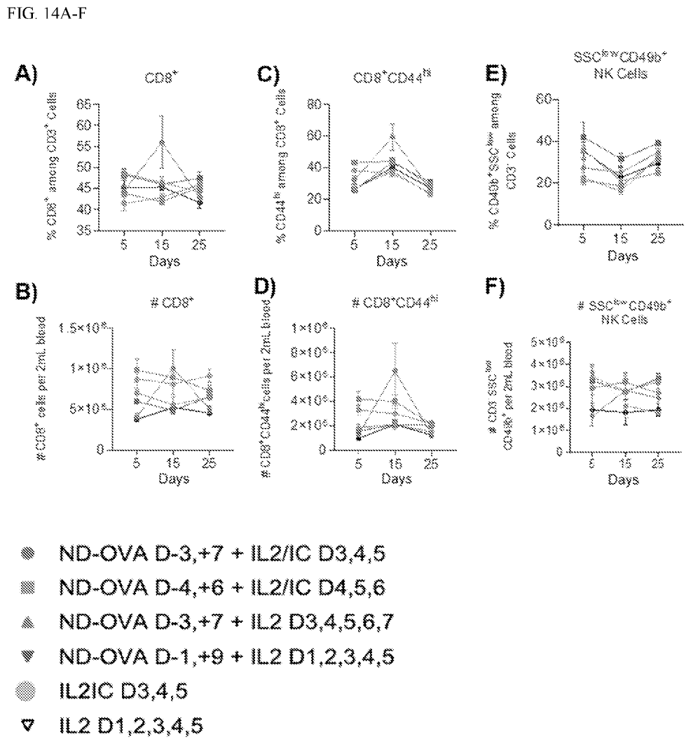

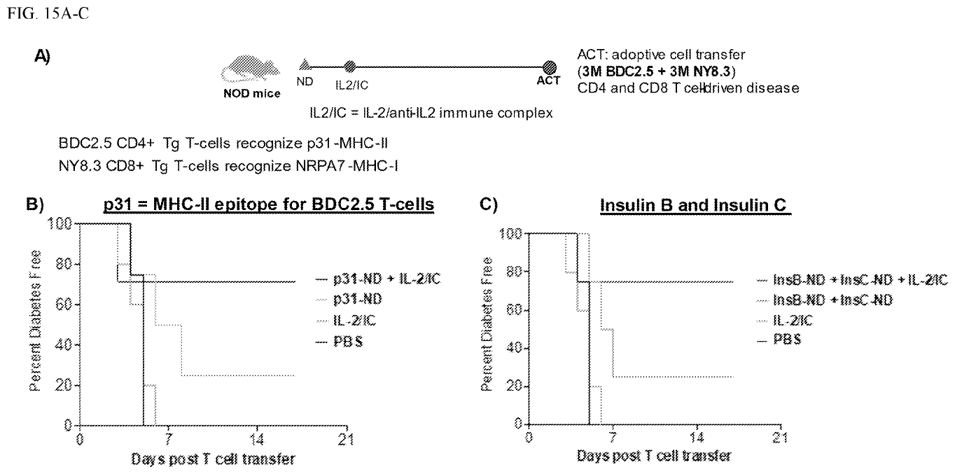

To examine the impact of the vaccine regimens on other immune cells subsets, we measured the frequency and absolute numbers of CD8+ T cells, activated CD8+CD44hi T cells, and NK cells among the PBMCs (FIG. 14). The frequencies of CD8+ cells among groups were similar, ranging from 40-50% on average from day 5 to 25 (FIG. 14a). On day 15, two of four mice from the IL-2/IC alone group displayed significantly elevated CD8+ T cells above 64%, while the other two mice had frequencies of CD8+ T cells in the average range. Absolute numbers of CD8+ T cells were between 0.38-1.0×106 per 2 mL of blood from day 5 to 25 (FIG. 14b). Frequencies of activated CD8+CD44hi were found to be between 24-60% of total CD8+ T cells, while absolute numbers were between 0.10-0.33×106 per 2 mL of blood from day 5 to 25 (FIG. 14c,d). Lastly, the frequency and absolute number of NK cells ranged from 16-42% of CD3-CD45+ cells and 1.6-2.8×106 per 2 mL of blood, respectively (FIG. 14e,f).

These results emphasize three important findings: (1) the addition of ND to IL-2/IC therapy can increase the frequency of total T_{regs} compared to IL-2/IC alone (FIG. 13b), (2) IL-2 protein fails to expand total T_{regs} and antigen-specific T_{regs} (FIG. 13b,d), and (3) Delaying IL-2/IC injection from D3,4,5 to D4,5,6 post ND-OVA vaccination does not significantly impact the expansion of total T_{regs} or antigen-specific T_{regs}.

### Example IV

### Materials and Methods

### Preparation of Antigen-Loaded Nanodiscs

DMPC(1,2-dimyristoyl-sn-glycero-3-phosphocholine) and DOPE-MAL (N-(3-Maleimide-1-oxopropyl)-L-α-phosphatidylethanolamine) were purchased from NOF AMERICA CORPORATION. DMPC and ApoA1-mimetic peptide 22A (PVLDLFRELLBELLEALKQKLK) powder were mixed and hydrated in 10 mM sodium phosphate buffer (DMPC: 22A=2:1, mass ratio), which was then subjected to heating and cooling cycles to obtain blank synthetic HDL Nanodisc, followed by sonication for 5 min at room temperature. To load antigen peptide into blank Nanodisc, cysteine-terminated antigen peptide was first conjugated with DOPE-MAL (antigen peptide:DOPE-MAL=2:1, molar ratio). Then DOPE-peptide was added to blank Nanodisc (22A:antigen peptide=5:1, mass ratio) and incubated at room temperature with gentle shaking on an orbital shaker for 1 h. Unreacted antigen peptides were removed by using Zeba Spin desalting columns (Pierce). Antigen peptide used in this study were p31 peptide (YVRPLWVRME), InsB9-23 peptide, and the full length C-peptide. The peptide loading efficiency was measured by LC-MS. Nanodiscs was analyzed by gel permeation chromatography (GPC) equipped with TSKgel G3000SWxl column (7.8 mm ID×30 cm, Tosoh Bioscience LLC). The hydrodynamic sizes and zeta potentials of Nanodisc samples were measured by dynamic light scattering (DLS, Zetasizer Nano ZSP).

### Preparation of Mouse IL-2:Anti-IL-2 Immune Complex (IL-2/IC)

IL-2/anti-IL-2 mAb immune complex (IL-2/IC) was prepared as previously reported [15]. 0.1 mg anti-IL-2 mAb (JES6-1A12, InVivoMAb, BioX-Cell, Lebanon, NH) was mixed in vitro with recombinant murine IL-2 (20 μg, PeproTech, Rocky Hill, NJ) in 2 mL HBSS (Mediatech, Herndon, VA) and incubated at room temperature for 10 min. Then the resulting IL-2/IC was administered in 0.1 mL single injection dose containing 1 μg murine IL-2 and 5 g IL-2 mAb.

### Animal Study

Animals were cared for following federal, state, and local guidelines. All work performed on animals was in accordance with and approved by Institutional Animal Care & Use Committee (IACUC) at University of Michigan, Ann Arbor. Seven-to-Nine-week-old male NOD mice were randomly divided into 4 groups with 5-10 mice in each group. On Day 0, mice in groups 1 and 2 were injected subcutaneously with 0.1 mL Nanodisc-p31 (containing 0.1 mg p31 peptide) at the tail base. In addition, group 1 received intraperitoneal (i.p.) administration of 0.1 mL as-prepared IL-2/IC solution (described above) on days 3, 4, and 5 after each Nanodisc-p31 injection. Mice in group 2 received Nanodisc-p31 treatments alone, and Mice in group 3 and group 4 received IL-2/IC and PBS as control groups, respectively. On day 11, mice were intravenously injected with 2 million preactivated BDC2.5 splenocytes and 2 million preactivated NY8.3 splenocytes, respectively. The mice were monitored for diabetes incidence thereafter using OneTouch Ultra2 glucose meter) via tail vein and animals with blood glucose levels above 250 mg/dl on two consecutive measurements were considered diabetic.

### Results

### Therapeutic Efficacy of ND and IL-2/IC Combo in Transferred T1D Models.

NOD mice were treated with Nanodisc-p31 (ND-p31) subcutaneously followed by IL-2/IC via i.p. route using the regimen shown in FIG. 15A. Control groups included either PBS or IL-2/IC or ND alone. On day 6 post the last IL-2/IC treatment, mice were transferred with 3 million preactivated BDC2.5 splenocytes and 3 million preactivated NY8.3 splenocytes via retro-orbital injection (FIG. 15A). In mice treated with IL-2/IC, transfer of diabetogenic T cells resulted in diabetes onset as fast as those in PBS group (FIG. 15B). In addition, one mouse in p31-ND treatment alone was protected from diabetes. In stark contrast, more than 70% of the mice receiving p31-ND+IL-2/IC combo were protected from the disease.

We also evaluated the therapeutic efficacy of ND+IL-2/IC combo using InsB9-23 and Ins-C peptide using the same experiment setting (FIG. 15A). In mice treated with IL-2/IC or PBS, transfer of diabetogenic BDC2.5 and NY8.3 T cells resulted in hyperglycemia within a week (FIG. 15C). One mouse in InsB-ND+InsC-ND treatment was protected from diabetes. In stark contrast, more than 70% of the mice receiving InsB-ND+InsC-ND+IL-2/IC combo were free of diabetes.

Taken together, these results show that a single round of ND therapy in combination with IL-2/IC results in potent bystander suppression and robust efficacy against the disease in the adoptive transfer model of T1D.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein is incorporated by reference for all purposes. The following references are herein incorporated by reference in their entireties:
1. Sakaguchi, S., Yamaguchi, T., Nomura, T. & Ono, M. Regulatory T cells and immune tolerance. cell 133, 775-787, 2008.
2. Shevach, E. M. Application of IL-2 therapy to target T regulatory cell function. Trends in immunology 33, 626-632, 2012.
3. Hirakawa, M., Matos, T., Liu, H., Koreth, J., Kim, H. T., Paul, N. E., Murase, K., Whangbo, J., Alho, A. C. & Nikiforow, S. Low-dose IL-2 selectively activates subsets of CD4+ Tregs and NK cells. JCI insight 12016.
4. Rosenzwajg, M., Lorenzon, R., Cacoub, P., Pham, H. P., Pitoiset, F., El Soufi, K., RIbet, C., Bernard, C., Aractingi, S. & Banneville, B. Immunological and clinical effects of low-dose interleukin-2 across 11 autoimmune diseases in a single, open clinical trial. Annals of the rheumatic diseases 78. 209-217, 2019.
5. Higginson-Scott, N., Otipoby, K. L. & Viney, J. PT101: A Treg selective agonist IL-2 mutein therapy for autoimmunity. (Am Assoc Immnol, 2020).
6. Ghelani, A., Bates, D., Conner, K., Wu, M.-Z., Lu, J., Hu, Y.-L., L₁, C.-M., Chaudhry, A. & Sohn, S. J. Defining the Threshold IL-2 Signal Required for Induction of Selective Treg Cell Responses Using Engineered IL-2 Muteins. Frontiers in Immunology 11, 1106, 2020.
7. Peterson, L. B., Bell, C. J., Howlett, S. K., Pekalski, M. L., Brady, K., Hinton, H., Sauter, D., Todd, J. A., Umana, P. & Ast, O. A long-lived IL-2 mutein that selectively activates and expands regulatory T cells as a therapy for autoimmune disease. Journal of autoimmunity 95, 1-14, 2018.
8. Khoryati, L., Pham, M. N., Sherve, M., Kumari, S., Cook, K., Pearson, J., Bogdani, M., Campbell, D. J. & Gavin, M. A. An IL-2 mutein engineered to promote expansion of regulatory T cells arrests ongoing autoimmunity in mice. Science immunology 52020.
9. globenewswire.com/en/news-release/2021/01/04/2152594/0/en/Pandion-Therapeutics-Announces-Positive-Top-Line-Phase-1a-Clinical-Data-Showing-PT101-was-Well-Tolerated-and-Selectively-Expanded-Regulatory-T-cells.html. 1/20/2022, 2022.
10. Mills, K. H. & McGuirk, P. Antigen-specific regulatory T cells—their induction and role in infection. Seminars in immunology.
11. Legoux, F. P., Lim, J.-B., Cauley, A. W., Dikiy, S., Ertelt, J., Mariani, T. J., Sparwasser, T., Way, S. S. & Moon, J. J. CD4+ T cell tolerance to tissue-restricted self antigens is mediated by antigen-specific regulatory T cells rather than deletion. Immunity 43, 896-908, 2015.
12. Tang, Q., Henriksen, K. J., Bi, M., Finger, E. B., Szot, G., Ye, J., Masteller, E. L., McDevitt, H., Bonyhadi, M. & Bluestone, J. A. In vitro-expanded antigen-specific regulatory T cells suppress autoimmune diabetes. The Journal of experimental medicine 199, 1455-1465, 2004.
13. Spence, A., Purtha, W., Tam, J., Dong, S., Kim, Y., Ju, C.-H., Sterling, T., Nakayama, M., Robinson, W. H. & Bluestone, J. A. Revealing the specificity of regulatory T cells in murine autoimmune diabetes. Proceedings of the National Academy of Sciences 115, 5265-5270, 2018.
14. Webster, K. E., Walters, S., Kohler, R. E., Mrkvan, T., Boyman, O., Surh, C. D., Grey, S. T. & Sprent, J. In vivo expansion of T reg cells with IL-2-mAb complexes: induction of resistance to EAE and long-term acceptance of islet allografts without immunosuppression. Journal of Experimental Medicine 206, 751-760, 2009.
15. Manirarora, J. N. & Wei, C.-H. Combination therapy using IL-2/IL-2 monoclonal antibody complexes, rapamycin, and islet autoantigen peptides increases regulatory T cell frequency and protects against spontaneous and induced type 1 diabetes in nonobese diabetic mice. The Journal of Immunology 195, 5203-5214, 2015.
16. Izquierdo, C., Ortiz, A. Z., Presa, M., Malo, S., Montoya, A., Garabatos, N., Mora, C., Verdaguer, J. & Stratmann, T. Treatment of T1D via optimized expansion of antigen-specific Tregs induced by IL-2/anti-IL-2 monoclonal antibody complexes and peptide/MHC tetramers. Scientific reports 8, 1-14, 2018.
17. Kuai, R., L₁, D., Chen, Y. E., Moon, J. J. & Schwendeman, A. High-density lipoproteins: nature's multifunctional nanoparticles. ACS Nano 10, 3015-3041, 2016.
18. Kuai, R., Ochyl, L. J., Bahjat, K. S., Schwendeman, A. & Moon, J. J. Designer vaccine nanodiscs for personalized cancer immunotherapy. Nat Mater 16, 489-496, 2017.

1. A method comprising administering to a subject:
a composition comprising a nanoparticle; and
a composition comprising an immunomodulatory agent capable of expanding regulatory T cells (Tregs) within the subject.

2. The method of claim 1,
wherein the nanoparticle is associated with one or more tolerogenic antigens,
wherein administration of the composition comprising a nanoparticle associated with one or more tolerogenic antigens followed by administration of the composition comprising an immunomodulatory agent capable of expanding Tregs results in one or more of the following:
treating, preventing, and/or attenuating one or more disorders in the subject;
facilitating a strong immune tolerance to antigens associated with an autoimmune disease within the subject;
in vivo amplification of Tregs (e.g., CD3+FOXP3+ cells) within the subject; and
increasing the ratio of CD3+FOXP3+ cells to CD3+FOXP3− cells within the population of T cells within the subject; and
wherein the antigen is specific to the one or more tolerogenic antigens associated with the nanoparticle associated with one or more tolerogenic antigens.

3. The method of claim 2, wherein the composition comprising an immunomodulatory agent capable of expanding Tregs is comprised within a nanoparticle such that the nanoparticle is associated with the immunomodulatory agent capable of expanding Tregs.

4. The method of claim 2-3, wherein the composition comprising a nanoparticle associated with one or more tolerogenic antigens is administered prior to and not simultaneously with (e.g., 1 second prior, 2 seconds prior, 1 minute prior, 1 hour prior, 1 day prior, 1 week prior, 1 month prior, 1 year prior, etc.) administration of the composition comprising an immunomodulatory agent capable of expanding Tregs.

5. The method of claim 4, wherein the composition comprising an immunomodulatory agent capable of expanding Tregs is administered within thirty days of the administration of the composition comprising a nanoparticle associated with one or more tolerogenic antigens.

6. The method of claim 1 or 2 or 3, wherein the nanoparticle is an sHDL nanoparticle.

7. The method of claim 6, wherein the sHDL nanoparticle comprises a mixture of at least one phospholipid and at least one HDL apolipoprotein or apolipoprotein mimetic.

8. The method of claim 7, wherein the phospholipid is selected from the group consisting of 1,2-dilauroyl-sn-glycero-3-phosphocholine; 1,2-dimyristoyl-sn-glycero-3-phosphocholine; 1,2-dipalmitoyl-sn-glycero-3-phosphocholine; 1,2-distearoyl-sn-glycero-3-phosphocholine; 1,2-diarachidoyl-sn-glycero-3-phosphocholine; 1,2-dibehenoyl-sn-glycero-3-phosphocholine; 1,2-dilignoceroyl-sn-glycero-3-phosphocholine; 1,2-dimyristoleoyl-sn-glycero-3-phosphocholine; 1,2-dimyristelaidoyl-sn-glycero-3-phosphocholine; 1,2-dipalmitoleoyl-sn-glycero-3-phosphocholine; 1,2-dipalmitelaidoyl-sn-glycero-3-phosphocholine; 1,2-dipetroselenoyl-sn-glycero-3-phosphocholine; 1,2-dioleoyl-sn-glycero-3-phosphocholine; 1,2-dielaidoyl-sn-glycero-3-phosphocholine; 1,2-dieicosenoyl-sn-glycero-3-phosphocholine; 1,2-dinervonoyl-sn-glycero-3-phosphocholine; 1,2-dilauroyl-sn-glycero-3-phosphoethanolamine; 1,2-dimyristoyl-sn-glycero-3-phosphoethanolamine; 1,2-dipentadecanoyl-sn-glycero-3-phosphoethanolamine; 1,2-dipalmitoyl-sn-glycero-3-phosphoethanolamine; 1,2-distearoyl-sn-glycero-3-phosphoethanolamine; 1,2-dipalmitoleoyl-sn-glycero-3-phosphoethanolamine; 1,2-dielaidoyl-sn-glycero-3-phosphoethanolamine; 1,2-dioleoyl-sn-glycero-3-phosphoethanolamine; dioleoyl-sn-glycero-3-phosphoethanolamine-N-[3-(2-pyridyldithio) propionate]; 1,2-dipalmitoyl-sn-glycero-3-phosphothioethanol; 1,2-di-(9Z-octadecenoyl)-sn-glycero-3-phosphoethanolamine-N-[4-(p-maleimidophenyl)butyramide]; 1,2-dihexadecanoyl-sn-glycero-3-phosphoethanolamine-N-[4-(p-maleimidophenyl)butyramide]; 1,2-dihexadecanoyl-sn-glycero-3-phosphoethanolamine-N-[4-(p-maleimidomethyl)cyclohexane-carboxamide]; 1,2-di-(9Z-octadecenoyl)-sn-gly cero-3-phosphoethanolamine-N-[4-(p-maleimidomethyl)cyclohexane-carboxamide]; N-[(3-Maleimide-1-oxopropyl)aminopropyl polyethyleneglycol-carbamyl]distearoylphosphatidyl-ethanolamine; N-[(3-Maleimide-1-oxopropyl)aminopropyl polyethyleneglycol-carbamyl]distearoylphosphatidyl-ethanolamine; N-(3-Maleimide-1-oxopropyl)-L-α-phosphatidylethanolamine, Distearoyl; N-[(3-Maleimide-1-oxopropyl)aminopropyl polyethyleneglycol-carbamyl]distearoylphosphatidyl-ethanolamine; N-(3-Maleimide-1-oxopropyl)-L-α-phosphatidylethanolamine, Dimyristoy; N-(3-Maleimide-1-oxopropyl)-L-α-phosphatidylethanolamine, Dioleoyl; N-(3-Maleimide-1-oxopropyl)-L-α-phosphatidylethanolamine, Dipalmitoyl; N-(3-Maleimide-1-oxopropyl)-L-α-phosphatidylethanolamine, 1-Palmitoyl-2-oleoyl; phosphatidylcholine; phosphatidylinositol; phosphatidylserine; phosphatidylethanolamine; N-(Succinimidyloxy-glutaryl)-L-α-phosphatidylethanolamine, Distearoyl; N-(Succinimidyloxy-glutaryl)-L-α-phosphatidylethanolamine, Dioleoyl; N-(Succinimidyloxy-glutaryl)-L-α-phosphatidylethanolamine, 1-Palmitoyl-2-oleoyl; N-(Succinimidyloxy-glutaryl)-L-α-phosphatidylethanolamine, Dipalmitoyl; N-(Succinimidyloxy-glutaryl)-L-α-phosphatidylethanolamine, Dimyristoyl; 3-(N-succinimidyloxyglutaryl)aminopropyl, and polyethyleneglycol-carbamyl distearoylphosphatidyl-ethanolamine; N-(3-oxopropoxy polyethylenegly col)carbamyl-distearoyl-ethanolamine.

9. The method of claim 7, wherein the HDL apolipoprotein component is selected from the group consisting of apolipoprotein A-I (apoA-I), apolipoprotein A-II (apoA-II), apolipoprotein A-II xxx (apoA-II-xxx), apolipoprotein A4 (apoA4), apolipoprotein Cs (apoCs), apolipoprotein E (apoE), apolipoprotein A-I milano (apoA-I-milano), apolipoprotein A-I paris (apoA-I-paris), apolipoprotein M (apoM), an HDL apolipoprotein mimetic, preproapoliprotein, preproApoA-I, proApoA I, preproApoA-II, proApoA II, preproApoA-IV, proApoA-IV, ApoA-V, preproApoE, proApoE, preproApoA I_{Milano}, proApOA-I_{Milano}, preproApoA-I_{Paris}, proApoA-I_{Paris}, and mixtures thereof.

10. The method of claim 7, wherein the apolipoprotein mimetic is described by any of SEQ ID NOs: 1-336 and WDRVKLDLATVYVDVLKDSGRDYVSQF (SEQ ID NO:341), LIKLLDNWDSVTSTFSKLREOL (SEQ ID NO:342), PVTOEFWDNLEKETEGLROEMS (SEQ ID NO:343), KDLEEVKAKVQ (SEQ ID NO: 344), KDLEEVKAKVO (SEQ ID NO: 345), PYLDDFQKKWQEEMIELYRQKVE (SEQ ID NO: 346), PLRAELQEGARQKLHELOEKLS (SEQ ID NO: 347), PLGEEMRDRARAHVDALRTHLA (SEQ ID NO: 348), PYSDELRQRLAARLEALKENGG (SEQ ID NO: 349), ARLAEYHAKATEHLSTLSEKAK (SEQ ID NO: 350), PALEDLROGLL (SEQ ID NO: 351), PVLESFKVSFLSALEFYTKKLN (SEQ ID NO:352), PVLESFVSFLSA LEYTKIKLN (SFQ ID NO:353), PVLESFKVSFLSALEEYTKKLN (SEQ ID NO:352), TVLLLTICSLEGALVRRQAKEPCV (SEQ ID NO: 354) QTVTDYGKDLME (SEQ ID NO:355), KVKSPELOAEAKSYFEK_SKE (SEQ ID NO:356), VLTLALVAVAGAPRAEVSADOVATV (SEQ ID NO:357), NNAKEAVEHLOKSELTOOLNAL (SEQ ID NO:358), LPVLVWLSIVLEGPAPAOGTPDVSS (SEQ ID NO:359), LPVLVVVLSIVLEGPAPAQGTPDVSS (SEQ ID NO:360), ALDKLKEFGNTLEDKARELIS (SEQ ID NO: 361), VVALLALLASARASEAEDASLL (SEQ ID NO:362), HLRKLRKRLLRDADDLQKRLAVYOA (SEQ ID NO:363), AQAWGERLRARIMEEMGSRTRDR (SEQ ID NO:364), LDEVKEQV AEVRAKLEEQAQ (SEQ ID NO:365), DWLKAFYDKV AEKLKEAF (SEQ ID NO:236), DWLKAFYDKVAEKLKEAFPDWAKAAYDKAAEKAKEAA (SEQ ID NO:366), PVLDLFRELLNELLEALKQKL (SEQ ID NO:367), PVLDLFRELLNELLEALKQKLA (SEQ ID NO:368), PVLDLFRELLNELLEALKQKLK (SEQ ID NO:4), PVLDLFRELLNELLEALKQKLA (SEQ ID NO:369), PVLDLFRELLNELLEALKKLLK (SEQ ID NO:370), PVLDLFRELLNELLEALKKLLA (SEQ ID NO:371), PLLDLFRELLNELLEALKKLLA (SEQ ID NO:372), and EVRSKLEEWFAAFREFAEEFLARLKS (SEQ ID NO: 373).

11. The method of claim 2-10, wherein the one or more tolerogenic antigens are tolerogenic antigens comprising between 3 amino acids and 50 amino acids in length.

12. The method of claim 2-10, wherein the one or more tolerogenic antigens are tolerogenic antigens comprising a polypeptide comprising an amino acid sequence of any one of SEQ ID NOs: 375-796.

13. The method of claim 2-10, wherein the one or more tolerogenic antigens are human allograft transplantation antigens.

14. The method of claim 13, wherein the human allograft transplantation antigens are selected from subunits of the various MHC class I and MHC class II haplotype proteins, and single-amino-acid polymorphisms on minor blood group antigens including RhCE, Kell, Kidd, Duffy and Ss.

15. The method of claim 2-10, wherein the one or more tolerogenic antigens are specific for type 1 diabetes mellitus.

16. The method of claim 15, wherein the tolerogenic antigens are selected from insulin, proinsulin, preproinsulin, glutamic acid decarboxylase-65 (GAD-65), GAD-67, insulinoma-associated protein 2 (IA-2), insulinoma-associated protein 2β (IA-2β), ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, S100P, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5.

17. The method of claim 2-10, wherein the tolerogenic antigens are specific for one or more of the following disorders: rheumatoid arthritis, multiple sclerosis, primary biliary cholangitis, primary sclerosing cholangitis, MOG antibody disease, diabetes, autoimmune diseases of the thyroid, thyroid-associated ophthalmopathy and dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutières syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polvarthritis/fetal and neonatal anemia, Sepsis, inflammatory bowel disease, transplantation related disorders, one or more allergies, a respiratory condition (e.g., asthma), and GVHD.

18. The method of claim 2-10, wherein the one or more tolerogenic antigens comprises one or more of tolerogenic antigens selected from thyroglobulin (TG), thyroid peroxidase (TPO), thyrotropin receptor (TSHR), sodium iodine symporter (NIS), megalin, thyroid autoantigens including TSHR, insulin-like growth factor 1 receptor, calcium sensitive receptor, 21-hydroxylase, 17α-hydroxylase, and P450 side chain cleavage enzyme (P450scc), ACTH receptor, P450c21, P450c17, FSH receptor, α-enolase, pituitary gland-specific protein factor (PGSF) Ta and 2, and type 2 iodothyronine deiodinase, myelin basic protein, myelin oligodendrocyte glycoprotein, proteolipid protein, collagen II, H⁺, K⁺-ATPase, tissue transglutaminase and gliadin, tyrosinase, tyrosinase related protein 1 and 2, acetylcholine receptor, desmoglein 3, 1 and 4, pemphaxin, desmocollins, plakoglobin, perplakin, desmoplakins, acetylcholine receptor, BP180, BP230, plectin, laminin 5, endomysium, tissue transglutaminase, collagen VII, matrix metalloproteinase 1 and 3, the collagen-specific molecular chaperone heat-shock protein 47, fibrillin-1, PDGF receptor, Scl-70, U1 RNP, Th/To, Ku, Jol, NAG-2, centromere proteins, topoisomerase I, nucleolar proteins, RNA polymerase I, II and III, PM-Slc, fibrillarin, B23, U1snRNP, nuclear antigens SS-A and SS-B, fodrin, poly(ADP-ribose) polymerase, topoisomerase, nuclear proteins including SS-A, high mobility group box 1 (HMGB1), nucleosomes, histone proteins, double-stranded DNA, glomerular basement membrane proteins including collagen IV, cardiac myosin, aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and type 1 interferons interferon alpha, beta and omega.

19. The method of claim 2-10, wherein the one or more tolerogenic antigens are specific for celiac disease.

20. The method of claim 19, wherein the tolerogenic antigens are selected from gliadin, glutenin, and fragments thereof capable of inducing an immune response.

21. The method of claim 20, wherein the tolerogenic antigens are selected from gliadin or fragments thereof.

22. The method of claim 21, wherein the tolerogenic antigens are selected from the group consisting of α, γ, and ω gliadins or fragments thereof.

23. The method of claim 21 or 22, wherein the tolerogenic antigen comprises a polypeptide having at least 90% sequence identity to the polypeptide sequence of any one of SEQ ID NOs: 375-580.

24. The method of claim 23, wherein the tolerogenic antigen comprises a polypeptide having at least 90% sequence identity to any one of the polypeptide sequences of SEQ ID NOs: 375-580.

25. The method of claim 24, wherein the tolerogenic antigen comprises a polypeptide having the polypeptide sequence of any one of SEQ ID NOs: 375-580.

26. method of claim 25, wherein the tolerogenic antigen comprises two or more polypeptide sequences having the sequence of any one of SEQ ID NOs: 375-580.

27. The method of claim 2-10, wherein the tolerogenic antigens are multimeric tolerogenic antigens comprising the following N-terminal-to-C-terminal structure
(P₄-L₄)ₙ₄-(P₃-L₃)n₃-P₂-(L₁-P₁)n
wherein P₁, P₂, P₃, and P₄ are each independently a tolerogenic antigen;
L₁, L₃, and L₄ are each independently a linker; and
n₁, n₃, and n₄ are each independently 0 or 1, wherein at least one of n₁, n₃, and n₄ are 1.

28. The composition of claim 27, wherein n₁ is 1, n₃ is 0, and n₄ is 0, and the tolerogenic antigen comprises the following N-terminal-to-C-terminal structure:
P₂-L₁-P₁.

29. The method of claim 27-28, wherein L₁ is a peptide linker comprising between 2 and 200 amino acids.

30. The method of claim 27-29, wherein L₁ is a peptide linker comprising between 5 and 50 amino acids.

31. The method of claim 27-30, wherein L₁ is a peptide linker comprising glycine (G) and serine (S) residues.

32. The method of claim 27-31, wherein L₁ is a peptide linker comprising the amino acid sequence of (GS)ₓ, (GGS)ₓ, or (GGGGS)ₓ, wherein x is an integer from 1 to 10.

33. The method of claim 27-32, wherein P₁ and P₂ each comprise different tolerogenic antigens.

34. The method of claim 27-32, wherein P₁ and P₂ each comprise identical tolerogenic antigens.

35. The composition of claim 27, wherein n₁ is 1, n₃ is 1, and n₄ is 0, and the tolerogenic antigen comprises the following N-terminal-to-C-terminal structure:
P₃-L₃-P₂-L₁-P₁.

36. The composition of claim 27-32, wherein L₁ and L₃ are each an independently selected peptide linker comprising between 2 and 200 amino acids.

37. The method of claim 27, wherein L₁ and L₃ are each an independently selected peptide linker comprising between 5 and 50 amino acids.

38. The method of claim 27, wherein L₁ and L₃ are each an independently selected peptide linker comprising glycine (G) and serine (S) residues.

39. The method of claim 35, wherein L₁ and L₃ are each an independently selected peptide linker comprising the amino acid sequence of (GS)ₓ, (GGS)ₓ, or (GGGGS)ₓ, wherein x is an integer from 1 to 10.

40. The method of claim 35, wherein P₁, P₂, and/or P₃ each comprise different tolerogenic antigens.

41. The method of claim 34, wherein P₁, P₂, and P₃ each comprise identical tolerogenic antigens.

42. The method of claim 27, wherein n₁ is 1, n₃ is 1, and n₄ is 1, and the tolerogenic antigen comprises the following N-terminal-to-C-terminal structure:
P₄-L₄-P₃-L₃-P₂-L₁-P₁.

43. The method of claim 42, wherein L₁ and L₂ are each an independently selected peptide linker comprising between 2 and 200 amino acids.

44. The method of claim 43, wherein L₁, L₂, and L₃ are each an independently selected peptide linker comprising between 5 and 50 amino acids.

45. The method of claim 43, wherein L₁, L₂, and L₃ are each an independently selected peptide linker comprising glycine (G) and serine (S) residues.

46. The method of claim 42, wherein L₁, L₂, and L₃ are each an independently selected peptide linker comprising the amino acid sequence of (GS)ₓ, (GGS)ₓ, or (GGGGS (SEQ ID NO: 219))ₓ, wherein x is an integer from 1 to 10.

47. The method of any one of claim 42, wherein P₁, P₂, P₃, and/or P₄ each comprise different tolerogenic antigens.

48. The method of claim 42, wherein P₁, P₂, P₃, and P₄ each comprise identical tolerogenic antigens.

49. The method of claim 2-10, wherein the number of tolerogenic antigens associated with a specific nanoparticle includes a population of between 1 and 30 tolerogenic antigens per nanoparticle.

50. The method of claim 49, wherein the number of tolerogenic antigens associated with a specific nanoparticle includes a population of between 1 and 10 tolerogenic antigens per particle.

51. The method of claim 49, wherein the number of tolerogenic antigens associated with a specific nanoparticle includes a population of 6 tolerogenic antigens per particle.

52. The method of claim 49, wherein the number of tolerogenic antigens associated with a specific nanoparticle includes a population of 8 tolerogenic antigens per particle.

53. The method of claim 50, wherein the population of tolerogenic antigens associated with a specific nanoparticle are the same tolerogenic antigen.

54. The method of claim 49, where the population of tolerogenic antigens associated with a specific nanoparticle comprises between 1 and 5 different tolerogenic antigens.

55. The method of claim 48, wherein the population of tolerogenic antigens associated with a specific nanoparticle include 3 to 4 different tolerogenic antigens.

56. The method of claim 54, wherein the population of tolerogenic antigens are specific to between 1 and 3 different diseases.

57. The method of claim 54, wherein the population of tolerogenic antigens are specific to the same disease.

58. The method of claim 2, wherein the population of tolerogenic antigens associated with a specific nanoparticle comprises (i) a first polypeptide population comprising the amino acid sequence of any one of SEQ ID NOs: 406-588, or a biologically active fragment or variant thereof, (ii) a second polypeptide population comprising the amino acid sequence of any one of SEQ ID NOs: 406-588, or biologically active fragment or variant thereof, and (iii) a third polypeptide population comprising the amino acid sequence of any one of SEQ ID NOs: 406-588, or a biologically active fragment or variant thereof.

59. The method of claim 58, wherein the first polypeptide population comprises the amino acid sequence of SEQ ID NO: 474, or a biologically active fragment or variant thereof, (ii) the second polypeptide population comprises the amino acid sequence of any one of SEQ ID NOs: 406-588, or biologically active fragment or variant thereof, and (iii) the third polypeptide population comprises the amino acid sequence of any one of SEQ ID NOs: 406-588, or a biologically active fragment or variant thereof.

60. The method of claim 58, wherein the population of tolerogenic antigens associated with a specific nanoparticle comprises (i) the first polypeptide population comprises the amino acid sequence of SEQ ID NO: 474, or a biologically active fragment or variant thereof, (ii) the second polypeptide population comprises the amino acid sequence of SEQ ID NO: 475, or biologically active fragment or variant thereof, and (iii) the third polypeptide population comprises the amino acid sequence of any one of SEQ ID NOs: 406-588, or a biologically active fragment or variant thereof.

61. The method of claim 60, wherein the third polypeptide population comprises the amino acid sequence of SEQ ID NO: 476, or a biologically active fragment or variant thereof.

62. The method of claim 59, wherein the second polypeptide population comprises the amino acid sequence of SEQ ID NO: 477, or a biologically active fragment or variant thereof, and/or the third polypeptide population comprises the amino acid sequence of SEQ ID NO: 478, or a biologically active fragment or variant thereof.

63. The method of claim 58, wherein the first polypeptide population comprises the amino acid sequence of SEQ ID NO: 506, or a biologically active fragment or variant thereof, (ii) the second polypeptide population comprises the amino acid sequence of any one of SEQ ID NOs: 406-588, or biologically active fragment or variant thereof, and (iii) the third polypeptide population comprises the amino acid sequence of any one of SEQ ID NOs: 406-588, or a biologically active fragment or variant thereof.

64. The method of claim 63, wherein the population of tolerogenic antigens associated with a specific nanoparticle comprises (i) the first polypeptide population comprises the amino acid sequence of SEQ ID NO: 506, or a biologically active fragment or variant thereof, (ii) the second polypeptide population comprises the amino acid sequence of SEQ ID NO: 507, or biologically active fragment or variant thereof, and (iii) the third polypeptide population comprises the amino acid sequence of any one of SEQ ID NOs: 406-588, or a biologically active fragment or variant thereof.

65. The method of claim 64, wherein the third polypeptide population comprises the amino acid sequence of SEQ ID NO: 508, or a biologically active fragment or variant thereof.

66. The method of claim 23, wherein the tolerogenic antigen comprises a polypeptide having at least 90% sequence identity to the polypeptide sequence of SEQ ID NO: 374.

67. The method of claim 66, wherein the tolerogenic antigen comprises a polypeptide having at least 95% sequence identity to the polypeptide of SEQ ID NO: 374.

68. The method of claim 67, wherein the tolerogenic antigen comprises a polypeptide having at least 97% sequence identity to the polypeptide of SEQ ID NO: 374.

69. The method of claim 68, wherein the tolerogenic antigen comprises a polypeptide sequence of SEQ ID NO: 374.

70. The method of claim 69, wherein the tolerogenic antigen comprises a fragment of SEQ ID NO: 374, comprising 6-12 amino acid residues in length.

71. The method of claim 2, wherein the tolerogenic antigen comprises an amide group at the C-terminus.

72. The method of claim 2, wherein the tolerogenic antigen comprises a pyroglutamic acid residue at the N-terminus.

73. The method of claim 2, wherein the tolerogenic antigen comprises an acetyl group at the N-terminus.

74. The method of claim 2, wherein the tolerogenic antigen comprises a pyroglutamic acid residue at the N-terminus and an amide group at the C-terminus.

75. The method of claim 2, wherein the tolerogenic antigen comprises an acetyl group at the N-terminus and an amide group at the C-terminus.

76. The method of claim 2, wherein the tolerogenic antigen comprises an N-terminus or a C-terminus modified with a cysteine residue bound to a linker.

77. The method of claim 2, wherein the tolerogenic antigen comprises an N-terminus and a C-terminus modified with cysteine residues bound to a linker

78. The method of claim 7, wherein the one or more tolerogenic antigens are conjugated with the nanoparticle phospholipid.

79. The method of claim 7, wherein the one or more tolerogenic antigens are conjugated with the nanoparticle phospholipid between each tolerogenic antigen and the nanoparticle phospholipid.

80. The method of claim 79, wherein the nanoparticle phospholipid is selected from
N-(3-Maleimide-1-oxopropyl)-L-α-phosphatidylethanolamine, Dioleoyl;
N-(3-Maleimide-1-oxopropyl)-L-α-phosphatidylethanolamine, Distearoyl;
N-(3-Maleimide-1-oxopropyl)-L-α-phosphatidylethanolamine, 1-Palmitoyl-2-oleoyl;
N-(3-Maleimide-1-oxopropyl)-L-α-phosphatidylethanolamine, Dipalmitoyl; and
N-(3-Maleimide-1-oxopropyl)-L-α-phosphatidylethanolamine, Dimyristoy.

81. The method of claim 7, wherein the one or more tolerogenic antigens are conjugated with the nanoparticle phospholipid via an amine-mediated interaction.

82. The method of claim 81, wherein the nanoparticle phospholipid is N-(Succinimidyloxy-glutaryl)-L-α-phosphatidylethanolamine, Dioleoyl (DOPE-NHS).

83. The method of claim 81, wherein the amine-mediated interaction is through an amine-reactive phospholipid with self-immolative linkage.

84. The method of claim 1 or 2,
wherein the nanoparticle is associated with an immunomodulatory agent, and is not associated with a tolerogenic antigen; or
wherein the nanoparticle is associated with a tolerogenic antigen and is further associated with an immunomodulatory agent.

85. The method of claim 84, wherein the immunomodulatory agent is one or more of fingolimod; rapamycin; 2-(1′H-indole-3′-carbonyl)-thiazole-4-carboxylic acid methyl ester (ITE) or related ligands; Trichostatin A; Suberoylanilide hydroxamic acid (SAHA); statins; mTOR inhibitors; TGF-0 signaling agents; TGF-β receptor agonists; histone deacetylase inhibitors; corticosteroids; inhibitors of mitochondrial function; NF-κβ inhibitors; adenosine receptor agonists; prostaglandin E2 agonists (PGE2; phosphodiesterase inhibitors; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors; autophagy inhibitors; aryl hydrocarbon receptor inhibitors; proteasome inhibitor I (PSI); oxidized ATPs IDO; vitamin D3; cyclosporins; aryl hydrocarbon receptor inhibitors; resveratrol; azathiopurine (Aza); 6-mercaptopurine (6-MP); 6-thioguanine (6-TG); FK506; sanglifehrin A; salmeterol; mycophenolate mofetil (MMF); aspirin and other COX inhibitors; niflumic acid; estriol; triptolide; OPN-305, OPN-401; Eritoran (E5564); TAK-242; Cpn10; NI-0101; 1A6; AV411; IRS-954 (DV-1079); IMO-3100; CPG-52363; CPG-52364; OPN-305; ATNC05; NI-0101; IMO-8400; Hydroxychloroquine; CU-CPT22; C29; Ortho-vanillin; SSL3 protein; OPN-305; 5 SsnB; Vizantin; (+)-N-phenethylnoroxymorphone; VB3323; Monosaccharide 3; (+)-Naltrexone and (+)-naloxone; HT52; HTB2; Compound 4a; CNT02424; TH1020; INH-ODN; E6446; AT791; CpG ODN 2088; ODN TTAGGG; COV08-0064; 2R9; GpG oligonucleotides; 2-aminopurine; Amlexanox; Bay11-7082; BX795; CH-223191; Chloroquine; CLI-095; CU-CPT9a; Cyclosporin A; CTY387; Gefitnib; Glybenclamide; H-89; H-131; Isoliquiritigenin; MCC950; MRT67307; OxPAPC; Parthenolide; Pepinh-MYD; Pepinh-TRIF; Polymyxin B; R406; RU.521; VX-765; YM201636; Z-VAD-FMK; and AHR-specific ligands; including but not limited to 2,3,7,8-tetrachloro-dibenzo-p-dioxin (TCDD); tryptamine (TA); and 6 formylindolo[3,2 b]carbazole (FICZ).

86. The method of claim 84, wherein the immunomodulatory agent is a cytokine.

87. The method of claim 86, wherein the cytokine is a human cytokine.

88. The method of claim 86, wherein the cytokine is selected from TGFβ, IL-1, IL-2, IL-4, IL-6, IL-7, IL-10, IL-12A, IL12B, IL-15, IL-21 and IL-18, and any variation/mutein thereof.

89. The method of claim 84, wherein the immunomodulatory agent is selected from a human IL-2 or a variant thereof, a low dose IL-2 or a variant thereof, PT101 or a variant thereof, an IL-2 mutein, and an IL-2:anti-IL-2 antibody immune complex (IL-2/IC).

90. The method of claim 84, wherein the immunomodulatory agent is selected from the group consisting of: an extended pharmacokinetic (PK) IL-2, an extended-PK IL-2 comprising a fusion protein (e.g., an immunoglobulin fragment, human serum albumin, Fn3), an IL-2 moiety operably linked to an immunoglobulin Fc domain, an IL-2 moiety conjugated to a non-protein polymer (e.g., polyethylene glycol), and an IL-2 mutein functioning as a high affinity CD25 binder.

91. The method of claim 2, wherein the one or more disorders are selected from transplantation related disorders,
one or more allergies,
a respiratory condition (e.g., asthma), GVHD, and
autoimmune disorders selected from rheumatoid arthritis, multiple sclerosis, primary biliary cholangitis, primary sclerosing cholangitis, MOG antibody disease, diabetes, autoimmune diseases of the thyroid, thyroid-associated ophthalmopathy, thyroid-associated dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutieres syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polvarthritis/fetal and neonatal anemia, Sepsis, and inflammatory bowel disease.

92. The method of claim 91, wherein the one or more autoimmune disorders is a single autoimmune disorder.

93. The method of claim 1, wherein the one or more immunomodulatory agents is selected from the group comprising fingolimod; rapamycin; 2-(1′H-indole-3′-carbonyl)-thiazole-4-carboxylic acid methyl ester (ITE) or related ligands; Trichostatin A; Suberoylanilide hydroxamic acid (SAHA); statins; mTOR inhibitors; TGF-0 signaling agents; TGF-β receptor agonists; histone deacetylase inhibitors; corticosteroids; inhibitors of mitochondrial function; NF-κβ inhibitors; adenosine receptor agonists; prostaglandin E2 agonists (PGE2; phosphodiesterase inhibitors; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors; autophagy inhibitors; aryl hydrocarbon receptor inhibitors; proteasome inhibitor I (PSI); oxidized ATPs IDO; vitamin D3; cyclosporins; aryl hydrocarbon receptor inhibitors; resveratrol; azathiopurine (Aza); 6-mercaptopurine (6-MP); 6-thioguanine (6-TG); FK506; sanglifehrin A; salmeterol; mycophenolate mofetil (MMF); aspirin and other COX inhibitors; niflumic acid; estriol; triptolide; OPN-305, OPN-401; Eritoran (E5564); TAK-242; Cpnl0; NI-0101; 1A6; AV411; IRS-954 (DV-1079); IMO-3100; CPG-52363; CPG-52364; OPN-305; ATNC05; NI-0101; IMO-8400; Hydroxychloroquine; CU-CPT22; C29; Ortho-vanillin; SSL3 protein; OPN-305; 5 SsnB; Vizantin; (+)-N-phenethylnoroxymorphone; VB3323; Monosaccharide 3; (+)-Naltrexone and (+)-naloxone; HT52; HTB2; Compound 4a; CNT02424; TH1020; INH-ODN; E6446; AT791; CpG ODN 2088; ODN TTAGGG; COV08-0064; 2R9; GpG oligonucleotides; 2-aminopurine; Amlexanox; Bay11-7082; BX795; CH-223191; Chloroquine; CLI-095; CU-CPT9a; Cyclosporin A; CTY387; Gefitnib; Glybenclamide; H-89; H-131; Isoliquiritigenin; MCC950; MRT67307; OxPAPC; Parthenolide; Pepinh-MYD; Pepinh-TRIF; Polymyxin B; R406; RU.521; VX-765; YM201636; Z-VAD-FMK; and AHR-specific ligands; including but not limited to 2,3,7,8-tetrachloro-dibenzo-p-dioxin (TCDD); tryptamine (TA); and 6 formylindolo[3,2 b]carbazole (FICZ).

94. The method of claim 1, wherein the immunomodulatory agent is a cytokine.

95. The method of claim 94, wherein the cytokine is a human cytokine.

96. The method of claim 94, wherein the cytokine is selected from TGFβ, IL-1, IL-2, IL-4, IL-6, IL-7, IL-10, IL-12A, IL12B, IL-15, IL-21 and IL-18, and any variation and/or mutein thereof.

97. The method of claim 1, wherein the immunomodulatory agent is selected from human IL-2 or a variant thereof, low dose IL-2 or a variant thereof, PT101 or a variant thereof, or mutein IL-2 or a variant thereof.

98. The method of claim 1, wherein the immunomodulatory agent is an IL-2:anti-IL-2 antibody immune complex (IL-2/IC).

99. The method of claim 1, wherein the composition comprising one or more immunomodulatory agents capable of expanding Tregs is administered after (e.g., after 1 second, 2 seconds, 1 minute, 1 hour, 1 day, 1 week, 1 month, 1 year, etc.) administration of the composition comprising a nanoparticle.

100. The method of claim 1, wherein the subject is a human subject.

101. The method of claim 1, wherein the subject is a human subject suffering from or at risk of suffering from an autoimmune disorder.

102. The method of claim 1, wherein the amount of CD3+FOXP3+ cells is lower than the amount of CD3+FOXP3− cells within the population of T cells within the subject.

103. The method of claim 2 or 3,
wherein the treating, preventing, and/or attenuating one or more autoimmune disorders in the subject is specific to a specific tissue region;
wherein facilitating a strong immune tolerance to antigens associated with an autoimmune disease within the subject is specific to a specific tissue region;
wherein in vivo amplification of Tregs (e.g., CD3+FOXP3+ cells) within the subject is specific to a specific tissue region; and
wherein increasing the ratio of CD3+FOXP3+ cells to CD3+FOXP3− cells within the population of T cells within the subject is specific to a specific tissue region.

104. The method of claim 103, wherein the specific tissue region is associated with one or more autoimmune disorders.

105. The method of claim 103 or 104,
wherein the administration of the composition comprising a nanoparticle associated with one or more tolerogenic antigens followed by the administration of the composition comprising an immunomodulatory agent capable of expanding Tregs is further followed administration of one or more tolerogenic antigens and/or nanoparticles associated with tolerogenic antigens to the specific tissue region.

106. The method of claim 105, wherein the administration of one or more tolerogenic antigens to the specific tissue region is by one or more administration techniques selected from: injection, topical administration, subcutaneous, oral, intranasal, inhalation, rectal, and transcutaneous.

107. The method of claim 105 or 106, wherein the administration of one or more tolerogenic antigens to the specific tissue region prevents immune tolerance within the specific tissue region.

108. The method of claim 1, wherein the composition comprising an immunomodulatory agent capable of expanding Tregs is capable of expanding antigen-specific Tregs.

109. A kit comprising
a composition comprising a nanoparticle as recited in claim 1; and
a composition comprising an immunomodulatory agent capable of expanding Tregs as recited in claim 1.

110. A kit comprising
a composition comprising a nanoparticle as recited in claim 1;
a composition comprising an immunomodulatory agent capable of expanding Tregs as recited in claim 1; and
one or more tolerogenic antigens configured for topical administration and/or injection administration.


SEQUENCE LISTING (829 sequences)

SEQ ID NO: 1 - protein, 22 aa, synthetic construct
PVLDLFRELLNELLEZLKQKLK

SEQ ID NO: 2 - protein, 23 aa, synthetic construct
GVLDLFRELLNELLEALKQKLKK

SEQ ID NO: 3 - protein, 22 aa, synthetic construct
PVLDLFRELLNELLEWLKQKLK

SEQ ID NO: 4 - protein, 22 aa, synthetic construct
PVLDLFRELLNELLEALKQKLK

SEQ ID NO: 5 - protein, 23 aa, synthetic construct
PVLDLFRELLNELLEALKQKLKK

SEQ ID NO: 6 - protein, 22 aa, synthetic construct
PVLDLFRELLNEXLEALKQKLK

SEQ ID NO: 7 - protein, 22 aa, synthetic construct
PVLDLFKELLNELLEALKQKLK

SEQ ID NO: 8 - protein, 22 aa, synthetic construct
PVLDLFRELLNEGLEALKQKLK

SEQ ID NO: 9 - protein, 22 aa, synthetic construct
PVLDLFRELGNELLEALKQKLK

SEQ ID NO: 10 - protein, 22 aa, synthetic construct
PVLDLFRELLNELLEAZKQKLK

SEQ ID NO: 11 - protein, 22 aa, synthetic construct
PVLDLFKELLQELLEALKQKLK

SEQ ID NO: 12 - protein, 22 aa, synthetic construct
PVLDLFRELLNELLEAGKQKLK

SEQ ID NO: 13 - protein, 22 aa, synthetic construct
GVLDLFRELLNEGLEALKQKLK

SEQ ID NO: 14 - protein, 22 aa, synthetic construct
PVLDLFRELLNELLEALOQOLO

SEQ ID NO: 15 - protein, 22 aa, synthetic construct
PVLDLFRELWNELLEALKQKLK

SEQ ID NO: 16 - protein, 22 aa, synthetic construct
PVLDLLRELLNELLEALKQKLK

SEQ ID NO: 17 - protein, 22 aa, synthetic construct
PVLELFKELLQELLEALKQKLK

SEQ ID NO: 18 - protein, 22 aa, synthetic construct
GVLDLFRELLNELLEALKQKLK

SEQ ID NO: 19 - protein, 22 aa, synthetic construct
PVLDLFRELLNEGLEALKQKLK

SEQ ID NO: 20 - protein, 22 aa, synthetic construct
PVLDLFREGLNELLEALKQKLK

SEQ ID NO: 21 - protein, 22 aa, synthetic construct
PVLDLFRELLNELLEALKQKLK

SEQ ID NO: 22 - protein, 22 aa, synthetic construct
PVLDLFRELLNELLEGLKQKLK

SEQ ID NO: 23 - protein, 22 aa, synthetic construct
PLLELFKELLQELLEALKQKLK

SEQ ID NO: 24 - protein, 22 aa, synthetic construct
PVLDLFRELLNELLEALQKKLK

SEQ ID NO: 25 - protein, 22 aa, synthetic construct
PVLDFFRELLNEXLEALKQKLK

SEQ ID NO: 26 - protein, 22 aa, synthetic construct
PVLDLFRELLNELLELLKQKLK

SEQ ID NO: 27 - protein, 22 aa, synthetic construct
PVLDLFRELLNELZEALKQKLK

SEQ ID NO: 28 - protein, 22 aa, synthetic construct
PVLDLFRELLNELWEALKQKLK

SEQ ID NO: 29 - protein, 22 aa, synthetic construct
AVLDLFRELLNELLEALKQKLK

SEQ ID NO: 30 - protein, 22 aa, synthetic construct
PVLDLPRELLNELLEALKQKLK

SEQ ID NO: 31 - protein, 22 aa, synthetic construct
PVLDLFLELLNEXLEALKQKLK

SEQ ID NO: 32 - protein, 22 aa, synthetic construct
XVLDLFRELLNELLEALKQKLK

SEQ ID NO: 33 - protein, 22 aa, synthetic construct
PVLDLFREKLNELLEALKQKLK

SEQ ID NO: 34 - protein, 22 aa, synthetic construct
PVLDZFRELLNELLEALKQKLK

SEQ ID NO: 35 - protein, 22 aa, synthetic construct
PVLDWFRELLNELLEALKQKLK

SEQ ID NO: 36 - protein, 22 aa, synthetic construct
PLLELLKELLQELLEALKQKLK

SEQ ID NO: 37 - protein, 22 aa, synthetic construct
PVLDLFREWLNELLEALKQKLK

SEQ ID NO: 38 - protein, 22 aa, synthetic construct
PVLDLFRELLNEXLEAWKQKLK

SEQ ID NO: 39 - protein, 22 aa, synthetic construct
PVLDLFRELLEELLKALKKKLK

SEQ ID NO: 40 - protein, 22 aa, synthetic construct
PVLDLFNELLRELLEALQKKLK

SEQ ID NO: 41 - protein, 22 aa, synthetic construct
PVLDLWRELLNEXLEALKQKLK

SEQ ID NO: 42 - protein, 22 aa, synthetic construct
PVLDEFREKLNEXWEALKQKLK

SEQ ID NO: 43 - protein, 22 aa, synthetic construct
PVLDEFREKLWEXLEALKQKLK

SEQ ID NO: 44 - protein, 22 aa, synthetic construct
PVLDEFREKLNEXLEALKQKLK

SEQ ID NO: 45 - protein, 22 aa, synthetic construct
PVLDEFREKLNEXLEALKQKLK

SEQ ID NO: 46 - protein, 22 aa, synthetic construct
PVLDLFREKLNEXLEALKQKLK

SEQ ID NO: 47 - protein, 21 aa, synthetic construct
VLDLFRELLNEGLEALKQKLK

SEQ ID NO: 48 - protein, 22 aa, synthetic construct
PVLDLFRELLNELLEALKQKLK

SEQ ID NO: 49 - protein, 22 aa, synthetic construct
PVLDLFRNLLEKLLEALEQKLK

SEQ ID NO: 50 - protein, 22 aa, synthetic construct
PVLDLFRELLWEXLEALKQKLK

SEQ ID NO: 51 - protein, 22 aa, synthetic construct
PVLDLFWELLNEXLEALKQKLK

SEQ ID NO: 52 - protein, 22 aa, synthetic construct
PVWDEFREKLNEXLEALKQKLK

SEQ ID NO: 53 - protein, 22 aa, synthetic construct
VVLDLFRELLNELLEALKQKLK

SEQ ID NO: 54 - protein, 22 aa, synthetic construct
PVLDLFRELLNEWLEALKQKLK

SEQ ID NO: 55 - protein, 19 aa, synthetic construct
PLFRELLNELLEALKQKLK

SEQ ID NO: 56 - protein, 22 aa, synthetic construct
PVLDLFRELLNELLEALKQKKK

SEQ ID NO: 57 - protein, 22 aa, synthetic construct
PVLDLFRNLLEELLKALEQKLK

SEQ ID NO: 58 - protein, 21 aa, synthetic construct
PVLDEFREKLNEXLEALKQKL

SEQ ID NO: 59 - protein, 22 aa, synthetic construct
LVLDLFRELLNELLEALKQKLK

SEQ ID NO: 60 - protein, 19 aa, synthetic construct
PVLDLFRELLNELLEALKQ

SEQ ID NO: 61 - protein, 22 aa, synthetic construct
PVLDEFRWKLNEXLEALKQKLK

SEQ ID NO: 62 - protein, 22 aa, synthetic construct
PVLDEWREKLNEXLEALKQKLK

SEQ ID NO: 63 - protein, 22 aa, synthetic construct
PVLDFFREKLNEXLEALKQKLK

SEQ ID NO: 64 - protein, 22 aa, synthetic construct
PWLDEFREKLNEXLEALKQKLK

SEQ ID NO: 65 - protein, 21 aa, synthetic construct
VLDEFREKLNEXLEALKQKLK

SEQ ID NO: 66 - protein, 22 aa, synthetic construct
PVLDLFRNLLEELLEALQKKLK

SEQ ID NO: 67 - protein, 21 aa, synthetic construct
VLDLFRELLNELLEALKQKLK

SEQ ID NO: 68 - protein, 22 aa, synthetic construct
PVLDEFRELLKEXLEALKQKLK

SEQ ID NO: 69 - protein, 22 aa, synthetic construct
PVLDEFRKKLNEXLEALKQKLK

SEQ ID NO: 70 - protein, 22 aa, synthetic construct
PVLDEFRELLYEXLEALKQKLK

SEQ ID NO: 71 - protein, 22 aa, synthetic construct
PVLDEFREKLNELXEALKQKLK

SEQ ID NO: 72 - protein, 22 aa, synthetic construct
PVLDLFRELLNEXLWALKQKLK

SEQ ID NO: 73 - protein, 22 aa, synthetic construct
PVLDEFWEKLNEXLEALKQKLK

SEQ ID NO: 74 - protein, 22 aa, synthetic construct
PVLDKFREKLNEXLEALKQKLK

SEQ ID NO: 75 - protein, 22 aa, synthetic construct
PVLDEFREKLNEELEALKQKLK

SEQ ID NO: 76 - protein, 22 aa, synthetic construct
PVLDEFRELLFEXLEALKQKLK

SEQ ID NO: 77 - protein, 22 aa, synthetic construct
PVLDEFREKLNKXLEALKQKLK

SEQ ID NO: 78 - protein, 22 aa, synthetic construct
PVLDEFRDKLNEXLEALKQKLK

SEQ ID NO: 79 - protein, 22 aa, synthetic construct
PVLDEFRELLNELLEALKQKLK

SEQ ID NO: 80 - protein, 22 aa, synthetic construct
PVLDLFERLLNELLEALQKKLK

SEQ ID NO: 81 - protein, 22 aa, synthetic construct
PVLDEFREKLNWXLEALKQKLK

SEQ ID NO: 82 - protein, 20 aa, synthetic construct
LDEFREKLNEXLEALKQKLK

SEQ ID NO: 83 - protein, 22 aa, synthetic construct
PVLDEFREKLNEXLEALWQKLK

SEQ ID NO: 84 - protein, 22 aa, synthetic construct
PVLDEFREKLNELLEALKQKLK

SEQ ID NO: 85 - protein, 21 aa, synthetic construct
PLDLFRELLNELLEALKQKLK

SEQ ID NO: 86 - protein, 22 aa, synthetic construct
PVLELFERLLDELLNALQKKLK

SEQ ID NO: 87 - protein, 22 aa, synthetic construct
PLLELLKELLQELLEALKQKLK

SEQ ID NO: 88 - protein, 22 aa, synthetic construct
PVLDKFRELLNEXLEALKQKLK

SEQ ID NO: 89 - protein, 22 aa, synthetic construct
PVLDEFREKLNEXLWALKQKLK

SEQ ID NO: 90 - protein, 19 aa, synthetic construct
DEFREKLNEXLEALKQKLK

SEQ ID NO: 91 - protein, 22 aa, synthetic construct
PVLDEFRELLNEXLEALKQKLK

SEQ ID NO: 92 - protein, 22 aa, synthetic construct
PVLDEFRELYNEXLEALKQKLK

SEQ ID NO: 93 - protein, 22 aa, synthetic construct
PVLDEFREKLNEXLKALKQKLK

SEQ ID NO: 94 - protein, 22 aa, synthetic construct
PVLDEFREKLNEALEALKQKLK

SEQ ID NO: 95 - protein, 22 aa, synthetic construct
PVLDLFRELLNLXLEALKQKLK

SEQ ID NO: 96 - protein, 22 aa, synthetic construct
PVLDLFRELLNEXLEALKQKLK

SEQ ID NO: 97 - protein, 15 aa, synthetic construct
PVLDLFRELLNELLE

SEQ ID NO: 98 - protein, 22 aa, synthetic construct
PVLDLFRELLNEELEALKQKLK

SEQ ID NO: 99 - protein, 22 aa, synthetic construct
KLKQKLAELLENLLERFLDLVP

SEQ ID NO: 100 - protein, 22 aa, synthetic construct
PVLDLFRELLNELLEALKQKLK

SEQ ID NO: 101 - protein, 22 aa, synthetic construct
PVLDLFRELLNWXLEALKQKLK

SEQ ID NO: 102 - protein, 22 aa, synthetic construct
PVLDLFRELLNLXLEALKEKLK

SEQ ID NO: 103 - protein, 22 aa, synthetic construct
PVLDEFRELLNEELEALKQKLK

SEQ ID NO: 104 - protein, 15 aa, synthetic construct
PLLNELLEALKQKLK

SEQ ID NO: 105 - protein, 22 aa, synthetic construct
PAADAFREAANEAAEAAKQKAK

SEQ ID NO: 106 - protein, 22 aa, synthetic construct
PVLDLFREKLNEELEALKQKLK

SEQ ID NO: 107 - protein, 22 aa, synthetic construct
KLKQKLAELLENLLERFLDLVP

SEQ ID NO: 108 - protein, 22 aa, synthetic construct
PVLDLFRWLLNEXLEALKQKLK

SEQ ID NO: 109 - protein, 22 aa, synthetic construct
PVLDEFREKLNERLEALKQKLK

SEQ ID NO: 110 - protein, 22 aa, synthetic construct
PVLDEFREKLNEXXEALKQKLK

SEQ ID NO: 111 - protein, 22 aa, synthetic construct
PVLDEFREKLWEXWEALKQKLK

SEQ ID NO: 112 - protein, 22 aa, synthetic construct
PVLDEFREKLNEXSEALKQKLK

SEQ ID NO: 113 - protein, 22 aa, synthetic construct
PVLDEFREKLNEPLEALKQKLK

SEQ ID NO: 114 - protein, 22 aa, synthetic construct
PVLDEFREKLNEXMEALKQKLK

SEQ ID NO: 115 - protein, 22 aa, synthetic construct
PKLDEFREKLNEXLEALKQKLK

SEQ ID NO: 116 - protein, 22 aa, synthetic construct
PHLDEFREKLNEXLEALKQKLK

SEQ ID NO: 117 - protein, 22 aa, synthetic construct
PELDEFREKLNEXLEALKQKLK

SEQ ID NO: 118 - protein, 22 aa, synthetic construct
PVLDEFREKLNEXLEALEQKLK

SEQ ID NO: 119 - protein, 22 aa, synthetic construct
PVLDEFREKLNEELEAXKQKLK

SEQ ID NO: 120 - protein, 22 aa, synthetic construct
PVLDEFREKLNEELEXLKQKLK

SEQ ID NO: 121 - protein, 22 aa, synthetic construct
PVLDEFREKLNEELEALWQKLK

SEQ ID NO: 122 - protein, 22 aa, synthetic construct
PVLDEFREKLNEELEWLKQKLK

SEQ ID NO: 123 - protein, 22 aa, synthetic construct
QVLDLFRELLNELLEALKQKLK

SEQ ID NO: 124 - protein, 22 aa, synthetic construct
PVLDLFOELLNELLEALOQOLO

SEQ ID NO: 125 - protein, 22 aa, synthetic construct
NVLDLFRELLNELLEALKQKLK

SEQ ID NO: 126 - protein, 22 aa, synthetic construct
PVLDLFRELLNELGEALKQKLK

SEQ ID NO: 127 - protein, 22 aa, synthetic construct
PVLDLFRELLNELLELLKQKLK

SEQ ID NO: 128 - protein, 22 aa, synthetic construct
PVLDLFRELLNELLEFLKQKLK

SEQ ID NO: 129 - protein, 22 aa, synthetic construct
PVLELFNDLLRELLEALQKKLK

SEQ ID NO: 130 - protein, 22 aa, synthetic construct
PVLELFNDLLRELLEALKQKLK

SEQ ID NO: 131 - protein, 22 aa, synthetic construct
PVLELFKELLNELLDALRQKLK

SEQ ID NO: 132 - protein, 22 aa, synthetic construct
PVLDLFRELLENLLEALQKKLK

SEQ ID NO: 133 - protein, 22 aa, synthetic construct
PVLELFERLLEDLLQALNKKLK

SEQ ID NO: 134 - protein, 22 aa, synthetic construct
PVLELFERLLEDLLKALNOKLK

SEQ ID NO: 135 - protein, 22 aa, synthetic construct
DVLDLFRELLNELLEALKQKLK

SEQ ID NO: 136 - protein, 22 aa, synthetic construct
PALELFKDLLQELLEALKQKLK

SEQ ID NO: 137 - protein, 22 aa, synthetic construct
PVLDLFRELLNEGLEAZKQKLK

SEQ ID NO: 138 - protein, 22 aa, synthetic construct
PVLDLFRELLNEGLEWLKQKLK

SEQ ID NO: 139 - protein, 22 aa, synthetic construct
PVLDLFRELWNEGLEALKQKLK

SEQ ID NO: 140 - protein, 22 aa, synthetic construct
PVLDLFRELLNEGLEALOQOLO

SEQ ID NO: 141 - protein, 22 aa, synthetic construct
PVLDFFRELLNEGLEALKQKLK

SEQ ID NO: 142 - protein, 22 aa, synthetic construct
PVLELFRELLNEGLEALKQKLK

SEQ ID NO: 143 - protein, 22 aa, synthetic construct
PVLDLFRELLNEGLEALKQKLK

SEQ ID NO: 144 - protein, 22 aa, synthetic construct
PVLELFENLLERLLDALQKKLK

SEQ ID NO: 145 - protein, 22 aa, synthetic construct
GVLELFENLLERLLDALQKKLK

SEQ ID NO: 146 - protein, 22 aa, synthetic construct
PVLELFENLLERLLDALQKKLK

SEQ ID NO: 147 - protein, 22 aa, synthetic construct
PVLELFENLLERLFDALQKKLK

SEQ ID NO: 148 - protein, 22 aa, synthetic construct
PVLELFENLLERLGDALQKKLK

SEQ ID NO: 149 - protein, 22 aa, synthetic construct
PVLELFENLWERLLDALQKKLK

SEQ ID NO: 150 - protein, 22 aa, synthetic construct
PLLELFENLLERLLDALQKKLK

SEQ ID NO: 151 - protein, 22 aa, synthetic construct
PVLELFENLGERLLDALQKKLK

SEQ ID NO: 152 - protein, 22 aa, synthetic construct
PVFELFENLLERLLDALQKKLK

SEQ ID NO: 153 - protein, 22 aa, synthetic construct
AVLELFENLLERLLDALQKKLK

SEQ ID NO: 154 - protein, 22 aa, synthetic construct
PVLELFENLLERGLDALQKKLK

SEQ ID NO: 155 - protein, 22 aa, synthetic construct
PVLELFLNLWERLLDALQKKLK

SEQ ID NO: 156 - protein, 22 aa, synthetic construct
PVLELFLNLLERLLDALQKKLK

SEQ ID NO: 157 - protein, 22 aa, synthetic construct
PVLEFFENLLERLLDALQKKLK

SEQ ID NO: 158 - protein, 22 aa, synthetic construct
PVLELFLNLLERLLDWLQKKLK

SEQ ID NO: 159 - protein, 22 aa, synthetic construct
PVLDLFENLLERLLDALQKKLK

SEQ ID NO: 160 - protein, 22 aa, synthetic construct
PVLELFENLLERLLDWLQKKLK

SEQ ID NO: 161 - protein, 22 aa, synthetic construct
PVLELFENLLERLLEALQKKLK

SEQ ID NO: 162 - protein, 22 aa, synthetic construct
PVLELFENWLERLLDALQKKLK

SEQ ID NO: 163 - protein, 22 aa, synthetic construct
PVLELFENLLERLWDALQKKLK

SEQ ID NO: 164 - protein, 22 aa, synthetic construct
PVLELFENLLERLLDAWQKKLK

SEQ ID NO: 165 - protein, 22 aa, synthetic construct
PVLELFENLLERLLDLLQKKLK

SEQ ID NO: 166 - protein, 22 aa, synthetic construct
PVLELFLNLLEKLLDALQKKLK

SEQ ID NO: 167 - protein, 22 aa, synthetic construct
PVLELFENGLERLLDALQKKLK

SEQ ID NO: 168 - protein, 22 aa, synthetic construct
PVLELFEQLLEKLLDALQKKLK

SEQ ID NO: 169 - protein, 22 aa, synthetic construct
PVLELFENLLEKLLDALQKKLK

SEQ ID NO: 170 - protein, 22 aa, synthetic construct
PVLELFENLLEOLLDALQOOLO

SEQ ID NO: 171 - protein, 22 aa, synthetic construct
PVLELFENLLEKLLDLLQKKLK

SEQ ID NO: 172 - protein, 22 aa, synthetic construct
PVLELFLNLLERLGDALQKKLK

SEQ ID NO: 173 - protein, 22 aa, synthetic construct
PVLDLFDNLLDRLLDLLNKKLK

SEQ ID NO: 174 - protein, 22 aa, synthetic construct
PVLELFENLLERLLDALQKKLK

SEQ ID NO: 175 - protein, 22 aa, synthetic construct
PVLELFENLLERLLELLNKKLK

SEQ ID NO: 176 - protein, 22 aa, synthetic construct
PVLELWENLLERLLDALQKKLK

SEQ ID NO: 177 - protein, 22 aa, synthetic construct
GVLELFLNLLERLLDALQKKLK

SEQ ID NO: 178 - protein, 22 aa, synthetic construct
PVLELFDNLLEKLLEALQKKLR

SEQ ID NO: 179 - protein, 22 aa, synthetic construct
PVLELFDNLLERLLDALQKKLK

SEQ ID NO: 180 - protein, 22 aa, synthetic construct
PVLELFDNLLDKLLDALQKKLR

SEQ ID NO: 181 - protein, 22 aa, synthetic construct
PVLELFENLLERWLDALQKKLK

SEQ ID NO: 182 - protein, 22 aa, synthetic construct
PVLELFENLLEKLLEALQKKLK

SEQ ID NO: 183 - protein, 22 aa, synthetic construct
PLLELFENLLEKLLDALQKKLK

SEQ ID NO: 184 - protein, 22 aa, synthetic construct
PVLELFLNLLERLLDAWQKKLK

SEQ ID NO: 185 - protein, 22 aa, synthetic construct
PVLELFENLLERLLDALQOOLO

SEQ ID NO: 186 - protein, 22 aa, synthetic construct
PVLELFEQLLERLLDALQKKLK

SEQ ID NO: 187 - protein, 22 aa, synthetic construct
PVLELFENLLERLLDALNKKLK

SEQ ID NO: 188 - protein, 22 aa, synthetic construct
PVLELFENLLDRLLDALQKKLK

SEQ ID NO: 189 - protein, 22 aa, synthetic construct
DVLELFENLLERLLDALQKKLK

SEQ ID NO: 190 - protein, 22 aa, synthetic construct
PVLEFWDNLLDKLLDALQKKLR

SEQ ID NO: 191 - protein, 18 aa, synthetic construct
PVLDLLRELLEELKQKLK

SEQ ID NO: 192 - protein, 18 aa, synthetic construct
PVLDLFKELLEELKQKLK

SEQ ID NO: 193 - protein, 18 aa, synthetic construct
PVLDLFRELLEELKQKLK

SEQ ID NO: 194 - protein, 18 aa, synthetic construct
PVLELFRELLEELKQKLK

SEQ ID NO: 195 - protein, 18 aa, synthetic construct
PVLELFKELLEELKQKLK

SEQ ID NO: 196 - protein, 18 aa, synthetic construct
PVLDLFRELLEELKNKLK

SEQ ID NO: 197 - protein, 18 aa, synthetic construct
PLLDLFRELLEELKQKLK

SEQ ID NO: 198 - protein, 18 aa, synthetic construct
GVLDLFRELLEELKQKLK

SEQ ID NO: 199 - protein, 18 aa, synthetic construct
PVLDLFRELWEELKQKLK

SEQ ID NO: 200 - protein, 18 aa, synthetic construct
NVLDLFRELLEELKQKLK

SEQ ID NO: 201 - protein, 18 aa, synthetic construct
PLLDLFKELLEELKQKLK

SEQ ID NO: 202 - protein, 18 aa, synthetic construct
PALELFKDLLEELRQKLR

SEQ ID NO: 203 - protein, 18 aa, synthetic construct
AVLDLFRELLEELKQKLK

SEQ ID NO: 204 - protein, 18 aa, synthetic construct
PVLDFFRELLEELKQKLK

SEQ ID NO: 205 - protein, 18 aa, synthetic construct
PVLDLFREWLEELKQKLK

SEQ ID NO: 206 - protein, 18 aa, synthetic construct
PLLELLKELLEELKQKLK

SEQ ID NO: 207 - protein, 18 aa, synthetic construct
PVLELLKELLEELKQKLK

SEQ ID NO: 208 - protein, 18 aa, synthetic construct
PALELFKDLLEELRQRLK

SEQ ID NO: 209 - protein, 18 aa, synthetic construct
PVLDLFRELLNELLQKLK

SEQ ID NO: 210 - protein, 18 aa, synthetic construct
PVLDLFRELLEELKQKLK

SEQ ID NO: 211 - protein, 18 aa, synthetic construct
PVLDLFRELLEELOQOLO

SEQ ID NO: 212 - protein, 18 aa, synthetic construct
PVLDLFOELLEELOQOLK

SEQ ID NO: 213 - protein, 18 aa, synthetic construct
PALELFKDLLEEFRQRLK

SEQ ID NO: 214 - protein, 18 aa, synthetic construct
PVLDLFRELLEELKQKLK

SEQ ID NO: 215 - protein, 18 aa, synthetic construct
PVLDLFRELLEEWKQKLK

SEQ ID NO: 216 - protein, 18 aa, synthetic construct
PVLELFKELLEELKQKLK

SEQ ID NO: 217 - protein, 18 aa, synthetic construct
PVLDLFRELLELLKQKLK

SEQ ID NO: 218 - protein, 18 aa, synthetic construct
PVLDLFRELLNELLQKLK

SEQ ID NO: 219 - protein, 18 aa, synthetic construct
PVLDLFRELLNELWQKLK

SEQ ID NO: 220 - protein, 18 aa, synthetic construct
PVLDLFRELLEELQKKLK

SEQ ID NO: 221 - protein, 18 aa, synthetic construct
DVLDLFRELLEELKQKLK

SEQ ID NO: 222 - protein, 18 aa, synthetic construct
PVLDAFRELLEALLQLKK

SEQ ID NO: 223 - protein, 18 aa, synthetic construct
PVLDAFRELLEALAQLKK

SEQ ID NO: 224 - protein, 18 aa, synthetic construct
PVLDLFREGWEELKQKLK

SEQ ID NO: 225 - protein, 18 aa, synthetic construct
PVLDAFRELAEALAQLKK

SEQ ID NO: 226 - protein, 18 aa, synthetic construct
PVLDAFRELGEALLQLKK

SEQ ID NO: 227 - protein, 18 aa, synthetic construct
PVLDLFRELGEELKQKLK

SEQ ID NO: 228 - protein, 18 aa, synthetic construct
PVLDLFREGLEELKQKLK

SEQ ID NO: 229 - protein, 18 aa, synthetic construct
PVLDLFRELLEEGKQKLK

SEQ ID NO: 230 - protein, 18 aa, synthetic construct
PVLELFERLLEDLQKKLK

SEQ ID NO: 231 - protein, 18 aa, synthetic construct
PVLDLFRELLEKLEQKLK

SEQ ID NO: 232 - protein, 18 aa, synthetic construct
PLLELFKELLEELKQKLK

SEQ ID NO: 233 - protein, 18 aa, synthetic construct
LDDLLQKWAEAFNQLLKK

SEQ ID NO: 234 - protein, 18 aa, synthetic construct
EWLKAFYEKVLEKLKELF

SEQ ID NO: 235 - protein, 18 aa, synthetic construct
EWLEAFYKKVLEKLKELF

SEQ ID NO: 236 - protein, 18 aa, synthetic construct
DWLKAFYDKVAEKLKEAF

SEQ ID NO: 237 - protein, 18 aa, synthetic construct
DWFKAFYDKVFEKFKEFF

SEQ ID NO: 238 - protein, 18 aa, synthetic construct
GIKKFLGSIWKFIKAFVG

SEQ ID NO: 239 - protein, 18 aa, synthetic construct
DWFKAFYDKVAEKFKEAF

SEQ ID NO: 240 - protein, 18 aa, synthetic construct
DWLKAFYDKVAEKLKEAF

SEQ ID NO: 241 - protein, 18 aa, synthetic construct
DWLKAFYDKVFEKFKEFF

SEQ ID NO: 242 - protein, 18 aa, synthetic construct
EWLEAFYKKVLEKLKELP

SEQ ID NO: 243 - protein, 18 aa, synthetic construct
DWFKAFYDKFFEKFKEFF

SEQ ID NO: 244 - protein, 18 aa, synthetic construct
EWLKAFYEKVLEKLKELF

SEQ ID NO: 245 - protein, 18 aa, synthetic construct
EWLKAEYEKVEEKLKELF

SEQ ID NO: 246 - protein, 18 aa, synthetic construct
EWLKAEYEKVLEKLKELF

SEQ ID NO: 247 - protein, 18 aa, synthetic construct
EWLKAFYKKVLEKLKELF

SEQ ID NO: 248 - protein, 15 aa, synthetic construct
PVLDLFRELLEQKLK

SEQ ID NO: 249 - protein, 16 aa, synthetic construct
PVLDLFRELLEELKQK

SEQ ID NO: 250 - protein, 16 aa, synthetic construct
PVLDLFRELLEKLKQK

SEQ ID NO: 251 - protein, 15 aa, synthetic construct
PVLDLFRELLEKLQK

SEQ ID NO: 252 - protein, 16 aa, synthetic construct
PVLDLFRELLEALKQK

SEQ ID NO: 253 - protein, 16 aa, synthetic construct
PVLDLFENLLERLKQK

SEQ ID NO: 254 - protein, 16 aa, synthetic construct
PVLDLFRELLNELKQK

SEQ ID NO: 255 - protein, 18 aa, synthetic construct
DWLKAFYDKVAEKLKEAF

SEQ ID NO: 256 - protein, 18 aa, synthetic construct
DWLKAFYDKVAEKLKEAF

SEQ ID NO: 257 - protein, 18 aa, synthetic construct
DWLKAFYDKVAEKLKEAF

SEQ ID NO: 258 - protein, 18 aa, synthetic construct
DWFKAFYDKVAEKLKEAF

SEQ ID NO: 259 - protein, 18 aa, synthetic construct
DWLKAFYDKVAEKFKEAF

SEQ ID NO: 260 - protein, 18 aa, synthetic construct
DWFKAFYDKVAEKFKEAF

SEQ ID NO: 261 - protein, 18 aa, synthetic construct
DWLKAFYDKVFEKFKEFF

SEQ ID NO: 262 - protein, 18 aa, synthetic construct
DWLKAFYDKFFEKFKEFF

SEQ ID NO: 263 - protein, 18 aa, synthetic construct
DWFKAFYDKFFEKFKEFF

SEQ ID NO: 264 - protein, 18 aa, synthetic construct
DWLKAFYDKVAEKLKEFF

SEQ ID NO: 265 - protein, 18 aa, synthetic construct
DWLKAFYDKVFEKFKEAF

SEQ ID NO: 266 - protein, 18 aa, synthetic construct
DWLKAFYDKVFEKLKEFF

SEQ ID NO: 267 - protein, 18 aa, synthetic construct
DWLKAFYDKVAEKFKEFF

SEQ ID NO: 268 - protein, 18 aa, synthetic construct
DWLKAFYDKVFEKFKEFF

SEQ ID NO: 269 - protein, 18 aa, synthetic construct
EWLKLFYEKVLEKFKEAF

SEQ ID NO: 270 - protein, 18 aa, synthetic construct
EWLKAFYDKVAEKFKEAF

SEQ ID NO: 271 - protein, 18 aa, synthetic construct
EWLKAFYDKVAEKLKEFF

SEQ ID NO: 272 - protein, 18 aa, synthetic construct
EWLKAFYDKVFEKFKEAF

SEQ ID NO: 273 - protein, 18 aa, synthetic construct
EWLKAFYDKVFEKLKEFF

SEQ ID NO: 274 - protein, 18 aa, synthetic construct
EWLKAFYDKVAEKFKEFF

SEQ ID NO: 275 - protein, 18 aa, synthetic construct
EWLKAFYDKVFEKFKEFF

SEQ ID NO: 276 - protein, 14 aa, synthetic construct
AFYDKVAEKLKEAF

SEQ ID NO: 277 - protein, 14 aa, synthetic construct
AFYDKVAEKFKEAF

SEQ ID NO: 278 - protein, 14 aa, synthetic construct
AFYDKVAEKFKEAF

SEQ ID NO: 279 - protein, 14 aa, synthetic construct
AFYDKFFEKFKEFF

SEQ ID NO: 280 - protein, 14 aa, synthetic construct
AFYDKFFEKFKEFF

SEQ ID NO: 281 - protein, 14 aa, synthetic construct
AFYDKVAEKFKEAF

SEQ ID NO: 282 - protein, 14 aa, synthetic construct
AFYDKVAEKLKEFF

SEQ ID NO: 283 - protein, 14 aa, synthetic construct
AFYDKVFEKFKEAF

SEQ ID NO: 284 - protein, 14 aa, synthetic construct
AFYDKVFEKLKEFF

SEQ ID NO: 285 - protein, 14 aa, synthetic construct
AFYDKVAEKFKEFF

SEQ ID NO: 286 - protein, 14 aa, synthetic construct
KAFYDKVFEKFKEF

SEQ ID NO: 287 - protein, 14 aa, synthetic construct
LFYEKVLEKFKEAF

SEQ ID NO: 288 - protein, 14 aa, synthetic construct
AFYDKVAEKFKEAF

SEQ ID NO: 289 - protein, 14 aa, synthetic construct
AFYDKVAEKLKEFF

SEQ ID NO: 290 - protein, 14 aa, synthetic construct
AFYDKVFEKFKEAF

SEQ ID NO: 291 - protein, 14 aa, synthetic construct
AFYDKVFEKLKEFF

SEQ ID NO: 292 - protein, 14 aa, synthetic construct
AFYDKVAEKFKEFF

SEQ ID NO: 293 - protein, 14 aa, synthetic construct
AFYDKVFEKFKEFF

SEQ ID NO: 294 - protein, 18 aa, synthetic construct
DWLKALYDKVAEKLKEAL

SEQ ID NO: 295 - protein, 18 aa, synthetic construct
DWFKAFYEKVAEKLKEFF

SEQ ID NO: 296 - protein, 18 aa, synthetic construct
DWFKAFYEKFFEKFKEFF

SEQ ID NO: 297 - protein, 18 aa, synthetic construct
EWLKALYEKVAEKLKEAL

SEQ ID NO: 298 - protein, 18 aa, synthetic construct
EWLKAFYEKVAEKLKEAF

SEQ ID NO: 299 - protein, 18 aa, synthetic construct
EWFKAFYEKVAEKLKEFF

SEQ ID NO: 300 - protein, 18 aa, synthetic construct
EWLKAFYEKVFEKFKEFF

SEQ ID NO: 301 - protein, 18 aa, synthetic construct
EWLKAFYEKFFEKFKEFF

SEQ ID NO: 302 - protein, 18 aa, synthetic construct
EWFKAFYEKFFEKFKEFF

SEQ ID NO: 303 - protein, 18 aa, synthetic construct
DFLKAWYDKVAEKLKEAW

SEQ ID NO: 304 - protein, 18 aa, synthetic construct
EFLKAWYEKVAEKLKEAW

SEQ ID NO: 305 - protein, 18 aa, synthetic construct
DFWKAWYDKVAEKLKEWW

SEQ ID NO: 306 - protein, 18 aa, synthetic construct
EFWKAWYEKVAEKLKEWW

SEQ ID NO: 307 - protein, 18 aa, synthetic construct
DKLKAFYDKVFEWAKEAF

SEQ ID NO: 308 - protein, 18 aa, synthetic construct
DKWKAVYDKFAEAFKEFL

SEQ ID NO: 309 - protein, 18 aa, synthetic construct
EKLKAFYEKVFEWAKEAF

SEQ ID NO: 310 - protein, 18 aa, synthetic construct
EKWKAVYEKFAEAFKEFL

SEQ ID NO: 311 - protein, 18 aa, synthetic construct
DWLKAFVDKFAEKFKEAY

SEQ ID NO: 312 - protein, 18 aa, synthetic construct
EKWKAVYEKFAEAFKEFL

SEQ ID NO: 313 - protein, 18 aa, synthetic construct
DWLKAFVYDKVFKLKEFF

SEQ ID NO: 314 - protein, 18 aa, synthetic construct
EWLKAFVYEKVFKLKEFF

SEQ ID NO: 315 - protein, 18 aa, synthetic construct
DWLRAFYDKVAEKLKEAF

SEQ ID NO: 316 - protein, 18 aa, synthetic construct
EWLRAFYEKVAEKLKEAF

SEQ ID NO: 317 - protein, 18 aa, synthetic construct
DWLKAFYDRVAEKLKEAF

SEQ ID NO: 318 - protein, 18 aa, synthetic construct
EWLKAFYERVAEKLKEAF

SEQ ID NO: 319 - protein, 18 aa, synthetic construct
DWLKAFYDKVAERLKEAF

SEQ ID NO: 320 - protein, 18 aa, synthetic construct
EWLKAFYEKVAERLKEAF

SEQ ID NO: 321 - protein, 18 aa, synthetic construct
DWLKAFYDKVAEKLREAF

SEQ ID NO: 322 - protein, 18 aa, synthetic construct
EWLKAFYEKVAEKLREAF

SEQ ID NO: 323 - protein, 18 aa, synthetic construct
DWLKAFYDRVAERLKEAF

SEQ ID NO: 324 - protein, 18 aa, synthetic construct
EWLKAFYERVAERLKEAF

SEQ ID NO: 325 - protein, 18 aa, synthetic construct
DWLRAFYDKVAEKLREAF

SEQ ID NO: 326 - protein, 18 aa, synthetic construct
EWLRAFYEKVAEKLREAF

SEQ ID NO: 327 - protein, 18 aa, synthetic construct
DWLRAFYDRVAEKLKEAF

SEQ ID NO: 328 - protein, 18 aa, synthetic construct
EWLRAFYERVAEKLKEAF

SEQ ID NO: 329 - protein, 18 aa, synthetic construct
DWLKAFYDKVAERLREAF

SEQ ID NO: 330 - protein, 18 aa, synthetic construct
EWLKAFYEKVAERLREAF

SEQ ID NO: 331 - protein, 18 aa, synthetic construct
DWLRAFYDKVAERLKEAF

SEQ ID NO: 332 - protein, 18 aa, synthetic construct
EWLRAFYEKVAERLKEAF

SEQ ID NO: 333 - protein, 18 aa, synthetic construct
FAEKFKEAVKDYFAKFWD

SEQ ID NO: 334 - protein, 18 aa, synthetic construct
DWFKAFYDKVAEKFKEAF

SEQ ID NO: 335 - protein, 18 aa, synthetic construct
DWLKAFYDKVAEKLKEAF

SEQ ID NO: 336 - protein, 34 aa, synthetic construct
PALEDLRQGLLPVLESFKVFLSALEEYTKKLNTQ

SEQ ID NO: 337-340 - skipped (4 entries)

SEQ ID NO: 341 - protein, 26 aa, synthetic construct
WDRVKDLATVYVDVLKDSGRDYVSQF

SEQ ID NO: 342 - protein, 21 aa, synthetic construct
LKLLDNWDSVTSTFSKLREOL

SEQ ID NO: 343 - protein, 22 aa, synthetic construct
PVTOEFWDNLEKETEGLROEMS

SEQ ID NO: 344 - protein, 11 aa, synthetic construct
KDLEEVKAKVQ

SEQ ID NO: 345 - protein, 11 aa, synthetic construct
KDLEEVKAKVO

SEQ ID NO: 346 - protein, 22 aa, synthetic construct
PYLDDFQKKWQEEMELYRQKVE

SEQ ID NO: 347 - protein, 22 aa, synthetic construct
PLRAELQEGARQKLHELOEKLS

SEQ ID NO: 348 - protein, 22 aa, synthetic construct
PLGEEMRDRARAHVDALRTHLA

SEQ ID NO: 349 - protein, 22 aa, synthetic construct
PYSDELRQRLAARLEALKENGG

SEQ ID NO: 350 - protein, 22 aa, synthetic construct
ARLAEYHAKATEHLSTLSEKAK

SEQ ID NO: 351 - protein, 11 aa, synthetic construct
PALEDLROGLL

SEQ ID NO: 352 - protein, 22 aa, synthetic construct
PVLESFKVSFLSALEEYTKKLN

SEQ ID NO: 353 - protein, 21 aa, synthetic construct
PVLESFVSFLSALEEYTKKLN

SEQ ID NO: 354 - protein, 24 aa, synthetic construct
TVLLLTICSLEGALVRRQAKEPCV

SEQ ID NO: 355 - protein, 12 aa, synthetic construct
QTVTDYGKDLME

SEQ ID NO: 356 - protein, 20 aa, synthetic construct
KVKSPELOAEAKSYFEKSKE

SEQ ID NO: 357 - protein, 24 aa, synthetic construct
VLTLALVAVAGARAEVSADOVATV

SEQ ID NO: 358 - protein, 22 aa, synthetic construct
NNAKEAVEHLOKSELTOOLNAL

SEQ ID NO: 359 - protein, 25 aa, synthetic construct
LPVLVWLSIVLEGPAPAOGTPDVSS

SEQ ID NO: 360 - protein, 26 aa, synthetic construct
LPVLVVVLSIVLEGPAPAQGTPDVSS

SEQ ID NO: 361 - protein, 21 aa, synthetic construct
ALDKLKEFGNTLEDKARELIS

SEQ ID NO: 362 - protein, 22 aa, synthetic construct
VVALLALLASARASEAEDASLL

SEQ ID NO: 363 - protein, 25 aa, synthetic construct
HLRKLRKRLLRDADDLQKRLAVYOA

SEQ ID NO: 364 - protein, 22 aa, synthetic construct
AQAWGERLRARMEEMGSRTRDR

SEQ ID NO: 365 - protein, 20 aa, synthetic construct
LDEVKEQVAEVRAKLEEQAQ

SEQ ID NO: 366 - protein, 37 aa, synthetic construct
DWLKAFYDKVAEKLKEAFPDWAKAAYDKAAEKAKEAA

SEQ ID NO: 367 - protein, 21 aa, synthetic construct
PVLDLFRELLNELLEALKQKL

SEQ ID NO: 368 - protein, 22 aa, synthetic construct
PVLDLFRELLNELLEALKQKLA

SEQ ID NO: 369 - protein, 22 aa, synthetic construct
PVLDLFRELLNELLEALKQKLA

SEQ ID NO: 370 - protein, 22 aa, synthetic construct
PVLDLFRELLNELLEALKKLLK

SEQ ID NO: 371 - protein, 22 aa, synthetic construct
PVLDLFRELLNELLEALKKLLA

SEQ ID NO: 372 - protein, 22 aa, synthetic construct
PLLDLFRELLNELLEALKKLLA

SEQ ID NO: 373 - protein, 26 aa, synthetic construct
EVRSKLEEWFAAFREFAEEFLARLKS

SEQ ID NO: 374 - protein, 33 aa, synthetic construct
LQLQPFPQPELPYPQPELPYPQPELPYPQPQPF

SEQ ID NO: 375 - protein, 9 aa, synthetic construct
PFPQPELPY

SEQ ID NO: 376 - protein, 9 aa, synthetic construct
PYPQPELPY

SEQ ID NO: 377 - protein, 9 aa, synthetic construct
PQPELPYPQ

SEQ ID NO: 378 - protein, 9 aa, synthetic construct
FRPEQPYPQ

SEQ ID NO: 379 - protein, 9 aa, synthetic construct
PQQSFPEQQ

SEQ ID NO: 380 - protein, 9 aa, synthetic construct
IQPEQPAQL

SEQ ID NO: 381 - protein, 9 aa, synthetic construct
QQPEQPYPQ

SEQ ID NO: 382 - protein, 9 aa, synthetic construct
SQPEQEFPQ

SEQ ID NO: 383 - protein, 9 aa, synthetic construct
PQPEQEFPQ

SEQ ID NO: 384 - protein, 9 aa, synthetic construct
QQPEQPFPQ

SEQ ID NO: 385 - protein, 9 aa, synthetic construct
PQPEQPFCQ

SEQ ID NO: 386 - protein, 9 aa, synthetic construct
QQPFPEQPQ

SEQ ID NO: 387 - protein, 9 aa, synthetic construct
PFPQPEQPF

SEQ ID NO: 388 - protein, 9 aa, synthetic construct
PQPEQPFPW

SEQ ID NO: 389 - protein, 9 aa, synthetic construct
PFSEQEQPV

SEQ ID NO: 390 - protein, 9 aa, synthetic construct
FSQQQESPF

SEQ ID NO: 391 - protein, 9 aa, synthetic construct
QQPIPEQPQ

SEQ ID NO: 392 - protein, 9 aa, synthetic construct
PQPEQPFPQ

SEQ ID NO: 393 - protein, 9 aa, synthetic construct
PIPEQPQPY

SEQ ID NO: 394 - protein, 9 aa, synthetic construct
EQPIPEQPQ

SEQ ID NO: 395 - protein, 9 aa, synthetic construct
PQPEQPFPQ

SEQ ID NO: 396 - protein, 9 aa, synthetic construct
PYPEQEEPF

SEQ ID NO: 397 - protein, 9 aa, synthetic construct
PYPEQEQPF

SEQ ID NO: 398 - protein, 9 aa, synthetic construct
PFSEQEQPV

SEQ ID NO: 399 - protein, 9 aa, synthetic construct
EGSFQPSQE

SEQ ID NO: 400 - protein, 9 aa, synthetic construct
EQPQQPFPQ

SEQ ID NO: 401 - protein, 9 aa, synthetic construct
EQPQQPYPE

SEQ ID NO: 402 - protein, 9 aa, synthetic construct
QGYYPTSPQ

SEQ ID NO: 403 - protein, 9 aa, synthetic construct
EGSFQPSQE

SEQ ID NO: 404 - protein, 9 aa, synthetic construct
PQQSFPEQE

SEQ ID NO: 405 - protein, 9 aa, synthetic construct
QGYYPTSPQ

SEQ ID NO: 406 - protein, 12 aa, synthetic construct
QPFPQPQQPFPW

SEQ ID NO: 407 - protein, 12 aa, synthetic construct
QPFPQPQQPIPV

SEQ ID NO: 408 - protein, 12 aa, synthetic construct
QPFPQPEQPFPW

SEQ ID NO: 409 - protein, 11 aa, synthetic construct
PFPQPEQPIPV

SEQ ID NO: 410 - protein, 12 aa, synthetic construct
QPFPQPELPFPQ

SEQ ID NO: 411 - protein, 12 aa, synthetic construct
LPYPQPQLPYPQ

SEQ ID NO: 412 - protein, 12 aa, synthetic construct
LPYPQPELPYPQ

SEQ ID NO: 413 - protein, 12 aa, synthetic construct
QPFPQPQLPYPQ

SEQ ID NO: 414 - protein, 12 aa, synthetic construct
QPFPQPELPYPQ

SEQ ID NO: 415 - protein, 12 aa, synthetic construct
QPFPQPQQPFSQ

SEQ ID NO: 416 - protein, 12 aa, synthetic construct
QPFPQPEQPFSQ

SEQ ID NO: 417 - protein, 12 aa, synthetic construct
QPFPQPQQPFCQ

SEQ ID NO: 418 - protein, 12 aa, synthetic construct
QPFPQPEQPFCQ

SEQ ID NO: 419 - protein, 12 aa, synthetic construct
QPFPQPQLPYSQ

SEQ ID NO: 420 - protein, 12 aa, synthetic construct
QPFPQPELPYSQ

SEQ ID NO: 421 - protein, 16 aa, synthetic construct
LQQQCSPVAMPQRLAR

SEQ ID NO: 422 - protein, 12 aa, synthetic construct
QPFPQPQLPYLQ

SEQ ID NO: 423 - protein, 12 aa, synthetic construct
QPFPQPELPYLQ

SEQ ID NO: 424 - protein, 12 aa, synthetic construct
QQFIQPQQPFPQ

SEQ ID NO: 425 - protein, 12 aa, synthetic construct
QQFIQPEQPFPQ

SEQ ID NO: 426 - protein, 12 aa, synthetic construct
LERPWQQQPLPP

SEQ ID NO: 427 - protein, 12 aa, synthetic construct
LERPWQEQPLPP

SEQ ID NO: 428 - protein, 12 aa, synthetic construct
PIPQQPEQPFPL

SEQ ID NO: 429 - protein, 16 aa, synthetic construct
QGQQGYYPISPQQSGQ

SEQ ID NO: 430 - protein, 16 aa, synthetic construct
QGQPGYYPTSPQQIGQ

SEQ ID NO: 431 - protein, 16 aa, synthetic construct
PGQGQSGYYPTSPQQS

SEQ ID NO: 432 - protein, 12 aa, synthetic construct
PQQTFPQQPQLP

SEQ ID NO: 433 - protein, 12 aa, synthetic construct
PQQTFPEQPQLP

SEQ ID NO: 434 - protein, 16 aa, synthetic construct
GQGQSGYYPTSPQQSG

SEQ ID NO: 435 - protein, 16 aa, synthetic construct
QYEVIRSLVLRTLPNM

SEQ ID NO: 436 - protein, 12 aa, synthetic construct
QVDPSGQVQWPQ

SEQ ID NO: 437 - protein, 12 aa, synthetic construct
QVDPSGEVQWPQ

SEQ ID NO: 438 - protein, 12 aa, synthetic construct
QPFPQPQQPFPL

SEQ ID NO: 439 - protein, 12 aa, synthetic construct
QPFPQPEQPFPL

SEQ ID NO: 440 - protein, 12 aa, synthetic construct
QPFPQPQQPIPY

SEQ ID NO: 441 - protein, 12 aa, synthetic construct
QPFPQPEQPIPY

SEQ ID NO: 442 - protein, 12 aa, synthetic construct
PQQPVPQQPQPY

SEQ ID NO: 443 - protein, 12 aa, synthetic construct
PQQPVPEQPQPY

SEQ ID NO: 444 - protein, 16 aa, synthetic construct
PQPFPQQPIPQQPQPY

SEQ ID NO: 445 - protein, 11 aa, synthetic construct
QQPIPQQPQPY

SEQ ID NO: 446 - protein, 11 aa, synthetic construct
QQPIPEQPQPY

SEQ ID NO: 447 - protein, 12 aa, synthetic construct
QQFPQPQQPFPQ

SEQ ID NO: 448 - protein, 12 aa, synthetic construct
QQFPQPEQPFPQ

SEQ ID NO: 449 - protein, 16 aa, synthetic construct
PQQPIPQQPQPYPQQP

SEQ ID NO: 450 - protein, 16 aa, synthetic construct
QQPFPQQPFPQQPQPY

SEQ ID NO: 451 - protein, 12 aa, synthetic construct
QPFPQPQQPFSW

SEQ ID NO: 452 - protein, 12 aa, synthetic construct
QPFPQPEQPFSW

SEQ ID NO: 453 - protein, 16 aa, synthetic construct
PQQPFPQQPQPYPQQP

SEQ ID NO: 454 - protein, 12 aa, synthetic construct
QPFPQPQQPIPQ

SEQ ID NO: 455 - protein, 12 aa, synthetic construct
QPFPQPEQPIPQ

SEQ ID NO: 456 - protein, 12 aa, synthetic construct
QPFPQPQQPFPQ

SEQ ID NO: 457 - protein, 12 aa, synthetic construct
QPFPQPEQPFPQ

SEQ ID NO: 458 - protein, 12 aa, synthetic construct
QPFPQPQQPTPI

SEQ ID NO: 459 - protein, 12 aa, synthetic construct
QPFPQPEQPTPI

SEQ ID NO: 460 - protein, 12 aa, synthetic construct
PAPIQPQQPFPQ

SEQ ID NO: 461 - protein, 12 aa, synthetic construct
PAPIQPEQPFPQ

SEQ ID NO: 462 - protein, 12 aa, synthetic construct
PQQPFPQQPEQI

SEQ ID NO: 463 - protein, 12 aa, synthetic construct
PQQPFPEQPEQI

SEQ ID NO: 464 - protein, 12 aa, synthetic construct
PQQPFPQQPQQI

SEQ ID NO: 465 - protein, 12 aa, synthetic construct
PQQPFPEQPQQI

SEQ ID NO: 466 - protein, 12 aa, synthetic construct
PFPQQPEQIISQ

SEQ ID NO: 467 - protein, 12 aa, synthetic construct
PFPQQPEQIISQ

SEQ ID NO: 468 - protein, 12 aa, synthetic construct
PFPQQPEQIIPQ

SEQ ID NO: 469 - protein, 12 aa, synthetic construct
PFPQQPEQIIPQ

SEQ ID NO: 470 - protein, 12 aa, synthetic construct
QPFPQPQQQLPL

SEQ ID NO: 471 - protein, 12 aa, synthetic construct
QPFPQPEQQLPL

SEQ ID NO: 472 - protein, 11 aa, synthetic construct
LFPLPQQPFPQ

SEQ ID NO: 473 - protein, 11 aa, synthetic construct
LFPLPEQPFPQ

SEQ ID NO: 474 - protein, 15 aa, synthetic construct
LQPFPQPELPYPQPQ

SEQ ID NO: 475 - protein, 14 aa, synthetic construct
QPFPQPEQPFPWQP

SEQ ID NO: 476 - protein, 15 aa, synthetic construct
PEQPIPEQPQPYPQQ

SEQ ID NO: 477 - protein, 16 aa, synthetic construct
PQQPFPQPEQPFPWQP

SEQ ID NO: 478 - protein, 16 aa, synthetic construct
FPEQPIPEQPQPYPQQ

SEQ ID NO: 479 - protein, 15 aa, synthetic construct
PEQPIPEQPQPYPQQ

SEQ ID NO: 480 - protein, 12 aa, synthetic construct
PQPFLPQLPYPQ

SEQ ID NO: 481 - protein, 12 aa, synthetic construct
QAFPQPQQTFPH

SEQ ID NO: 482 - protein, 11 aa, synthetic construct
TPIQPQQPFPQ

SEQ ID NO: 483 - protein, 12 aa, synthetic construct
PFPLQPQQPFPQ

SEQ ID NO: 484 - protein, 11 aa, synthetic construct
PFTQPQQPTPI

SEQ ID NO: 485 - protein, 12 aa, synthetic construct
QPFPQLQQPQQP

SEQ ID NO: 486 - protein, 16 aa, synthetic construct
VAHAIIMHQQQQQQQE

SEQ ID NO: 487 - protein, 12 aa, synthetic construct
SYPVQPQQPFPQ

SEQ ID NO: 488 - protein, 16 aa, synthetic construct
PQQPQPFPQQPVPQQP

SEQ ID NO: 489 - protein, 12 aa, synthetic construct
PFPWQPQQPFPQ

SEQ ID NO: 490 - protein, 12 aa, synthetic construct
PFPLQPQQPFPQ

SEQ ID NO: 491 - protein, 12 aa, synthetic construct
QQPFQPQQPFPQ

SEQ ID NO: 492 - protein, 16 aa, synthetic construct
NPLQPQQPFPLQPQPP

SEQ ID NO: 493 - protein, 16 aa, synthetic construct
PLQPQQPFPLQPQPPQ

SEQ ID NO: 494 - protein, 13 aa, synthetic construct
PNPLQPQQPFPLQ

SEQ ID NO: 495 - protein, 12 aa, synthetic construct
TIPQQPQQPFPL

SEQ ID NO: 496 - protein, 12 aa, synthetic construct
SFSQQPQQPFPL

SEQ ID NO: 497 - protein, 12 aa, synthetic construct
SFSEQPQQPFPL

SEQ ID NO: 498 - protein, 12 aa, synthetic construct
YSPYQPQQPFPQ

SEQ ID NO: 499 - protein, 12 aa, synthetic construct
QLPLQPQQPFPQ

SEQ ID NO: 500 - protein, 16 aa, synthetic construct
QQPQQPFPLQPQQPVP

SEQ ID NO: 501 - protein, 12 aa, synthetic construct
IIPQQPQQPFPL

SEQ ID NO: 502 - protein, 12 aa, synthetic construct
PEQIIPQQPQQP

SEQ ID NO: 503 - protein, 11 aa, synthetic construct
FLLQPQQPFSQ

SEQ ID NO: 504 - protein, 12 aa, synthetic construct
IISQQPQQPFPL

SEQ ID NO: 505 - protein, 12 aa, synthetic construct
PFPQRPQQPFPQ

SEQ ID NO: 506 - protein, 16 aa, synthetic construct
ELQPFPQPELPYPQPQ

SEQ ID NO: 507 - protein, 15 aa, synthetic construct
EQPFPQPEQPFPWQP

SEQ ID NO: 508 - protein, 16 aa, synthetic construct
EPEQPIPEQPQPYPQQ

SEQ ID NO: 509 - protein, 16 aa, synthetic construct
QLQPFPQPELPYPQPQ

SEQ ID NO: 510 - protein, 15 aa, synthetic construct
QQPFPQPEQPFPWQP

SEQ ID NO: 511 - protein, 16 aa, synthetic construct
FPEQPIPEQPQPYPQQ

SEQ ID NO: 512 - protein, 4 aa, synthetic construct
PELP

SEQ ID NO: 513 - protein, 7 aa, synthetic construct
QPELPYP

SEQ ID NO: 514 - protein, 7 aa, synthetic construct
PQPELPY

SEQ ID NO: 515 - protein, 7 aa, synthetic construct
FPQPELP

SEQ ID NO: 516 - protein, 8 aa, synthetic construct
PELPYPQP

SEQ ID NO: 517 - protein, 8 aa, synthetic construct
QPELPYPQ

SEQ ID NO: 518 - protein, 8 aa, synthetic construct
PQPELPYP

SEQ ID NO: 519 - protein, 8 aa, synthetic construct
FPQPELPY

SEQ ID NO: 520 - protein, 8 aa, synthetic construct
PFPQPELP

SEQ ID NO: 521 - protein, 9 aa, synthetic construct
PELPYPQPQ

SEQ ID NO: 522 - protein, 9 aa, synthetic construct
QPELPYPQP

SEQ ID NO: 523 - protein, 9 aa, synthetic construct
FPQPELPYP

SEQ ID NO: 524 - protein, 9 aa, synthetic construct
PFPQPELPY

SEQ ID NO: 525 - protein, 9 aa, synthetic construct
QPFPQPELP

SEQ ID NO: 526 - protein, 10 aa, synthetic construct
QPELPYPQPQ

SEQ ID NO: 527 - protein, 10 aa, synthetic construct
PQPELPYPQP

SEQ ID NO: 528 - protein, 10 aa, synthetic construct
FPQPELPYPQ

SEQ ID NO: 529 - protein, 10 aa, synthetic construct
PFPQPELPYP

SEQ ID NO: 530 - protein, 10 aa, synthetic construct
QPFPQPELPY

SEQ ID NO: 531 - protein, 10 aa, synthetic construct
LQPFPQPELP

SEQ ID NO: 532 - protein, 11 aa, synthetic construct
PQPELPYPQPQ

SEQ ID NO: 533 - protein, 11 aa, synthetic construct
FPQPELPYPQP

SEQ ID NO: 534 - protein, 11 aa, synthetic construct
PFPQPELPYPQ

SEQ ID NO: 535 - protein, 11 aa, synthetic construct
QPFPQPELPYP

SEQ ID NO: 536 - protein, 11 aa, synthetic construct
LQPFPQPELPY

SEQ ID NO: 537 - protein, 12 aa, synthetic construct
FPQPELPYPQPQ

SEQ ID NO: 538 - protein, 12 aa, synthetic construct
PFPQPELPYPQP

SEQ ID NO: 539 - protein, 12 aa, synthetic construct
LQPFPQPELPYP

SEQ ID NO: 540 - protein, 13 aa, synthetic construct
PFPQPELPYPQPQ

SEQ ID NO: 541 - protein, 13 aa, synthetic construct
LQPFPQPELPYPQ

SEQ ID NO: 542 - protein, 6 aa, synthetic construct
QPEQPF

SEQ ID NO: 543 - protein, 7 aa, synthetic construct
QPEQPFP

SEQ ID NO: 544 - protein, 7 aa, synthetic construct
PQPEQPF

SEQ ID NO: 545 - protein, 8 aa, synthetic construct
QPEQPFPW

SEQ ID NO: 546 - protein, 8 aa, synthetic construct
PQPEQPFP

SEQ ID NO: 547 - protein, 8 aa, synthetic construct
FPQPEQPF

SEQ ID NO: 548 - protein, 9 aa, synthetic construct
QPEQPFPWQ

SEQ ID NO: 549 - protein, 9 aa, synthetic construct
FPQPEQPFP

SEQ ID NO: 550 - protein, 10 aa, synthetic construct
QPEQPFPWQP

SEQ ID NO: 551 - protein, 10 aa, synthetic construct
PQPEQPFPWQ

SEQ ID NO: 552 - protein, 10 aa, synthetic construct
FPQPEQPFPW

SEQ ID NO: 553 - protein, 10 aa, synthetic construct
PFPQPEQPFP

SEQ ID NO: 554 - protein, 10 aa, synthetic construct
QPFPQPEQPF

SEQ ID NO: 555 - protein, 11 aa, synthetic construct
PQPEQPFPWQP

SEQ ID NO: 556 - protein, 11 aa, synthetic construct
FPQPEQPFPWQ

SEQ ID NO: 557 - protein, 11 aa, synthetic construct
PFPQPEQPFPW

SEQ ID NO: 558 - protein, 11 aa, synthetic construct
QPFPQPEQPFP

SEQ ID NO: 559 - protein, 12 aa, synthetic construct
FPQPEQPFPWQP

SEQ ID NO: 560 - protein, 12 aa, synthetic construct
PFPQPEQPFPWQ

SEQ ID NO: 561 - protein, 13 aa, synthetic construct
PFPQPEQPFPWQP

SEQ ID NO: 562 - protein, 13 aa, synthetic construct
QPFPQPEQPFPWQ

SEQ ID NO: 563 - protein, 7 aa, synthetic construct
PIPEQPQ

SEQ ID NO: 564 - protein, 8 aa, synthetic construct
PIPEQPQP

SEQ ID NO: 565 - protein, 8 aa, synthetic construct
QPIPEQPQ

SEQ ID NO: 566 - protein, 9 aa, synthetic construct
QPIPEQPQP

SEQ ID NO: 567 - protein, 10 aa, synthetic construct
PIPEQPQPYP

SEQ ID NO: 568 - protein, 10 aa, synthetic construct
QPIPEQPQPY

SEQ ID NO: 569 - protein, 10 aa, synthetic construct
EQPIPEQPQP

SEQ ID NO: 570 - protein, 10 aa, synthetic construct
PEQPIPEQPQ

SEQ ID NO: 571 - protein, 12 aa, synthetic construct
PIPEQPQPYPQQ

SEQ ID NO: 572 - protein, 12 aa, synthetic construct
QPIPEQPQPYPQ

SEQ ID NO: 573 - protein, 12 aa, synthetic construct
EQPIPEQPQPYP

SEQ ID NO: 574 - protein, 12 aa, synthetic construct
PEQPIPEQPQPY

SEQ ID NO: 575 - protein, 13 aa, synthetic construct
QPIPEQPQPYPQQ

SEQ ID NO: 576 - protein, 13 aa, synthetic construct
EQPIPEQPQPYPQ

SEQ ID NO: 577 - protein, 13 aa, synthetic construct
PEQPIPEQPQPYP

SEQ ID NO: 578 - protein, 14 aa, synthetic construct
EQPIPEQPQPYPQQ

SEQ ID NO: 579 - protein, 14 aa, synthetic construct
PEQPIPEQPQPYPQ

SEQ ID NO: 580 - protein, 4 aa, synthetic construct
PDLP

SEQ ID NO: 581 - protein, 7 aa, synthetic construct
PELPYPQ

SEQ ID NO: 582 - protein, 13 aa, synthetic construct
QPFPQPELPYPQP

SEQ ID NO: 583 - protein, 14 aa, synthetic construct
QPFPQPELPYPQPQ

SEQ ID NO: 584 - protein, 14 aa, synthetic construct
LQPFPQPELPYPQP

SEQ ID NO: 585 - protein, 11 aa, synthetic construct
PIPEQPQPYPQ

SEQ ID NO: 586 - protein, 11 aa, synthetic construct
QPIPEQPQPYP

SEQ ID NO: 587 - protein, 11 aa, synthetic construct
EQPIPEQPQPY

SEQ ID NO: 588 - protein, 11 aa, synthetic construct
PEQPIPEQPQP

SEQ ID NO: 589 - protein, 9 aa, synthetic construct
SQQPYLQLQ

SEQ ID NO: 590 - protein, 9 aa, synthetic construct
ALALVRMLI

SEQ ID NO: 591 - protein, 10 aa, synthetic construct
YLLPRRGPRL

SEQ ID NO: 592 - protein, 9 aa, synthetic construct
AISPRTLNA

SEQ ID NO: 593 - protein, 9 aa, synthetic construct
VMAPRTLIL

SEQ ID NO: 594 - protein, 9 aa, synthetic construct
SQAPLPCVL

SEQ ID NO: 595 - protein, 9 aa, synthetic construct
QMRPVSRVL

SEQ ID NO: 596 - protein, 16 aa, synthetic construct
HAVSEGTKAVTKYTSS

SEQ ID NO: 597 - protein, 16 aa, synthetic construct
PAETATPAPVEKSPAK

SEQ ID NO: 598 - protein, 16 aa, synthetic construct
AYVRLAPDYDALDVAN

SEQ ID NO: 599 - protein, 16 aa, synthetic construct
HAVSEGTKAVTKYTSA

SEQ ID NO: 600 - protein, 16 aa, synthetic construct
AVSDGVIKVFNDMKVR

SEQ ID NO: 601 - protein, 16 aa, synthetic construct
HAVSEGTKAVTKYTSS

SEQ ID NO: 602 - protein, 16 aa, synthetic construct
QLLQANPILEAFGNAK

SEQ ID NO: 603 - protein, 16 aa, synthetic construct
KSADTLWDIQKDLKDL

SEQ ID NO: 604 - protein, 16 aa, synthetic construct
AYVRLAPDYDALDVAN

SEQ ID NO: 605 - protein, 16 aa, synthetic construct
HAVSEGTKAVTKYTSA

SEQ ID NO: 606 - protein, 16 aa, synthetic construct
TGLIKGSGTAEVELKK

SEQ ID NO: 607 - protein, 16 aa, synthetic construct
VSDGVIKVFNDMKVRK

SEQ ID NO: 608 - protein, 16 aa, synthetic construct
ASGNYATVISHNPETK

SEQ ID NO: 609 - protein, 16 aa, synthetic construct
TAEILELAGNAARDNK

SEQ ID NO: 610 - protein, 16 aa, synthetic construct
HAVSEGTKAVTKYTSA

SEQ ID NO: 611 - protein, 15 aa, synthetic construct
PAPVEKSPAKKKATK

SEQ ID NO: 612 - protein, 15 aa, synthetic construct
SADTLWDIQKDLKDL

SEQ ID NO: 613 - protein, 15 aa, synthetic construct
TGLIKGSGTAEVELK

SEQ ID NO: 614 - protein, 15 aa, synthetic construct
KSADTLWGIQKELQF

SEQ ID NO: 615 - protein, 15 aa, synthetic construct
HGSYEDAVHSGALND

SEQ ID NO: 616 - protein, 15 aa, synthetic construct
SDGVIKVFNDMKVRK

SEQ ID NO: 617 - protein, 15 aa, synthetic construct
AGNLGGGVVTIERSK

SEQ ID NO: 618 - protein, 15 aa, synthetic construct
AQAAAPASVPAQAPK

SEQ ID NO: 619 - protein, 15 aa, synthetic construct
PRKIEEIKDFLLTAR

SEQ ID NO: 620 - protein, 14 aa, synthetic construct
SEGTKAVTKYTSSK

SEQ ID NO: 621 - protein, 14 aa, synthetic construct
VLKQVHPDTGISSK

SEQ ID NO: 622 - protein, 14 aa, synthetic construct
SWTAADTAAQITQR

SEQ ID NO: 623 - protein, 14 aa, synthetic construct
FISVGYVDDTQFVR

SEQ ID NO: 624 - protein, 14 aa, synthetic construct
NIDDGTSDRPYSHA

SEQ ID NO: 625 - protein, 14 aa, synthetic construct
VLKQVHPDTGISSK

SEQ ID NO: 626 - protein, 14 aa, synthetic construct
RKTVTAMDVVYALK

SEQ ID NO: 627 - protein, 14 aa, synthetic construct
SADTLWGIQKELQF

SEQ ID NO: 628 - protein, 14 aa, synthetic construct
ASAETVDPASLWEY

SEQ ID NO: 629 - protein, 13 aa, synthetic construct
TVVNKDVFRDPAL

SEQ ID NO: 630 - protein, 13 aa, synthetic construct
KTVTAMDVVYALK

SEQ ID NO: 631 - protein, 13 aa, synthetic construct
EGIPALDNFLDKL

SEQ ID NO: 632 - protein, 13 aa, synthetic construct
RVTIMPKDIQLAR

SEQ ID NO: 633 - protein, 13 aa, synthetic construct
PVAVMAESAFSFK

SEQ ID NO: 634 - protein, 13 aa, synthetic construct
QTVAVGVIKAVDK

SEQ ID NO: 635 - protein, 13 aa, synthetic construct
ILELAGNAARDNK

SEQ ID NO: 636 - protein, 12 aa, synthetic construct
GTGASGSFKLNK

SEQ ID NO: 637 - protein, 12 aa, synthetic construct
KQVHPDTGISSK

SEQ ID NO: 638 - protein, 12 aa, synthetic construct
VGGTSDVEVNEK

SEQ ID NO: 639 - protein, 12 aa, synthetic construct
NSVVEASEAAYK

SEQ ID NO: 640 - protein, 12 aa, synthetic construct
ALRYPMAVGLNK

SEQ ID NO: 641 - protein, 12 aa, synthetic construct
SLVSKGTLVQTK

SEQ ID NO: 642 - protein, 11 aa, synthetic construct
PELAKSAPAPK

SEQ ID NO: 643 - protein, 11 aa, synthetic construct
SEMEVQDAELK

SEQ ID NO: 644 - protein, 11 aa, synthetic construct
QTYSTEPNNLK

SEQ ID NO: 645 - protein, 11 aa, synthetic construct
PMFIVNTNVPR

SEQ ID NO: 646 - protein, 10 aa, synthetic construct
AGFAGDDAPR

SEQ ID NO: 647 - protein, 10 aa, synthetic construct
RVNAGTLAVL

SEQ ID NO: 648 - protein, 9 aa, synthetic construct
IGQSKVFFR

SEQ ID NO: 649 - protein, 16 aa, synthetic construct
TAEILELAGNAARDNK

SEQ ID NO: 650 - protein, 13 aa, synthetic construct
ILELAGNAARDNK

SEQ ID NO: 651 - protein, 17 aa, synthetic construct
ALAGCHLEDTQRKLQKG

SEQ ID NO: 652 - protein, 16 aa, synthetic construct
MQLITRGKGAGTPNLI

SEQ ID NO: 653 - protein, 15 aa, synthetic construct
KMKLRNTVHLSYLTV

SEQ ID NO: 654 - protein, 15 aa, synthetic construct
CRASQTISSYLDWYQ

SEQ ID NO: 655 - protein, 13 aa, synthetic construct
PAALTNKGNTVFA

SEQ ID NO: 656 - protein, 13 aa, synthetic construct
WTPGPSAGVTGIA

SEQ ID NO: 657 - protein, 10 aa, synthetic construct
ILRTIGKEAF

SEQ ID NO: 658 - protein, 9 aa, synthetic construct
RSCGYACTA

SEQ ID NO: 659 - protein, 9 aa, synthetic construct
FPNGFSFIH

SEQ ID NO: 660 - protein, 9 aa, synthetic construct
SHGPYIKLI

SEQ ID NO: 661 - protein, 15 aa, synthetic construct
AQAAAPASVPAQAPK

SEQ ID NO: 662 - protein, 17 aa, synthetic construct
AYVRLAPDYDALDVANK

SEQ ID NO: 663 - protein, 16 aa, synthetic construct
AYVRLAPDYDALDVAN

SEQ ID NO: 664 - protein, 16 aa, synthetic construct
ASGNYATVISHNPETK

SEQ ID NO: 665 - protein, 15 aa, synthetic construct
AGNLGGGVVTIERSK

SEQ ID NO: 666 - protein, 15 aa, synthetic construct
PRKIEEIKDFLLTAR

SEQ ID NO: 667 - protein, 14 aa, synthetic construct
NIDDGTSDRPYSHA

SEQ ID NO: 668 - protein, 13 aa, synthetic construct
TVVNKDVFRDPAL

SEQ ID NO: 669 - protein, 12 aa, synthetic construct
ALRYPMAVGLNK

SEQ ID NO: 670 - protein, 11 aa, synthetic construct
QTYSTEPNNLK

SEQ ID NO: 671 - protein, 11 aa, synthetic construct
PELAKSAPAPK

SEQ ID NO: 672 - protein, 12 aa, synthetic construct
KQVHPDTGISSK

SEQ ID NO: 673 - protein, 14 aa, synthetic construct
VLKQVHPDTGISSK

SEQ ID NO: 674 - protein, 14 aa, synthetic construct
SEGTKAVTKYTSSK

SEQ ID NO: 675 - protein, 16 aa, synthetic construct
HAVSEGTKAVTKYTSA

SEQ ID NO: 676 - protein, 16 aa, synthetic construct
HAVSEGTKAVTKYTSS

SEQ ID NO: 677 - protein, 17 aa, synthetic construct
HAVSEGTKAVTKYTSAK

SEQ ID NO: 678 - protein, 17 aa, synthetic construct
HAVSEGTKAVTKYTSSK

SEQ ID NO: 679 - protein, 16 aa, synthetic construct
HAVSEGTKAVTKYTSA

SEQ ID NO: 680 - protein, 10 aa, synthetic construct
AGFAGDDAPR

SEQ ID NO: 681 - protein, 9 aa, synthetic construct
SQAPLPCVL

SEQ ID NO: 682 - protein, 9 aa, synthetic construct
VMAPRTLFL

SEQ ID NO: 683 - protein, 13 aa, synthetic construct
PKKTESHHKAKGK

SEQ ID NO: 684 - protein, 7 aa, synthetic construct
AAVLEYL

SEQ ID NO: 685 - protein, 25 aa, synthetic construct
AQAAAPASVPAQAPKRTQAPTKASE

SEQ ID NO: 686 - protein, 19 aa, synthetic construct
KLEKEEEEGISQESSEEEQ

SEQ ID NO: 687 - protein, 19 aa, synthetic construct
GDRSEDFGVNEDLADSDAR

SEQ ID NO: 688 - protein, 18 aa, synthetic construct
VAPEEHPVLLTEAPLNPK

SEQ ID NO: 689 - protein, 16 aa, synthetic construct
STAGDTHLGGEDFDNR

SEQ ID NO: 690 - protein, 15 aa, synthetic construct
KVPQVSTPTLVEVSR

SEQ ID NO: 691 - protein, 15 aa, synthetic construct
PDPAKSAPAPKKGSK

SEQ ID NO: 692 - protein, 12 aa, synthetic construct
LQAEIEGLKGQR

SEQ ID NO: 693 - protein, 11 aa, synthetic construct
PDPAKSAPAPK

SEQ ID NO: 694 - protein, 11 aa, synthetic construct
PELAKSAPAPK

SEQ ID NO: 695 - protein, 11 aa, synthetic construct
PEPVKSAPVPK

SEQ ID NO: 696 - protein, 9 aa, synthetic construct
AAPATRAAL

SEQ ID NO: 697 - protein, 9 aa, synthetic construct
SAPSRATAL

SEQ ID NO: 698 - protein, 8 aa, synthetic construct
ILNFPPPP

SEQ ID NO: 699 - protein, 8 aa, synthetic construct
IAPTGHSL

SEQ ID NO: 700 - protein, 8 aa, synthetic construct
ISPHGNAL

SEQ ID NO: 701 - protein, 15 aa, synthetic construct
PDPAKSAPAPKKGSK

SEQ ID NO: 702 - protein, 11 aa, synthetic construct
PDPAKSAPAPK

SEQ ID NO: 703 - protein, 11 aa, synthetic construct
PELAKSAPAPK

SEQ ID NO: 704 - protein, 11 aa, synthetic construct
PEPVKSAPVPK

SEQ ID NO: 705 - protein, 18 aa, synthetic construct
VAPEEHPVLLTEAPLNPK

SEQ ID NO: 706 - protein, 13 aa, synthetic construct
PLLALLALWGPDP

SEQ ID NO: 707 - protein, 11 aa, synthetic construct
PKTRREAEVGQ

SEQ ID NO: 708 - protein, 11 aa, synthetic construct
RREAEDLQGSL

SEQ ID NO: 709 - protein, 11 aa, synthetic construct
RREAEDLEGSL

SEQ ID NO: 710 - protein, 13 aa, synthetic construct
DLQVGQVELGGGP

SEQ ID NO: 711 - protein, 13 aa, synthetic construct
DLQVGEVELGGGP

SEQ ID NO: 712 - protein, 13 aa, synthetic construct
CGSHLVEALYLVC

SEQ ID NO: 713 - protein, 11 aa, synthetic construct
MNILLEYVVKS

SEQ ID NO: 714 - protein, 17 aa, synthetic construct
NMFTYEIAPVFVLLEYV

SEQ ID NO: 715 - protein, 17 aa, synthetic construct
NVCFWYIPPSLRTLEDN

SEQ ID NO: 716 - protein, 17 aa, synthetic construct
FNQLSTGLDMVGLAADW

SEQ ID NO: 717 - protein, 17 aa, synthetic construct
GRTGTYILIDMVLNRMA

SEQ ID NO: 718 - protein, 17 aa, synthetic construct
PKAARPPVTPVLLEKKS

SEQ ID NO: 719 - protein, 11 aa, synthetic construct
LLALLALWGPD

SEQ ID NO: 720 - protein, 21 aa, synthetic construct
KKKKYVSIDVTLQQLESHKKK

SEQ ID NO: 721 - protein, 15 aa, synthetic construct
GLKMFPDLTKVYSTD

SEQ ID NO: 722 - protein, 15 aa, synthetic construct
PRHRDTGILDSIGRF

SEQ ID NO: 723 - protein, 17 aa, synthetic construct
ENPVVHFFKNIVTPRTP

SEQ ID NO: 724 - protein, 15 aa, synthetic construct
ASDYKSAHKGFKGVD

SEQ ID NO: 725 - protein, 15 aa, synthetic construct
GFKGVDAQGTLSKIF

SEQ ID NO: 726 - protein, 14 aa, synthetic construct
TQQIRLQAEIFQAR

SEQ ID NO: 727 - protein, 14 aa, synthetic construct
AQQIRLQAEAFQAR

SEQ ID NO: 728 - protein, 14 aa, synthetic construct
TAQIRLQAEIFQAR

SEQ ID NO: 729 - protein, 14 aa, synthetic construct
TQAIRLQAEIFQAR

SEQ ID NO: 730 - protein, 14 aa, synthetic construct
TQQARLQAEIFQAR

SEQ ID NO: 731 - protein, 14 aa, synthetic construct
TQQIALQAEIFQAR

SEQ ID NO: 732 - protein, 15 aa, synthetic construct
QTQQIRLQAEIFQAR

SEQ ID NO: 733 - protein, 13 aa, synthetic construct
QQIRLQAEIFQAR

SEQ ID NO: 734 - protein, 9 aa, synthetic construct
NIDALNENK

SEQ ID NO: 735 - protein, 11 aa, synthetic construct
NVLVLDTDYKK

SEQ ID NO: 736 - protein, 15 aa, synthetic construct
NTPEVDDEALEKFDK

SEQ ID NO: 737 - protein, 15 aa, synthetic construct
LEDARRLKAIYEKKK

SEQ ID NO: 738 - protein, 13 aa, synthetic construct
HSLGKWLGHPDKF

SEQ ID NO: 739 - protein, 14 aa, synthetic construct
NTWTTCQSIAFPSK

SEQ ID NO: 740 - protein, 16 aa, synthetic construct
IAATYNFAVLKLMGRG

SEQ ID NO: 741 - protein, 13 aa, synthetic construct
VHFFKNIVTPRTP

SEQ ID NO: 742 - protein, 15 aa, synthetic construct
DEGGYTCFFRDHSYQ

SEQ ID NO: 743 - protein, 9 aa, synthetic construct
HLVEALYLV

SEQ ID NO: 744 - protein, 9 aa, synthetic construct
LNIDLLWSV

SEQ ID NO: 745 - protein, 9 aa, synthetic construct
VLFGLGFAI

SEQ ID NO: 746 - protein, 9 aa, synthetic construct
VYLKTNVFL

SEQ ID NO: 747 - protein, 9 aa, synthetic construct
KYNKANAFL

SEQ ID NO: 748 - protein, 9 aa, synthetic construct
KYNIANVFL

SEQ ID NO: 749 - protein, 9 aa, synthetic construct
KYNKANVFL

SEQ ID NO: 750 - protein, 9 aa, synthetic construct
FQDENYLYL

SEQ ID NO: 751 - protein, 9 aa, synthetic construct
LYLVCGERG

SEQ ID NO: 752 - protein, 10 aa, synthetic construct
VMNILLQYVV

SEQ ID NO: 753 - protein, 10 aa, synthetic construct
RMMEYGTTMV

SEQ ID NO: 754 - protein, 10 aa, synthetic construct
NLAQTDLATV

SEQ ID NO: 755 - protein, 9 aa, synthetic construct
QLARQQVHV

SEQ ID NO: 756 - protein, 9 aa, synthetic construct
SLSPLQAEL

SEQ ID NO: 757 - protein, 9 aa, synthetic construct
SLAAGVKLL

SEQ ID NO: 758 - protein, 9 aa, synthetic construct
VIVMLTPLV

SEQ ID NO: 759 - protein, 9 aa, synthetic construct
KLQVFLIVL

SEQ ID NO: 760 - protein, 9 aa, synthetic construct
FLIVLSVAL

SEQ ID NO: 761 - protein, 9 aa, synthetic construct
FLWSVFMLI

SEQ ID NO: 762 - protein, 9 aa, synthetic construct
NLFLFLFAV

SEQ ID NO: 763 - protein, 9 aa, synthetic construct
FLFAVGFYL

SEQ ID NO: 764 - protein, 9 aa, synthetic construct
YLLLRVLNI

SEQ ID NO: 765 - protein, 9 aa, synthetic construct
RLLCALTSL

SEQ ID NO: 766 - protein, 9 aa, synthetic construct
ALWMRLLPL

SEQ ID NO: 767 - protein, 9 aa, synthetic construct
LWMRLLPLL

SEQ ID NO: 768 - protein, 9 aa, synthetic construct
RLLPLLALL

SEQ ID NO: 769 - protein, 10 aa, synthetic construct
HLCGSHLVEA

SEQ ID NO: 770 - protein, 9 aa, synthetic construct
ALYLVCGER

SEQ ID NO: 771 - protein, 10 aa, synthetic construct
LYLVCGERGF

SEQ ID NO: 772 - protein, 9 aa, synthetic construct
LVCGERGFF

SEQ ID NO: 773 - protein, 10 aa, synthetic construct
VCGERGFFYT

SEQ ID NO: 774 - protein, 8 aa, synthetic construct
GERGFFYT

SEQ ID NO: 775 - protein, 9 aa, synthetic construct
ERGFFYTPK

SEQ ID NO: 776 - protein, 9 aa, synthetic construct
FYTPKTRRE

SEQ ID NO: 777 - protein, 11 aa, synthetic construct
TPKTRREAEDL

SEQ ID NO: 778 - protein, 9 aa, synthetic construct
SLQPLALEG

SEQ ID NO: 779 - protein, 9 aa, synthetic construct
ALEGSLQKR

SEQ ID NO: 780 - protein, 10 aa, synthetic construct
SLQKRGIVEQ

SEQ ID NO: 781 - protein, 10 aa, synthetic construct
GIVEQCCTSI

SEQ ID NO: 782 - protein, 9 aa, synthetic construct
IVEQCCTSI

SEQ ID NO: 783 - protein, 9 aa, synthetic construct
SLYQLENYC

SEQ ID NO: 784 - protein, 9 aa, synthetic construct
SLLLELEEV

SEQ ID NO: 785 - protein, 9 aa, synthetic construct
LMWAKIGPV

SEQ ID NO: 786 - protein, 9 aa, synthetic construct
VLFSSDFRI

SEQ ID NO: 787 - protein, 9 aa, synthetic construct
SLSRFSWGA

SEQ ID NO: 788 - protein, 9 aa, synthetic construct
KVEDPFYWV

SEQ ID NO: 789 - protein, 10 aa, synthetic construct
RTFDPHFLRV

SEQ ID NO: 790 - protein, 9 aa, synthetic construct
FLRVPCWKI

SEQ ID NO: 791 - protein, 10 aa, synthetic construct
KITLFVIVPV

SEQ ID NO: 792 - protein, 9 aa, synthetic construct
VLGPLVALI

SEQ ID NO: 793 - protein, 9 aa, synthetic construct
TLFVIVPVL

SEQ ID NO: 794 - protein, 10 aa, synthetic construct
RLAGQFLEEL

SEQ ID NO: 795 - protein, 9 aa, synthetic construct
FLYGALLLA

SEQ ID NO: 796 - protein, 15 aa, synthetic construct
FIEWNKLRFRQGLEW

SEQ ID NO: 797 - protein, 5 aa, synthetic construct
GGGGS

SEQ ID NO: 798 - protein, 4 aa, synthetic construct
GGSG

SEQ ID NO: 799 - protein, 4 aa, synthetic construct
SGGG

SEQ ID NO: 800 - protein, 4 aa, synthetic construct
GSGS

SEQ ID NO: 801 - protein, 6 aa, synthetic construct
GSGSGS

SEQ ID NO: 802 - protein, 8 aa, synthetic construct
GSGSGSGS

SEQ ID NO: 803 - protein, 10 aa, synthetic construct
GSGSGSGSGS

SEQ ID NO: 804 - protein, 12 aa, synthetic construct
GSGSGSGSGSGS

SEQ ID NO: 805 - protein, 6 aa, synthetic construct
GGSGGS

SEQ ID NO: 806 - protein, 9 aa, synthetic construct
GGSGGSGGS

SEQ ID NO: 807 - protein, 12 aa, synthetic construct
GGSGGSGGSGGS

SEQ ID NO: 808 - protein, 4 aa, synthetic construct
GGSG

SEQ ID NO: 809 - protein, 8 aa, synthetic construct
GGSGGGSG

SEQ ID NO: 810 - protein, 12 aa, synthetic construct
GGSGGGSGGGSG

SEQ ID NO: 811 - protein, 15 aa, synthetic construct
GGGGSGGGGSGGGGS

SEQ ID NO: 812 - protein, 20 aa, synthetic construct
SGGGSGGGSGGGSGGGSGGG

SEQ ID NO: 813 - protein, 4 aa, synthetic construct
GGGG

SEQ ID NO: 814 - protein, 8 aa, synthetic construct
GGGGGGGG

SEQ ID NO: 815 - protein, 12 aa, synthetic construct
GGGGGGGGGGGG

SEQ ID NO: 816 - protein, 16 aa, synthetic construct
GGGGGGGGGGGGGGGG

SEQ ID NO: 817 - protein, 20 aa, synthetic construct
GGGGGGGGGGGGGGGGGGGG

SEQ ID NO: 818 - protein, 5 aa, synthetic construct
GGGGG

SEQ ID NO: 819 - protein, 10 aa, synthetic construct
GGGGGGGGGG

SEQ ID NO: 820 - protein, 15 aa, synthetic construct
GGGGGGGGGGGGGGG

SEQ ID NO: 821 - protein, 20 aa, synthetic construct
GGGGGGGGGGGGGGGGGGGG

SEQ ID NO: 822 - protein, 20 aa, synthetic construct
GGGGGGGGGGGGGGGGGGGG

SEQ ID NO: 823 - protein, 10 aa, synthetic construct
GENLYFQSGG

SEQ ID NO: 824 - protein, 8 aa, synthetic construct
SACYCELS

SEQ ID NO: 825 - protein, 5 aa, synthetic construct
RSIAT

SEQ ID NO: 826 - protein, 17 aa, synthetic construct
RPACKIPNDLKQKVMNH

SEQ ID NO: 827 - protein, 36 aa, synthetic construct
GGSAGGSGSGSSGGSSGASGTGTAGGTGSGSGTGSG

SEQ ID NO: 828 - protein, 17 aa, synthetic construct
AAANSSIDLISVPVDSR

SEQ ID NO: 829 - protein, 36 aa, synthetic construct
GGSGGGSEGGGSEGGGSEGGGSEGGGSEGGGSGGGS